(19) United States
(12) Patent Application Publication
(10) Pub. No.: US 2026/0248850 A1
(43) Pub. Date: Aug. 27, 2026

(54) IMMUNE CELLS WITH CHIMERIC ANTIGEN RECEPTORS OR CHIMERIC AUTOANTIBODY RECEPTORS

(71) Applicant: Saliogen Therapeutics, Inc., Lexington, MA (US)

(72) Inventors: Joseph J. HIGGINS, Lexington, MA (US); Ray TABIBIAZAR, Lexington, MA (US); Omid F. HARANDI, Lexington, MA (US); Francisco NAVARRO, Lexington, MA (US); James B. HEMPHILL, Lexington, MA (US); Joshua LAMORA, Lexington, MA (US)

(21) Appl. No.: 18/706,018

(22) PCT Filed: Nov. 4, 2022
(86) PCT No.: PCT/US2022/079288

§ 371 Date: Apr. 30, 2024

Related U.S. Application Data
(60) Provisional application No. 63/275,777, filed on Nov. 4, 2021.

(51) Int. Cl. A61K 35/17; A61K 31/7105; A61K 35/15; A61K 38/00; A61K 40/11; A61K 40/15; A61K 40/17; A61K 40/31; A61K 40/41; A61K 48/00; C07K 14/705; C12N 5/0783; C12N 5/0786; C12N 15/52; C12N 15/88

(52) U.S. Cl. CPC A61K 35/17; A61K 31/7105; A61K 35/15; A61K 38/00; A61K 40/11; A61K 40/15; A61K 40/17; A61K 40/31; A61K 40/416; A61K 48/00; C07K 14/7051; C12N 5/0636; C12N 5/0645; C12N 5/0646; C12N 15/52; C12N 15/88; A61K 2239/13; A61K 2239/15; C07K 2319/02; C07K 2319/03; C12N 2510/00

(57) ABSTRACT

Methods of making a chimeric antigen receptor (CAR)-immune cell or chimeric autoantibody receptor (CAAR)-immune cell, e.g., with an enzyme capable of performing targeted genomic integration, are provided.

### CROSS-REFERENCE TO RELATED APPLICATIONS

This application is a national stage filing under 35 U.S.C. § 371 of International PCT Application No. PCT/US2022/079288, filed Nov. 4, 2022, which claims priority under 35 U.S.C. § 119(e) to U.S. Provisional Patent Application No. 63/275,777, filed on Nov. 4, 2021, the entire content of each of which is hereby incorporated herein by reference in its entirety.

### FIELD

The present disclosure relates, in part, to a method making a chimeric antigen receptor (CAR)-immune cell or chimeric autoantibody receptor (CAAR)-immune cell, e.g., using an enzyme capable of performing targeted genomic integration, such as a mobile element enzyme.

### SEQUENCE LISTING

The contents of the text file submitted electronically herewith are incorporated herein by reference in their entirety: A computer readable format copy of the Sequence Listing (filename: “Sequence_Listing_SAL-009PC_126933-5009.xml”; date recorded: Nov. 4, 2022; file size: 962,560 bytes).

### BACKGROUND

Two types of T cells work together to protect the body from abnormal attacks against self and infectious pathogens. The first type is called effector T cells and eliminates pathogens. The secFond type, called regulatory T (Treg) cells, functions to prevent an immune response against self. Although effector T cell responses are generally potent, a subset of infectious diseases and tumors have evolved a large variety of escape mechanisms to bypass T cell control. Similarly, the incidence of autoimmune diseases, such as type 1 diabetes, highlights that Treg cells are not always successful in preventing aberrant immune responses. Moreover, in organ transplantation, Treg cells often fail to protect life-saving tissues from immune rejection.

Chimeric antigen receptors (CARs) technology has emerged as a promising approach to re-program T cells to overcome the barriers that confront naturally occurring T cells. Because CARs alter how T cells recognize antigen by directly binding to cell surface proteins without requiring peptide presentation by MHC molecules, there are fewer available targets for CARs to recognize relative to TCRs. However, CAR targeting has more specificity, no HLA restriction, and avoid many of the T cell escape mechanisms that are used by infectious agents and tumors are no longer effective against CAR T cells. Although clinical success is relatively new to the CAR T cell field, the concept first emerged in the 1990s when investigators showed that T cell specificity could be redirected by fusing a targeting moiety that recognizes a cell surface protein with a T cell activation domain such as the CD3ζ cytoplasmic tail. The first example of this technology fused CD4 to the CD3ζ chain (CD4ζCAR). When expressed in effector T cells, this construct redirected T cell specificity to HIV-infected cells by taking advantage of the interaction between HIV envelope protein (Env) and CD42. This concept was brought to the clinic in the late 1990s; although it was shown to be safe and feasible, durable control of virus infection was not observed. In the intervening years, our understanding of how to engineer potent effector CAR T cells to target tumors has increased. In addition, pharmaceutical companies have promoted the transition of effector CAR T cell therapy from a boutique Phase I single center clinical trial to a Food and Drug Administration (FDA)-approved therapy that is capable of treating thousands of patients across the United States and elsewhere.

As T cells have pivotal roles in controlling infectious disease and autoimmunity, many in the field are considering how CAR T cell therapy could provide long-term solutions to diseases outside of cancer in which traditional medical approaches have not provided a cure.

Chimeric auto-antibody receptor T (CAAR-T) cells are the modified form of CAR-T cells which identify cells secreting antibodies like autoreactive B cells and bring similar excitement and limitations as CAR-T cells.

Accordingly, there is a need for improved approach to development of CAR-immune cells and CAAR-immune cells.

### SUMMARY

In aspects, there is provided a method of making a chimeric antigen receptor (CAR)-immune cell, the method comprising: obtaining an immune cell from a sample obtained from a subject, the sample comprising immune cells or immune cell progenitors; and transfecting the immune cell with a first nucleic acid encoding an enzyme capable of performing targeted genomic integration, optionally wherein the first nucleic acid is RNA, and a second, non-viral nucleic acid encoding a donor DNA comprising a chimeric CAR and flanked by ends recognized by the enzyme capable of performing targeted genomic integration, e.g., without limitation, a mobile element enzyme, to thereby create a transfected CAR-immune cell, wherein the immune cell is selected from a T cell, macrophage, and a natural killer (NK) cell.

In aspects, there is provided a method of making a chimeric antigen receptor (CAR)-immune cell, the method comprising: administering to a subject with a first nucleic acid encoding an enzyme capable of performing targeted genomic integration, wherein the first nucleic acid is RNA, and a second, non-viral nucleic acid encoding a donor DNA comprising a chimeric CAR and flanked by ends recognized by the enzyme capable of performing targeted genomic integration, e.g., without limitation, a mobile element enzyme, wherein the administration uses lipid nanoparticles (LNPs) capable of directing the first and second nucleic acids to an immune cell, to thereby create a CAR-immune cell, wherein the immune cell is selected from a T cell, macrophage, and a natural killer (NK) cell. In embodiments, the administration is to the skin cell in a subject.

In embodiments, the immune cell is a T cell. In embodiments, the transfected CAR-immune cell is a CAR-T cell, optionally selected from an allogeneic CAR-T cell or an allogeneic CAR-T cell. In embodiments, the immune cell is a macrophage. In embodiments, the transfected CAR-immune cell is a CAR-M cell, optionally selected from an allogeneic CAR-M cell or an allogeneic CAR-M cell. In embodiments, the immune cell is a natural killer (NK). In embodiments, the transfected CAR-immune cell is a CAR-NK cell, optionally selected from an allogeneic CAR-NK cell or an allogeneic CAR-NK cell.

In embodiments, the donor DNA comprises or further comprises one or more of a nucleic acid sequence of a transmembrane domain, a nucleic acid sequence of an intracellular domain of a costimulatory molecule, and a nucleic acid sequence of a signaling domain. In embodiments, the donor DNA comprises or further comprises a nucleic acid sequence of a CD8 alpha chain signal peptide. In embodiments, the nucleic acid sequence of the transmembrane domain encodes an CD8 alpha chain hinge and a transmembrane domain. In embodiments, the donor DNA further comprises a nucleic acid sequence of a peptide linker. In embodiments, the nucleic acid sequence of the intracellular signaling domain comprises a nucleic acid sequence encoding a CD3 zeta signaling domain. In embodiments, the second nucleic acid encodes an extracellular single-chain variable fragment (scFv) antibody to CD19 fused to a T cell cytoplasmic signaling domain.

In embodiments, the CAR comprises: a) an extracellular domain that binds an antigen selected from alpha folate receptor, 5T4, αvβ6 integrin, BCMA, B7-H3, B7-H6, CAIX, CD19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, GD2, GD3, Glypican-3 (GPC3), HLA-A1+MAGE1, HLA-A2+MAGE1, HLA-A3+MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, and VEGFR2; b) a transmembrane domain derived from a polypeptide selected from CD8α; CD4, CD28, CD45, PD-1, and CD152; c) one or more intracellular costimulatory signaling domains selected from CD28, CD54 (ICAM), CD134 (OX40), CD137 (41BB), IL-2Rβ, CD152 (CTLA4), CD273 (PD-L2), CD274 (PD-L1), and CD278 (ICOS); and/or d) a CD3ζ signaling domain.

In aspects, there is provided a method of making a chimeric autoantibody receptor (CAAR)-immune cell, the method comprising: obtaining a cell from a sample obtained from a subject, the sample comprising T cells or Tcell progenitors; and transfecting the cell with a first nucleic acid encoding an enzyme capable of performing targeted genomic integration, wherein the first nucleic acid is RNA, and a second, non-viral nucleic acid encoding a donor DNA comprising a chimeric CAAR comprising an autoantigen or a fragment thereof, and flanked by ends recognized by the enzyme capable of performing targeted genomic integration, e.g., without limitation, a mobile element enzyme, to thereby create a transfected CAAR-immune cell.

In aspects, there is provided a method of making a method of making a chimeric autoantibody receptor (CAAR)-immune cell, the method comprising: administering to a skin cell in a subject with a first nucleic acid encoding an enzyme capable of performing targeted genomic integration, wherein the first nucleic acid is RNA, and a second, non-viral nucleic acid encoding a donor DNA comprising a chimeric CAAR comprising an autoantigen or a fragment thereof, and flanked by ends recognized by the enzyme capable of performing targeted genomic integration, e.g., without limitation, a mobile element enzyme, wherein the administration is performed via intradermal or subcutaneous delivery and using lipid nanoparticles (LNPs) capable of directing the first and second nucleic acids to an immune cell, to thereby create a CAR-immune cell.

In embodiments, the immune cell is a T cell. In embodiments, the transfected CAAR-immune cell is a CAAR-T cell, optionally selected from an allogeneic CAAR-T cell or an allogeneic CAAR-T cell. In embodiments, the immune cell is a macrophage. In embodiments, the transfected CAAR-immune cell is a CAAR-M cell, optionally selected from an allogeneic CAAR-M cell or an allogeneic CAAR-M cell. In embodiments, the immune cell is a natural killer (NK). In embodiments, the transfected CAAR-immune cell is a CAAR-NK cell, optionally selected from an allogeneic CAAR-NK cell or an allogeneic CAAR-NK cell.

In embodiments, the autoantigen or a fragment thereof is an extracellular domain for targeting of an autoantibody or a B-cell receptor (BCR). In embodiments, the extracellular domain is an AChR autoantigen. In embodiments, the AChR autoantigen comprises domains E2-E6, and wherein the AChR autoantigen is fused to a CD8 alpha chain transmembrane domain and CD137-CD3z cytoplasmic signaling domains. In embodiments, the extracellular domain is a Muscle Specific Kinase (MuSK) extracellular domain. In embodiments, the extracellular domain is a desmoglein-3 (Dsg3). In embodiments, the extracellular domain is a desmoglein-1 (Dsg1).

In embodiments, the enzyme capable of performing targeted genomic integration is a recombinase, e.g., an integrase or a mobile element enzyme. In embodiments, the enzyme is a mobile element enzyme, e.g., derived from, or an engineered version of a mobile element enzyme of, Bombyx mori, Xenopus tropicalis, Trichoplusia ni, Myotis lucifugus, Rhinolophus ferrumequinum, Rousettus aegyptiacus, Phyllostomus discolor, Myotis myotis, Pteropus vampyrus, Pipistrellus kuhlii, Molossus molossus, Pan troglodytes, or Homo sapiens, e.g., one or more of the Tn1, Tn2, Tn3, Tn5, Tn7, Tn9, Tn10, Tn552, Tn903, Tn1000/Gamma-delta, Tn/0, tnsA, tnsB, tnsC, tniQ, IS10, ISS, 1S911, Minos, Sleeping beauty, piggyBac, Tol2, Mos1, Himar1, Hermes, Tol2, Minos, Tel, P-element, MuA, Ty1, Chapaev, transib, Tc1/mariner, or Tc3 donor DNA system, or biologically active fragments variants thereof, inclusive of hyperactive variants. In embodiments, the mobile element enzyme has the amino acid sequence of SEQ ID NO: 1, or a variant thereof, e.g., having an amino acid other than serine at the position corresponding to position 2 of SEQ ID NO: 1 (e.g., selected from G, A, V, L, I and P, optionally A), not having additional residues at the C terminus relative to SEQ ID NO: 1, and/or having one or more mutations which confer hyperactivity (e.g., of TABLE 1) and/or having one or more mutations which modulation integration (e.g., of TABLE 2A or TABLE 2B). In embodiments, the mobile element enzyme having at least about 90% identity to the amino acid sequence of SEQ ID NO: 1, SEQ ID NO: 3, SEQ ID NO: 4, SEQ ID NO: 6, SEQ ID NO: 7, SEQ ID NO: 8, SEQ ID NO: 9, SEQ ID NO: 10, SEQ ID NO: 11, or SEQ ID NO: 430, or a variant thereof, e.g., having one or more mutations which confer hyperactivity (e.g., of TABLE 1) and/or having one or more mutations which modulation integration (e.g., of TABLE 2A or TABLE 2B). In embodiments, the mobile element enzyme has gene cleavage activity (Exc+) and/or gene integration activity (Int+). In embodiments, the mobile element enzyme has gene cleavage activity (Exc+) and/or lacks gene integration activity (Int−).

In embodiments, the enzyme comprises a targeting element, and an enzyme that is capable of inserting the donor DNA comprising a chimeric CAR or chimeric CAAR, optionally at a TA dinucleotide site or a TTAA (SEQ ID NO: 440) tetranucleotide site, optionally in a genomic safe harbor site (GSHS). In embodiments, the mobile element enzyme is a chimeric mobile element enzyme. In embodiments, the targeting element comprises one or more of a gRNA, optionally associated with a Cas enzyme, which is optionally catalytically inactive, transcription activator-like effector (TALE), Zinc finger, catalytically inactive transcription factor, nickase, a transcriptional activator, a transcriptional repressor, a recombinase, a DNA methyltransferase, a histone methyltransferase, a paternally expressed gene 10 (PEG10), and a TnsD.

In embodiments, the GSHS is in an open chromatin location in a chromosome. In embodiments, the GSHS is selected from adeno-associated virus site 1 (AAVS1), chemokine (C—C motif) receptor 5 (CCR5) gene, HIV-1 coreceptor, and human Rosa26 locus. In embodiments, the GSHS is located on human chromosome 2, 4, 6, 10, 11, 17, 22, or X. In embodiments, the GSHS is selected from TALC1, TALC2, TALC3, TALC4, TALC5, TALC7, TALC8, AVS1, AVS2, AVS3, ROSA1, ROSA2, TALER1, TALER2, TALER3, TALER4, TALER5, SHCHR2-1, SHCHR2-2, SHCHR2-3, SHCHR2-4, SHCHR4-1, SHCHR4-2, SHCHR4-3, SHCHR6-1, SHCHR6-2, SHCHR6-3, SHCHR6-4, SHCHR10-1, SHCHR10-2, SHCHR10-3, SHCHR10-4, SHCHR10-5, SHCHR11-1, SHCHR11-2, SHCHR11-3, SHCHR17-1, SHCHR17-2, SHCHR17-3, and SHCHR17-4.

In embodiments, the disclosure provides a CAR-immune cell or a CAAR-immune cell generated by a method described herein.

In embodiments, the disclosure provides a method of delivering a CAR-immune cell or a CAAR-immune cell therapy, comprising administering to a patient in need thereof the CAR-immune cell or a CAAR-immune cell generated by a method described herein.

In embodiments, the disclosure provides a method of treating a disease or condition using a CAR-immune cell or a CAAR-immune cell therapy, comprising administering to a patient in need thereof the CAR-immune cell or a CAAR-immune cell generated by a method described herein.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs.

### BRIEF DESCRIPTION OF DRAWINGS

FIGS. 1A-D depict a non-limiting representation of a system in accordance with embodiments of the present disclosure comprising a nucleic acid (e.g., helper RNA or DNA) encoding an enzyme capable of performing targeted genomic integration and a nucleic acid encoding a mobile element enzyme (donor DNA). The chimeric, monomer or head-to-tail dimer mobile element enzymes are designed to target human genomic safe harbor site (GSHS) using Zinc Finger proteins (ZnF), TALE and Cas9/guide RNA DNA binders. Both DNA and RNA constructs are shown in FIGS. 1A-B.

FIG. 1A and FIG. 1C show DNA helper constructs while FIG. 1B shows RNA helper constructs. All DNA binding proteins are designed to target a TTAA site within 100 base pairs, 200 base pairs in either the sense or anti-sense orientation from the TTAA sites. ZnF sequences are based on rational design by using structure-based (Elrod-Erickson M, Pabo C. 1999 J Biol Chem 274:19281-19285) and database-guided (Desjarlais J R, Berg J M.; 1992 Proteins 12:101-104) rules that govern these discriminating DNA binding (Choo Y, Klug A. 1994 Proc Natl Acad Sci USA 91:11163-11172; Choo Y, Klug A. 1997 Curr Opin Struct Biol 7:117-125). TALEs include nuclear localization signals (NLS) and an activation domain (AD) to function as transcriptional activators. The DNA binding domain has approximately 16.5 repeats of 33-34 amino acids with a residual variable di-residue (RVD) at bases of the DNA leading strand are shown. FIGS. 1A-B show chimeric mobile element enzyme constructs comprising a ZnF, TALE DNA-binding protein, or dCas with guide RNAs fused thereto by a linker that is greater than 23 amino acids in length or spliced internally to the N-terminus of the mobile element enzyme by an intein comprises either a DNA or RNA chimeric mobile element enzyme construct. FIG. 1C shows a DNA donor construct flanked by two recognition ends or ITRs that depicts a gene of interest driven by a promoter. FIG. 1D is a non-limiting representation of a system in accordance with embodiments of the present disclosure comprising a nucleic acid (e.g., helper RNA or DNA) encoding an enzyme capable of performing targeted genomic integration and a nucleic acid encoding a mobile element enzyme (donor DNA). The helper RNA or DNA is translated into a bioengineered enzyme (e.g., integrase, recombinase, or mobile element enzyme) that recognizes specific ends and seamlessly inserts the donor DNA into the human genome in a site-specific manner without a footprint. Chimeric mobile element enzymes form dimers or tetramers at open chromatin to insert donor DNA at TTAA (SEQ ID NO: 440) recognition sites near DNA binding regions targeted by ZnF, dCas9/gRNA or TALEs. Binding of the ZnF, TALE, or Cas9/gRNA to genomic safe harbor site (GSHS) physically sequesters the mobile element enzyme as a monomer or dimer to the same location and promotes transposition to the nearby TTAA (SEQ ID NO: 440) sequences near repeat variable di-residues (RVD) nucleotide sequences.

FIG. 2 depicts a schematic diagram of present embodiments of the disclosure to produce CAR-immune (e.g., CAR-T) cells.

FIG. 3 depicts a schematic diagram of chimeric autoantibody receptor (CAAR) with the autoantigen AchR as the CAAR extracellular domain. A panel of AchR CAARs was designed for expression in primary human T-cells using AchR domains (E2-E6) as the extracellular domain, fused to a dimerization-competent CD8a transmembrane and CD137-CD3z cytoplasmic signaling domains which were used successfully in CD19 clinical trials.

FIG. 4 depicts a schematic representation of the experimental design of the T cell process for generating either a GFP or CD19-CAR-T cells from three different healthy donors for ex-vivo analysis.

FIGS. 5A-B depict results showing that nanoplasmid donor-DNA outperforms plasmid and dbDNA backbones in T cells with >50% integration >90% viability at harvest. FIG. 5A depicts GFP transgene expression by flow cytometry at days 1 and 11 post-electroporation. FIG. 5B shows % integration efficiency and % Live for human primary T cells at Day 15.

FIGS. 6A-C depicts results showing that several mRNA can achieve ~50% expression, >60% integration, and 59-75% viability in T-cells at harvest. FIG. 6A depicts % GFP transgene expression at day 14 post-electroporation. FIG. 6B depicts % integration efficiency at day 14 post-electroporation. FIG. 6C depicts representative flow plots at day 14 post-electroporation for CleanCap (34A) mRNA.

FIGS. 7A-C depicts results showing that donor:mRNA ratio of 1:2 and 1:5 show the highest GFP+ cells at Day 11 Post EP. FIG. 7A depicts GFP transgene expression at day 11 post-electroporation. FIG. 7B depicts T cell viability at day 11 post-electroporation. FIG. 7C depicts GFP Mean Fluorescence Intensity (MFI) at day 11 post-electroporation. The results indicate that the highest number of GFP+ cells were in the 2 ug donor.

FIG. 8A-B depict results showing that 1 μg and 2 ug donor show similar % integration at higher mRNA ratios at Day 11 Post EP. FIG. 8A depicts representative flow plots at day 11 post-electroporation. FIG. 8B depicts % integration efficiency at day 11 post-electroporation. The results suggest that the next step should focus on the ratio of 1:5.

FIGS. 9A-C depicts results showing that efficient engineering of T cells by MLT transposase shows stable GFP and CAR expression as well demonstrating good safety profile. FIG. 9A depicts GFP transgene expression at days 1 and 12 post-electroporation. FIG. 9B depicts CD19-CAR expression at day 14 post-electroporation. FIG. 9C depicts number of viable cells over time.

FIGS. 10A-B depict an ex vivo efficacy of CD19-CAR-T cells from three healthy donors generated by MLT transposase that efficiently kill CD19-expressing tumor target cells. FIG. 10A depicts CD19− data which shows Cytotoxicity of control and CD19-CAR T cells against CD19 negative K562 erythroleukemia cells. FIG. 10B depicts cytotoxicity of control and CD19-CAR T cells against CD19 positive B cell leukemia and lymphoma cells.

FIGS. 11A-B depict ex vivo efficacy by showing high levels of proinflammatory cytokine release by CD19 CAR-T cells upon recognition of CD19 expressing tumor targets. FIG. 11A depicts levels of IFNg and TNFa released into the supernatant upon co-culturing control or CD19-CAR T cells with CD19− or CD19+ leukemia cells. FIG. 11B depicts levels of GZMB released into the supernatant upon co-culturing control or CD19-CAR T cells with CD19− or CD19+ leukemia cells.

FIGS. 12A-B depict CD19-CAR expression of 21-26% at Day 14 (without enrichment) and >95% viability. FIG. 12A depicts CAR expression by flow cytometry of untransfected and CD19-CAR T cells at time of collection (D14). FIG. 12B depicts viability of CD19-CAR T cells at time of collection (D14).

FIGS. 13A-B depict results showing that CD19 CAR⁺ T Cells exhibit a CD4:CD8 ratio similar to those of CAR-T Cells. FIG. 13A depicts CD4 and CD8 expression by flow cytometry of control and CD19-CAR T cells. FIG. 13B depicts percentages of CD4 and CD8 populations shown in FIG. 13A.

FIGS. 14A-B depict results showing that CD19-CAR⁺ T Cells exhibit a favorable memory phenotype similar to CAR-T Cells. The favorable memory phenotype exhibits are mostly that of T_{CM} and T_{SCM}. FIG. 14A depicts a schematic diagram of different stages of memory T cell differentiation. FIG. 14B depicts Representative flow plots and percentages of T_{scm}, T_{cm}, Tₑₘ, and T_{eff} populations.

FIGS. 15A-B depict results showing that CD19-CAR⁺ T cells exhibit non-exhaustion phenotype with low expression of two exhaustion markers. The exhaustion markers are PD-1 and LAG-3. FIG. 15A depicts expression of exhaustion markers PD-1, LAG-3, and TIM-3 by flow cytometry. FIG. 15B depicts percentages of CD19-CAR T cells that express PD-1, LAG-3, or TIM-3.

### DETAILED DESCRIPTION

The present disclosure is based, in part, on the discovery that CAR-immune cell or a CAAR-immune cell generation can be made more efficient with the use of enzymatic transposition.

In aspects, there is provided a method of making a chimeric antigen receptor (CAR)-immune cell, the method comprising: obtaining an immune cell from a sample obtained from a subject, the sample comprising immune cells or immune cell progenitors; and transfecting the immune cell with a first nucleic acid encoding an enzyme capable of performing targeted genomic integration, optionally wherein the first nucleic acid is RNA, and a second, non-viral nucleic acid encoding a donor DNA comprising a chimeric CAR and flanked by ends recognized by the enzyme capable of performing targeted genomic integration, e.g., without limitation, a mobile element enzyme, to thereby create a transfected CAR-immune cell, wherein the immune cell is selected from a T cell, a macrophage, and an NK cell.

In aspects, there is provided a method of making a chimeric antigen receptor (CAR)-immune cell, the method comprising: administering to a subject with a first nucleic acid encoding an enzyme capable of performing targeted genomic integration, wherein the first nucleic acid is RNA, and a second, non-viral nucleic acid encoding a donor DNA comprising a chimeric CAR and flanked by ends recognized by the enzyme capable of performing targeted genomic integration, e.g., without limitation, a mobile element enzyme, wherein the administration uses lipid nanoparticles (LNPs) capable of directing the first and second nucleic acids to an immune cell, to thereby create a CAR-immune cell, wherein the immune cell is selected from a T cell, a macrophage, and an NK cell.

In aspects, there is provided a method of making a chimeric autoantibody receptor (CAAR)-immune cell, the method comprising: obtaining a cell from a sample obtained from a subject, the sample comprising T cells or T cell progenitors; and transfecting the cell with a first nucleic acid encoding an enzyme capable of performing targeted genomic integration, wherein the first nucleic acid is RNA, and a second, non-viral nucleic acid encoding a donor DNA comprising a chimeric CAAR comprising an autoantigen or a fragment thereof, and flanked by ends recognized by enzyme capable of performing targeted genomic integration, e.g., without limitation, a mobile element enzyme, to thereby create a transfected CAAR-immune cell.

In aspects, there is provided a method of making a chimeric autoantibody receptor (CAAR)-immune cell, the method comprising: administering to a skin cell in a subject with a first nucleic acid encoding an enzyme capable of performing targeted genomic integration, wherein the first nucleic acid is RNA, and a second, non-viral nucleic acid encoding a donor DNA comprising a chimeric CAAR comprising an autoantigen or a fragment thereof, and flanked by ends recognized by the enzyme capable of performing targeted genomic integration, e.g., without limitation, a mobile element enzyme, wherein the administration is performed via intradermal or subcutaneous delivery and using lipid nanoparticles (LNPs) capable of directing the first and second nucleic acids to an immune cell, to thereby create a CAR-immune cell.

In embodiments, the enzyme that is capable of performing targeted genomic integration inserts transgene(s) into a TCR, MHC class I, and MHC class II. In embodiments, the enzyme is capable of performing targeted genomic integration in a T-cell receptor (TCR) (e.g., without limitation, TRAC). In embodiments, the enzyme is capable of performing targeted genomic integration in a major histocompatibility complex (MHC) class I (e.g., without limitation, B2M). In embodiments, the enzyme is capable of performing targeted genomic integration in an MHC class II (e.g., without limitation, CIITA).

In embodiments, the transgene is a T cell receptor alpha constant (TRAC) gene. In embodiments, the transgene is a 12 microglobulin (B2M) gene. In embodiments, the transgene is a CIITA gene. In embodiments, the transgene is a PDL gene, optionally a programmed cell death ligand-1 (PDL-1) gene.

In embodiments, the enzyme is capable of targeting the genes, optionally a TRAC gene, associated with the TCR. In embodiments, the enzyme is capable of targeting the genes, optionally a B2M gene, associated with MHC class I. In embodiments, the enzyme is capable of targeting the genes, optionally a CIITA gene, associated with MHC class II.

### Generation of CAR-Immune Cells, CAAR-Immune Cells, and the Like

In aspects, the present disclosure relates to the production of CAR-immune cells and/or CAAR-immune cells via an enzyme capable of performing targeted genomic integration. In embodiments, the methods are conducted ex vivo or in vivo.

In embodiments, the immune cell is selected from a T cell, a macrophage, and an NK cell.

In embodiments, the immune cell is a T cell. In embodiments, the T cells comprise primary T cells. In embodiments, the transfected CAR-immune cell is a CAR-T cell, optionally selected from an allogeneic CAR-T cell or an allogeneic CAR-T cell.

In embodiments, the immune cell is a macrophage. In embodiments, the macrophages comprise primary macrophages.

In embodiments, the transfected CAR-immune cell is a CAR-M cell, optionally selected from an allogeneic CAR-M cell or an allogeneic CAR-M cell.

In embodiments, the immune cell is a natural killer (NK) cell. In embodiments, the NK cells comprise primary NK cells.

In embodiments, the transfected CAR-immune cell is a CAR-NK cell, optionally selected from an allogeneic CAR-NK cell or an allogeneic CAR-NK cell.

In embodiments, the immune cell is a T cell. In embodiments, the T cells comprise primary T cells. In embodiments, the transfected CAAR-immune cell is a CAAR-T cell, optionally selected from an allogeneic CAAR-T cell or an allogeneic CAAR-T cell.

In embodiments, the immune cell is a macrophage. In embodiments, the macrophages comprise primary macrophages.

In embodiments, the transfected CAAR-immune cell is a CAAR-M cell, optionally selected from an allogeneic CAAR-M cell or an allogeneic CAAR-M cell.

In embodiments, the immune cell is a natural killer (NK) cell. In embodiments, the NK cells comprise primary NK cells.

In embodiments, the transfected CAAR-immune cell is a CAAR-NK cell, optionally selected from an allogeneic CAAR-NK cell or an allogeneic CAAR-NK cell.

In embodiments, the method comprises activating the cell before transfection or administration, wherein the activation optionally comprises contacting the cell with an anti-CD3 antibody or CD3-binding fragment thereof.

In embodiments, the method comprises stimulating the cell before transfection or administration to create a population of cells, wherein the stimulation optionally comprises contacting the cell with an anti-CD28 antibody or a CD28-binding fragment thereof, B7-1 or a CD28-binding fragment thereof, or B7-2 or a CD28-binding fragment thereof.

In embodiments, the chimeric CAAR comprises an autoantigen or a fragment thereof. In embodiments, the autoantigen or a fragment thereof is an extracellular domain for targeting of an autoantibody or a B-cell receptor (BCR). In embodiments, the extracellular domain is an AChR autoantigen. In embodiments, the AChR autoantigen comprises domains E2-E6, and wherein the AChR autoantigen is fused to a CD8 alpha chain transmembrane domain and CD137-CD3z cytoplasmic signaling domains. In embodiments, the extracellular domain is a Muscle Specific Kinase (MuSK) extracellular domain. In embodiments, the extracellular domain is a desmoglein-3 (Dsg3). In embodiments, the extracellular domain is a desmoglein-1 (Dsg1). In embodiments, the autoantigen or a fragment thereof is allogeneic. In embodiments, the autoantigen or a fragment thereof is xenogeneic. In embodiments, the method comprises culturing the transfected CAAR-immune cells in a medium that selectively enhances proliferation of CAAR-immune cells. In embodiments, the transfected CAAR-immune cell is created in about 1 day or about 2 days. In embodiments, the transfected CAAR-immune cell is created in less than 2 days. In embodiments, the method obviates the use of ex vivo CAAR-immune cell expansion.

In embodiments, the CAAR comprises an extracellular domain that binds an autoantibody expressed on a B cell, a transmembrane domain, and/or an intracellular signaling domain. In embodiments, the extracellular domain that binds an autoantibody expressed on a B cell comprises Dsg1, Dsg3, or a fragment thereof. In embodiments, the CAAR comprises a transmembrane domain, such as those disclosed elsewhere herein. In embodiments, the CAAR comprises a transmembrane domain, such as, but not limited to, a human T cell surface glycoprotein CD8 alpha chain hinge and/or transmembrane domain. In embodiments, the CAAR comprises an intracellular signaling domain, such as those disclosed elsewhere herein. In embodiments, the CAAR comprises an intracellular signaling domain such as the cytoplasmic portion of the T cell receptor (TCR) and co-receptors that act in concert to initiate signal transduction following antigen receptor engagement, as well as any derivative or variant thereof.

In embodiments, the donor DNA comprises or further comprises one or more of a nucleic acid sequence of a transmembrane domain, a nucleic acid sequence of an intracellular domain of a costimulatory molecule, and a nucleic acid sequence of a signaling domain. In embodiments, the donor DNA comprises or further comprises a nucleic acid sequence of a CD8 alpha chain signal peptide. In embodiments, the nucleic acid sequence of the transmembrane domain encodes a CD8 alpha chain hinge and a transmembrane domain. In embodiments, the donor DNA further comprises a nucleic acid sequence of a peptide linker. In embodiments, the nucleic acid sequence of the intracellular signaling domain comprises a nucleic acid sequence encoding a CD3 zeta (CD3ζ) signaling domain. In embodiments, the second nucleic acid encodes an extracellular single-chain variable fragment (scFv) antibody to CD19 fused to a T cell cytoplasmic signaling domain. In embodiments, the second nucleic acid encodes a single-chain Fv domain (scFv) comprising a VL linked to a VH of a specific antibody by a flexible linker, at least a part of a transmembrane domain, at least a part of a cytoplasmic, optionally an extracellular, domain of an endogenous protein. In embodiments, the scFv domain is an scFv domain of an antibody against a tumor cell or tumor cell marker. In embodiments, the scFv domain is an scFv domain of an antibody against a virus.

In embodiments, the endogenous protein is a lymphocyte receptor chain, a polypeptide of the TCR/CD3 complex, or a subunit of the Fc or IL-2 receptor. In embodiments, the second nucleic acid encodes the α, β, γ, or δ chain of an antigen-specific T cell receptor.

In embodiments, the donor DNA comprises a nucleic acid encoding an extracellular domain, a transmembrane domain, and an intracellular domain of the CAR.

In embodiments, the intracellular domain comprises a costimulatory signaling region comprising an intracellular domain of a costimulatory molecule selected from CD27, CD28, 4-1BB, IL-2Rβ, OX40, CD30, GITR, TIM3, DAP10, CD40, PD-1, ICOS, lymphocyte function-associated antigen-1 (LFA-1), CD2, CD7, LIGHT, NKG2C, B7-H3, and any combination thereof.

In embodiments, the CAR comprises an extracellular domain that binds an antigen selected from alpha folate receptor, 5T4, αvβ6 integrin, BCMA, B7-H3, B7-H6, CAIX, CD19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, GD2, GD3, Glypican-3 (GPC3), HLA-A1+MAGE1, HLA-A2+MAGE1, HLA-A3+MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, and VEGFR2.

In embodiments, the CAR comprises a transmembrane domain derived from a polypeptide selected from CD8α; CD4, CD28, CD45, PD-1, and CD152.

In embodiments, the CAR comprises one or more intracellular costimulatory signaling domains selected from CD28, CD54 (ICAM), CD134 (OX40), CD137 (41BB), IL-2Rβ, CD152 (CTLA4), CD273 (PD-L2), CD274 (PD-L1), and CD278 (ICOS).

In embodiments, the CAR comprises a CD3 zeta (CD3ζ) signaling domain.

In embodiments, the CAR comprises an extracellular domain that binds an antigen selected from alpha folate receptor, 5T4, αvβ6 integrin, BCMA, B7-H3, B7-H6, CAIX, CD19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, GD2, GD3, Glypican-3 (GPC3), HLA-A1+MAGE1, HLA-A 2+MAGE1, HLA-A3+MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, and VEGFR2; a transmembrane domain derived from a polypeptide selected from CD8α; CD4, CD28, CD45, PD-1, and CD152; one or more intracellular costimulatory signaling domains selected from CD28, CD54 (ICAM), CD134 (OX40), CD137 (41BB), IL-2Rβ, CD152 (CTLA4), CD273 (PD-L2), CD274 (PD-L1), and CD278 (ICOS); and a CD3 zeta (CD3ζ) signaling domain.

In embodiments, the CARs to be used in the method of the present disclosure are recombinant chimeric receptors comprising: (i) an antigen-specific targeting domain; (ii) a spacer domain; (iii) a transmembrane domain; (iv) at least one costimulatory domain; and/or (v) an intracellular signaling domain.

In embodiments, the extracellular domain of the CAR to be used in the method of the present disclosure comprises an antigen-specific targeting domain that has the function of binding to the target antigen of interest. In embodiments, the antigen-specific targeting domain may be any naturally occurring, synthetic, semi-synthetic, or a molecule produced recombinant technology, protein, peptide or oligo peptide that specifically binds to the target antigen. Examples of possible antigen-specific targeting domains include antibodies or antibody fragments or derivatives, synthetic or naturally occurring ligands of the targeted receptor including molecules, binding or extracellular domains of receptors or binding proteins. In embodiments, the antigen-specific targeting domain is, or is derived from, an antibody. An antibody is a protein, or a polypeptide sequence derived from an immunoglobulin able to bind with an antigen. Antibody as herein used includes polyclonal or monoclonal, multiple or single chain antibodies as well as immunoglobulins, whether deriving from natural or recombinant source. An antibody-derived targeting domain can be a fragment of an antibody or a genetically engineered product of one or more fragments of the antibody, which fragment is involved in binding with the antigen. Examples include a variable region (Fv), a complementarity determining region (CDR), a Fab, a single chain antibody (scFv), a heavy chain variable region (VFI), a light chain variable region (VL) and a camelid antibody (VHH). In embodiments, the binding domain is a single chain antibody (scFv). The scFv may be murine, human, or humanized scFv.

In embodiments, the CAR to be used in the method of the disclosure comprises an extracellular spacer domain that connects the antigen-specific targeting domain to the transmembrane domain. The most common sequence used as spacer is the constant immunoglobulin IgG1 hinge-CH2-CH3 Fc domain. Mutants and or variants of this spacer may be employed, e.g., those which reduce Fc Receptor binding.

In embodiments, the CAR to be used in the method of the disclosure comprises a transmembrane domain between the spacer domain and the signaling domain. In embodiments, the transmembrane domain may be derived either from a natural or from a synthetic source. In embodiments, the domain deriving from natural sources may comprise the transmembrane sequence from any membrane-bound or transmembrane protein including any of the type I, type II, or type Ill transmembrane proteins. In embodiments, the transmembrane regions that may be used in the CAR may be derived from the alpha, beta, or zeta chain of the T-cell receptor, CD28, CD3 epsilon, CD45, CD4, CD5, CD8, CD9, CD16, CD22, CD33, CD37, CD64, CD80, CD86, CD134, CD137, CD154, CD244 (2B4), DAP10, or DAP12. In embodiments, the domain deriving from synthetic source will comprise predominantly hydrophobic sequence including residues such as leucine and valine.

In embodiments, the CAR used in the method of the present disclosure may include, e.g., in the cytoplasmic tail, one or more costimulatory domains. Such domains may consist of the intracellular signaling domain of one or more costimulatory protein receptors (e.g., CD28, 41BB, ICOS). In embodiments, the costimulatory domain provides additional signals to the cells thus enhancing cell expansion, cell survival and development of memory cells. In embodiments, the CAR used in the method of the present disclosure may comprise one or more costimulatory domain selected from the group consisting of the intracellular domain of members of the TNFR super family, CD28, CD137 (4-1BB), CD134 (OX40), Dap10, CD27, CD2, CD5, ICAM-1, LFA-1, Lck, TNFR-1, TNFR-II, Fas, CD30, CD40, CD244 (2B4), DAP12, or combinations thereof. Further examples of costimulatory domains may be employed by the skilled in the art in the CAR.

In embodiments, the CAR used in the method of the disclosure may also comprise an intracellular signaling domain. This domain may be cytoplasmic, transmits the activation signal and direct the cell to perform its specialized function. Examples of intracellular signaling domains include, but are not limited to, z chain of the T-cell receptor or any of its homologs (e.g., h chain, FcRly and b chains, MB1 (Iga) chain, B29 (IgP) chain, etc.), CD3 polypeptides (D, d and e), syk family tyrosine kinases (Syk, ZAP 70, etc.), src family tyrosine kinases (Lck, Fyn, Lyn, etc.) and other molecules involved in T-cell signal transduction, such as CD2, CD5 and CD28. In embodiments, the intracellular signaling domain may be human CD3 zeta chain, FcγRIII, FcsRI, cytoplasmic tails of Fc receptors, immunoreceptor tyrosine-based activation motif (ITAM) bearing cytoplasmic receptors or combinations thereof. In embodiments, the signaling domain comprises the intracellular signaling domain of human CD3 zeta chain.

In embodiments, the method comprises culturing the transfected CAR-immune cell in a medium that selectively enhances proliferation of CAR-immune cells or CAAR-immune cells.

In embodiments, the CAR-immune cell or CAAR-immune cell is created in about 1 day or about 2 days. In embodiments, the CAR-immune cell or CAAR-immune cell is created in less than about 2 days, or less than about 3 days, or less than about 7 days, or less than about 14 days.

In embodiments, the method obviates a use of ex vivo expansion of CAR-immune cells or CAAR-immune cells.

In embodiments, the skin cell is epidermis or dermis skin cell.

In embodiments, the extracellular domain comprises an antibody or antigen binding fragment that binds an antigen.

In embodiments, the cell is transfected by electroporation, nucleofection, hydrodynamic delivery, or calcium phosphate precipitation and/or is carried out using a lipid vehicle, optionally N-[1-(2,3-dioleoyloxy)propyl]-N,N,N-trimethylammonium chloride (DOTMA), 1,2-bis(oleoyloxy)-3-3-(trimethylammonia) propane (DOTAP), or 1,2-dioleoyl-3-dimethylammonium-propane (DODAP), dioleoylphosphatidylethanolamine (DOPE), cholesterol, LIPOFECTIN (cationic liposome formulation), LIPOFECTAMINE (cationic liposome formulation), LIPOFECTAMINE 2000 (cationic liposome formulation), LIPOFECTAMINE 3000 (cationic liposome formulation), TRANSFECTAM (cationic liposome formulation), a lipid nanoparticle, or a liposome and combinations thereof.

In embodiments, the transfecting of the cell is carried out using electroporation, or calcium phosphate precipitation.

In embodiments, the transfecting of the cell is carried out using a lipid vehicle, optionally N-[1-(2,3-dioleoyloxy)propyl]-N,N,N-trimethylammonium chloride (DOTMA), 1,2-bis(oleoyloxy)-3-3-(trimethylammonia) propane (DOTAP), or 1,2-dioleoyl-3-dimethylammonium-propane (DODAP), dioleoylphosphatidylethanolamine (DOPE), cholesterol, LIPOFECTIN (cationic liposome formulation), LIPOFECTAMINE (cationic liposome formulation), LIPOFECTAMINE 2000 (cationic liposome formulation), LIPOFECTAMINE 3000 (cationic liposome formulation), TRANSFECTAM (cationic liposome formulation), a lipid nanoparticle, or a liposome and combinations thereof.

In embodiments, the transfecting of the cell is carried out using a lipid selected from one or more of the following categories: cationic lipids; anionic lipids; neutral lipids; multi-valent charged lipids; and zwitterionic lipids. In embodiments, a cationic lipid may be used to facilitate a charge-charge interaction with nucleic acids. In embodiments, the lipid is a neutral lipid. In embodiments, the neutral lipid is dioleoylphosphatidylethanolamine (DOPE), 1,2-Dioleoyl-sn-glycero-3-phosphocholine (DOPC), or cholesterol. In embodiments, cholesterol is derived from plant sources. In other embodiments, cholesterol is derived from animal, fungal, bacterial or archaeal sources. In embodiments, the lipid is a cationic lipid. In embodiments, the cationic lipid is N-[1-(2,3-dioleoyloxy)propyl]-N,N,N-trimethylammonium chloride (DOTMA), 1,2-bis(oleoyloxy)-3-3-(trimethylammonia) propane (DOTAP), or 1,2-dioleoyl-3-dimethylammonium-propane (DODAP). In embodiments, one or more of the phospholipids 18:0 PC, 18:1 PC, 18:2 PC, DMPC, DSPE, DOPE, 18:2 PE, DMPE, or a combination thereof are used as lipids. In embodiments, the lipid is DOTMA and DOPE, optionally in a ratio of about 1:1. In embodiments, the lipid is DHDOS and DOPE, optionally in a ratio of about 1:1. In embodiments, the lipid is a commercially available product (e.g., LIPOFECTIN (cationic liposome formulation), LIPOFECTAMINE (cationic liposome formulation), LIPOFECTAMINE 2000 (cationic liposome formulation), LIPOFECTAMINE 3000 (cationic liposome formulation) (Life Technologies)).

In embodiments, the transfecting of the cell is carried out using a cationic vehicle, optionally LIPOFECTIN or TRANSFECTAM.

In embodiments, the transfecting of the cell is carried out using a lipid nanoparticle, or a liposome.

In embodiments, the method is helper virus-free.

In embodiments, the second nucleic acid is included in an expression vector. In embodiments, the expression vector comprises a plasmid. In embodiments, the expression vector includes a neomycin phosphotransferase gene.

In embodiments, the second nucleic acid is DNA, optionally cDNA. In embodiments, the second nucleic acid is a plasmid, optionally a nanoplamid.

In embodiments, the second nucleic acid has at least one chromatin element, wherein the at least one chromatin element is optionally a Matrix Attachment Region (MAR) element.

Epigenetic regulatory elements can be used to protect a transgene from unwanted epigenetic effects when placed near the transgene on a vector including the transgene. See Ley et al., PloS One vol. 8,4 e62784. 30 Apr. 2013, doi:10.1371/journal.pone.0062784. For example, MARs were shown to increase genomic integration and integration of a transgene while preventing heterochromatin silencing, as exemplified by the human MAR 1-68. See id.; see also Grandjean et al., Nucleic Acids Res. 2011 August; 39(15):e104. MARs can also act as insulators and thereby prevent the activation of neighboring cellular genes. Gaussin et al., Gene Ther. 2012 January; 19(1):15-24. It has been shown that a piggyBac donor DNA containing human MARs in CHO cells mediated efficient and sustained expression from a few transgene copies, using cell populations generated without an antibiotic selection procedure. See Ley et al. (2013).

In embodiments, the cell is further transfected with a third nucleic acid having at least one chromatin element, wherein the at least one chromatin element is optionally a Matrix Attachment Region (MAR) element. MARs are expression enhancing, epigenetic regulator elements which are used to enhance and/or facilitate transgene expression, as described, for example, in PCT/IB2010/002337 (WO2011033375) which is incorporated by reference herein in its entirety. A MAR element can be located in cis or trans to the donor DNA.

In embodiments, the donor DNA has a size of about 100,000 bases or less, e.g., about 100,000 bases, or about 50,000 bases, or about 30,000 bases, or about 10,000 bases, or about 5,000 bases, or about 10,000 to about 100,000 bases, or about 30,000 to about 100,000 bases, or about 50,000 to about 100,000 bases, or about 10,000 to about 50,000 bases, or about 10,000 to about 30,000 bases, or about 30,000 to about 50,000 bases.

In embodiments, the donor DNA has a size of about 200,000 bases or less, e.g., about 200,000 bases, or about 10,000 to about 200,000 bases, or about 30,000 to about 200,000 bases, or about 50,000 to about 200,000 bases, or about 100,000 to about 200,000 bases, or about 150,000 to about 200,000 bases.

In embodiments, the enzyme and the donor DNA are transfected at a donor DNA to enzyme ratio of about 1 to about 5, or about 1 to about 4, or about 1 to about 3, or about 1 to about 2, or about 1 to about 1.

In embodiments, the amount of donor DNA transfected is about 1 μg to about 10 μg, or about 1 μg to about 8 μg, or about 1 μg to about 6 μg, or about 1 μg to about 4 μg, or about 1 μg, or about 2 μg, or about 5 μg, or about 10 μg.

In embodiments, the amount of donor DNA transfected is about 1 μg.

In embodiments, the amount of donor DNA transfected is about 2 μg.

In embodiments, the amount of donor DNA transfected is about 1 μg and the amount of an enzyme RNA transfected is about 2 μg, or about 5 μg, or about 10 μg.

In embodiments, the amount of donor DNA transfected is about 2 μg and the amount of an enzyme RNA transfected is about 4 μg, or about 8 μg, or about 10 μg, or about 20 μg.

In embodiments, the present method comprises culturing for about 10, or about 12, or about 14, or about 16 days after transfection. In embodiments, the resultant cells have a viability of at least about 90%, or at least about 95%, or at least about 99%.

In embodiments, the present method yields CAR T-cells that are cytotoxic to target, e.g., tumor, cells. In embodiments, the present method yields CAR T-cells that cause release of cytokines upon target recognition on a tumor cell. In embodiments, the present method yields CAR T-cells that cause release of one or more of IFN gamma, TNF alpha, and granzyme B upon target recognition on a tumor cell. In embodiments, the present method yields CAR T-cells which are not exhausted. In embodiments, the present method yields CAR T-cells with low expression of PD-1 and/or LAG-3. In embodiments, the present method yields CAR T-cells which are memory competent.

### Enzymes

In embodiments, an enzyme capable of performing targeted genomic integration is any type of an enzyme that cause a donor DNA to be inserted from one location (e.g., without limitation, donor DNA) to a specific site and/or locus in a subject's genome.

In embodiments, the enzyme capable of performing targeted genomic integration is a recombinase.

In embodiments, the recombinase is an integrase. In embodiments, the enzyme is a mobile element enzyme. In embodiments, the recombinase is an integrase or a mobile element enzyme.

In embodiments, the mobile element enzyme is an engineered mammalian mobile element enzyme. In embodiments, the mobile element enzyme is a mammal-derived, helper RNA mobile element enzyme. Messenger RNA (mRNA) is an effective alternative to DNA as a source of a mobile element enzyme for targeting somatic cells and tissues, given that RNA is a safer alternative to DNA as a source of a mobile element enzyme for somatic gene therapy applications. See, e.g., Wilber et al., Mol. Ther. 13, 625-630 (2006). Successful use of in vitro-transcribed mRNA as a transient source of mobile element enzyme and subsequent transposition in cultured human cells and in live mice was previously reported for Sleeping Beauty mobile element enzyme. See id. It was demonstrated that in vitro-transcribed, UTR-stabilized mobile element enzyme-encoding mRNA can be used as a source of mobile element enzyme for Sleeping Beauty-mediated transposition in cultured somatic cells. Id. Also, Hoerr et al. reported that a specific cytotoxic T cell response and circulating antigen-specific antibodies were detected after administration of in vitro-transcribed, UTR-stabilized, and protamine-condensed bacterial lacZ mRNA into the ear pinna of Balb/C mice. Hoerr et al., Eur. J. Immunol. 2000; 30: 1-7; see also Wilber et al. (2006).

In embodiments, the mobile element enzyme is a mammal-derived, DNA mobile element enzyme. In embodiments, the mobile element enzyme is a chimeric mobile element enzyme.

In embodiments, the enzyme capable of performing targeted genomic integration is a mobile element enzyme, and the mobile element enzyme comprises (a) a targeting element which is or comprises a gene-editing system, and (b) a mobile element enzyme that is capable of inserting the donor DNA comprising a transgene, a chimeric CAR, or chimeric CAAR, optionally at a TA dinucleotide site or a TTAA (SEQ ID NO: 440) tetranucleotide site, optionally in a genomic safe harbor site (GSHS), as described elsewhere herein.

In embodiments, the enzyme is derived from Bombyx mori, Xenopus tropicalis, Trichoplusia ni, Myotis lucifugus, Rhinolophus ferrumequinum, Rousettus aegyptiacus, Phyllostomus discolor, Myotis myotis, Pteropus vampyrus, Pipistrellus kuhlii, Molossus molossus, Pan troglodytes, or Homo sapiens. In embodiments, the enzyme is an engineered version, including but not limited to hyperactive forms, of an enzyme derived from Bombyx mori, Xenopus tropicalis, Trichoplusia ni, Myotis lucifugus, Rhinolophus ferrumequinum, Rousettus aegyptiacus, Phyllostomus discolor, Myotis myotis, Pteropus vampyrus, Pipistrellus kuhlii, Molossus molossus, Pan troglodytes, or Homo sapiens.

In embodiments, the enzyme is a mobile element enzyme derived from Bombyx mori, Xenopus tropicalis, Trichoplusia ni, Myotis lucifugus, Rhinolophus ferrumequinum, Rousettus aegyptiacus, Phyllostomus discolor, Myotis myotis, Pteropus vampyrus, Pipistrellus kuhlii, Molossus molossus, Pan troglodytes, or Homo sapiens. In embodiments, the enzyme is an engineered version, including but not limited to hyperactive forms, of a mobile element enzyme derived from Bombyx mori, Xenopus tropicalis, Trichoplusia ni, Myotis lucifugus, Rhinolophus ferrumequinum, Rousettus aegyptiacus, Phyllostomus discolor, Myotis myotis, Pteropus vampyrus, Pipistrellus kuhlii, Molossus molossus, Pan troglodytes, or Homo sapiens.

In embodiments, the mobile element enzyme is from one or more of the Tn1, Tn2, Tn3, Tn5, Tn7, Tn9, Tn10, Tn552, Tn903, Tn1000/Gamma-delta, Tn/O, tnsA, tnsB, tnsC, tniQ, IS10, ISS, IS911, Minos, Sleeping beauty, piggyBac, Tol2, Mos1, Himar1, Hermes, Tol2, Minos, Tel, P-element, MuA, Ty1, Chapaev, transib, Tc1/mariner, or Tc3 donor DNA system, or biologically active fragments variants thereof, inclusive of hyperactive mutants (e.g., without limitation selected from TABLE 1, or equivalents thereof).

In embodiments, the mobile element enzyme is from a MLT donor DNA system that is based on a cut-and-paste MLT element obtained from the little brown bat (Myotis lucifugus) or other bat mobile element enzymes, such as Rhinolophus ferrumequinum, Rousettus aegyptiacus, Phyllostomus discolor, Myotis myotis, Pipistrellus kuhlii, and Molossus molossus. See Mitra et al., Proc Natl Acad Sci USA. 2013 Jan. 2; 110(1):234-9; Jebb et al., Nature, volume 583, pages 578-584 (2020), which are incorporated by reference herein in their entireties. In embodiments, hyperactive forms of a bat mobile element enzyme are used. The MLT mobile element enzyme has been shown to be capable of transposition in bat, human, and yeast cells. The hyperactive forms of the MLT mobile element enzyme enhance the transposition process. In addition, chimeric MLT mobile element enzymes are capable of site-specific excision without genomic integration.

In embodiments, the mobile element enzyme is a Myotis lucifugus mobile element enzyme (MLT), which is either the wild type, monomer, dimer, tetramer (or another multimer), hyperactive, an Int-mutant, or of any other form.

In embodiments, the MLT mobile element enzyme has an amino acid sequence of SEQ ID NO: 1, or a variant having at least about 80%, at least about 90%, or at least about 93%, or at least about 95%, or at least about 97%, or at least about 98%, or at least about 99% identity thereto, and one or more mutations selected from L573X, E574X, and S2X, wherein X is any amino acid or no amino acid, optionally X is A, G, or a deletion, optionally the mutations are L573del E574del, and S2A). In embodiments, the MLT mobile element enzyme has the nucleotide sequence of SEQ ID NO: 2 (which is a codon-optimized form of MLT), or a nucleotide sequence having at least about 90%, or at least about 93%, or at least about 95%, or at least about 97%, or at least about 98%, or at least about 99% identity thereto.

SEQ ID NO: 1 is:

MAQHSDYSDDEFCADKLSNYSCDSDLENASTSDEDSSDDEVMVRP
RTLRRRRISSSSSDSESDIEGGREEWSHVDNPPVLEDFLGHQGLN
TDAVINNIEDAVKLFIGDDFFEFLVEESNRYYNQNRNNFKLSKKS
LKWKDITPQEMKKFLGLIVLMGQVRKDRRDDYWTTEPWTETPYFG
KTMTRDRFRQIWKAWHENNNADIVNESDRLCKVRPVLDYFVPKFI
NIYKPHQQLSLDEGIVPWRGRLFFRVYNAGKIVKYGILVRLLCES
DTGYICNMEIYCGEGKRLLETIQTVVSPYTDSWYHIYMDNYYNSV
ANCEALMKNKFRICGTIRKNRGIPKDFQTISLKKGETKFIRKNDI
LLQVWQSKKPVYLISSIHSAEMEESQNIDRISKKKIVKPNALIDY
NKHMKGVDRADQYLSYYSILRRTVKWTKRLAMYMINCALFNSYAV
YKSVRQRKMGFKMFLKQTAIHWLTDDIPEDMDIVPDLQPVPSTSG
MRAKPPTSDPPCRLSMDMRKHTLQAIVGSGKKKNILRRCRVCSVH
KLRSETRYMCKFCNIPLHKGACFEKYHTLKNY

In embodiments, the MLT mobile element enzyme has an amino acid sequence of SEQ ID NO: 1 or a variant having at least about 80%, at least about 90%, or at least about 93%, or at least about 95%, or at least about 97%, or at least about 98%, or at least about 99% identity thereto and comprises an amino acid other than serine at the position corresponding to position 2 of SEQ ID NO: 1. In embodiments, the amino acid is a non-polar aliphatic amino acid, optionally a non-polar aliphatic amino acid optionally selected from G, A, V, L, I and P, optionally A. In embodiments, the mobile element enzyme does not have additional residues at the C terminus relative to SEQ ID NO: 1.

In embodiments, the MLT mobile element enzyme has a nucleotide sequence of SEQ ID NO: 2 (which is codon-optimized) and an amino acid sequence SEQ ID NO: 1, respectively. In embodiments, the MLT mobile element enzyme has a nucleotide sequence of SEQ ID NO: 2, or a nucleotide sequence having at least about 90%, or at least about 93%, or at least about 95%, or at least about 97%, or at least about 98%, or at least about 99% identity thereto, or a codon-optimized form thereof. In embodiments, the MLT mobile element enzyme has an amino acid sequence SEQ ID NO: 1, or an amino acid sequence having at least about 90%, or at least about 93%, or at least about 95%, or at least about 97%, or at least about 98%, or at least about 99% identity thereto.

In embodiments, the mobile element enzyme can act on an MLT left terminal end, or a sequence having at least about 90%, or at least about 93%, or at least about 95%, or at least about 97%, or at least about 98%, or at least about 99% identity thereto, wherein the nucleotide sequence of the MLT left terminal end (5′ to 3′) is as follows:

(SEQ ID NO: 21)
ttaacacttggattgcgggaaacgagttaagtcggctcgcgtgaa
ttgcgcgtactccgcgggagccgtcttaactcggttcatatagat
ttgcggtggagtgcgggaaacgtgtaaactcgggccgattgtaac
tgcgtattaccaaatatttgtt

In embodiments, the mobile element enzyme can act on an MLT right terminal end, or a sequence having at least about 90%, or at least about 93%, or at least about 95%, or at least about 97%, or at least about 98%, or at least about 99% identity thereto, wherein the nucleotide sequence of the MLT right terminal end (5′ to 3′) is as follows:

(SEQ ID NO: 22)
aattatttatgtactgaatagataaaaaaatgtctgtgattgaat
aaattttcattttttacacaagaaaccgaaaatttcatttcaatc
gaacccatacttcaaaagatataggcattttaaactaactctgat
tttgcgcgggaaacctaaataattgcccgcgccatcttatatttt
ggQgggaaattcacccgacaccgtAgtgttaa

In embodiments, the donor DNA is flanked by one or more terminal ends. In embodiments, the donor DNA is or comprises a gene encoding a compete polypeptide. In embodiments, the donor DNA is or comprises a gene which is defective or substantially absent in a disease state.

In embodiments, the enzyme (e.g., without limitation, a mobile element enzyme, e.g., without limitation, MLT mobile element enzyme), inclusive of any described herein has one or more mutations which confer hyperactivity.

In embodiments, the enzyme (e.g., without limitation, a mobile element enzyme, e.g., without limitation, MLT mobile element enzyme) has gene cleavage activity (Exc+) and/or gene integration activity (Int+) activity. In embodiments, the enzyme (e.g., without limitation, a mobile element enzyme, e.g., without limitation, MLT mobile element enzyme) has gene cleavage activity (Exc+) and/or a lack of gene integration activity (Int−).

In embodiments, the mobile element enzyme, e.g., without limitation, MLT mobile element enzyme includes a hyperactive mutation, e.g., about 1, or about 2, or about 3, or about 4, or about 5 hyperactive mutations or combinations thereof. In embodiments, the mobile element enzyme can include any number of any of the hyperactive mutations, or equivalents thereof, described herein.

In embodiments, the MLT mobile element enzyme includes a hyperactive mutation, e.g., about 1, or about 2, or about 3, or about 4, or about 5 hyperactive mutations, or combinations thereof. In embodiments, the mobile element enzyme can include any number of any of the hyperactive mutations, or equivalents thereof, described herein.

In embodiments, the enzyme comprises one or more mutations corresponding to TABLE 1, or positions corresponding thereto, which, without being bound by theory, provides hyperactive mutations. Numbering relative to the amino acid sequence of protein of SEQ ID NO: 1, and nucleic acid sequence of SEQ ID NO: 2.

TABLE 1

| Nucleotide Change | Amino Acid Change |
|---|---|
| T13C | S5P |
| T22C | S8P |
| T22C/T37C | S8P/C13R |
| A26G | D9G |
| A29G | D10G |
| A32G | E11G |
| T37C | C13R |
| C41T | A14V |
| A106G | S36G |
| G161A | S54N |
| T375G | N125K |
| A389C | K130T |
| G715A | G239S |
| A880G | T294A |
| A898G | T300A |
| A1033G | I345V |
| G1280A | R427H |
| A1424G | D475G |
| A1441G | M481V |
| C1472A | P491Q |
| G1558A | A520T |
| G1681A | A561T |

In embodiments, the MLT mobile element enzyme has one or more amino acid substitutions selected from S8X1, C13X2 and/or N125X3, at positions corresponding to SEQ ID NO: 1, wherein X1 is selected from G, A, V, L, I and P, X2 is selected from K, R, and H, and X3 is selected from K, R, and H, or wherein: X1 is P, X2 is R, and/or X3 is K.

In embodiments, the MLT mobile element enzyme has S8X1, C13X2 and N125X3 substitutions, at positions corresponding to SEQ ID NO: 1, wherein X1 is selected from G, A, V, L, I and P, X2 is selected from K, R, and H, and X3 is selected from K, R, and H, or wherein: X1 is P, X2 is R, and/or X3 is K.

In embodiments, the MLT mobile element enzyme has S8X1 and C13X2 substitutions, at positions corresponding to SEQ ID NO: 1, wherein X1 is selected from G, A, V, L, I and P, X2 is selected from K, R, and H, and X3 is selected from K, R, and H, or wherein: X1 is P, X2 is R, and/or X3 is K.

In embodiments, the MLT mobile element enzyme has S8X1 and N125X3 substitutions, at positions corresponding to SEQ ID NO: 1, wherein X1 is selected from G, A, V, L, I and P, X2 is selected from K, R, and H, and X3 is selected from K, R, and H, or wherein: X1 is P, X2 is R, and/or X3 is K.

In embodiments, the MLT mobile element enzyme has C13X2 and N125X3 substitutions, at positions corresponding to SEQ ID NO: 1, wherein X1 is selected from G, A, V, L, I and P, X2 is selected from K, R, and H, and X3 is selected from K, R, and H, or wherein: X1 is P, X2 is R, and/or X3 is K.

In embodiments, the MLT mobile element enzyme has an amino acid sequence of SEQ ID NO: 1, or a variant thereof, and S8P and C13R mutations (SEQ ID NO: 11). In embodiments, the MLT mobile element enzyme has an amino acid sequence having mutations at positions which correspond to at least one of S8P and C13R mutations relative to the amino acid of SEQ ID NO: 1 or a functional equivalent thereof. In embodiments, the MLT mobile element enzyme has an amino acid sequence having mutations at positions which correspond to S8P and C13R mutations relative to the amino acid of SEQ ID NO: 1 or a functional equivalent thereof.

In embodiments, the MLT mobile element enzyme has an amino acid sequence of SEQ ID NO: 1, or a variant thereof, and S8P, C13R, and N125K mutations (SEQ ID NO: 10).

In embodiments, a MLT mobile element enzyme comprising the amino acid sequence of SEQ ID NO: 1, or a variant thereof, and includes one or more hyperactive mutations selected from a substitution or deletion at one or more of positions S5, S8, D9, D10, E11, C13, A14, S36, S54, N125, K130, G239, T294, T300, I345, R427, D475, M481, P491, A520, and A561, or positions corresponding thereto.

In embodiments, a MLT mobile element enzyme comprising the amino acid sequence of SEQ ID NO: 1, or a variant thereof, and includes one or more hyperactive mutations selected from S5P, S8P, S8P/C13R, D9G, D10G, E11G, C13R, A14V, S36G, S54N, N125K, K130T, G239S, T294A, T300A, I345V, R427H, D475G, M481V, P491Q, A520T, and A561T, or positions corresponding thereto.

In embodiments, the MLT mobile element enzyme comprises one or more of hyperactive mutants selected from S8X₁, C13X₂ and/or N125X₃ (e.g., all of S8X₁, C13X₂ and N125X₃, S8X₁ and C13X₂, S8X₁ and N125X₃, and C13X₂ and N125X₃), where X₁, X₂, and X₃ is each independently any amino acid, or X₁ is a non-polar aliphatic amino acid, selected from G, A, V, L, I and P, X₂ is a positively charged amino acid selected from K, R, and H, and/or X₃ is a positively charged amino acid selected from K, R, and H. In embodiments, X₁ is P, X₂ is R, and/or X₃ is K.

In embodiments, the enzyme (e.g., without limitation, a mobile element enzyme, e.g., without limitation, MLT mobile element enzyme) has gene cleavage activity (Exc+) and/or gene integration activity (Int+). In embodiments, the enzyme (e.g., without limitation, a mobile element enzyme) has gene cleavage activity (Exc+) and/or a lack of gene integration activity (Int−). In embodiments, the MLT mobile element enzyme has gene cleavage activity (Exc+) and/or gene integration activity (Int+). In embodiments, the MLT mobile element enzyme has gene cleavage activity (Exc+) and/or a lack of gene integration activity (Int−).

In embodiments, the mobile element enzyme, e.g., without limitation, MLT mobile element enzyme includes an integration reduced or deficient mutation, e.g., about 1, or about 2, or about 3, or about 4, or about 5 integration reduced or deficient mutations or combinations thereof. In embodiments, the mobile element enzyme can include any number of any of the integration reduced or deficient mutations, or equivalents thereof, described herein.

In embodiments, the MLT mobile element enzyme includes an integration reduced or deficient mutations, e.g., about 1, or about 2, or about 3, or about 4, or about 5 integration reduced or deficient mutations, or combinations thereof. In embodiments, the mobile element enzyme can include any number of any of the integration reduced or deficient mutations, or equivalents thereof, described herein.

In embodiments, the enzyme comprises one or more mutations corresponding to TABLE 2A, or positions corresponding thereto, which, without being bound by theory, provides integration reduced or deficient mutations. Numbering relative to the amino acid sequence of protein of SEQ ID NO: 1, and nucleic acid sequence of SEQ ID NO: 2.

TABLE 2A
Amino Acid Change

Y281A
C282A
G283A
E284A
G285A
N310A
G330A
T331A
I332A
R333A
K334A
N335A
R336A
G337A
I338A
P339A
D416A
K286A
R287A
N310A
K286A/R287A
K286A/N310A
K286A/K369A
R287A/N310A
R287A/K369A
N310A/K369A
R287A/N310A

In embodiments, the enzyme comprises one or more mutations corresponding to TABLE 2B, or positions corresponding thereto, which, without being bound by theory, provides excision positive and integration deficient mutations. Numbering relative to the amino acid sequence of protein of SEQ ID NO: 1, and nucleic acid sequence of SEQ ID NO: 2.

TABLE 2B

| MLT Backbone | MLT Mutant 1 | MLT Mutant 2 | MLT Mutant 3 |
|---|---|---|---|
| S8P/C13R | R164N | 0 | 0 |
| S8P/C13R | W168V | 0 | 0 |
| S8P/C13R | W168V | K369A | 0 |
| S8P/C13R | M278A | 0 | 0 |
| S8P/C13R | K286A | 0 | 0 |
| S8P/C13R | K286A | R287 | |
| S8P/C13R | K286A | N310N | |
| S8P/C13R | K286A | K369A | |
| S8P/C13R | R287A | 0 | 0 |
| S8P/C13R | R287A | N310A | |
| S8P/C13R | R287A | K369A | |
| S8P/C13R | R287A | N310A | K369A |
| S8P/C13R | N310A | K369A | |
| S8P/C13R | R333A | 0 | 0 |
| S8P/C13R | R333A | E284A | 0 |
| S8P/C13R | R333A | E284A | R336A |
| S8P/C13R | K334A | 0 | 0 |
| S8P/C13R | N335A | 0 | 0 |
| S8P/C13R | K349A | 0 | 0 |
| S8P/C13R | K350A | 0 | 0 |
| S8P/C13R | K368A | 0 | 0 |
| S8P/C13R | K369A | 0 | 0 |
| S8P/C13R | D416N | 0 | 0 |
| S8P/C13R | D416N | K286A | 0 |
| S8P/C13R | D416N | R287A | 0 |
| S8P/C13R | D416N | R333A | 0 |
| S8P/C13R | D416N | K334A | 0 |
| S8P/C13R | D416N | R336A | 0 |
| S8P/C13R | D416N | K349A | 0 |
| S8P/C13R | D416N | K350A | 0 |
| S8P/C13R | D416N | K368A | 0 |
| S8P/C13R | D416N | K369A | 0 |
| S8P/C13R | D416N | N310A | 0 |

In embodiments, a MLT mobile element enzyme comprising the amino acid sequence of SEQ ID NO: 1, or a variant thereof, and includes one or more mutations selected from S8P and/or C13R and one of R164N, W168V, M278A, K286A, R287A, R333A, K334A, N335A, K349A, K350A, K368A, K369A, and D416N and/or one or more of E284A, K286A, R287A, N310A, R333A, K334A, R336A, K349A, K350A, K368A, and K369A and/or one R336A.

In embodiments, the mobile element enzyme is or is derived from any of Bombyx mori, Xenopus tropicalis, Trichoplusia ni, Rhinolophus ferrumequinum, Rousettus aegyptiacus, Phyllostomus discolor, Myotis myotis, Myotis lucifugus, Pipistrellus kuhilii, Pteropus vampyrus, and Molossus molossus. In embodiments, the mobile element enzyme is or is derived from any of Trichoplusia ni (SEQ ID NO: 433), Myotis myotis (SEQ ID NO: 435, SEQ ID NO: 436, SEQ ID NO: 438, or SEQ ID NO: 439), or Pteropus vampyrus (SEQ ID NO: 434). In embodiments, the mobile element enzymes have one or more hyperactive and/or integration deficient mutations selected from TABLE 1, TABLE 2A, and/or TABLE 2B, or equivalents thereof. One skilled in the art can correspond such mutants to mobile element enzymes from any of Trichoplusia ni (SEQ ID NO: 433), Myotis lucifugus (SEQ ID NO: 437), Myotis myotis (SEQ ID NO: 435, SEQ ID NO: 436, SEQ ID NO: 438, or SEQ ID NO: 439), or Pteropus vampyrus (SEQ ID NO: 434), e.g.:

```
Trichnoplusia ni
(SEQ ID NO: 433)
    1 MGSSLDDEHI LSALLQSDDE LVGEDSDSEI SDHVSEDDVQ SDTEEAFIDE VHEVQPTSSG
  61 SEILDEQNVI EQPGSSLASN KILTLPQRTI RGKNKHCWST SKSTRRSRVS ALNIVRSQRG
121 PTRMCRNIYD PLLCFKLFFT DEIISEIVKW TNAEISLKRR ESMTGATFRD TNEDEIYAFF
181 GILVMTAVRK DNHMSTDDLF DRSLSMVYVS VMSRDREDFL IRCLRMDDKS IRPTLRENDV
241 FTPVRKIWDL FIHQCIQNYT PGAHLTIDEQ LLGFRGRCPF RMYIPNKPSK YGIKILMMCD
301 SGTKYMINGM PYLGRGTQTN GVPLGEYYVK ELSKPVRGSC RNITCDNWFT SIPLAKNLLQ
361 EPYKLTIVGT VRSNKREIPE VLKNSRSRPV GTSMFCFDGP LTLVSYKPKP AKMVYLLSSC
421 DEDASINEST GKPQMVMYYN QTKGGVDILD QMCSVMTCSR KTNRWPMALL YGMINIACIN
481 SFIIYSHNVS SKGEKVQSRK KFMRNLYMSL TSSFMRKRLE APTLKRYLRD NISNILPNEV
541 PGTSDDSTEE PVTKKRTYCT YCPSKIRRKA NASCKKCKKV ICREHNIDMC QSCF
Pteropus vampyrus
(SEQ ID NO: 434)
    1 MSNPRKRSIP TCDVNFVLEQ LLAEDSFDES DFSEIDDSDD FSDSASEDYT VRPPSDSESD
  61 GNSPTSADSG RALKWSTRVM IPRQRYDFTG TPGRKVDVSD TTDPLQYFEL FFTEELVSKI
121 TSEMNAQAAL LASKPPGPKG FSRMDKWKDT DNDELKVFFA VMLLQGIVQK PELEMFWSTR
181 PLLDIPYLRQ IMTGERFLLL LRCLHFVNNS SISAGQSKAQ ISLQKIKPVF DFLVNKFSTV
241 YTPNRNIAVD ESLMLFKGRL AMKQYIPTKM NLKDSADGLK
Myotis myotis (“2a”)
(SEQ ID NO: 435)
    1 MDLRCQHTVL SIRESRGLLP NLKMKTSRMK KGDIIFSRKG DILLLAWKDK RVVRMISIHD
  61 TSVSTTGKKN RKTGENIVKP ACIKEYNAHM KGVDRADQFL SCCSILRKMM KWTKKVVLYL
121 INCGLENSFR VYNVLNPQAK MKYKQFLLSV ARDWIMDDNN EGSPEPETNL SSPSPGGARR
181 APRKDPPKRL SGDMKQHEPT CIPASGKKKF PTRACRVCAH GKRSESRYLC KFCLVPLHRG
241 KCFTQYHTLK KY
Myotis myotis (“1”)
(SEQ ID NO: 436)
    1 MKAFLGVILN MGVLNHPNLQ SYWSMDFESH IPFFRSVFKR ERFLQIFWML HLKNDQKSSK
  61 DLRTRTEKVN CFLSYLEMKF RERFCPGREI AVDEAVVGFK GKIHFITYNP KKPTKWGIRL
121 YVLSDSKCGY VHSFVPYYGG ITSETLVRPD LPFTSRIVLE LHERLKNSVP GSQGYHFFTD
181 RYYTSVTLAK ELFKEKTHLT GTIMPNRKDN PPVIKHQKLK KGEIVAFRDE NVMLLAWKDK
241 RIVTLSTWDS ETESVERRVG GGKEIVLKPK VVTNYTKFMG GVDIADYTST YCFMRKTLKW
301 WRTLFFWGLE VSVVNSYILY KECQKRKNEK PITHVKFIRK LVHDLVGEFR DGTLTSRGRL
361 LSTNLEQRLD GKLHIITPHP NKKHKDCVVC SNRKIKGGRR ETIYICETCE CKPGLHVGEC
421 FKKYHTMKNY RD
Myotis lucifugus (“2”)
(SEQ ID NO: 437)
    1 MPSLRKRKET NETDTLPEVF NDNLSDIPSE IEDADDCFDD SGDDSTDSTD SEIIRPVRKR
  61 KVAVLSSDSD TDEATDNCWS EIDTPPRLQM FEGHAGVTTF PSQCDSVPSV TNLFFGDELF
121 EMLCKELSNY HDQTAMKRKT PSRTLKWSPV TQKDIKKFLG LIILMGQTRK DSLKDYWSTD
181 PLICTPIFPQ TMSRHRFEQI WTFWHENDNA KMDSRSGRLF KIQPVLDYFL HKFRTIYKPK
241 QQLSLDEGMI PWRGRFKFRT YNPAKITKYG LLVRMVCESD TGYICSMEIY TAEGRKLQET
301 VLSVLGPYLG IWHHIYQDNY YNATSTAELL LQNKTRVCGT IRESRGLPPN LEMKTSRMKK
361 GDIIFSRKGD ILLLAWKDKR VVRMISTIHD TSVSTTGKKN RKTGENIVKP TCIKEYNAHM
421 KGVDRADQFL SCCSILRKTM KWIKKVVLYL INCGLENSER VYNVLNPQAK MKYKQFLLSV
481 ARDWITDDNN EGSPEPETNL SSPSPGGARR APRKDPPKRL SGDMKQHEPT CIPASGKKKE
541 PTRACRVCAA HGKRSESRYL CKFCLVPLHR GKCFTQYHTL KKYMDLRCQH TVLSTVGRGY
601 SVLARFKPRT NERTGSSHCH VQVPAGGQGP PSTIIANGCG CKLEPMVRTR SPTCLVIEFG
661 CM
Myotis myotis (“2”)
(SEQ ID NO: 438)
    1 MPSLRKRKET NETDTLPEVF NDNLSDIPSE IEDADDCEDD SGDDSIDSTE SEIIRPVRKR
  61 KVAVLSSDSN TDEATDNCWS EIDTPPRLQM FEGHAGVTTF PSQCDSVPSV TNLFFGDELF
121 EMLCKELSNY HDQTAMKRKT PSRTLKWSPV TQKDIKKELG LIILMGQTRK DSWKDYWSTD
181 PLICTPIFPQ TMSRHRFEQI WTFWHENDNA KMDSCSGRLF KIQPVLDYFL HKFRTIYKPK
241 QQLSLDEGMI PWRGRLKFTY NPAITKYGLL VRMVCESDTG YICNMEIYTA ERKKLQETVL
301 SVLGPYLGIW HHIYQDNYYN ATSTAELLLQ NKTRVCGTIR ESRGLPPNLK MKTSRMKKGD
361 IIFSRKGDIL LLAWKDKRVV RMISTIHDTS VSTTGKKNRK TGENIVKPTC IKEYNAHMKG
421 VDRADQFLSC CSILRKTTKW TKKVVLYLIN CGLENSFRVY NILNPQAKMK YKQFLLSVAR
481 DWITDDNNEG SPEPETNLSS PSSGGARRAP RKDQPKRLSG DMKQHEPTCI PASGKKKFPT
541 ACRVCAAHGK RSESRYLRKF CFVPLRGKCF MYHTLKKYSE LFSLIVVSKI QNVIIYKTTK
601 VYMRYVMRSH CPLSELVFAP SVKDRSRVFS FFTRHLLWTL DVNTLSCPHR MKRSHWWKPC
661 RSIYEKLYNC TNP
Myotis myotis (“2b”)
(SEQ ID NO: 439)
    1 MDLRCQHTVL SIRESRGLPP NLKMKTSRMK KGDIIFSRKG DILLLAWKDK RVVRMISTIH
  61 DTSVSTTGKK NRKTGENIVK PACIKEYNAH MKGVDRADQF LSCCSILRKT MKWTKKVVLY
121 LINCGLENSF RVYNVLNPQA KMKYKQFLLS VARDWITDDN NEGSPEPETN LSSPSPGGAR
181 RAPRKDPPKR LSGDMKQHEP TCIPASGKKK FPTRACRVCA AHGKRSESRY LCKFCLVPLH
241 RGKCFTQYHT LKKY
```

In embodiments, the mobile element enzyme is derived from Bombyx mori, Xenopus tropicalis, or Trichoplusia ni. In embodiments, the mobile element enzyme is an engineered version of a mobile element enzyme, including but not limited to monomers, dimers, tetramers, hyperactive, or Int-forms, derived from Bombyx mori, Xenopus tropicalis, or Trichoplusia ni.

In embodiments, the mobile element enzyme is derived from Bombyx mori, Xenopus tropicalis, Trichoplusia ni, or Myotis lucifugus. In embodiments, the mobile element enzyme is an engineered version, including but not limited to a mobile element enzyme that is a monomer, dimer, tetramer (or another multimer), hyperactive, or has a reduced interaction with non-TTAA (SEQ ID NO: 440) recognitions sites (Int−), derived from Bombyx mori, Xenopus tropicalis, Trichoplusia ni, or Myotis lucifugus. In embodiments, the mobile element enzymes have one or more hyperactive and/or integration deficient mutations selected from TABLE 1, TABLE 2A, and TABLE 2B, or equivalents thereof.

In embodiments, one skilled in the art can correspond such mutants to mobile element enzymes from any of Bombyx mori, Xenopus tropicalis, Trichoplusia ni, Rhinolophus ferrumequinum, Rousettus aegyptiacus, Phyllostomus discolor, Myotis myotis, Myotis lucifugus, Pipistrellus kuhlii, Pteropus vampyrus, Pan troglodytes, and Molossus molossus.

In embodiments, the mobile element enzyme has a nucleotide sequence having at least about 90%, or at least about 93%, or at least about 95%, or at least about 97%, or at least about 98%, or at least about 99% identity to a nucleotide sequence of any of Rhinolophus ferrumequinum, Rousettus aegyptiacus, Phyllostomus discolor, Myotis myotis, Myotis lucifugus, Pteropus vampyrus, Pipistrellus kuhliim, Pan troglodytes, and Molossus molossus. In embodiments, the mobile element enzyme has an amino acid sequence having at least about 90%, or at least about 93%, or at least about 95%, or at least about 97%, or at least about 98%, or at least about 99% identity to an amino acid sequence of any of Rhinolophus ferrumequinum, Rousettus aegyptiacus, Phyllostomus discolor, Myotis myotis, Myotis lucifugus, Pteropus vampyrus, Pipistrellus kuhlii, and Molossus molossus. See Jebb, et al. (2020).

In embodiments, the enzyme (e.g., without limitation, a mobile element enzyme) is derived from Bombyx mori, Xenopus tropicalis, Trichoplusia ni, Myotis lucifugus, Rhinolophus ferrumequinum, Rousettus aegyptiacus, Phyllostomus discolor, Myotis myotis, Pteropus vampyrus, Pipistrellus kuhlii, Molossus molossus, Pan troglodytes, or Homo sapiens.

In embodiments, the enzyme (e.g., without limitation, a mobile element enzyme) is an engineered version, including but not limited to hyperactive forms, of a mobile element enzyme derived from Bombyx mori, Xenopus tropicalis, Trichoplusia ni, Myotis lucifugus, Rhinolophus ferrumequinum, Rousettus aegyptiacus, Phyllostomus discolor, Myotis myotis, Pteropus vampyrus, Pipistrellus kuhlii, Molossus molossus, Pan troglodytes, or Homo sapiens. The enzyme is either the wild type, monomer, dimer, tetramer, hyperactive, or an Int-mutant. In embodiments, the mobile element enzymes have one or more hyperactive and/or integration deficient mutations selected from TABLE 1, TABLE 2A, and/or TABLE 2B, or equivalents thereof.

In embodiments, the mobile element enzyme has a nucleotide sequence having at least about 90%, or at least about 93%, or at least about 95%, or at least about 97%, or at least about 98%, or at least about 99% identity to a nucleotide sequence of any of Rhinolophus ferrumequinum, Rousettus aegyptiacus, Phyllostomus discolor, Myotis myotis, Pteropus vampyrus, Pipistrellus kuhlii, Molossus molossus, and Pan troglodytes. In embodiments, the mobile element enzyme has an amino acid sequence having at least about 90%, or at least about 93%, or at least about 95%, or at least about 97%, or at least about 98%, or at least about 99% identity to an amino acid sequence of any of Rhinolophus ferrumequinum, Rousettus aegyptiacus, Phyllostomus discolor, Myotis myotis, Pteropus vampyrus, Pipistrellus kuhlii, Molossus molossus, Pan troglodytes, and Homo sapiens.

In embodiments, the mobile element enzyme is an engineered version, including but not limited to a mobile element enzyme that is a monomer, dimer, tetramer, hyperactive, or has a reduced interaction with non-TTAA (SEQ ID NO: 440) recognitions sites (Int−), derived from any of Bombyx mori, Xenopus tropicalis, Trichoplusia ni, Rhinolophus ferrumequinum, Rousettus aegyptiacus, Phyllostomus discolor, Myotis myotis, Myotis lucifugus, Pipistrellus kuhlii, Pteropus vampyrus, and Molossus molossus Bombyx mori, Xenopus tropicalis, Trichoplusia ni, Pan troglodytes, Myotis lucifugus, and Homo sapiens. The mobile element enzyme is either the wild type, monomer, dimer, tetramer or another multimer, hyperactive, or a an Int-mutant.

In embodiments, the mobile element enzyme is from a Tc1/mariner donor DNA system. See, e.g., Plasterk et al. Trends in Genetics. 1999; 15(8):326-32.

In embodiments, the mobile element enzyme is from a Sleeping Beauty donor DNA system (see, e.g., Cell. 1997; 91:501-510), e.g., a hyperactive form of Sleeping Beauty (hypSB), e.g., SB100X (see Gene Therapy volume 18, pages 849-856(2011), or a piggyBac (PB) donor DNA system (see, e.g., Trends Biotechnol. 2015 September; 33(9):525-33, which is incorporated herein by reference in its entirety), e.g., a hyperactive form of PB mobile element enzyme (hypPB), e.g., with seven amino acid substitutions (e.g., I30V, S103P, G165S, M282V, S509G, N570S, N538K on mPB, or functional equivalents in non-mPB, see Mol Ther Nucleic Acids. 2012 October; 1(10): e50, which is incorporated herein by reference in its entirety); see also Yusa et al., PNAS Jan. 25, 2011 108 (4) 1531-1536; Voigt et al., Nature Communications volume 7, Article number: 11126 (2016).

The piggyBac mobile element enzymes belong to the IS4 mobile element enzyme family. De Palmenaer et al., BMC Evolutionary Biology. 2008; 8:18. doi: 10.1186/1471-2148-8-18. The piggyBac family includes a large diversity of donor DNAs, and any of these donor DNAs can be used in embodiments of the present disclosure. See, e.g., Bouallègue et al., Genome Biol Evol. 2017; 9(2):323-339. The founding member of the piggyBac (super)family, insect piggyBac, was originally identified in the cabbage looper moth (Trichoplusiani ni) and studied both in vivo and in vitro. Insect piggyBac is known to transpose by a canonical cut-and-paste mechanism promoted by an element-encoded mobile element enzyme with a catalytic site resembling the RNase H fold shared by many recombinases. The insect piggyBac donor DNA system has been shown to be highly active in a wide range of animals, including Drosophila and mice, where it has been developed as a powerful tool for gene tagging and genome engineering. Other donor DNAs affiliated to the piggyBac superfamily are common in arthropods and vertebrates including Xenopus and Bombyx. Mammalian piggyBac donor DNAs and mobile element enzymes, including hyperactive mammalian piggyBac variants, which can be used in embodiments of the present disclosure, are described, e.g., in International Application WO2010085699, which is incorporated herein by reference in its entirety.

In embodiments, the mobile element enzyme is from a LEAP-IN 1 type or LEAP-IN donor DNA system (Biotechnol J. 2018 October; 13(10):e1700748. doi: 10.1002/biot.201700748. Epub 2018 Jun. 11). The LEAPIN mobile element enzyme system includes a mobile element enzyme (e.g., without limitation, a mobile element enzyme mRNA) and a vector containing one or more genes of interest (donor DNAs), selection markers, regulatory elements, insulators, etc., flanked by the donor DNA cognate inverted terminal ends and the transposition recognition motif (TTAT). Upon co-transfection of vector DNA and mobile element enzyme mRNA, the transiently expressed enzyme catalyzes high-efficiency and precise integration of a single copy of the donor DNA cassette (all sequences between the terminal ends) at one or more sites across the genome of the host cell. Hottentot et al. In Genotyping: Methods and Protocols. White S J, Cantsilieris S, eds: 185-196. (New York, NY: Springer): 2017. pp. 185-196. The LEAPIN mobile element enzyme generates stable transgene integrants with various advantageous characteristics, including single copy integrations at multiple genomic loci, primarily in open chromatin segments; no payload limit, so multiple independent transcriptional units may be expressed from a single construct; the integrated transgenes maintain their structural and functional integrity; and maintenance of transgene integrity ensures the desired chain ratio in every recombinant cell.

In embodiments, the mobile element enzyme is an engineered form of a mobile element enzyme reconstructed from Homo sapiens or a predecessor thereof.

Donor DNAs in Humans have 5 inactive elements, designated PiggyBac domain (PGBD)1, PGBD2, PGBD3, PGBD4, and PGBD5. PGBD1, PGBD2, and PGBD3 have multiple coding exons, but in each case the mobile element enzyme-related sequence is encoded by a single uninterrupted 3′ terminal exon. Thus, PGBD1 and PGBD2 may resemble the PGBD3 donor DNA in which the mobile element enzyme ORF is flanked upstream by a 3′ splice site and downstream by a polyadenylation site. See Newman et al., PLoS Genet 2008; 4:e1000031. PLoS Genet 4(3): e1000031. https://doi.org/10.1371/journal.pgen.1000031; Gray et al., PLoS Genet 8(9): e1002972. https://doi.org/10.1371/journal.pgen.1002972.

The PGBD5 inactive mobile element enzyme sequence belongs to the RNase H clan of Pfam structures, while PGBD3 has sustained only a single D to N mutation in the essential catalytic triad DDD(D) and retains the ability to bind the upstream piggyBac terminal inverted repeat. Bailey et al., DNA Repair (Amst) 2012; 11:488-501. The PGBD5 mobile element enzyme does not retain the catalytic DDD (D) motif found in active elements, and the mobile element enzyme is not only inactive but fails to associate with either DNA or chromatin in vivo. Pavelitz et al., Mob DNA 2013; 4:23. However, in vitro studies showed that it is transpositionally active in HEK293 cells. See Henssen et al., Elife 2015; 4. PGBD1 and PGBD2 are thought to be present in the common ancestor of mammals, while PGBD3 and PGBD4 are restricted to primates. See Sarkar et al., Mol Genet Genomics 2003; 270:173-80. The Pteropus vampyrus mobile element enzyme is closely related to PGBD4 and shares DDD catalytic domain and the C-terminal region that are involved in excision mechanisms. See Mitra et al., EMBO J 2008; 27:1097-109.

A mammalian mobile element enzyme, which has gene cleavage and/or gene integration activity, can be constructed based on alignment of the amino acid sequence of Pteropus vampyrus mobile element enzyme to PGBD1, PGBD2, PGBD3, PGBD4, and PGBD5 sequences. Also, in embodiments, the mammalian mobile element enzyme has mutations that confers hyperactivity to a recombinant mammalian mobile element enzyme. Accordingly, in embodiments, the mobile element enzyme has gene cleavage activity (Exc+) and/or gene integration activity (Int+). In embodiments, the mobile element enzyme has gene cleavage activity (Exc+) and/or lacks gene integration activity (Int−).

In some aspects, an enzyme capable of performing targeted genomic integration is a recombinant mammalian mobile element enzyme that was derived by, in part, aligning several inactive mobile element enzyme sequences from a human genome to Pteropus vampyrus mobile element enzyme sequence. In embodiments, the Pteropus vampyrus mobile element enzyme has an amino acid sequence having at least 90% identity (e.g., a sequence having at least about 90%, or at least about 93%, or at least about 95%, or at least about 97%, or at least about 98%, or at least about 99% identity) to SEQ ID NO: 430 (or a functional equivalent thereof. In embodiments, the Pteropus vampyrus mobile element enzyme has an amino acid sequence of SEQ ID NO: 430, or a functional equivalent thereof. In embodiments, the Pteropus vampyrus mobile element enzyme has a nucleotide sequence having at least 90% identity (e.g., a sequence having at least about 90%, or at least about 93%, or at least about 95%, or at least about 97%, or at least about 98%, or at least about 99% identity) to SEQ ID NO: 429 or a codon-optimized variant thereof.

In embodiments, the mobile element enzyme is a mammalian mobile element enzyme, such as a mobile element enzyme from a bat, e.g., without limitation, Pteropus vampyrus.

In embodiments, the mobile element enzyme is an engineered form that is based on a mobile element enzyme reconstructed from Homo sapiens or a predecessor thereof. In embodiments, the mobile element enzyme includes but is not limited to an engineered version that is a monomer, dimer, tetramer (or another multimer), hyperactive, or has a reduced interaction with non-TTAA (SEQ ID NO: 440) recognitions sites (Int−), of an engineered version of a mobile element enzyme reconstructed from Homo sapiens or a predecessor thereof.

In embodiments, the mobile element enzyme is an engineered form that is based on a mobile element enzyme reconstructed from mammalian species. In embodiments, the mobile element enzyme includes but is not limited to an engineered that is a monomer, dimer, tetramer (or another multimer), hyperactive, or has a reduced interaction with non-TTAA (SEQ ID NO: 440) recognitions sites (Int−), of a mobile element enzyme reconstructed from mammalian species.

In embodiments, the donor DNA is included in a vector comprising left and right end sequences recognized by the enzyme capable of performing targeted genomic integration, e.g., without limitation, a mobile element enzyme.

In embodiments, the end sequences are selected from MER, MER75A, MER75B, and MER85.

In embodiments, the end sequences are selected from nucleotide sequences of SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 441, and SEQ ID NO: 22, or a nucleotide sequence having at least about 90% identity (e.g., a sequence having at least about 90%, or at least about 93%, or at least about 95%, or at least about 97%, or at least about 98%, or at least about 99% identity) thereto. In embodiments, one or more of the end sequences are optionally flanked by a TTAA (SEQ ID NO: 440) sequence.

In embodiments, the end sequences include at least one repeat from a nucleotide sequence having at least about 90% (e.g., a sequence having at least about 90%, or at least about 93%, or at least about 95%, or at least about 97%, or at least about 98%, or at least about 99% identity) identity to the nucleotide sequence of SEQ ID NO: 12, and wherein the at least one repeat from the nucleotide sequence having at least about 90% identity (e.g., a sequence having at least about 90%, or at least about 93%, or at least about 95%, or at least about 97%, or at least about 98%, or at least about 99% identity) to the nucleotide sequence of SEQ ID NO: 12 is positioned at the 5′ end of the donor DNA. The end sequences can further include at least one repeat from a nucleotide sequence having at least about 90% identity (e.g., a sequence having at least about 90%, or at least about 93%, or at least about 95%, or at least about 97%, or at least about 98%, or at least about 99% identity to the nucleotide sequence of SEQ ID NO: 17, and wherein the at least one repeat from the nucleotide sequence having at least about 90% identity (e.g., a sequence having at least about 90%, or at least about 93%, or at least about 95%, or at least about 97%, or at least about 98%, or at least about 99% identity) to the nucleotide sequence of SEQ ID NO: 17 is positioned at the 3′ end of the donor DNA. The end sequences, which can be from, e.g., Pteropus vampyrus, are optionally flanked by a TTAA (SEQ ID NO: 440) sequence.

In embodiments, the end sequences include at least one repeat from a nucleotide sequence having at least about 90% identity (e.g., a sequence having at least about 90%, or at least about 93%, or at least about 95%, or at least about 97%, or at least about 98%, or at least about 99% identity) to the nucleotide sequence of SEQ ID NO: 13, and wherein the at least one repeat from the nucleotide sequence having at least about 90% identity (e.g., a sequence having at least about 90%, or at least about 93%, or at least about 95%, or at least about 97%, or at least about 98%, or at least about 99% identity) to the nucleotide sequence of SEQ ID NO: 13 is positioned at the 5′ end of the donor DNA. The end sequences can further include at least one repeat from a nucleotide sequence having at least about 90% identity (e.g., a sequence having at least about 90%, or at least about 93%, or at least about 95%, or at least about 97%, or at least about 98%, or at least about 99% identity) to the nucleotide sequence of SEQ ID NO: 18, and wherein the at least one repeat from the nucleotide sequence having at least about 90% identity (e.g., a sequence having at least about 90%, or at least about 93%, or at least about 95%, or at least about 97%, or at least about 98%, or at least about 99% identity) to the nucleotide sequence of SEQ ID NO: 18 is positioned at the 3′ end of the donor DNA. The end sequences, which can be, e.g., PGBD4, are optionally flanked by a TTAA (SEQ ID NO: 440) sequence.

In embodiments, the end sequences include at least one repeat from a nucleotide sequence having at least about 90% identity (e.g., a sequence having at least about 90%, or at least about 93%, or at least about 95%, or at least about 97%, or at least about 98%, or at least about 99% identity) to the nucleotide sequence of SEQ ID NO: 14, wherein the at least one repeat from the nucleotide sequence having at least about 90% identity (e.g., a sequence having at least about 90%, or at least about 93%, or at least about 95%, or at least about 97%, or at least about 98%, or at least about 99% identity) to the nucleotide sequence of SEQ ID NO: 14 is positioned at the 5′ end of the donor DNA. The end sequences include at least one repeat from a nucleotide sequence having at least about 90% identity (e.g., a sequence having at least about 90%, or at least about 93%, or at least about 95%, or at least about 97%, or at least about 98%, or at least about 99% identity) to the nucleotide sequence of SEQ ID NO: 18, wherein the at least one repeat from the nucleotide sequence having at least about 90% identity (e.g., a sequence having at least about 90%, or at least about 93%, or at least about 95%, or at least about 97%, or at least about 98%, or at least about 99% identity) to the nucleotide sequence of SEQ ID NO: 19 is positioned at the 3′ end of the donor DNA. The end sequences, which can be, e.g., MER75, are optionally flanked by a TTAA (SEQ ID NO: 440) sequence.

In embodiments, the end sequences include at least one repeat from a nucleotide sequence having at least about 90% identity (e.g., a sequence having at least about 90%, or at least about 93%, or at least about 95%, or at least about 97%, or at least about 98%, or at least about 99% identity) to the nucleotide sequence of SEQ ID NO: 15, wherein the at least one repeat from the nucleotide sequence having at least about 90% identity (e.g., a sequence having at least about 90%, or at least about 93%, or at least about 95%, or at least about 97%, or at least about 98%, or at least about 99% identity) to the nucleotide sequence of SEQ ID NO: 15 is positioned at the 5′ end of the donor DNA. The end sequences include at least one repeat from a nucleotide sequence having at least about 90% identity (e.g., a sequence having at least about 90%, or at least about 93%, or at least about 95%, or at least about 97%, or at least about 98%, or at least about 99% identity) to the nucleotide sequence of SEQ ID NO: 20, wherein the at least one repeat from the nucleotide sequence having at least about 90% identity (e.g., a sequence having at least about 90%, or at least about 93%, or at least about 95%, or at least about 97%, or at least about 98%, or at least about 99% identity) to the nucleotide sequence of SEQ ID NO: 20 is positioned at the 3′ end of the donor DNA. The end sequences, which can be, e.g., MER75B, are optionally flanked by a TTAA (SEQ ID NO: 440) sequence.

In embodiments, the end sequences include at least one repeat from a nucleotide sequence having at least about 90% (e.g., a sequence having at least about 90%, or at least about 93%, or at least about 95%, or at least about 97%, or at least about 98%, or at least about 99% identity) identity to the nucleotide sequence of SEQ ID NO: 16, wherein the at least one repeat from the nucleotide sequence having at least about 90% identity (e.g., a sequence having at least about 90%, or at least about 93%, or at least about 95%, or at least about 97%, or at least about 98%, or at least about 99% identity) to the nucleotide sequence of SEQ ID NO: 16 is positioned at the 5′ end of the donor DNA. The end sequences include at least one repeat from a nucleotide sequence having at least about 90% identity (e.g., a sequence having at least about 90%, or at least about 93%, or at least about 95%, or at least about 97%, or at least about 98%, or at least about 99% identity) to the nucleotide sequence of SEQ ID NO: 21 or SEQ ID NO: 441, wherein the at least one repeat from the nucleotide sequence having at least about 90% identity (e.g., a sequence having at least about 90%, or at least about 93%, or at least about 95%, or at least about 97%, or at least about 98%, or at least about 99% identity) to the nucleotide sequence of SEQ ID NO: 21 or SEQ ID NO: 441 is positioned at the 3′ end of the donor DNA. The end sequences, which can be, e.g., MER75A, are optionally flanked by a TTAA (SEQ ID NO: 440) sequence.

In embodiments, a donor DNA is or comprises a vector comprising a donor DNA comprising one or more end sequences recognized by an enzyme such as, for example a mobile element enzyme. In embodiments, the end sequences are selected from Pteropus vampyrus, MER75, MER75A, and MER75B. MERs contain end sequences with similarity to piggyBac-like mobile elements and exhibit duplications of their presumed TTAA (SEQ ID NO: 440) target sites. In embodiments, the end sequences are selected from nucleotide sequences of SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 441, and SEQ ID NO: 22, or a nucleotide sequence having at least about 90% identity (e.g., a sequence having at least about 90%, or at least about 93%, or at least about 95%, or at least about 97%, or at least about 98%, or at least about 99% identity) thereto.

In embodiments, the mobile element enzyme has an amino acid sequence of SEQ ID NO: 3, SEQ ID NO: 4, SEQ ID NO: 6, SEQ ID NO: 7, SEQ ID NO: 8, or SEQ ID NO: 9, or a variant sequence having at least about 90%, or at least about 93%, or at least about 95%, or at least about 97%, or at least about 98%, or at least about 99% identity thereto, or at least about 10 mutations, or at least about 9 mutations, or at least about 8 mutations, or at least about 7 mutations, or at least about 6 mutations, or at least about 5 mutations, or at least about 4 mutations, or at least about 3 mutations, or at least about 2 mutations, or at least about 1 mutation.

In embodiments, the mobile element enzyme has an amino acid sequence having S8P, G17R, and/or K134K mutation relative to the amino acid sequence of SEQ ID NO: 4 or a functional equivalent thereof.

In embodiments, the mobile element enzyme has an amino acid sequence having S8P, G17R, and/or K134K mutation relative to the amino acid sequence of SEQ ID NO: 5 or a functional equivalent thereof.

In embodiments, the mobile element enzyme has an amino acid sequence having 183P and/or V118R mutation relative to the amino acid sequence of SEQ ID NO: 6 or a functional equivalent thereof.

In embodiments, the mobile element enzyme has an amino acid sequence having S20P and/or A29R mutation relative to the amino acid sequence of SEQ ID NO: 7 or a functional equivalent thereof.

In embodiments, the mobile element enzyme has an amino acid sequence having T4P and/or L13R mutation relative to the amino acid sequence of SEQ ID NO: 8 or a functional equivalent thereof.

In embodiments, the mobile element enzyme has an amino acid sequence having A12P and/or 128R mutation and/or R152K mutation relative to the amino acid sequence of SEQ ID NO: 9 or a functional equivalent thereof.

In embodiments, the enzyme capable of performing targeted genomic integration (e.g., without limitations, a mobile element enzyme) is in a monomeric or dimeric form. In embodiments, the enzyme capable of performing targeted genomic integration (e.g., without limitations, a mobile element enzyme) is in a multimeric form.

In embodiments, the enzyme (e.g., without limitation, a mobile element enzyme) is an engineered version, including but not limited to a mobile element enzyme that is a monomer, dimer, tetramer, hyperactive, or has a reduced interaction with non-TTAA (SEQ ID NO: 440) recognitions sites (Int−), and is derived from any of Bombyx mori, Xenopus tropicalis, Trichoplusia ni, Myotis lucifugus, Rhinolophus ferrumequinum, Rousettus aegyptiacus, Phyllostomus discolor, Myotis myotis, Pteropus vampyrus, Pipistrellus kuhlii, Pan troglodytes, Molossus molossus, or Homo sapiens.

In embodiments, the mobile element enzyme is either the wild type, monomer, dimer, tetramer or another multimer, hyperactive, or an Int-mutant.

### Targeting Chimeric Constructs

In aspects, the present disclosure provides for targeted chimeras, e.g., in embodiments, the enzyme, without limitation, a mobile element enzyme, comprises a targeting element.

in embodiments, the enzyme, without limitation, a mobile element enzyme, associated with the targeting element, is capable of inserting the donor DNA comprising a chimeric CAR or chimeric CAAR, optionally at a TA dinucleotide site or a TTAA (SEQ ID NO: 440) tetranucleotide site in a genomic safe harbor site (GSHS).

In embodiments, the enzyme, without limitation, a mobile element enzyme, associated with the targeting element has one or more mutations which confer hyperactivity.

In embodiments, the enzyme, without limitation, a mobile element enzyme, associated with the targeting element has gene cleavage activity (Exc+) and/or gene integration activity (Int+).

In embodiments, the enzyme, without limitation, a mobile element enzyme, associated with the targeting element has gene cleavage activity (Exc+) and/or reduced integration activity (Int−).

In embodiments, the targeting element comprises one or more proteins or nucleic acids that are capable of binding to a nucleic acid.

In embodiments, the targeting element comprises one or more of a of a gRNA, optionally associated with a Cas enzyme, which is optionally catalytically inactive, transcription activator-like effector (TALE), Zinc finger, catalytically inactive transcription factor, nickase, a transcriptional activator, a transcriptional repressor, a recombinase, a DNA methyltransferase, a histone methyltransferase, paternally expressed gene 10 (PEG10), and TnsD.

In embodiments, the targeting element comprises a transcription activator-like effector (TALE) DNA binding domain (DBD).

In embodiments, the TALE DBD comprises one or more repeat sequences. In embodiments, the TALE DBD comprises about 14, or about 15, or about, 16, or about 17, or about 18, or about 18.5 repeat sequences. In embodiments, the TALE DBD repeat sequences comprise 33 or 34 amino acids. In embodiments, the TALE DBD repeat sequences comprise a repeat variable di-residue (RVD) at residue 12 or 13 of the 33 or 34 amino acids. In embodiments, the RVD recognizes one base pair in the nucleic acid molecule. In embodiments, the RVD recognizes a C residue in the nucleic acid molecule and is selected from HD, N(gap), HA, ND, and HI. In embodiments, the RVD recognizes a G residue in the nucleic acid molecule and is selected from NN, NH, NK, HN, and NA. In embodiments, the RVD recognizes an A residue in the nucleic acid molecule and is selected from NI and NS. In embodiments, the RVD recognizes a T residue in the nucleic acid molecule and is selected from NG, HG, H(gap), and IG. In embodiments, the GSHS is in an open chromatin location in a chromosome. In embodiments, the GSHS is selected from adeno-associated virus site 1 (AAVS1), chemokine (C—C motif) receptor 5 (CCR5) gene, HIV-1 coreceptor, and human Rosa26 locus. In embodiments, the GSHS is located on human chromosome 2, 4, 6, 10, 11, 17, 22, or X. In embodiments, the GSHS is selected from TALC1, TALC2, TALC3, TALC4, TALC5, TALC7, TALC8, AVS1, AVS2, AVS3, ROSA1, ROSA2, TALER1, TALER2, TALER3, TALER4, TALER5, SHCHR2-1, SHCHR2-2, SHCHR2-3, SHCHR2-4, SHCHR4-1, SHCHR4-2, SHCHR4-3, SHCHR6-1, SHCHR6-2, SHCHR6-3, SHCHR6-4, SHCHR10-1, SHCHR10-2, SHCHR10-3, SHCHR10-4, SHCHR10-5, SHCHR11-1, SHCHR11-2, SHCHR11-3, SHCHR17-1, SHCHR17-2, SHCHR17-3, and SHCHR17-4.

In embodiments, the targeting element comprises a Cas9 enzyme guide RNA complex. In embodiments, the Cas9 enzyme guide RNA complex comprises a nuclease-deficient dCas9 guide RNA complex. In embodiments, the targeting element comprises a Cas12 enzyme guide RNA complex. In embodiments, the targeting element comprises a nuclease-deficient dCas12 guide RNA complex, optionally dCas12j guide RNA complex or dCas12a guide RNA complex. In embodiments, the targeting element comprises a Cas12k enzyme guide RNA complex. In embodiments, the targeting element comprises a nuclease-deficient dCas12 guide RNA complex, optionally dCas12k guide RNA complex.

In embodiments, a targeting chimeric system or construct, having a DBD fused to a mobile element enzyme, directs binding of an enzyme capable of performing targeted genomic integration (e.g., without limitation, a mobile element enzyme) to a specific sequence (e.g., transcription activator-like effector proteins (TALE) repeat variable di-residues (RVD) or gRNA) near an enzyme recognition site. The enzyme is thus prevented from binding to random recognition sites. In embodiments, the targeting chimeric construct binds to human GSHS. In embodiments, dCas9 (i.e., deficient for nuclease activity) is programmed with gRNAs directed to bind at a desired sequence of DNA in GSHS.

In embodiments, TALEs described herein can physically sequester the enzyme such as, e.g., a mobile element enzyme, to GSHS and promote transposition to nearby TTAA (SEQ ID NO: 440) sequences in close proximity to the RVD TALE nucleotide sequences. GSHS in open chromatin sites are specifically targeted based on the predilection for mobile element enzymes to insert into open chromatin.

In embodiments, an enzyme capable of performing targeted genomic integration (e.g., without limitation, a recombinase, integrase, or a mobile element enzyme such as, without limitation, a mammalian mobile element enzyme) is linked to or fused with a TALE DNA binding domain (DBD) or a Cas-based gene-editing system, such as, e.g., Cas9 or a variant thereof.

In embodiments, the targeting element targets the enzyme to a locus of interest. In embodiments, the targeting element comprises CRISPR (Clustered Regularly Interspaced Short Palindromic Repeat) associated protein 9 (Cas9), or a variant thereof. A CRISPR/Cas9 tool only requires Cas9 nuclease for DNA cleavage and a single-guide RNA (sgRNA) for target specificity. See Jinek et al. (2012) Science 337, 816-821; Chylinski et al. (2014) Nucleic Acids Res 42, 6091-6105. The inactivated form of Cas9, which is a nuclease-deficient (or inactive, or “catalytically dead” Cas9, is typically denoted as “dCas9,” has no substantial nuclease activity. Qi, L. S. et al. (2013). Cell 152, 1173-1183. CRISPR/dCas9 binds precisely to specific genomic sequences through targeting of guide RNA (gRNA) sequences. See Dominguez et al., Nat Rev Mol Cell Biol. 2016; 17:5-15; Wang et al., Annu Rev Biochem. 2016; 85:227-64. dCas9 is utilized to edit gene expression when applied to the transcription binding site of a desired site and/or locus in a genome. When the dCas9 protein is coupled to guide RNA (gRNA) to create dCas9 guide RNA complex, dCas9 prevents the proliferation of repeating codons and DNA sequences that might be harmful to an organism's genome. Essentially, when multiple repeat codons are produced, it elicits a response, or recruits an abundance of dCas9 to combat the overproduction of those codons and results in the shut-down of transcription. Thus, dCas9 works synergistically with gRNA and directly affects the DNA polymerase II from continuing transcription.

In embodiments, the targeting element comprises a nuclease-deficient Cas enzyme guide RNA complex. In embodiments, the targeting element comprises a nuclease-deficient (or inactive, or “catalytically dead” Cas, e.g., Cas9, typically denoted as “dCas” or “dCas9”) guide RNA complex.

In embodiments, the dCas9/gRNA complex comprises a guide RNA selected from: GTTTAGCTCACCCGTGAGCC (SEQ ID NO: 91), CCCAATATTATTGTTCTCTG (SEQ ID NO: 92), GGGGTGGGATAGGGGATACG (SEQ ID NO: 93), GGATCCCCCTCTACATTTAA (SEQ ID NO: 94), GTGATCTTGTACAAATCATT (SEQ ID NO: 95), CTACACAGAATCTGTTAGAA (SEQ ID NO: 96), TAAGCTAGAGAATAGATCTC (SEQ ID NO: 97), and TCAATACACTTAATGATTTA (SEQ ID NO: 98), wherein the guide RNA directs the enzyme to a chemokine (C—C motif) receptor 5 (CCR5) gene.

In embodiments, the dCas9/gRNA complex comprises a guide RNA selected from:

(SEQ ID NO: 99)
CACCGGGAGCCACGAAAACAGATCC;
(SEQ ID NO: 100)
CACCGCGAAAACAGATCCAGGGACA;
(SEQ ID NO: 101)
CACCGAGATCCAGGGACACGGTGCT;
(SEQ ID NO: 102)
CACCGGACACGGTGCTAGGACAGTG;
(SEQ ID NO: 103)
CACCGGAAAATGACCCAACAGCCTC;
(SEQ ID NO: 104)
CACCGGCCTGGCCGGCCTGACCACT;
(SEQ ID NO: 105)
CACCGCTGAGCACTGAAGGCCTGGC;
(SEQ ID NO: 106)
CACCGTGGTTTCCACTGAGCACTGA;
(SEQ ID NO: 107)
CACCGGATAGCCAGGAGTCCTTTCG;
(SEQ ID NO: 108)
CACCGGCGCTTCCAGTGCTCAGACT;
(SEQ ID NO: 109)
CACCGCAGTGCTCAGACTAGGGAAG;
(SEQ ID NO: 110)
CACCGGCCCCTCCTCCTTCAGAGCC;
(SEQ ID NO: 111)
CACCGTCCTTCAGAGCCAGGAGTCC;
(SEQ ID NO: 112)
CACCGTGGTTTCCGAGCTTGACCCT;
(SEQ ID NO: 113)
CACCGCTGCAGAGTATCTGCTGGGG;
(SEQ ID NO: 114)
CACCGCGTTCCTGCAGAGTATCTGC;
(SEQ ID NO: 131)
TCCCCTCCCAGAAAGACCTG;
(SEQ ID NO: 132)
TGGGCTCCAAGCAATCCTGG;
(SEQ ID NO: 133)
GTGGCTCAGGAGGTACCTGG;
(SEQ ID NO: 134)
GAGCCACGAAAACAGATCCA;
(SEQ ID NO: 135)
AAGTGAACGGGGAAGGGAGG;
(SEQ ID NO: 136)
GACAAAAGCCGAAGTCCAGG;
(SEQ ID NO: 137)
GTGGTTGATAAACCCACGTG;
(SEQ ID NO: 138)
TGGGAACAGCCACAGCAGGG;
(SEQ ID NO: 139)
GCAGGGGAACGGGGATGCAG;
(SEQ ID NO: 140)
GAGATGGTGGACGAGGAAGG;
(SEQ ID NO: 141)
GAGATGGCTCCAGGAAATGG;
(SEQ ID NO: 142)
TAAGGAATCTGCCTAACAGG;
(SEQ ID NO: 143)
TCAGGAGACTAGGAAGGAGG;
(SEQ ID NO: 144)
TATAAGGTGGTCCCAGCTCG;
(SEQ ID NO: 145)
CTGGAAGATGCCATGACAGG;
(SEQ ID NO: 146)
GCACAGACTAGAGAGGTAAG;
(SEQ ID NO: 147)
ACAGACTAGAGAGGTAAGGG;
(SEQ ID NO: 148)
GAGAGGTGACCCGAATCCAC;
(SEQ ID NO: 149)
GCACAGGCCCCAGAAGGAGA;
(SEQ ID NO: 150)
CCGGAGAGGACCCAGACACG;
(SEQ ID NO: 151)
GAGAGGACCCAGACACGGGG;
(SEQ ID NO: 152)
GCAACACAGCAGAGAGCAAG;
(SEQ ID NO: 153)
GAAGAGGGAGTGGAGGAAGA;
(SEQ ID NO: 154)
AAGACGGAACCTGAAGGAGG;
(SEQ ID NO: 155)
AGAAAGCGGCACAGGCCCAG;
(SEQ ID NO: 156)
GGGAAACAGTGGGCCAGAGG;
(SEQ ID NO: 157)
GTCCGGACTCAGGAGAGAGA;
(SEQ ID NO: 158)
GGCACAGCAAGGGCACTCGG;
(SEQ ID NO: 159)
GAAGAGGGGAAGTCGAGGGA;
(SEQ ID NO: 160)
GGGAATGGTAAGGAGGCCTG;
(SEQ ID NO: 161)
GCAGAGTGGTCAGCACAGAG;
(SEQ ID NO: 162)
GCACAGAGTGGCTAAGCCCA;
(SEQ ID NO: 163)
GACGGGGTGTCAGCATAGGG;
(SEQ ID NO: 164)
GCCCAGGGCCAGGAACGACG;
(SEQ ID NO: 165)
GGTGGAGTCCAGCACGGCGC;
(SEQ ID NO: 166)
ACAGGCCGCCAGGAACTCGG;
(SEQ ID NO: 167)
ACTAGGAAGTGTGTAGCACC;
(SEQ ID NO: 168)
ATGAATAGCAGACTGCCCCG;
(SEQ ID NO: 169)
ACACCCCTAAAAGCACAGTG;
(SEQ ID NO: 170)
CAAGGAGTTCCAGCAGGTGG;
(SEQ ID NO: 171)
AAGGAGTTCCAGCAGGTGGG;
(SEQ ID NO: 172)
TGGAAAGAGGAGGGAAGAGG;
(SEQ ID NO: 173)
TCGAATTCCTAACTGCCCCG;
(SEQ ID NO: 174)
GACCTGCCCAGCACACCCTG;
(SEQ ID NO: 175)
GGAGCAGCTGCGGCAGTGGG;
(SEQ ID NO: 176)
GGGAGGGAGAGCTTGGCAGG;
(SEQ ID NO: 177)
GTTACGTGGCCAAGAAGCAG;
(SEQ ID NO: 178)
GCTGAACAGAGAAGAGCTGG;
(SEQ ID NO: 179)
TCTGAGGGTGGAGGGACTGG;
(SEQ ID NO: 180)
GGAGAGGTGAGGGACTTGGG;
(SEQ ID NO: 181)
GTGAACCAGGCAGACAACGA;
(SEQ ID NO: 182)
CAGGTACCTCCTGAGCCACG;
(SEQ ID NO: 183)
GGGGGAGTAGGGGCATGCAG;
(SEQ ID NO: 184)
GCAAATGGCCAGCAAGGGTG;
(SEQ ID NO: 309)
CAAATGGCCAGCAAGGGTGG;
(SEQ ID NO: 310)
GCAGAACCTGAGGATATGGA;
(SEQ ID NO: 311)
AATACACAGAATGAAAATAG;
(SEQ ID NO: 312)
CTGGTGACTAGAATAGGCAG;
(SEQ ID NO: 313)
TGGTGACTAGAATAGGCAGT;
(SEQ ID NO: 314)
TAAAAGAATGTGAAAAGATG;
(SEQ ID NO: 315)
TCAGGAGTTCAAGACCACCC;
(SEQ ID NO: 316)
TGTAGTCCCAGTTATGCAGG;
(SEQ ID NO: 317)
GGGTTCACACCACAAATGCA;
(SEQ ID NO: 318)
GGCAAATGGCCAGCAAGGGT;
(SEQ ID NO: 319)
AGAAACCAATCCCAAAGCAA;
(SEQ ID NO: 320)
GCCAAGGACACCAAAACCCA;
(SEQ ID NO: 321)
AGTGGTGATAAGGCAACAGT;
(SEQ ID NO: 322)
CCTGAGACAGAAGTATTAAG;
(SEQ ID NO: 323)
AAGGTCACACAATGAATAGG;
(SEQ ID NO: 324)
CACCATACTAGGGAAGAAGA;
(SEQ ID NO: 327)
CAATACCCTGCCCTTAGTGG;
(SEQ ID NO: 325)
AATACCCTGCCCTTAGTGGG;
(SEQ ID NO: 326)
TTAGTGGGGGGGGAGTGGG;
(SEQ ID NO: 328)
GTGGGGGGTGGAGTGGGGGG;
(SEQ ID NO: 329)
GGGGGGGGAGTGGGGGGTG;
(SEQ ID NO: 330)
GGGGTGGAGTGGGGGGTGGG;
(SEQ ID NO: 331)
GGGTGGAGTGGGGGGTGGGG;
(SEQ ID NO: 332)
GGGGGTGGGGAAAGACATCG;
(SEQ ID NO: 333)
GCAGCTGTGAATTCTGATAG;
(SEQ ID NO: 334)
GAGATCAGAGAAACCAGATG;
(SEQ ID NO: 335)
TCTATACTGATTGCAGCCAG;
(SEQ ID NO: 185)
CACCGAATCGAGAAGCGACTCGACA;
(SEQ ID NO: 186)
CACCGGTCCCTGGGCGTTGCCCTGC;
(SEQ ID NO: 187)
CACCGCCCTGGGCGTTGCCCTGCAG;
(SEQ ID NO: 188)
CACCGCCGTGGGAAGATAAACTAAT;
(SEQ ID NO: 189)
CACCGTCCCCTGCAGGGCAACGCCC;
(SEQ ID NO: 190)
CACCGGTCGAGTCGCTTCTCGATTA;
(SEQ ID NO: 191)
CACCGCTGCTGCCTCCCGTCTTGTA;
(SEQ ID NO: 192)
CACCGGAGTGCCGCAATACCTTTAT;
(SEQ ID NO: 193)
CACCGACACTTTGGTGGTGCAGCAA;
(SEQ ID NO: 194)
CACCGTCTCAAATGGTATAAAACTC;
(SEQ ID NO: 195)
CACCGAATCCCGCCCATAATCGAGA;
(SEQ ID NO: 196)
CACCGTCCCGCCCATAATCGAGAAG;
(SEQ ID NO: 197)
CACCGCCCATAATCGAGAAGCGACT;
(SEQ ID NO: 198)
CACCGGAGAAGCGACTCGACATGGA;
(SEQ ID NO: 199)
CACCGGAAGCGACTCGACATGGAGG;
(SEQ ID NO: 200)
CACCGGCGACTCGACATGGAGGCGA;
(SEQ ID NO: 201)
AAACTGTCGAGTCGCTTCTCGATTC;
(SEQ ID NO: 202)
AAACGCAGGGCAACGCCCAGGGACC;
(SEQ ID NO: 203)
AAACCTGCAGGGCAACGCCCAGGGC;
(SEQ ID NO: 204)
AAACATTAGTTTATCTTCCCACGGC;
(SEQ ID NO: 205)
AAACGGGCGTTGCCCTGCAGGGGAC;
(SEQ ID NO: 206)
AAACTAATCGAGAAGCGACTCGACC;
(SEQ ID NO: 207)
AAACTACAAGACGGGAGGCAGCAGC;
(SEQ ID NO: 208)
AAACATAAAGGTATTGCGGCACTCC;
(SEQ ID NO: 209)
AAACTTGCTGCACCACCAAAGTGTC;
(SEQ ID NO: 210)
AAACGAGTTTTATACCATTTGAGAC;
(SEQ ID NO: 211)
AAACTCTCGATTATGGGGGGGATTC;
(SEQ ID NO: 212)
AAACCTTCTCGATTATGGGGGGGAC;
(SEQ ID NO: 213)
AAACAGTCGCTTCTCGATTATGGGC;
(SEQ ID NO: 214)
AAACTCCATGTCGAGTCGCTTCTCC;
(SEQ ID NO: 215)
AAACCCTCCATGTCGAGTCGCTTCC;
(SEQ ID NO: 216)
AAACTCGCCTCCATGTCGAGTCGCC;
(SEQ ID NO: 217)
CACCGACAGGGTTAATGTGAAGTCC;
(SEQ ID NO: 218)
CACCGTCCCCCTCTACATTTAAAGT;
(SEQ ID NO: 219)
CACCGCATTTAAAGTTGGTTTAAGT;
(SEQ ID NO: 220)
CACCGTTAGAAAATATAAAGAATAA;
(SEQ ID NO: 221)
CACCGTAAATGCTTACTGGTTTGAA;
(SEQ ID NO: 222)
CACCGTCCTGGGTCCAGAAAAAGAT;
(SEQ ID NO: 223)
CACCGTTGGGTGGTGAGCATCTGTG;
(SEQ ID NO: 224)
CACCGCGGGGAGAGTGGAGAAAAAG;
(SEQ ID NO: 225)
CACCGGTTAAAACTCTTTAGACAAC;
(SEQ ID NO: 226)
CACCGGAAAATCCCCACTAAGATCC;
(SEQ ID NO: 227)
AAACGGACTTCACATTAACCCTGTC;
(SEQ ID NO: 228)
AAACACTTTAAATGTAGAGGGGGAC;
(SEQ ID NO: 229)
AAACACTTAAACCAACTTTAAATGC;
(SEQ ID NO: 230)
AAACTTATTCTTTATATTTTCTAAC;
(SEQ ID NO: 231)
AAACTTCAAACCAGTAAGCATTTAC;
(SEQ ID NO: 232)
AAACATCTTTTTCTGGACCCAGGAC;
(SEQ ID NO: 233)
AAACCACAGATGCTCACCACCCAAC;
(SEQ ID NO: 234)
AAACCTTTTTCTCCACTCTCCCCGC;
(SEQ ID NO: 235)
AAACGTTGTCTAAAGAGTTTTAACC;
(SEQ ID NO: 236)
AAACGGATCTTAGTGGGGATTTTCC;
(SEQ ID NO: 237)
AGTAGCAGTAATGAAGCTGG;
(SEQ ID NO: 238)
ATACCCAGACGAGAAAGCTG;
(SEQ ID NO: 239)
TACCCAGACGAGAAAGCTGA;
(SEQ ID NO: 240)
GGTGGTGAGCATCTGTGTGG;
(SEQ ID NO: 241)
AAATGAGAAGAAGAGGCACA;
(SEQ ID NO: 242)
CTTGTGGCCTGGGAGAGCTG;
(SEQ ID NO: 243)
GCTGTAGAAGGAGACAGAGC;
(SEQ ID NO: 244)
GAGCTGGTTGGGAAGACATG;
(SEQ ID NO: 245)
CTGGTTGGGAAGACATGGGG;
(SEQ ID NO: 246)
CGTGAGGATGGGAAGGAGGG;
(SEQ ID NO: 247)
ATGCAGAGTCAGCAGAACTG;
(SEQ ID NO: 248)
AAGACATCAAGCACAGAAGG;
(SEQ ID NO: 249)
TCAAGCACAGAAGGAGGAGG;
(SEQ ID NO: 250)
AACCGTCAATAGGCAAAGGG;
(SEQ ID NO: 251)
CCGTATTTCAGACTGAATGG;
(SEQ ID NO: 252)
GAGAGGACAGGTGCTACAGG;
(SEQ ID NO: 253)
AACCAAGGAAGGGCAGGAGG;
(SEQ ID NO: 254)
GACCTCTGGGTGGAGACAGA;
(SEQ ID NO: 255)
CAGATGACCATGACAAGCAG;
(SEQ ID NO: 256)
AACACCAGTGAGTAGAGCGG;
(SEQ ID NO: 257)
AGGACCTTGAAGCACAGAGA;
(SEQ ID NO: 258)
TACAGAGGCAGACTAACCCA;
(SEQ ID NO: 259)
ACAGAGGCAGACTAACCCAG;
(SEQ ID NO: 260)
TAAATGACGTGCTAGACCTG;
(SEQ ID NO: 261)
AGTAACCACTCAGGACAGGG;
(SEQ ID NO: 262)
ACCACAAAACAGAAACACCA;
(SEQ ID NO: 263)
GTTTGAAGACAAGCCTGAGG;
(SEQ ID NO: 264)
GCTGAACCCCAAAAGACAGG;
(SEQ ID NO: 265)
GCAGCTGAGACACACACCAG;
(SEQ ID NO: 266)
AGGACACCCCAAAGAAGCTG;
(SEQ ID NO: 267)
GGACACCCCAAAGAAGCTGA;
(SEQ ID NO: 268)
CCAGTGCAATGGACAGAAGA;
(SEQ ID NO: 269)
AGAAGAGGGAGCCTGCAAGT;
(SEQ ID NO: 270)
GTGTTTGGGCCCTAGAGCGA;
(SEQ ID NO: 271)
CATGTGCCTGGTGCAATGCA;
(SEQ ID NO: 272)
TACAAAGAGGAAGATAAGTG;
(SEQ ID NO: 273)
GTCACAGAATACACCACTAG;
(SEQ ID NO: 274)
GGGTTACCCTGGACATGGAA;
(SEQ ID NO: 275)
CATGGAAGGGTATTCACTCG;
(SEQ ID NO: 276)
AGAGTGGCCTAGACAGGCTG;
(SEQ ID NO: 277)
CATGCTGGACAGCTCGGCAG;
(SEQ ID NO: 278)
AGTGAAAGAAGAGAAAATTC;
(SEQ ID NO: 279)
TGGTAAGTCTAAGAAACCTA;
(SEQ ID NO: 280)
CCCACAGCCTAACCACCCTA;
(SEQ ID NO: 281)
AATATTTCAAAGCCCTAGGG;
(SEQ ID NO: 282)
GCACTCGGAACAGGGTCTGG;
(SEQ ID NO: 283)
AGATAGGAGCTCCAACAGTG;
(SEQ ID NO: 284)
AAGTTAGAGCAGCCAGGAAA;
(SEQ ID NO: 285)
TAGAGCAGCCAGGAAAGGGA;
(SEQ ID NO: 286)
TGAATACCCTTCCATGTCCA;
(SEQ ID NO: 287)
CCTGCATTGCACCAGGCACA;
(SEQ ID NO: 288)
TCTAGGGCCCAAACACACCT;
(SEQ ID NO: 289)
TCCCTCCATCTATCAAAAGG;
(SEQ ID NO: 290)
AGCCCTGAGACAGAAGCAGG;
(SEQ ID NO: 291)
GCCCTGAGACAGAAGCAGGT;
(SEQ ID NO: 292)
AGGAGATGCAGTGATACGCA;
(SEQ ID NO: 293)
ACAATACCAAGGGTATCCGG;
(SEQ ID NO: 294)
TGATAAAGAAAACAAAGTGA;
(SEQ ID NO: 295)
AAAGAAAACAAAGTGAGGGA;
(SEQ ID NO: 296)
GTGGCAAGTGGAGAAATTGA;
(SEQ ID NO: 297)
CAAGTGGAGAAATTGAGGGA;
(SEQ ID NO: 298)
GTGGTGATGATTGCAGCTGG;
(SEQ ID NO: 299)
CTATGTGCCTGACACACAGG;
(SEQ ID NO: 300)
GGGTTGGACCAGGAAAGAGG;
(SEQ ID NO: 301)
GATGCCTGGAAAAGGAAAGA;
(SEQ ID NO: 302)
TAGTATGCACCTGCAAGAGG;
(SEQ ID NO: 303)
TATGCACCTGCAAGAGGGGG;
(SEQ ID NO: 304)
AGGGGAAGAAGAGAAGCAGA;
(SEQ ID NO: 305)
GCTGAATCAAGAGACAAGCG;
(SEQ ID NO: 306)
AAGCAAATAAATCTCCTGGG;
(SEQ ID NO: 307)
AGATGAGTGCTAGAGACTGG;
and
(SEQ ID NO: 308)
CTGATGGTTGAGCACAGCAG.

In embodiments, the guide RNAs are: AATCGAGAAGCGACTCGACA (SEQ ID NO: 425), and tgccctgcaggggagtgagc (SEQ ID NO: 426). In embodiments, the guide RNAs are gaagcgactcgacatggagg (SEQ ID NO: 427) and cctgcaggggagtgagcagc (SEQ ID NO: 428).

In embodiments, guide RNAs (gRNAs) for targeting human genomic safe harbor sites using any of the gRNA-based targeting elements, e.g., without limitation dCas, in areas of open chromatin are as shown in TABLE 3A-3F.

In embodiments, guide RNAs (gRNAs) for targeting human genomic safe harbor sites using any of the gRNA-based targeting elements, e.g., without limitation dCas, in areas of open chromatin are as shown in TABLE 3A:

| GSHS | Identifier | Sequence |
|---|---|---|
| AAVS1 | 14F | ggagccacgaaaacagatcc |
| | | (SEQ ID NO: 800) |
| AAVS1 | 15F | cgaaaacagatccagggaca |
| | | (SEQ ID NO: 801) |
| AAVS1 | 16F | agatccagggacacggtgct |
| | | (SEQ ID NO: 802) |
| AAVS1 | 17F | gacacggtgctaggacagtg |
| | | (SEQ ID NO: 803) |
| AAVS1 | 18F | gaaaatgacccaacagcctc |
| | | (SEQ ID NO: 804) |
| AAVS1 | 19F | gcctggccggcctgaccact |
| | | (SEQ ID NO: 805) |
| AAVS1 | 20F | ctgagcactgaaggcctggc |
| | | (SEQ ID NO: 806) |
| AAVS1 | 21F | tggtttccactgagcactga |
| | | (SEQ ID NO: 807) |
| AAVS1 | 22F | gatagccaggagtcctttcg |
| | | (SEQ ID NO: 808) |
| AAVS1 | 23F | gogcttccagtgctcagact |
| | | (SEQ ID NO: 809) |
| AAVS1 | 24F | cagtgctcagactagggaag |
| | | (SEQ ID NO: 810) |
| AAVS1 | 25F | gcccctcctcottcagagcc |
| | | (SEQ ID NO: 811) |
| AAVS1 | 26F | tccttcagagccaggagtcc |
| | | (SEQ ID NO: 812) |
| AAVS1 | 27F | tggtttccgagcttgaccct |
| | | (SEQ ID NO: 813) |
| AAVS1 | 28F | ctgcagagtatctgctgggg |
| | | (SEQ ID NO: 814) |
| AAVS1 | 29F | cgttcctgcagagtatctgc |
| | | (SEQ ID NO: 815) |
| AAVS1 | 29R | AAACGCAGATACTCTGCAGGAACGC |
| | | (SEQ ID NO: 130) |
| AAVS1 | AAVS1 | TCCCCTCCCAGAAAGACCTG |
| | | (SEQ ID NO: 131) |
| AAVS1 | gAAVS2 | TGGGCTCCAAGCAATCCTGG |
| | | (SEQ ID NO: 132) |
| AAVS1 | gAAVS3 | GTGGCTCAGGAGGTACCTGG |
| | | (SEQ ID NO: 133) |
| AAVS1 | gAAVS4 | GAGCCACGAAAACAGATCCA |
| | | (SEQ ID NO: 134) |
| AAVS1 | gAAVS5 | AAGTGAACGGGGAAGGGAGG |
| | | (SEQ ID NO: 135) |
| AAVS1 | gAAVS6 | GACAAAAGCCGAAGTCCAGG |
| | | (SEQ ID NO: 136) |
| AAVS1 | gAAVS7 | GTGGTTGATAAACCCACGTG |
| | | (SEQ ID NO: 137) |
| AAVS1 | gAAVS8 | TGGGAACAGCCACAGCAGGG |
| | | (SEQ ID NO: 138) |
| AAVS1 | gAAVS9 | GCAGGGGAACGGGGATGCAG |
| | | (SEQ ID NO: 139) |
| AAVS1 | gAAVS10 | GAGATGGTGGACGAGGAAGG |
| | | (SEQ ID NO: 140) |
| AAVS1 | gAAVS11 | GAGATGGCTCCAGGAAATGG |
| | | (SEQ ID NO: 141) |
| AAVS1 | gAAVS12 | TAAGGAATCTGCCTAACAGG |
| | | (SEQ ID NO: 142) |
| AAVS1 | gAAVS13 | TCAGGAGACTAGGAAGGAGG |
| | | (SEQ ID NO: 143) |
| AAVS1 | gAAVS14 | TATAAGGTGGTCCCAGCTCG |
| | | (SEQ ID NO: 144) |
| AAVS1 | gAAVS15 | CTGGAAGATGCCATGACAGG |
| | | (SEQ ID NO: 145) |
| AAVS1 | gAAVS16 | GCACAGACTAGAGAGGTAAG |
| | | (SEQ ID NO: 146) |
| AAVS1 | gAAVS17 | ACAGACTAGAGAGGTAAGGG |
| | | (SEQ ID NO: 147) |
| AAVS1 | gAAVS18 | GAGAGGTGACCCGAATCCAC |
| | | (SEQ ID NO: 148) |
| AAVS1 | gAAVS19 | GCACAGGCCCCAGAAGGAGA |
| | | (SEQ ID NO: 149) |
| AAVS1 | gAAVS20 | CCGGAGAGGACCCAGACACG |
| | | (SEQ ID NO: 150) |
| AAVS1 | gAAVS21 | GAGAGGACCCAGACACGGGG |
| | | (SEQ ID NO: 151) |
| AAVS1 | gAAVS22 | GCAACACAGCAGAGAGCAAG |
| | | (SEQ ID NO: 152) |
| AAVS1 | gAAVS23 | GAAGAGGGAGTGGAGGAAGA |
| | | (SEQ ID NO: 153) |
| AAVS1 | gAAVS24 | AAGACGGAACCTGAAGGAGG |
| | | (SEQ ID NO: 154) |
| AAVS1 | gAAVS25 | AGAAAGCGGCACAGGCCCAG |
| | | (SEQ ID NO: 155) |
| AAVS1 | gAAVS26 | GGGAAACAGTGGGCCAGAGG |
| | | (SEQ ID NO: 156) |
| AAVS1 | gAAVS27 | GTCCGGACTCAGGAGAGAGA |
| | | (SEQ ID NO: 157) |
| AAVS1 | gAAVS28 | GGCACAGCAAGGGCACTCGG |
| | | (SEQ ID NO: 158) |
| AAVS1 | gAAVS29 | GAAGAGGGGAAGTCGAGGGA |
| | | (SEQ ID NO: 159) |
| AAVS1 | gAAVS30 | GGGAATGGTAAGGAGGCCTG |
| | | (SEQ ID NO: 160) |
| AAVS1 | gAAVS31 | GCAGAGTGGTCAGCACAGAG |
| | | (SEQ ID NO: 161) |
| AAVS1 | gAAVS32 | GCACAGAGTGGCTAAGCCCA |
| | | (SEQ ID NO: 162) |
| AAVS1 | gAAVS33 | GACGGGGTGTCAGCATAGGG |
| | | (SEQ ID NO: 163) |
| AAVS1 | gAAVS34 | GCCCAGGGCCAGGAACGACG |
| | | (SEQ ID NO: 164) |
| AAVS1 | gAAVS35 | GGTGGAGTCCAGCACGGCGC |
| | | (SEQ ID NO: 165) |
| AAVS1 | gAAVS36 | ACAGGCCGCCAGGAACTCGG |
| | | (SEQ ID NO: 166) |
| AAVS1 | gAAVS37 | ACTAGGAAGTGTGTAGCACC |
| | | (SEQ ID NO: 167) |
| AAVS1 | gAAVS38 | ATGAATAGCAGACTGCCCCG |
| | | (SEQ ID NO: 168) |
| AAVS1 | gAAVS39 | ACACCCCTAAAAGCACAGTG |
| | | (SEQ ID NO: 169) |
| AAVS1 | gAAVS40 | CAAGGAGTTCCAGCAGGTGG |
| | | (SEQ ID NO: 170) |
| AAVS1 | gAAVS41 | AAGGAGTTCCAGCAGGTGGG |
| | | (SEQ ID NO: 171) |
| AAVS1 | gAAVS42 | TGGAAAGAGGAGGGAAGAGG |
| | | (SEQ ID NO: 172) |
| AAVS1 | gAAVS43 | TCGAATTCCTAACTGCCCCG |
| | | (SEQ ID NO: 173) |
| AAVS1 | gAAVS44 | GACCTGCCCAGCACACCCTG |
| | | (SEQ ID NO: 174) |
| AAVS1 | gAAVS45 | GGAGCAGCTGCGGCAGTGGG |
| | | (SEQ ID NO: 175) |
| AAVS1 | gAAVS46 | GGGAGGGAGAGCTTGGCAGG |
| | | (SEQ ID NO: 176) |
| AAVS1 | gAAVS47 | GTTACGTGGCCAAGAAGCAG |
| | | (SEQ ID NO: 177) |
| AAVS1 | gAAVS48 | GCTGAACAGAGAAGAGCTGG |
| | | (SEQ ID NO: 178) |
| AAVS1 | gAAVS49 | TCTGAGGGTGGAGGGACTGG |
| | | (SEQ ID NO: 179) |
| AAVS1 | gAAVS50 | GGAGAGGTGAGGGACTTGGG |
| | | (SEQ ID NO: 180) |
| AAVS1 | gAAVS51 | GTGAACCAGGCAGACAACGA |
| | | (SEQ ID NO: 181) |
| AAVS1 | gAAVS52 | CAGGTACCTCCTGAGCCACG |
| | | (SEQ ID NO: 182) |
| AAVS1 | gAAVS53 | GGGGGAGTAGGGGCATGCAG |
| | | (SEQ ID NO: 183) |
| hROSA26 | gHROSA26-1 | GCAAATGGCCAGCAAGGGTG |
| | | (SEQ ID NO: 184) |
| hROSA26 | gHROSA26-2 | CAAATGGCCAGCAAGGGTGG |
| | | (SEQ ID NO: 309) |
| hROSA26 | gHROSA26-3 | GCAGAACCTGAGGATATGGA |
| | | (SEQ ID NO: 310) |
| hROSA26 | gHROSA26-3 | AATACACAGAATGAAAATAG |
| | | (SEQ ID NO: 311) |
| hROSA26 | gHROSA26-4 | CTGGTGACTAGAATAGGCAG |
| | | (SEQ ID NO: 312) |
| hROSA26 | gHROSA26-5 | TGGTGACTAGAATAGGCAGT |
| | | (SEQ ID NO: 313) |
| hROSA26 | gHROSA26-6 | TAAAAGAATGTGAAAAGATG |
| | | (SEQ ID NO: 314) |
| hROSA26 | gHROSA26-7 | TCAGGAGTTCAAGACCACCC |
| | | (SEQ ID NO: 315) |
| hROSA26 | gHROSA26-8 | TGTAGTCCCAGTTATGCAGG |
| | | (SEQ ID NO: 316) |
| hROSA26 | gHROSA26-9 | GGGTTCACACCACAAATGCA |
| | | (SEQ ID NO: 317) |
| hROSA26 | gHROSA26-10 | GGCAAATGGCCAGCAAGGGT |
| | | (SEQ ID NO: 318) |
| hROSA26 | gHROSA26-11 | AGAAACCAATCCCAAAGCAA |
| | | (SEQ ID NO: 319) |
| hROSA26 | gHROSA26-12 | GCCAAGGACACCAAAACCCA |
| | | (SEQ ID NO: 320) |
| hROSA26 | gHROSA26-13 | AGTGGTGATAAGGCAACAGT |
| | | (SEQ ID NO: 321) |
| hROSA26 | gHROSA26-14 | CCTGAGACAGAAGTATTAAG |
| | | (SEQ ID NO: 322) |
| hROSA26 | gHROSA26-15 | AAGGTCACACAATGAATAGG |
| | | (SEQ ID NO: 323) |
| hROSA26 | gHROSA26-16 | CACCATACTAGGGAAGAAGA |
| | | (SEQ ID NO: 324) |
| hROSA26 | gHROSA26-17 | CAATACCCTGCCCTTAGTGG |
| | | (SEQ ID NO: 327) |
| hROSA26 | gHROSA26-18 | AATACCCTGCCCTTAGTGGG |
| | | (SEQ ID NO: 325) |
| hROSA26 | gHROSA26-19 | TTAGTGGGGGGTGGAGTGGG |
| | | (SEQ ID NO: 326) |
| hROSA26 | gHROSA26-20 | GTGGGGGGTGGAGTGGGGGG |
| | | (SEQ ID NO: 328) |
| hROSA26 | gHROSA26-21 | GGGGGGTGGAGTGGGGGGTG |
| | | (SEQ ID NO: 329) |
| hROSA26 | gHROSA26-22 | GGGGTGGAGTGGGGGGTGGG |
| | | (SEQ ID NO: 330) |
| hROSA26 | gHROSA26-23 | GGGTGGAGTGGGGGGTGGGG |
| | | (SEQ ID NO: 331) |
| hROSA26 | gHROSA26-24 | GGGGGTGGGGAAAGACATCG |
| | | (SEQ ID NO: 332) |
| hROSA26 | gHROSA26-25 | GCAAATGGCCAGCAAGGGTG |
| | | (SEQ ID NO: 184) |
| hROSA26 | gHROSA26-26 | CAAATGGCCAGCAAGGGTGG |
| | | (SEQ ID NO: 309) |
| hROSA26 | gHROSA26-27 | GCAGAACCTGAGGATATGGA |
| | | (SEQ ID NO: 310) |
| hROSA26 | gHROSA26-28 | AATACACAGAATGAAAATAG |
| | | (SEQ ID NO: 311) |
| hROSA26 | gHROSA26-29 | CTGGTGACTAGAATAGGCAG |
| | | (SEQ ID NO: 312) |
| hROSA26 | gHROSA26-30 | TGGTGACTAGAATAGGCAGT |
| | | (SEQ ID NO: 313) |
| hROSA26 | gHROSA26-31 | TAAAAGAATGTGAAAAGATG |
| | | (SEQ ID NO: 314) |
| hROSA26 | gHROSA26-32 | TCAGGAGTTCAAGACCACCC |
| | | (SEQ ID NO: 315) |
| hROSA26 | gHROSA26-33 | TGTAGTCCCAGTTATGCAGG |
| | | (SEQ ID NO: 316) |
| hROSA26 | gHROSA26-34 | GGGTTCACACCACAAATGCA |
| | | (SEQ ID NO: 317) |
| hROSA26 | gHROSA26-35 | GGCAAATGGCCAGCAAGGGT |
| | | (SEQ ID NO: 318) |
| hROSA26 | gHROSA26-36 | AGAAACCAATCCCAAAGCAA |
| | | (SEQ ID NO: 319) |
| hROSA26 | gHROSA26-37 | GCCAAGGACACCAAAACCCA |
| | | (SEQ ID NO: 320) |
| hROSA26 | gHROSA26-38 | AGTGGTGATAAGGCAACAGT |
| | | (SEQ ID NO: 321) |
| hROSA26 | gHROSA26-39 | CCTGAGACAGAAGTATTAAG |
| | | (SEQ ID NO: 322) |
| hROSA26 | gHROSA26-40 | AAGGTCACACAATGAATAGG |
| | | (SEQ ID NO: 323) |
| hROSA26 | gHROSA26-41 | CACCATACTAGGGAAGAAGA |
| | | (SEQ ID NO: 324) |
| hROSA26 | gHROSA26-42 | CAATACCCTGCCCTTAGTGG |
| | | (SEQ ID NO: 327) |
| hROSA26 | gHROSA26-43 | AATACCCTGCCCTTAGTGGG |
| | | (SEQ ID NO: 325) |
| hROSA26 | gHROSA26-44 | TTAGTGGGGGGTGGAGTGGG |
| | | (SEQ ID NO: 326) |
| hROSA26 | gHROSA26-45 | GTGGGGGGTGGAGTGGGGGG |
| | | (SEQ ID NO: 328) |
| hROSA26 | gHROSA26-46 | GGGGGGTGGAGTGGGGGGTG |
| | | (SEQ ID NO: 329) |
| hROSA26 | gHROSA26-47 | GGGGTGGAGTGGGGGGTGGG |
| | | (SEQ ID NO: 330) |
| hROSA26 | gHROSA26-48 | GGGTGGAGTGGGGGGTGGGG |
| | | (SEQ ID NO: 331) |
| hROSA26 | gHROSA26-49 | GGGGGTGGGGAAAGACATCG |
| | | (SEQ ID NO: 332) |
| hROSA26 | gHROSA26-50 | GCAGCTGTGAATTCTGATAG |
| | | (SEQ ID NO: 333) |
| hROSA26 | gHROSA26-51 | GAGATCAGAGAAACCAGATG |
| | | (SEQ ID NO: 334) |
| hROSA26 | gHROSA26-52 | TCTATACTGATTGCAGCCAG |
| | | (SEQ ID NO: 335) |
| hROSA26 | gHROSA26-1 | GCAAATGGCCAGCAAGGGTG |
| | | (SEQ ID NO: 184) |
| hROSA26 | 44F | CACCGAATCGAGAAGCGACTCGACA |
| | | (SEQ ID NO: 185) |
| hROSA26 | 45F | CACCGGTCCCTGGGCGTTGCCCTGC |
| | | (SEQ ID NO: 186) |
| hROSA26 | 46F | CACCGCCCTGGGCGTTGCCCTGCAG |
| | | (SEQ ID NO: 187) |
| hROSA26 | 1nF | CACCGCCGTGGGAAGATAAACTAAT |
| | | (SEQ ID NO: 188) |
| hROSA26 | 2nF | CACCGTCCCCTGCAGGGCAACGCCC |
| | | (SEQ ID NO: 189) |
| hROSA26 | 3nF | CACCGGTCGAGTCGCTTCTCGATTA |
| | | (SEQ ID NO: 190) |
| hROSA26 | 4nF | CACCGCTGCTGCCTCCCGTCTTGTA |
| | | (SEQ ID NO: 191) |
| hROSA26 | 5nF | CACCGGAGTGCCGCAATACCTTTAT |
| | | (SEQ ID NO: 192) |
| hROSA26 | 6nF | CACCGACACTTTGGTGGTGCAGCAA |
| | | (SEQ ID NO: 193) |
| hROSA26 | 7nF | CACCGTCTCAAATGGTATAAAACTC |
| | | (SEQ ID NO: 194) |
| hROSA26 | 8nF | CACCGCCGTGGGAAGATAAACTAAT |
| | | (SEQ ID NO: 188) |
| hROSA26 | 9F | CACCGAATCCCGCCCATAATCGAGA |
| | | (SEQ ID NO: 195) |
| hROSA26 | 10F | CACCGTCCCGCCCATAATCGAGAAG |
| | | (SEQ ID NO: 196) |
| hROSA26 | 11F | CACCGCCCATAATCGAGAAGCGACT |
| | | (SEQ ID NO: 197) |
| hROSA26 | 12F | CACCGGAGAAGCGACTCGACATGGA |
| | | (SEQ ID NO: 198) |
| hROSA26 | 13F | CACCGGAAGCGACTCGACATGGAGG |
| | | (SEQ ID NO: 199) |
| hROSA26 | 14F | CACCGGCGACTCGACATGGAGGCGA |
| | | (SEQ ID NO: 200) |
| hROSA26 | 44F | AAACTGTCGAGTCGCTTCTCGATTC |
| | | (SEQ ID NO: 201) |
| hROSA26 | 45F | AAACGCAGGGCAACGCCCAGGGACC |
| | | (SEQ ID NO: 202) |
| hROSA26 | 46F | AAACCTGCAGGGCAACGCCCAGGGC |
| | | (SEQ ID NO: 203) |
| hROSA26 | 1nR | AAACATTAGTTTATCTTCCCACGGC |
| | | (SEQ ID NO: 204) |
| hROSA26 | 2nR | AAACGGGCGTTGCCCTGCAGGGGAC |
| | | (SEQ ID NO: 205) |
| hROSA26 | 3nR | AAACTAATCGAGAAGCGACTCGACC |
| | | (SEQ ID NO: 206) |
| hROSA26 | 4nR | AAACTACAAGACGGGAGGCAGCAGC |
| | | (SEQ ID NO: 207) |
| hROSA26 | 5nR | AAACATAAAGGTATTGCGGCACTCC |
| | | (SEQ ID NO: 208) |
| hROSA26 | 6nR | AAACTTGCTGCACCACCAAAGTGTC |
| | | (SEQ ID NO: 209) |
| hROSA26 | 7nR | AAACGAGTTTTATACCATTTGAGAC |
| | | (SEQ ID NO: 210) |
| hROSA26 | 8nR | AAACATTAGTTTATCTTCCCACGGC |
| | | (SEQ ID NO: 204) |
| hROSA26 | 9R | AAACTCTCGATTATGGGGGGATTC |
| | | (SEQ ID NO: 211) |
| hROSA26 | 10R | AAACCTTCTCGATTATGGGGGGGAC |
| | | (SEQ ID NO: 212) |
| hROSA26 | 11R | AAACAGTCGCTTCTCGATTATGGGC |
| | | (SEQ ID NO: 213) |
| hROSA26 | 12R | AAACTCCATGTCGAGTCGCTTCTCC |
| | | (SEQ ID NO: 214) |
| hROSA26 | 13R | AAACCCTCCATGTCGAGTCGCTTCC |
| | | (SEQ ID NO: 215) |
| hROSA26 | 14R | AAACTCGCCTCCATGTCGAGTCGCC |
| | | (SEQ ID NO: 216) |
| CCR5 | 1F | CACCGACAGGGTTAATGTGAAGTCC |
| | | (SEQ ID NO: 217) |
| CCR5 | 2F | CACCGTCCCCCTCTACATTTAAAGT |
| | | (SEQ ID NO: 218) |
| CCR5 | 3F | CACCGCATTTAAAGTTGGTTTAAGT |
| | | (SEQ ID NO: 219) |
| CCR5 | 4F | CACCGTTAGAAAATATAAAGAATAA |
| | | (SEQ ID NO: 220) |
| CCR5 | 5 | CACCGTAAATGCTTACTGGTTTGAA |
| | | (SEQ ID NO: 221) |
| CCR5 | 6F | CACCGTCCTGGGTCCAGAAAAAGAT |
| | | (SEQ ID NO: 222) |
| CCR5 | 7F | CACCGTTGGGTGGTGAGCATCTGTG |
| | | (SEQ ID NO: 223) |
| CCR5 | 8F | CACCGCGGGGAGAGTGGAGAAAAAG |
| | | (SEQ ID NO: 224) |
| CCR5 | 9F | CACCGGTTAAAACTCTTTAGACAAC |
| | | (SEQ ID NO: 225) |
| CCR5 | 10F | CACCGGAAAATCCCCACTAAGATCC |
| | | (SEQ ID NO: 226) |
| CCR5 | 1R | AAACGGACTTCACATTAACCCTGTC |
| | | (SEQ ID NO: 227) |
| CCR5 | 2R | AAACACTTTAAATGTAGAGGGGGAC |
| | | (SEQ ID NO: 228) |
| CCR5 | 3R | AAACACTTAAACCAACTTTAAATGC |
| | | (SEQ ID NO: 229) |
| CCR5 | 4R | AAACTTATTCTTTATATTTTCTAAC |
| | | (SEQ ID NO: 230) |
| CCR5 | 5R | AAACTTCAAACCAGTAAGCATTTAC |
| | | (SEQ ID NO: 231) |
| CCR5 | 6R | AAACATCTTTTTCTGGACCCAGGAC |
| | | (SEQ ID NO: 232) |
| CCR5 | 7R | AAACCACAGATGCTCACCACCCAAC |
| | | (SEQ ID NO: 233) |
| CCR5 | 8R | AAACCTTTTTCTCCACTCTCCCCGC |
| | | (SEQ ID NO: 234) |
| CCR5 | 9R | AAACGTTGTCTAAAGAGTTTTAACC |
| | | (SEQ ID NO: 235) |
| CCR5 | 10R | AAACGGATCTTAGTGGGGATTTTCC |
| | | (SEQ ID NO: 236) |
| CCR5 | gCCR5-1 | AGTAGCAGTAATGAAGCTGG |
| | | (SEQ ID NO: 237) |
| CCR5 | gCCR5-2 | ATACCCAGACGAGAAAGCTG |
| | | (SEQ ID NO: 238) |
| CCR5 | gCCR5-3 | TACCCAGACGAGAAAGCTGA |
| | | (SEQ ID NO: 239) |
| CCR5 | gCCR5-4 | GGTGGTGAGCATCTGTGTGG |
| | | (SEQ ID NO: 240) |
| CCR5 | gCCR5-5 | AAATGAGAAGAAGAGGCACA |
| | | (SEQ ID NO: 241) |
| CCR5 | gCCR5-6 | CTTGTGGCCTGGGAGAGCTG |
| | | (SEQ ID NO: 242) |
| CCR5 | gCCR5-7 | GCTGTAGAAGGAGACAGAGC |
| | | (SEQ ID NO: 243) |
| CCR5 | gCCR5-8 | GAGCTGGTTGGGAAGACATG |
| | | (SEQ ID NO: 244) |
| CCR5 | gCCR5-9 | CTGGTTGGGAAGACATGGGG |
| | | (SEQ ID NO: 245) |
| CCR5 | gCCR5-10 | CGTGAGGATGGGAAGGAGGG |
| | | (SEQ ID NO: 246) |
| CCR5 | gCCR5-11 | ATGCAGAGTCAGCAGAACTG |
| | | (SEQ ID NO: 247) |
| CCR5 | gCCR5-12 | AAGACATCAAGCACAGAAGG |
| | | (SEQ ID NO: 248) |
| CCR5 | gCCR5-13 | TCAAGCACAGAAGGAGGAGG |
| | | (SEQ ID NO: 249) |
| CCR5 | gCCR5-14 | AACCGTCAATAGGCAAAGGG |
| | | (SEQ ID NO: 250) |
| CCR5 | gCCR5-15 | CCGTATTTCAGACTGAATGG |
| | | (SEQ ID NO: 251) |
| CCR5 | gCCR5-16 | GAGAGGACAGGTGCTACAGG |
| | | (SEQ ID NO: 252) |
| CCR5 | gCCR5-17 | AACCAAGGAAGGGCAGGAGG |
| | | (SEQ ID NO: 253) |
| CCR5 | gCCR5-18 | GACCTCTGGGTGGAGACAGA |
| | | (SEQ ID NO: 254) |
| CCR5 | gCCR5-19 | CAGATGACCATGACAAGCAG |
| | | (SEQ ID NO: 255) |
| CCR5 | gCCR5-20 | AACACCAGTGAGTAGAGCGG |
| | | (SEQ ID NO: 256) |
| CCR5 | gCCR5-21 | AGGACCTTGAAGCACAGAGA |
| | | (SEQ ID NO: 257) |
| CCR5 | gCCR5-22 | TACAGAGGCAGACTAACCCA |
| | | (SEQ ID NO: 258) |
| CCR5 | gCCR5-23 | ACAGAGGCAGACTAACCCAG |
| | | (SEQ ID NO: 259) |
| CCR5 | gCCR5-24 | TAAATGACGTGCTAGACCTG |
| | | (SEQ ID NO: 260) |
| CCR5 | gCCR5-25 | AGTAACCACTCAGGACAGGG |
| | | (SEQ ID NO: 261) |
| chr2 | gchr2-1 | ACCACAAAACAGAAACACCA |
| | | (SEQ ID NO: 262) |
| chr2 | gchr2-2 | GTTTGAAGACAAGCCTGAGG |
| | | (SEQ ID NO: 263) |
| chr4 | gchr4-1 | GCTGAACCCCAAAAGACAGG |
| | | (SEQ ID NO: 264) |
| chr4 | gchr4-2 | GCAGCTGAGACACACACCAG |
| | | (SEQ ID NO: 265) |
| chr4 | gchr4-3 | AGGACACCCCAAAGAAGCTG |
| | | (SEQ ID NO: 266) |
| chr4 | gchr4-4 | GGACACCCCAAAGAAGCTGA |
| | | (SEQ ID NO: 267) |
| chr6 | gchr6-1 | CCAGTGCAATGGACAGAAGA |
| | | (SEQ ID NO: 268) |
| chr6 | gchr6-2 | AGAAGAGGGAGCCTGCAAGT |
| | | (SEQ ID NO: 269) |
| chr6 | gchr6-3 | GTGTTTGGGCCCTAGAGCGA |
| | | (SEQ ID NO: 270) |
| chr6 | gchr6-4 | CATGTGCCTGGTGCAATGCA |
| | | (SEQ ID NO: 271) |
| chr6 | gchr6-5 | TACAAAGAGGAAGATAAGTG |
| | | (SEQ ID NO: 272) |
| chr6 | gchr6-6 | GTCACAGAATACACCACTAG |
| | | (SEQ ID NO: 273) |
| chr6 | gchr6-7 | GGGTTACCCTGGACATGGAA |
| | | (SEQ ID NO: 274) |
| chr6 | gchr6-8 | CATGGAAGGGTATTCACTCG |
| | | (SEQ ID NO: 275) |
| chr6 | gchr6-9 | AGAGTGGCCTAGACAGGCTG |
| | | (SEQ ID NO: 276) |
| chr6 | gchr6-10 | CATGCTGGACAGCTCGGCAG |
| | | (SEQ ID NO: 277) |
| chr6 | gchr6-11 | AGTGAAAGAAGAGAAAATTC |
| | | (SEQ ID NO: 278) |
| chr6 | gchr6-12 | TGGTAAGTCTAAGAAACCTA |
| | | (SEQ ID NO: 279) |
| chr6 | gchr6-13 | CCCACAGCCTAACCACCCTA |
| | | (SEQ ID NO: 280) |
| chr6 | gchr6-14 | AATATTTCAAAGCCCTAGGG |
| | | (SEQ ID NO: 281) |
| chr6 | gchr6-15 | GCACTCGGAACAGGGTCTGG |
| | | (SEQ ID NO: 282) |
| chr6 | gchr6-16 | AGATAGGAGCTCCAACAGTG |
| | | (SEQ ID NO: 283) |
| chr6 | gchr6-17 | AAGTTAGAGCAGCCAGGAAA |
| | | (SEQ ID NO: 284) |
| chr6 | gchr6-18 | TAGAGCAGCCAGGAAAGGGA |
| | | (SEQ ID NO: 285) |
| chr6 | gchr6-19 | TGAATACCCTTCCATGTCCA |
| | | (SEQ ID NO: 286) |
| chr6 | gchr6-20 | CCTGCATTGCACCAGGCACA |
| | | (SEQ ID NO: 287) |
| chr6 | gchr6-21 | TCTAGGGCCCAAACACACCT |
| | | (SEQ ID NO: 288) |
| chr6 | gchr6-22 | TCCCTCCATCTATCAAAAGG |
| | | (SEQ ID NO: 289) |
| chr10 | gchr10-1 | AGCCCTGAGACAGAAGCAGG |
| | | (SEQ ID NO: 290) |
| chr10 | gchr10-2 | GCCCTGAGACAGAAGCAGGT |
| | | (SEQ ID NO: 291) |
| chr10 | gchr10-3 | AGGAGATGCAGTGATACGCA |
| | | (SEQ ID NO: 292) |
| chr10 | gchr10-4 | ACAATACCAAGGGTATCCGG |
| | | (SEQ ID NO: 293) |
| chr10 | gchr10-5 | TGATAAAGAAAACAAAGTGA |
| | | (SEQ ID NO: 294) |
| chr10 | gchr10-6 | AAAGAAAACAAAGTGAGGGA |
| | | (SEQ ID NO: 295) |
| chr10 | gchr10-7 | GTGGCAAGTGGAGAAATTGA |
| | | (SEQ ID NO: 296) |
| chr10 | gchr10-8 | CAAGTGGAGAAATTGAGGGA |
| | | (SEQ ID NO: 297) |
| chr10 | gchr10-9 | GTGGTGATGATTGCAGCTGG |
| | | (SEQ ID NO: 298) |
| chr11 | gchr11-1 | CTATGTGCCTGACACACAGG |
| | | (SEQ ID NO: 299) |
| chr11 | gchr11-2 | GGGTTGGACCAGGAAAGAGG |
| | | (SEQ ID NO: 300) |
| chr17 | gchr17-1 | GATGCCTGGAAAAGGAAAGA |
| | | (SEQ ID NO: 301) |
| chr17 | gchr17-2 | TAGTATGCACCTGCAAGAGG |
| | | (SEQ ID NO: 302) |
| chr17 | gchr17-3 | TATGCACCTGCAAGAGGCGG |
| | | (SEQ ID NO: 303) |
| chr17 | gchr17-4 | AGGGGAAGAAGAGAAGCAGA |
| | | (SEQ ID NO: 304) |
| chr17 | gchr17-5 | GCTGAATCAAGAGACAAGCG |
| | | (SEQ ID NO: 305) |
| chr17 | gchr17-6 | AAGCAAATAAATCTCCTGGG |
| | | (SEQ ID NO: 306) |
| chr17 | gchr17-7 | AGATGAGTGCTAGAGACTGG |
| | | (SEQ ID NO: 307) |
| chr17 | gchr17-8 | CTGATGGTTGAGCACAGCAG |
| | | (SEQ ID NO: 308) |

In embodiments, gRNAs for targeting human genomic safe harbor sites using any of the gRNA-based targeting elements, e.g., without limitation dCas, to the TTAA site in hROSA26 (e.g., hg38 chr3:9,396,133-9,396,305) are shown in TABLE 3B:

| HROSA26 GUIDE NO. | DNA SEQUENCE | SEQ ID NO: |
|---|---|---|
| GUIDE 44 | AATCGAGAAGCGACTCGACA | 425 |
| GUIDE 45-C | GTCCCTGGGCGTTGCCCTGC | 442 |
| GUIDE 46-C | CCCTGGGCGTTGCCCTGCAG | 443 |
| SPG GUIDE1-C | GAGTGAGCAGCTGTAAGATT | 444 |
| SPG GUIDE2-C | CAGGGGAGTGAGCAGCTGTA | 445 |
| SPG GUIDE3-C | CCTGCAGGGGAGTGAGCAGC | 428 |
| SPG GUIDE4-C | TGCCCTGCAGGGGAGTGAGC | 426 |
| SPG GUIDE5-C | CGTTGCCCTGCAGGGGAGTG | 446 |
| SPG GUIDE6-C | TGGGCGTTGCCCTGCAGGGG | 447 |
| SPG GUIDE7-C | TTGGTCCCTGGGCGTTGCCC | 448 |
| SPG GUIDE8 | AAGAATCCCGCCCATAATCG | 449 |
| SPG GUIDE9 | AATCCCGCCCATAATCGAGA | 450 |
| SPG GUIDE10 | TCCCGCCCATAATCGAGAAG | 451 |
| SPG GUIDE11 | CCCATAATCGAGAAGCGACT | 452 |
| SPG GUIDE12 | GAGAAGCGACTCGACATGGA | 453 |
| SPG GUIDE13 | GAAGCGACTCGACATGGAGG | 427 |
| SPG GUIDE14 | GCGACTCGACATGGAGGCGA | 454 |
| GUIDE N1 | CCGTGGGAAGATAAACTAAT | 455 |
| GUIDE N2 | TCCCCTGCAGGGCAACGCCC | 456 |
| GUIDE N3-C | GTCGAGTCGCTTCTCGATTA | 457 |
| GUIDE 012 | CGACACCAACTCTAGTCCGT | 458 |
| GUIDE 013 | CAGCTGCTCACTCCCCTGCA | 459 |
| GUIDE 014-C | AGTCGCTTCTCGATTATGGG | 460 |

In embodiments, gRNAs for targeting human genomic safe harbor sites using any of the gRNA-based targeting elements, e.g., without limitation dCas, to the AAVS1 (e.g., hg38 chr19:55,112,851-55,113,324) are shown in TABLE 3C:

| AAVS1 GUIDE NO. | DNA SEQUENCE | SEQ ID NO: |
|---|---|---|
| AAV GUIDE 12 | ACCCTTGGAAGGACCTGGCTGGG | 461 |
| AAV GUIDE 13c | TCCGAGCTTGACCCTTGGAA | 462 |
| AAV GUIDE 14 | GGAGCCACGAAAACAGATCCAGG | 463 |
| AAV GUIDE 14c | TGGTTTCCGAGCTTGACCCT | 112 |
| AAV GUIDE 15 | GGAGCCACGAAAACAGATCCAGG | 463 |
| AAV GUIDE 16 | AGATCCAGGGACACGGTGCTAGG | 464 |
| AAV GUIDE 17 | GACACGGTGCTAGGACAGTGGGG | 465 |
| AAV GUIDE 18 | GAAAATGACCCAACAGCCTCTGG | 466 |
| AAV GUIDE 19 | GCCTGGCCGGCCTGACCACTGGG | 467 |
| AAV GUIDE 20 | CTGAGCACTGAAGGCCTGGCCGG | 468 |
| AAV GUIDE 21 | TGGTTTCCACTGAGCACTGAAGG | 469 |
| AAV GUIDE 22 | GGTGCTTTCCTGAGGACCGATAG | 470 |
| AAV GUIDE 23 | GCGCTTCCAGTGCTCAGACTAGG | 471 |
| AAV GUIDE 24 | CAGTGCTCAGACTAGGGAAGAGG | 472 |
| AAV GUIDE 25 | GCCCCTCCTCCTTCAGAGCCAGG | 473 |
| AAV GUIDE 26 | TCCTTCAGAGCCAGGAGTCCTGG | 474 |
| AAV GUIDE 27 | CCAAGGGTCAAGCTCGGAAACCA | 475 |
| AAV GUIDE 28 | CTGCAGAGTATCTGCTGGGGTGG | 476 |
| AAV GUIDE 29 | CGTTCCTGCAGAGTATCTGCTGG | 477 |
| AAV GUIDE 30c | GTGGGGAAAATGACCCAACA | 478 |
| AAV GUIDE 31 | GAAGGCCTGGCCGGCCTGAC | 479 |
| AAV GUIDE 32c | ACTCCTGGCTCTGAAGGAGG | 480 |
| AAV GUIDE 33c | GGGCTGGGGGCCAGGACTCC | 481 |
| AAV GUIDE 34 | GTCCTTCCAAGGGTCAAGCT | 482 |
| AAV GUIDE 35 | TCAAGCTCGGAAACCACCCC | 483 |

In embodiments, gRNAs for targeting human genomic safe harbor sites using any of the gRNA-based targeting elements, e.g., without limitation dCas, to Chromosome 4 (e.g., hg38 chr4:30,793,534-30,875,476 or hg38 chr4:30,793,533-30,793,537 (9677); chr4:30,875,472-30,875,476 (8948)) are shown in TABLE 3D:

| CHR4 GUIDE NO. | DNA SEQUENCE | SEQ ID NO: |
|---|---|---|
| Guide C4-1 | ATTGTCTTCACTAAACCCGTTGG | 484 |
| Guide C4-2 | TAAACCCGTTGGGAATACAATGG | 485 |
| Guide C4-3 | TTGTCTTCACTAAACCCGTTGGG | 486 |
| Guide C4-4 | TGATTCATAGGAGTCTATTAAGG | 487 |
| Guide C4-5 | TTACATATGCTTCGAGTTTGTGG | 488 |
| Example 1 | ACTCTTAAGGTAGGACTAATTGG | 489 |
| Guide C4-6 | | |
| Guide C4-7 | TATGTGTGCAATAGCGTTAAAGG | 490 |
| Guide C4-8 | CGTTGGGAATACAATGGCTTAGG | 491 |
| Guide C4-9 | TCACAATGGAACTCTGCCTTTGG | 492 |
| Guide C4-10 | GACCACAAATCAATGCCCAAAGG | 493 |
| Guide C4-11 | CTAAGCCATTGTATTCCCAACGG | 494 |
| Guide C4-12 | AGCATTCTGGAGTGTCACAATGG | 495 |
| Guide C4-13 | CAATAGCCCACTTTAATACTAGG | 496 |
| Guide C4-14 | CTTTATCCAAGTGAATCCITTGG | 497 |
| Guide C4-15 | GGCATTGATTTGTGGTCATTTGG | 498 |
| Guide C4-16 | TAAGCCATTGTATTCCCAACGGG | 499 |
| Guide C4-17 | AATACAATCACTCTTAAGGTAGG | 500 |
| Guide C4-18 | GAAGTACCTTTCACTATTTTGGG | 501 |
| Guide C4-19 | CAAGCAACAAATGACTTCTAAGG | 502 |
| Guide C4-20 | TTTGAATACAATCACTCTTAAGG | 503 |
| Guide C4A1 | ACAAACGGACTACGTAAACTTGG | 504 |
| Guide C4A2 | ACAAGATGTGAACACGACGATGG | 505 |
| Guide C4A3 | GTTGCACCGTTGATTCCTTCAGG | 506 |
| Guide C4A4 | AGTAATATTGAATTAGGGCGTGG | 507 |
| Guide C4A5 | CCTGATGTTGGCTCGACATTAGG | 508 |
| Guide C4A6 | CTTTGTTGGGTCTTAGCTTAAGG | 509 |
| Guide C4A7 | TCGGAACAGCTCCTTCCTGAAGG | 510 |
| Guide C4A8 | AGTAGTTTCTGAGGTCATGTTGG | 511 |
| Guide C4A9 | CTTGAAAATACGATGATGTGAGG | 512 |
| Guide C4A10 | GCATTAATCTAGAGAGAGGGAGG | 513 |
| Guide C4A11 | GGGTCATGTTAGAATTCATGTGG | 514 |
| Guide C4A12 | TGATGCATTAATCTAGAGAGAGG | 515 |
| Guide C4A13 | ACATCATCGTATTTTCAAGTTGG | 516 |
| Guide C4A14 | CTAGCTGACAAACATGTGAGTGG | 517 |
| Guide C4A15 | AACATGACCCAAGTGAGTCCAGG | 518 |
| Guide C4A16 | GATTCCGTATTTGCTTTGTTGGG | 519 |
| Guide C4A17 | TACGATGATGTGAGGAAATAAGG | 520 |
| Guide C4A18 | GTAATATGTCTAAGTACTGATGG | 521 |
| Guide C4A19 | GTAAAGTGAGCTGGTTCATTAGG | 522 |
| Guide C4A20 | ACTAGAGTCCTTAAGAAGGGGGG | 523 |

In embodiments, gRNAs for targeting human genomic safe harbor sites using any of the gRNA-based targeting elements, e.g., without limitation dCas, to Chromosome 22 (e.g., hg38 chr22:35,370,000-35,380,000 or hg38 chr22:35,373,912-35,373,916 (861); chr22:35,377,843-35,377,847 (1153)) are shown in TABLE 3E:

| | | SEQ |
|---|---|---|
| CHR22 | | ID |
| GUIDE NO. | DNA SEQUENCE | NO: |
| Guide C22-1 | ATAACACGTGAGCCGTCCTAAGG | 524 |
| Guide C22-2 | GGAAGACTTTTCTCTATACGAGG | 525 |
| Guide C22-3 | GCATTCCTTTCATCCATGGCAGG | 526 |
| Guide C22-4 | GACATATGGTTATAAAAATCAGG | 527 |
| Guide C22-5 | GGAGTGCAGTCCCTGACATATGG | 528 |
| Guide C22-6 | GTGGGTTAGGGTGGTTAACTGGG | 529 |
| Guide C22-7 | AGGTGCAAAAAGGTTGCTGTGGG | 530 |
| Guide C22-8 | CGTGACAAGGCAAAGTGGCGTGG | 531 |
| Guide C22-9 | GAAGGACTGCCCCTGACGTCAGG | 532 |
| Guide C22-10 | CTGCCCCTGACGTCAGGAGTTGG | 533 |
| Guide C22-11 | TGTGGGTTAGGGTGGTTAACTGG | 534 |
| Guide C22-12 | ACCCTTTTAGAGTTTTCTGCTGG | 535 |
| Guide C22-13 | AACTTCCTGCCATGGATGAAAGG | 536 |
| Guide C22-14 | GCAAAAAGGTTGCTGTGGGTTGG | 537 |
| Guide C22-15 | AATTTGGGGGTAGATAGGCATGG | 538 |
| Guide C22-16 | AGAAAACTCTAAAAGGGTATAGG | 539 |
| Guide C22-17 | ATTAGCATTCCTTTCATCCATGG | 540 |
| Guide C22-18 | CCCAGCAGAAAACTCTAAAAGGG | 541 |
| Guide C22-19 | CAGGTGCAAAAAGGTTGCTGTGG | 542 |
| Guide C22-20 | GCAAGAGATGAAATTCCATATGG | 543 |
| Guide C22A1 | GGGCTGTTCTAACGAAGTCTGGG | 544 |
| Guide C22A2 | TGTCCATTCAGCGACCCTAGAGG | 545 |
| Guide C22A3 | GGCTGTTCTAACGAAGTCTGGGG | 546 |
| Guide C22A4 | GTCCATTCAGCGACCCTAGAGGG | 547 |
| Guide C22A5 | GGGGCTGTTCTAACGAAGTCTGG | 548 |
| Guide C22A6 | GGCTGAATCAGCATGCGAAAGGG | 549 |
| Guide C22A7 | TTCCAATGGGGGGCATAGCCTGG | 550 |
| Guide C22A8 | TACCCTCTAGGGTCGCTGAATGG | 551 |
| Guide C22A9 | ATCCTCTTGGGCCTTATAAGAGG | 552 |
| Guide C22A10 | GGCCAGGCTATGCCCCCCATTGG | 553 |
| Guide C22A11 | CTAGAGGACCAGAACAACTCTGG | 554 |
| Guide C22A12 | TCCCTCTTATAAGGCCCAAGAGG | 555 |
| Guide C22A13 | AGGCTGAATCAGCATGCGAAAGG | 556 |
| Guide C22A14 | GGACCAGAACAACTCTGGCCTGG | 557 |
| Guide C22A15 | GGGCTTTTATTTGGCCCAGCAGG | 558 |
| Guide C22A16 | GTCGCTGAATGGACAGACTCTGG | 559 |
| Guide C22A17 | CTCATGAGTTTTACCCTCTAGGG | 560 |
| Guide C22A18 | TCCTCTTGGGCCTTATAAGAGGG | 561 |
| Guide C22A19 | TCTTGGGCCTTATAAGAGGGAGG | 562 |
| Guide C22A20 | TAGAACAGCCCCCCACACAGTGG | 563 |

In embodiments, gRNAs for targeting human genomic safe harbor sites using any of the gRNA-based targeting elements, e.g., without limitation dCas, to Chromosome X (e.g., hg38 chrX:134,419,661-134,541,172 or hg38 chrX:134,476,304-134,476,307 (85); chrX:134,476,337-134,476,340 (51)) are shown in TABLE 3F:

| CHRX GUIDE NO. | DNA SEQUENCE | SEQ ID NO: |
|---|---|---|
| Guide CX-1 | GTTACGTTATGACTAATCTTTGG | 564 |
| Guide CX-2 | TACGTTATGACTAATCTTTGGGG | 565 |
| Guide CX-3 | GGAAGTAGTGTTATGATGTATGG | 566 |
| Guide CX-4 | GTTATGATGTATGGGCATAAAGG | 567 |
| Guide CX-5 | GAAGTAGTGTTATGATGTATGGG | 568 |
| Guide CX-6 | ATAGCTGCTGGCAGTATAACTGG | 569 |
| Guide CX-7 | GCATCACAACATTGACACTGTGG | 570 |
| Guide CX-8 | AAGGCGAGTTTCTACAAAGATGG | 571 |
| Guide CX-9 | TTACGTTATGACTAATCTTTGGG | 572 |
| Guide CX-10 | CAAGACTGATTAAGACTGATGGG | 573 |
| Guide CX-11 | AGCAGCAATGTATTAAAGGCTGG | 574 |
| Guide CX-12 | CTACAGGATTGATGTAAACATGG | 575 |
| Guide CX-13 | TGGGCATAAAGGGTTTTAATGGG | 576 |
| Guide CX-14 | ACATCAATCCTGTAGGTGATTGG | 577 |
| Guide CX-15 | ATTCTAGTCATTATAGCTGCTGG | 578 |
| Guide CX-16 | CATCAATCCTGTAGGTGATTGGG | 579 |
| Guide CX-17 | GTTATAAGATCAATTCTGAGTGG | 580 |
| Guide CX-18 | GGCAGACTGTGGATCAAAAGTGG | 581 |
| Guide CX-19 | ATGGCTGCCCAATCACCTACAGG | 582 |
| Guide CX-20 | TCAAAGCATGTACTTAGAGTTGG | 583 |

In embodiments, the gRNA comprises one or more of the sequences outlined herein or a variant sequence having at least about 10 mutations, or at least about 9 mutations, or at least about 8 mutations, or at least about 7 mutations, or at least about 6 mutations, or at least about 5 mutations, or at least about 4 mutations, or at least about 3 mutations, or at least about 2 mutations, or at least about 1 mutation.

In embodiments, a Cas-based targeting element comprises Cas12 or a variant thereof, e.g., without limitation, Cas12a (e.g., dCas12a), or Cas12j (e.g., dCas12j), or Cas12k (e.g., dCas12k). In embodiments, the targeting element comprises a Cas12 enzyme guide RNA complex. In embodiments, comprises a nuclease-deficient dCas12 guide RNA complex, optionally dCas12j guide RNA complex or dCas12a guide RNA complex.

In embodiments, the targeting element is selected from a zinc finger (ZF), transcription activator-like effector (TALE), meganuclease, and clustered regularly interspaced short palindromic repeat (CRISPR)-associated protein, any of which are, in embodiments, catalytically inactive. In embodiments, the CRISPR-associated protein is selected from Cas9, CasX, CasY, Cas12a (Cpf1), and gRNA complexes thereof. In embodiments, the CRISPR-associated protein is selected from Cas9, xCas9, Cas 6, Cas7, Cas8, Cas12a (Cpf1), Cas13a, Cas14, CasX, CasY, a Class 1 Cas protein, a Class 2 Cas protein, MAD7, MG1 nuclease, MG2 nuclease, MG3 nuclease, or catalytically inactive forms thereof, and gRNA complexes thereof.

In embodiments, the mobile element enzyme is capable of inserting a donor DNA at a TA dinucleotide site or a TTAA tetranucleotide site in a genomic safe harbor site (GSHS) of a nucleic acid molecule. The mobile element enzyme is suitable for causing insertion of the donor DNA in a GSHS when contacted with a biological cell.

In embodiments, the targeting element is suitable for directing the mobile element enzyme to the GSHS sequence.

In embodiments, the targeting element comprises transcription activator-like effector (TALE) DNA binding domain (DBD). The TALE DBD comprises one or more repeat sequences. For example, in embodiments, the TALE DBD comprises about 14, or about 15, or about, 16, or about 17, or about 18, or about 18.5 repeat sequences. In embodiments, the TALE DBD repeat sequences comprise 33 or 34 amino acids.

In embodiments, the one or more of the TALE DBD repeat sequences comprise a repeat variable di-residue (RVD) at residue 12 or 13 of the 33 or 34 amino acids.

In embodiments, the targeting element (e.g., TALE or Cas (e.g., Cas9 or Cas12, or variants thereof) DBDs cause the mammalian mobile element enzyme to bind specifically to human GSHS. In embodiments, the TALEs or Cas DBDs sequester the mobile element enzyme to GSHS and promote transposition to nearby TA dinucleotide or a TTAA tetranucleotide sites which can be located in proximity to the repeat variable di-residues (RVD) TALE or gRNA nucleotide sequences. The GSHS regions are located in open chromatin sites that are susceptible to mobile element enzyme activity. Accordingly, the mammalian mobile element enzyme does not only operate based on its ability to recognize TA or TTAA sites, but it also directs a donor DNA comprising a chimeric CAR or chimeric CAAR to specific locations in proximity to a TALE or Cas DBD. The chimeric mobile element enzyme in accordance with embodiments of the present disclosure has negligible risk of genotoxicity and exhibits superior features as compared to existing gene therapies.

In embodiments, a chimeric mobile element enzyme is mutated to be characterized by reduced or inhibited binding of off-target sequences and consequently reliant on a DBD fused thereto, such as a TALE or Cas DBD, for transposition.

The described cells, compositions, and methods allow reducing vector and transgene insertions that increase a mutagenic risk. The described cells and methods make use of a gene transfer system that reduces genotoxicity compared to viral- and nuclease-mediated gene therapies. The dual system is designed to avoid the persistence of an active mobile element enzyme and efficiently transfect human cell without significant cytotoxicity.

In embodiments, TALE or Cas DBDs are customizable, such as a TALE or Cas DBDs is selected for targeting a specific genomic location. In embodiments, the genomic location is in proximity to a TA dinucleotide site or a TTAA (SEQ ID NO: 440) tetranucleotide site.

Embodiments of the present disclosure make use of the ability of TALE or Cas or dCas9/gRNA DBDs to target specific sites in a host genome. The DNA targeting ability of a TALE or Cas DBD or dCas9/gRNA DBD is provided by TALE repeat sequences (e.g., modular arrays) or gRNA which are linked together to recognize flanking DNA sequences. Each TALE or gRNA can recognize certain base pair(s) or residue(s).

TALE nucleases (TALENs) are a known tool for genome editing and introducing targeted double-stranded breaks. TALENs comprise endonucleases, such as FokI nuclease domain, fused to a customizable DBD. This DBD is composed of highly conserved repeats from TALEs, which are proteins secreted by Xanthomonas bacteria to alter transcription of genes in host plant cells. The DBD includes a repeated highly conserved 33-34 amino acid sequence with divergent 12th and 13th amino acids. These two positions, referred to as the RVD, are highly variable and show a strong correlation with specific base pair or nucleotide recognition. This straightforward relationship between amino acid sequence and DNA recognition has allowed for the engineering of specific DBDs by selecting a combination of repeat segments containing the appropriate RVDs. Boch et al. Nature Biotechnology. 2011; 29 (2): 135-6.

Accordingly, TALENs can be readily designed using a “protein-DNA code” that relates modular DNA-binding TALE repeat domains to individual bases in a target-binding site. See Joung et al. Nat Rev Mol Cell Biol. 2013; 14(1):49-55. doi:10.1038/nrm3486. The following table, for example, shows such code:

| RVD | Nucleotide | RVD | Nucleotide |
|---|---|---|---|
| HD | C | NI | A |
| NH | G | NN | G, A |
| NK | G | NS | G, C, A |
| NG | T, mC | | |

It has been demonstrated that TALENs can be used to target essentially any DNA sequence of interest in human cell. Miller et al. Nat Biotechnol. 2011; 29:143-148. Guidelines for selection of potential target sites and for use of particular TALE repeat domains (harboring NH residues at the hypervariable positions) for recognition of G bases have been proposed. See Streubel et al. Nat Biotechnol. 2012; 30:593-595.

Accordingly, in embodiments, the TALE DBD comprises one or more repeat sequences. In embodiments, the TALE DBD comprises about 15, or about, 16, or about 17, or about 18, or about 18.5 repeat sequences. In embodiments, the TALE DBD repeat sequences comprise 33 or 34 amino acids.

In embodiments, the one or more of the TALE DBD repeat sequences comprise an RVD at residue 12 or 13 of the 33 or 34 amino acids. The RVD can recognize certain base pair(s) or residue(s). In embodiments, the RVD recognizes one base pair in the nucleic acid molecule. In embodiments, the RVD recognizes a C residue in the nucleic acid molecule and is selected from HD, N(gap), HA, ND, and HI. In embodiments, the RVD recognizes a G residue in the nucleic acid molecule and is selected from NN, NH, NK, HN, and NA. In embodiments, the RVD recognizes an A residue in the nucleic acid molecule and is selected from NI and NS. In embodiments, the RVD recognizes a T residue in the nucleic acid molecule and is selected from NG, HG, H(gap), and IG.

In embodiments, the GSHS is in an open chromatin location in a chromosome. In embodiments, the GSHS is selected from adeno-associated virus site 1 (AAVS1), chemokine (C—C motif) receptor 5 (CCR5) gene, HIV-1 coreceptor; and human Rosa26 locus. In embodiments, the GSHS is located on human chromosome 2, 4, 6, 10, 11, 17, 22, or X.

In embodiments, the GSHS is selected from TALC1, TALC2, TALC3, TALC4, TALC5, TALC7, TALC8, AVS1, AVS2, AVS3, ROSA1, ROSA2, TALER1, TALER2, TALER3, TALER4, TALER5, SHCHR2-1, SHCHR2-2, SHCHR2-3, SHCHR2-4, SHCHR4-1, SHCHR4-2, SHCHR4-3, SHCHR6-1, SHCHR6-2, SHCHR6-3, SHCHR6-4, SHCHR10-1, SHCHR10-2, SHCHR10-3, SHCHR10-4, SHCHR10-5, SHCHR11-1, SHCHR11-2, SHCHR11-3, SHCHR17-1, SHCHR17-2, SHCHR17-3, and SHCHR17-4.

In embodiments, the GSHS comprises one or more of TGGCCGGCCTGACCACTGG (SEQ ID NO: 23), TGAAGGCCTGGCCGGCCTG (SEQ ID NO: 24), TGAGCACTGAAGGCCTGGC (SEQ ID NO: 25), TCCACTGAGCACTGAAGGC (SEQ ID NO: 26), TGGTTTCCACTGAGCACTG (SEQ ID NO: 27), TGGGGAAAATGACCCAACA (SEQ ID NO: 28), TAGGACAGTGGGGAAAATG (SEQ ID NO: 29), TCCAGGGACACGGTGCTAG (SEQ ID NO: 30), TCAGAGCCAGGAGTCCTGG (SEQ ID NO: 31), TCCTTCAGAGCCAGGAGTC (SEQ ID NO: 32), TCCTCCTTCAGAGCCAGGA (SEQ ID NO: 33), TCCAGCCCCTCCTCCTTCA (SEQ ID NO: 34), TCCGAGCTTGACCCTTGGA (SEQ ID NO: 35), TGGTTTCCGAGCTTGACCC (SEQ ID NO: 36), TGGGGTGGTTTCCGAGCTT (SEQ ID NO: 37), TCTGCTGGGGTGGTTTCCG (SEQ ID NO: 38), TGCAGAGTATCTGCTGGGG (SEQ ID NO: 39), CCAATCCCCTCAGT (SEQ ID NO: 40), CAGTGCTCAGTGGAA (SEQ ID NO: 41), GAAACATCCGGCGACTCA (SEQ ID NO: 42), TCGCCCCTCAAATCTTACA (SEQ ID NO: 43), TCAAATCTTACAGCTGCTC (SEQ ID NO: 44), TCTTACAGCTGCTCACTCC (SEQ ID NO: 45), TACAGCTGCTCACTCCCCT (SEQ ID NO: 46), TGCTCACTCCCCTGCAGGG (SEQ ID NO: 47), TCCCCTGCAGGGCAACGCC (SEQ ID NO: 48), TGCAGGGCAACGCCCAGGG (SEQ ID NO: 49), TCTCGATTATGGGCGGGAT (SEQ ID NO: 50), TCGCTTCTCGATTATGGGC (SEQ ID NO: 51), TGTCGAGTCGCTTCTCGAT (SEQ ID NO: 52), TCCATGTCGAGTCGCTTCT (SEQ ID NO: 53), TCGCCTCCATGTCGAGTCG (SEQ ID NO: 54), TCGTCATCGCCTCCATGTC (SEQ ID NO: 55), TGATCTCGTCATCGCCTCC (SEQ ID NO: 56), GCTTCAGCTTCCTA (SEQ ID NO: 57), CTGTGATCATGCCA (SEQ ID NO: 58), ACAGTGGTACACACCT (SEQ ID NO: 59), CCACCCCCCACTAAG (SEQ ID NO: 60), CATTGGCCGGGCAC (SEQ ID NO: 61), GCTTGAACCCAGGAGA (SEQ ID NO: 62), ACACCCGATCCACTGGG (SEQ ID NO: 63), GCTGCATCAACCCC (SEQ ID NO: 64), GCCACAAACAGAAATA (SEQ ID NO: 65), GGTGGCTCATGCCTG (SEQ ID NO: 66), GATTTGCACAGCTCAT (SEQ ID NO: 67), AAGCTCTGAGGAGCA (SEQ ID NO: 68), CCCTAGCTGTCCC (SEQ ID NO: 69), GCCTAGCATGCTAG (SEQ ID NO: 70), ATGGGCTTCACGGAT (SEQ ID NO: 71), GAAACTATGCCTGC (SEQ ID NO: 72), GCACCATTGCTCCC (SEQ ID NO: 73), GACATGCAACTCAG (SEQ ID NO: 74), ACACCACTAGGGGT (SEQ ID NO: 75), GTCTGCTAGACAGG (SEQ ID NO: 76), GGCCTAGACAGGCTG (SEQ ID NO: 77), GAGGCATTCTTATCG (SEQ ID NO: 78), GCCTGGAAACGTTCC (SEQ ID NO: 79), GTGCTCTGACAATA (SEQ ID NO: 80), GTTTTGCAGCCTCC (SEQ ID NO: 81), ACAGCTGTGGAACGT (SEQ ID NO: 82), GGCTCTCTTCCTCCT (SEQ ID NO: 83), CTATCCCAAAACTCT (SEQ ID NO: 84), GAAAAACTATGTAT (SEQ ID NO: 85), AGGCAGGCTGGTTGA (SEQ ID NO: 86), CAATACAACCACGC (SEQ ID NO: 87), ATGACGGACTCAACT (SEQ ID NO: 88), CACAACATTTGTAA (SEQ ID NO: 89), and ATTTCCAGTGCACA (SEQ ID NO: 90).

In embodiments, the TALE DBD binds to one of TGGCCGGCCTGACCACTGG (SEQ ID NO: 23), TGAAGGCCTGGCCGGCCTG (SEQ ID NO: 24), TGAGCACTGAAGGCCTGGC (SEQ ID NO: 25), TCCACTGAGCACTGAAGGC (SEQ ID NO: 26), TGGTTTCCACTGAGCACTG (SEQ ID NO: 27), TGGGGAAAATGACCCAACA (SEQ ID NO: 28), TAGGACAGTGGGGAAAATG (SEQ ID NO: 29), TCCAGGGACACGGTGCTAG (SEQ ID NO: 30), TCAGAGCCAGGAGTCCTGG (SEQ ID NO: 31), TCCTTCAGAGCCAGGAGTC (SEQ ID NO: 32), TCCTCCTTCAGAGCCAGGA (SEQ ID NO: 33), TCCAGCCCCTCCTCCTTCA (SEQ ID NO: 34), TCCGAGCTTGACCCTTGGA (SEQ ID NO: 35), TGGTTTCCGAGCTTGACCC (SEQ ID NO: 36), TGGGGTGGTTTCCGAGCTT (SEQ ID NO: 37), TCTGCTGGGGTGGTTTCCG (SEQ ID NO: 38), TGCAGAGTATCTGCTGGGG (SEQ ID NO: 39), CCAATCCCCTCAGT (SEQ ID NO: 40), CAGTGCTCAGTGGAA (SEQ ID NO: 41), GAAACATCCGGCGACTCA (SEQ ID NO: 42), TCGCCCCTCAAATCTTACA (SEQ ID NO: 43), TCAAATCTTACAGCTGCTC (SEQ ID NO: 44), TCTTACAGCTGCTCACTCC (SEQ ID NO: 45), TACAGCTGCTCACTCCCCT (SEQ ID NO: 46), TGCTCACTCCCCTGCAGGG (SEQ ID NO: 47), TCCCCTGCAGGGCAACGCC (SEQ ID NO: 48), TGCAGGGCAACGCCCAGGG (SEQ ID NO: 49), TCTCGATTATGGGCGGGAT (SEQ ID NO: 50), TCGCTTCTCGATTATGGGC (SEQ ID NO: 51), TGTCGAGTCGCTTCTCGAT (SEQ ID NO: 52), TCCATGTCGAGTCGCTTCT (SEQ ID NO: 53), TCGCCTCCATGTCGAGTCG (SEQ ID NO: 54), TCGTCATCGCCTCCATGTC (SEQ ID NO: 55), TGATCTCGTCATCGCCTCC (SEQ ID NO: 56), GCTTCAGCTTCCTA (SEQ ID NO: 57), CTGTGATCATGCCA (SEQ ID NO: 58), ACAGTGGTACACACCT (SEQ ID NO: 59), CCACCCCCCACTAAG (SEQ ID NO: 60), CATTGGCCGGGCAC (SEQ ID NO: 61), GCTTGAACCCAGGAGA (SEQ ID NO: 62), ACACCCGATCCACTGGG (SEQ ID NO: 63), GCTGCATCAACCCC (SEQ ID NO: 64), GCCACAAACAGAAATA (SEQ ID NO: 65), GGTGGCTCATGCCTG (SEQ ID NO: 66), GATTTGCACAGCTCAT (SEQ ID NO: 67), AAGCTCTGAGGAGCA (SEQ ID NO: 68), CCCTAGCTGTCCC (SEQ ID NO: 69), GCCTAGCATGCTAG (SEQ ID NO: 70), ATGGGCTTCACGGAT (SEQ ID NO: 71), GAAACTATGCCTGC (SEQ ID NO: 72), GCACCATTGCTCCC (SEQ ID NO: 73), GACATGCAACTCAG (SEQ ID NO: 74), ACACCACTAGGGGT (SEQ ID NO: 75), GTCTGCTAGACAGG (SEQ ID NO: 76), GGCCTAGACAGGCTG (SEQ ID NO: 77), GAGGCATTCTTATCG (SEQ ID NO: 78), GCCTGGAAACGTTCC (SEQ ID NO: 79), GTGCTCTGACAATA (SEQ ID NO: 80), GTTTTGCAGCCTCC (SEQ ID NO: 81), ACAGCTGTGGAACGT (SEQ ID NO: 82), GGCTCTCTTCCTCCT (SEQ ID NO: 83), CTATCCCAAAACTCT (SEQ ID NO: 84), GAAAAACTATGTAT (SEQ ID NO: 85), AGGCAGGCTGGTTGA (SEQ ID NO: 86), CAATACAACCACGC (SEQ ID NO: 87), ATGACGGACTCAACT (SEQ ID NO: 88), CACAACATTTGTAA (SEQ ID NO: 89), and ATTTCCAGTGCACA (SEQ ID NO: 90).

In embodiments, the TALE DBD comprises one or more of:

NH NH HD HD NH NH HD HD NG NH NI HD HD NI HD NG NH NH,
NH NI NI NH NH HD HD NG NH NH HD HD NH NH HD HD NG NH,
NH NI NH HD NI HD NG NH NI NI NH NH HD HD NG NH NH HD,
HD HD NI HD NG NH NI NH HD NI HD NG NH NI NI NH NH HD,
NH NH NG NG NG HD HD NI HD NG NH NI NH HD NI HD NG NH,
NH NH NH NH NI NI NI NI NG NH NI HD HD HD NI NI HD NI,
NI NH NH NI HD NI NH NG NH NH NH NH NI NI NI NING NH,
HD HD NI NH NH NH NI HD NI HD NH NH NG NH HD NG NI NH,
HD NI NH NI NH HD HD NI NH NH NI NH NG HD HD NG NH NH,
HD HD NG NG HD NI NH NI NH HD HD NI NH NH NI NH NG HD,
HD HD NG HD HD NG NG HD NI NH NI NH HD HD NI NH NH NI,
HD HD NI NH HD HD HD HD NG HD HD NG HD HD NG NG HD NI,
HD HD NH NI NH HD NG NG NH NI HD HD HD NG NG NH NH NI,
NH NH NG NG NG HD HD NH NI NH HD NG NG NH NI HD HD HD,
NH NH NH NH NG NH NH NG NG NG HD HD NH NI NH HD NG NG,
HD NG NH HD NG NH NH NH NH NG NH NH NG NG NG HD HD NH,
NH HD NI NH NI NH NG NI NG HD NG NH HD NG NH NH NH NH,
HD HD NI NI NG HD HD HD HD NG HD NI NH NG,
HD NI NH NG NH HD NG HD NI NH NG NH NH NI NI,
NH NI NI NI HD NI NG HD HD NH NH HD NH NI HD NG HD NI,
HD NH HD HD HD HD NG HD NI NI NI NG HD NG NG NI HD NI,
HD NI NI NI NG HD NG NG NI HD NI NH HD NG NH HD NG HD,
HD NG NG NI HD NI NH HD NG NH HD NG HD NI HD NG HD HD,
NI HD NI NH HD NG NH HD NG HD NI HD NG HD HD HD HD NG,
NH HD NG HD NI HD NG HD HD HD HD NG NH HD NI NH NH NH,
HD HD HD HD NG NH HD NI NH NH NH HD NI NI HD NH HD HD,
NH HD NI NH NH NH HD NI NI HD NH HD HD HD NI NH NH NH,
HD NG HD NH NI NG NG NING NH NH NH HD NH NH NH NING,
HD NH HD NG NG HD NG HD NH NI NG NG NING NH NH NH HD,
NH NG HD NH NI NH NG HD NH HD NG NG HD NG HD NH NI NG,
HD HD NI NG NH NG HD NH NI NH NG HD NH HD NG NG HD NG,
HD NH HD HD NG HD HD NI NG NH NG HD NH NI NH NG HD NH,
HD NH NG HD NI NG HD NH HD HD NG HD HD NI NG NH NG HD,
NH NI NG HD NG HD NH NG HD NI NG HD NH HD HD NG HD HD,
NH HD NG NG HD NI NH HD NG NG HD HD NG NI,
HD NG NK NG NH NI NG HD NI NG NH HD HD NI,
NI HD NI NN NG NN NN NG NI HD NI HD NI HD HD NG,
HD HD NI HD HD HD HD HD HD NI HD NG NI NI NN,
HD NI NG NG NN NN HD HD NN NN NN HD NI HD,
NN HD NG NG NN NI NI HD HD HD NI NN NN NI NN NI,
NI HD NI HD HD HD NN NI NG HD HD NI HD NG NN NN NN,
NN HD NG NN HD NI NG HD NI NI HD HD HD HD,
NN NN HD NI HD NN NI NI NI HD NI HD HD HD NG HD HD,
NN NN NG NN NN HD NG HD NING NN HD HD NG NN,
NN NI NG NG NG NN HD NI HD NI NN HD NG HD NI NG,
NI NI NH HD NG HD NG NH NI NH NH NI NH HD,
HD HD HD NG NI NK HD NG NH NG HD HD HD HD,
NH HD HD NG NI NH HD NI NG NH HD NG NI NH,
NI NG NH NH NH HD NG NG HD NI HD NH NH NI NG,
NH NI NI NI HD NG NING NH HD HD NG NH HD,
NH HD NI HD HD NI NG NG NH HD NG HD HD HD,
NH NI HD NI NG NH HD NI NI HD NG HD NI NH,
NI HD NI HD HD NI HD NG NI NH NH NH NH NG,
NH NG HD NG NH HD NG NI NH NI HD NI NH NH,
NH NH HD HD NG NI NH NI HD NI NH NH HD NG NH,
NH NI NH NH HD NI NG NG HD NG NG NI NG HD NH,
NN HD HD NG NN NN NI NI NI HD NN NG NG HD HD,
NN NG NN HD NG HD NG NN NI HD NI NI NG NI,
NN NG NG NG NG NN HD NI NN HD HD NG HD HD,
NI HD NI NN HD NG NN NG NN NN NI NI HD NN NG,
HD NI NI NN NI HD HD NN NI NN HD NI HD NG NN HD NG NN,
HD NG NI NG HD HD HD NI NI NI NI HD NG HD NG,
NH NI NI NI NI NI HD NG NING NH NG NI NG,
NI NH NH HD NI NH NH HD NG NH NH NG NG NH NI,
HD NI NI NG NI HD NI NI HD HD NI HD NN HD,
NI NG NN NI HD NN NN NI HD NG HD NI NI HD NG,
HD NI HD NI NI HD NI NG NG NG NN NG NI NI,
and
NI NG NG NG HD HD NI NN NG NN HD NI HD NI.

In embodiments, the TALE DBD comprises one or more of the sequences outlined herein or a variant sequence having at least about 90%, or at least about 93%, or at least about 95%, or at least about 97%, or at least about 98%, or at least about 99% identity thereto, or at least about 10 mutations, or at least about 9 mutations, or at least about 8 mutations, or at least about 7 mutations, or at least about 6 mutations, or at least about 5 mutations, or at least about 4 mutations, or at least about 3 mutations, or at least about 2 mutations, or at least about 1 mutation.

In embodiments, the GSHS and the TALE DBD sequences are selected from:

(SEQ ID NO: 23)
TGGCCGGCCTGACCACTGG
and
NH NH HD HD NH NH HD HD NG NH NI HD HD NI HD NG NH
NH;
(SEQ ID NO: 24)
TGAAGGCCTGGCCGGCCTG
and
NH NI NI NH NH HD HD NG NH NH HD HD NH NH HD HD NG
NH;
(SEQ ID NO: 25)
TGAGCACTGAAGGCCTGGC
and
NH NI NH HD NI HD NG NH NI NI NH NH HD HD NG NH NH
HD;
(SEQ ID NO: 26)
TCCACTGAGCACTGAAGGC
and
HD HD NI HD NG NH NI NH HD NI HD NG NH NI NI NH NH
HD;
(SEQ ID NO: 27)
TGGTTTCCACTGAGCACTG
and
NH NH NG NG NG HD HD NI HD NG NH NI NH HD NI HD NG
NH;
(SEQ ID NO: 28)
TGGGGAAAATGACCCAACA
and
NH NH NH NH NI NI NI NING NH NI HD HD HD NI NI HD
NI;
(SEQ ID NO: 29)
TAGGACAGTGGGGAAAATG
and
NI NH NH NI HD NI NH NG NH NH NH NH NI NI NI NI NG
NH;
(SEQ ID NO: 30)
TCCAGGGACACGGTGCTAG
and
HD HD NI NH NH NH NI HD NI HD NH NH NG NH HD NG NI
NH;
(SEQ ID NO: 31)
TCAGAGCCAGGAGTCCTGG
and
HD NI NH NI NH HD HD NI NH NH NI NH NG HD HD NG NH
NH;
(SEQ ID NO: 32)
TCCTTCAGAGCCAGGAGTC
and
HD HD NG NG HD NI NH NI NH HD HD NI NH NH NI NH NG
HD;
(SEQ ID NO: 33)
TCCTCCTTCAGAGCCAGGA
and
HD HD NG HD HD NG NG HD NI NH NI NH HD HD NI NH NH
NI;
(SEQ ID NO: 34)
TCCAGCCCCTCCTCCTTCA
and
HD HD NI NH HD HD HD HD NG HD HD NG HD HD NG NG HD
NI;
(SEQ ID NO: 35)
TCCGAGCTTGACCCTTGGA
and
HD HD NH NI NH HD NG NG NH NI HD HD HD NG NG NH NH
NI;
(SEQ ID NO: 36)
TGGTTTCCGAGCTTGACCC
and
NH NH NG NG NG HD HD NH NI NH HD NG NG NH NI HD HD
HD;
(SEQ ID NO: 37)
TGGGGTGGTTTCCGAGCTT
and
NH NH NH NH NG NH NH NG NG NG HD HD NH NI
NH HD NG NG;
(SEQ ID NO: 38)
TCTGCTGGGGTGGTTTCCG
and
HD NG NH HD NG NH NH NH NH NG NH NH NG NG NG HD
HD NH;
(SEQ ID NO: 39)
TGCAGAGTATCTGCTGGGG
and
NH HD NI NH NI NH NG NI NG HD NG NH HD NG NH NH NH
NH;
(SEQ ID NO: 40)
CCAATCCCCTCAGT
and
HD HD NI NI NG HD HD HD HD NG HD NI NH
NG;
(SEQ ID NO: 41)
CAGTGCTCAGTGGAA
and
HD NI NH NG NH HD NG HD NI NH NG NH NH NI
NI;
(SEQ ID NO: 42)
GAAACATCCGGCGACTCA
and
NH NI NI NI HD NI NG HD HD NH NH HD NH NI HD NG HD
NI;
(SEQ ID NO: 43)
TCGCCCCTCAAATCTTACA
and
HD NH HD HD HD HD NG HD NI NI NI NG HD NG NG NI HD
NI;
(SEQ ID NO: 44)
TCAAATCTTACAGCTGCTC
and
HD NI NI NI NG HD NG NG NI HD NI NH HD NG NH HD NG
HD;
(SEQ ID NO: 45)
TCTTACAGCTGCTCACTCC
and
HD NG NG NI HD NI NH HD NG NH HD NG HD NI HD NG HD
HD;
(SEQ ID NO: 46)
TACAGCTGCTCACTCCCCT
and
NI HD NI NH HD NG NH HD NG HD NI HD NG HD HD HD HD
NG;
(SEQ ID NO: 47)
TGCTCACTCCCCTGCAGGG
and
NH HD NG HD NI HD NG HD HD HD HD NG NH HD NI NH NH
NH;
(SEQ ID NO: 48)
TCCCCTGCAGGGCAACGCC
and
HD HD HD HD NG NH HD NI NH NH NH HD NI NI HD NH HD
HD;
(SEQ ID NO: 49)
TGCAGGGCAACGCCCAGGG
and
NH HD NI NH NH NH HD NI NI HD NH HD HD HD NI NH NH
NH;
(SEQ ID NO: 50)
TCTCGATTATGGGGGGGAT
and
HD NG HD NH NI NG NG NING NH NH NH HD NH NH NH NI
NG;
(SEQ ID NO: 51)
TCGCTTCTCGATTATGGGC
and
HD NH HD NG NG HD NG HD NH NI NG NG NING NH NH NH
HD;
(SEQ ID NO: 52)
TGTCGAGTCGCTTCTCGAT
and
NH NG HD NH NI NH NG HD NH HD NG NG HD NG HD NH NI
NG;
(SEQ ID NO: 53)
TCCATGTCGAGTCGCTTCT
and
HD HD NI NG NH NG HD NH NI NH NG HD NH HD NG NG HD
NG;
(SEQ ID NO: 54)
TCGCCTCCATGTCGAGTCG
and
HD NH HD HD NG HD HD NI NG NH NG HD NH NI NH NG HD
NH
(SEQ ID NO: 55)
TCGTCATCGCCTCCATGTC
and
HD NH NG HD NI NG HD NH HD HD NG HD HD NI NG NH NG
HD;
(SEQ ID NO: 56)
TGATCTCGTCATCGCCTCC
and
NH NI NG HD NG HD NH NG HD NI NG HD NH HD HD NG HD
HD;
(SEQ ID NO: 57)
GCTTCAGCTTCCTA
and
NH HD NG NG HD NI NH HD NG NG HD HD NG
NI;
(SEQ ID NO: 58)
CTGTGATCATGCCA
and
HD NG NK NG NH NI NG HD NI NG NH HD HD
NI;
(SEQ ID NO: 59)
ACAGTGGTACACACCT
and
NI HD NI NN NG NN NN NG NI HD NI HD NI HD HD
NG;
(SEQ ID NO: 60)
CCACCCCCCACTAAG
and
HD HD NI HD HD HD HD HD HD NI HD NG NI NI
NN;
(SEQ ID NO: 61)
CATTGGCCGGGCAC
and
HD NI NG NG NN NN HD HD NN NN NN HD NI
HD;
(SEQ ID NO: 62)
GCTTGAACCCAGGAGA
and
NN HD NG NG NN NI NI HD HD HD NI NN NN NI NN
NI;
(SEQ ID NO: 63)
ACACCCGATCCACTGGG
and
NI HD NI HD HD HD NN NI NG HD HD NI HD NG NN NN
NN;
(SEQ ID NO: 64)
GCTGCATCAACCCC
and
NN HD NG NN HD NI NG HD NI NI HD HD HD
HD;
(SEQ ID NO: 65)
GCCACAAACAGAAATA
and
NN NN HD NI HD NN NI NI NI HD NI HD HD HD NG HD
HD;
(SEQ ID NO: 66)
GGTGGCTCATGCCTG
and
NN NN NG NN NN HD NG HD NI NG NN HD HD NG
NN;
(SEQ ID NO: 67)
GATTTGCACAGCTCAT
and
NN NI NG NG NG NN HD NI HD NI NN HD NG HD NI
NG;
(SEQ ID NO: 68)
AAGCTCTGAGGAGCA
and
NI NI NH HD NG HD NG NH NI NH NH NI NH
HD;
(SEQ ID NO: 69)
CCCTAGCTGTCCC
and
HD HD HD NG NI NK HD NG NH NG HD HD HD
HD;
(SEQ ID NO: 70)
GCCTAGCATGCTAG
and
NH HD HD NG NI NH HD NI NG NH HD NG NI
NH;
(SEQ ID NO: 71)
ATGGGCTTCACGGAT
and
NI NG NH NH NH HD NG NG HD NI HD NH NH NI
NG;
(SEQ ID NO: 72)
GAAACTATGCCTGC
and
NH NI NI NI HD NG NING NH HD HD NG NH
HD;
(SEQ ID NO: 73)
GCACCATTGCTCCC
and
NH HD NI HD HD NI NG NG NH HD NG HD HD
HD;
(SEQ ID NO: 74)
GACATGCAACTCAG
and
NH NI HD NI NG NH HD NI NI HD NG HD NI
NH;
(SEQ ID NO: 75)
ACACCACTAGGGGT
and
NI HD NI HD HD NI HD NG NI NH NH NH NH
NG;
(SEQ ID NO: 76)
GTCTGCTAGACAGG
and
NH NG HD NG NH HD NG NI NH NI HD NI NH
NH;
(SEQ ID NO: 77)
GGCCTAGACAGGCTG
and
NH NH HD HD NG NI NH NI HD NI NH NH HD NG
NH;
(SEQ ID NO: 78)
GAGGCATTCTTATCG
and
NH NI NH NH HD NI NG NG HD NG NG NI NG HD
NH;
(SEQ ID NO: 79)
GCCTGGAAACGTTCC
and
NN HD HD NG NN NN NI NI NI HD NN NG NG HD
HD;
(SEQ ID NO: 80)
GTGCTCTGACAATA
and
NN NG NN HD NG HD NG NN NI HD NI NI NG
NI;
(SEQ ID NO: 81)
GTTTTGCAGCCTCC
and
NN NG NG NG NG NN HD NI NN HD HD NG HD
HD;
(SEQ ID NO: 82)
ACAGCTGTGGAACGT
and
NI HD NI NN HD NG NN NG NN NN NI NI HD NN
NG;
(SEQ ID NO: 83)
GGCTCTCTTCCTCCT
and
HD NI NI NN NI HD HD NN NI NN HD NI HD NG NN HD NG
NN;
(SEQ ID NO: 84)
CTATCCCAAAACTCT
and
HD NG NI NG HD HD HD NI NI NI NI HD NG HD
NG;
(SEQ ID NO: 85)
GAAAAACTATGTAT
and
NH NI NI NI NI NI HD NG NING NH NG NI
NG;
(SEQ ID NO: 86)
AGGCAGGCTGGTTGA
and
NI NH NH HD NI NH NH HD NG NH NH NG NG NH
NI;
(SEQ ID NO: 87)
CAATACAACCACGC
and
HD NI NI NG NI HD NI NI HD HD NI HD NN
HD;
(SEQ ID NO: 88)
ATGACGGACTCAACT
and
NI NG NN NI HD NN NN NI HD NG HD NI NI HD
NG;
and
(SEQ ID NO: 89)
CACAACATTTGTAA
and
HD NI HD NI NI HD NI NG NG NG NN NG NI NI.

In embodiments, the GSHS is within about 25, or about 50, or about 100, or about 150, or about 200, or about 300, or about 500 nucleotides of the TA dinucleotide site or TTAA (SEQ ID NO: 440) tetranucleotide site.

Illustrative DNA binding codes for human genomic safe harbor in areas of open chromatin via TALEs, encompassed by various embodiments are provided in TABLE 4A-4F. In embodiments, there is provided a variant of the TALEs, encompassed by various embodiments are provided in TABLE 4A-4F, e.g., having a sequence having at least about 90%, or at least about 93%, or at least about 95%, or at least about 97%, or at least about 98%, or at least about 99% identity to any of the sequences in TABLE 4A-4F.

Illustrative DNA binding codes for human genomic safe harbor in areas of open chromatin via TALEs, encompassed by various embodiments are provided in TABLE 4A:

| GSHS | ID | Sequence | TALE (DNA binding code) |
|---|---|---|---|
| AAVS1 | 1 | TGGCCGGCCTGACCACTGG | NH NH HD HD NH NH HD HD NG |
| | | (SEQ ID NO: 23) | NH NI HD HD NI HD NG NH NH |
| AAVS1 | 2 | TGAAGGCCTGGCCGGCCTG | NH NI NI NH NH HD HD NG NH NH |
| | | (SEQ ID NO: 24) | HD HD NH NH HD HD NG NH |
| AAVS1 | 3 | TGAGCACTGAAGGCCTGGC | NH NI NH HD NI HD NG NH NI NI |
| | | (SEQ ID NO: 25) | NH NH HD HD NG NH NH HD |
| AAVS1 | 4 | TCCACTGAGCACTGAAGGC | HD HD NI HD NG NH NI NH HD NI |
| | | (SEQ ID NO: 26) | HD NG NH NI NI NH NH HD |
| AAVS1 | 5 | TGGTTTCCACTGAGCACTG | NH NH NG NG NG HD HD NI HD |
| | | (SEQ ID NO: 27) | NG NH NI NH HD NI HD NG NH |
| AAVS1 | 6 | TGGGGAAAATGACCCAACA | NH NH NH NH NI NI NI NI NG NH |
| | | (SEQ ID NO: 28) | NI HD HD HD NI NI HD NI |
| AAVS1 | 7 | TAGGACAGTGGGGAAAATG | NI NH NH NI HD NI NH NG NH NH |
| | | (SEQ ID NO: 29) | NH NH NI NI NI NING NH |
| AAVS1 | 8 | TCCAGGGACACGGTGCTAG | HD HD NI NH NH NH NI HD NI HD |
| | | (SEQ ID NO: 30) | NH NH NG NH HD NG NI NH |
| AAVS1 | 9 | TCAGAGCCAGGAGTCCTGG | HD NI NH NI NH HD HD NI NH NH |
| | | (SEQ ID NO: 31) | NI NH NG HD HD NG NH NH |
| AAVS1 | 10 | TCCTTCAGAGCCAGGAGTC | HD HD NG NG HD NI NH NI NH HD |
| | | (SEQ ID NO: 32) | HD NI NH NH NI NH NG HD |
| AAVS1 | 11 | TCCTCCTTCAGAGCCAGGA | HD HD NG HD HD NG NG HD NI |
| | | (SEQ ID NO: 33) | NH NI NH HD HD NI NH NH NI |
| AAVS1 | 12 | TCCAGCCCCTCCTCCTTCA | HD HD NI NH HD HD HD HD NG |
| | | (SEQ ID NO: 34) | HD HD NG HD HD NG NG HD NI |
| AAVS1 | 13 | TCCGAGCTTGACCCTTGGA | HD HD NH NI NH HD NG NG NH NI |
| | | (SEQ ID NO: 35) | HD HD HD NG NG NH NH NI |
| AAVS1 | 14 | TGGTTTCCGAGCTTGACCC | NH NH NG NG NG HD HD NH NI |
| | | (SEQ ID NO: 36) | NH HD NG NG NH NI HD HD HD |
| AAVS1 | 15 | TGGGGTGGTTTCCGAGCTT | NH NH NH NH NG NH NH NG NG |
| | | (SEQ ID NO: 37) | NG HD HD NH NI NH HD NG NG |
| AAVS1 | 16 | TCTGCTGGGGTGGTTTCCG | HD NG NH HD NG NH NH NH NH |
| | | (SEQ ID NO: 38) | NG NH NH NG NG NG HD HD NH |
| AAVS1 | 17 | TGCAGAGTATCTGCTGGGG | NH HD NI NH NI NH NG NING HD |
| | | (SEQ ID NO: 39) | NG NH HD NG NH NH NH NH |
| AAVS1 | AVS1 | CCAATCCCCTCAGT (SEQ ID | HD HD NI NI NG HD HD HD HD NG |
| | | NO: 40) | HD NI NH NG |
| AAVS1 | AVS2 | CAGTGCTCAGTGGAA (SEQ | HD NI NH NG NH HD NG HD NI NH |
| | | ID NO: 41) | NG NH NH NI NI |
| AAVS1 | AVS3 | GAAACATCCGGCGACTCA | NH NI NI NI HD NI NG HD HD NH |
| | | (SEQ ID NO: 42) | NH HD NH NI HD NG HD NI |
| hROSA26 | 1F | TCGCCCCTCAAATCTTACA | HD NH HD HD HD HD NG HD NI NI |
| | | (SEQ ID NO: 43) | NI NG HD NG NG NI HD NI |
| hROSA26 | 2F | TCAAATCTTACAGCTGCTC | HD NI NI NI NG HD NG NG NI HD |
| | | (SEQ ID NO: 44) | NI NH HD NG NH HD NG HD |
| hROSA26 | 3F | TCTTACAGCTGCTCACTCC | HD NG NG NI HD NI NH HD NG NH |
| | | (SEQ ID NO: 45) | HD NG HD NI HD NG HD HD |
| hROSA26 | 4F | TACAGCTGCTCACTCCCCT | NI HD NI NH HD NG NH HD NG HD |
| | | (SEQ ID NO: 46) | NI HD NG HD HD HD HD NG |
| hROSA26 | 5F | TGCTCACTCCCCTGCAGGG | NH HD NG HD NI HD NG HD HD |
| | | (SEQ ID NO: 47) | HD HD NG NH HD NI NH NH NH |
| hROSA26 | 6F | TCCCCTGCAGGGCAACGCC | HD HD HD HD NG NH HD NI NH |
| | | (SEQ ID NO: 48) | NH NH HD NI NI HD NH HD HD |
| hROSA26 | 7F | TGCAGGGCAACGCCCAGGG | NH HD NI NH NH NH HD NI NI HD |
| | | (SEQ ID NO: 49) | NH HD HD HD NI NH NH NH |
| hROSA26 | 8R | TCTCGATTATGGGCGGGAT | HD NG HD NH NI NG NG NING |
| | | (SEQ ID NO: 50) | NH NH NH HD NH NH NH NI NG |
| hROSA26 | 9R | TCGCTTCTCGATTATGGGC | HD NH HD NG NG HD NG HD NH |
| | | (SEQ ID NO: 51) | NI NG NG NI NG NH NH NH HD |
| hROSA26 | 10R | TGTCGAGTCGCTTCTCGAT | NH NG HD NH NI NH NG HD NH |
| | | (SEQ ID NO: 52) | HD NG NG HD NG HD NH NI NG |
| hROSA26 | 11R | TCCATGTCGAGTCGCTTCT | HD HD NI NG NH NG HD NH NI NH |
| | | (SEQ ID NO: 53) | NG HD NH HD NG NG HD NG |
| hROSA26 | 12R | TCGCCTCCATGTCGAGTCG | HD NH HD HD NG HD HD NI NG |
| | | (SEQ ID NO: 54) | NH NG HD NH NI NH NG HD NH |
| hROSA26 | 13R | TCGTCATCGCCTCCATGTC | HD NH NG HD NI NG HD NH HD |
| | | (SEQ ID NO: 55) | HD NG HD HD NING NH NG HD |
| hROSA26 | 14R | TGATCTCGTCATCGCCTCC | NH NI NG HD NG HD NH NG HD NI |
| | | (SEQ ID NO: 56) | NG HD NH HD HD NG HD HD |
| hROSA26 | ROSA1 | GCTTCAGCTTCCTA (SEQ ID | NH HD NG NG HD NI NH HD NG |
| | | NO: 57) | NG HD HD NG NI |
| hROSA26 | ROSA2 | CTGTGATCATGCCA (SEQ ID | HD NG NK NG NH NI NG HD NI NG |
| | | NO: 58) | NH HD HD NI |
| hROSA26 | TALER2 | ACAGTGGTACACACCT (SEQ | NI HD NI NN NG NN NN NG NI HD |
| | | ID NO: 59) | NI HD NI HD HD NG |
| hROSA26 | TALER3 | CCACCCCCCACTAAG (SEQ | HD HD NI HD HD HD HD HD HD NI |
| | | ID NO: 60) | HD NG NI NI NN |
| hROSA26 | TALER4 | CATTGGCCGGGCAC (SEQ | HD NI NG NG NN NN HD HD NN |
| | | ID NO: 61) | NN NN HD NI HD |
| hROSA26 | TALER5 | GCTTGAACCCAGGAGA | NN HD NG NG NN NI NI HD HD HD |
| | | (SEQ ID NO: 62) | NI NN NN NI NN NI |
| CCR5 | TALC3 | ACACCCGATCCACTGGG | NI HD NI HD HD HD NN NI NG HD |
| | | (SEQ ID NO: 63) | HD NI HD NG NN NN NN |
| CCR5 | TALC4 | GCTGCATCAACCCC (SEQ ID | NN HD NG NN HD NI NG HD NI NI |
| | | NO: 64) | HD HD HD HD |
| CCR5 | TALC5 | GCCACAAACAGAAATA (SEQ | NN NN HD NI HD NN NI NI NI HD |
| | | ID NO: 65) | NI HD HD HD NG HD HD |
| CCR5 | TALC7 | GGTGGCTCATGCCTG (SEQ | NN NN NG NN NN HD NG HD NI |
| | | ID NO: 66) | NG NN HD HD NG NN |
| CCR5 | TALC8 | GATTTGCACAGCTCAT (SEQ | NN NI NG NG NG NN HD NI HD NI |
| | | ID NO: 67) | NN HD NG HD NI NG |
| Chr 2 | SHCHR2-1 | AAGCTCTGAGGAGCA (SEQ | NI NI NH HD NG HD NG NH NI NH |
| | | ID NO: 68) | NH NI NH HD |
| Chr 2 | SHCHR2-2 | CCCTAGCTGTCCC (SEQ ID | HD HD HD NG NI NK HD NG NH |
| | | NO: 69) | NG HD HD HD HD |
| Chr 2 | SHCHR2-3 | GCCTAGCATGCTAG (SEQ ID | NH HD HD NG NI NH HD NI NG NH |
| | | NO: 70) | HD NG NI NH |
| Chr 2 | SHCHR2-4 | ATGGGCTTCACGGAT (SEQ | NI NG NH NH NH HD NG NG HD NI |
| | | ID NO: 71) | HD NH NH NI NG |
| Chr 4 | SHCHR4-1 | GAAACTATGCCTGC (SEQ ID | NH NI NI NI HD NG NI NG NH HD |
| | | NO: 72) | HD NG NH HD |
| Chr 4 | SHCHR4-2 | GCACCATTGCTCCC (SEQ ID | NH HD NI HD HD NI NG NG NH HD |
| | | NO: 73) | NG HD HD HD |
| Chr 4 | SHCHR4-3 | GACATGCAACTCAG (SEQ ID | NH NI HD NI NG NH HD NI NI HD |
| | | NO: 74) | NG HD NI NH |
| Chr 6 | SHCHR6-1 | ACACCACTAGGGGT (SEQ | NI HD NI HD HD NI HD NG NI NH |
| | | ID NO: 75) | NH NH NH NG |
| Chr 6 | SHCHR6-2 | GTCTGCTAGACAGG (SEQ | NH NG HD NG NH HD NG NI NH NI |
| | | ID NO: 76) | HD NI NH NH |
| Chr 6 | SHCHR6-3 | GGCCTAGACAGGCTG (SEQ | NH NH HD HD NG NI NH NI HD NI |
| | | ID NO: 77) | NH NH HD NG NH |
| Chr 6 | SHCHR6-4 | GAGGCATTCTTATCG (SEQ | NH NI NH NH HD NING NG HD NG |
| | | ID NO: 78) | NG NI NG HD NH |
| Chr 10 | SHCHR10- | GCCTGGAAACGTTCC (SEQ | NN HD HD NG NN NN NI NI NI HD |
| | 1 | ID NO: 79) | NN NG NG HD HD |
| Chr 10 | SHCHR10- | GTGCTCTGACAATA (SEQ ID | NN NG NN HD NG HD NG NN NI |
| | 2 | NO: 80) | HD NI NI NG NI |
| Chr 10 | SHCHR10- | GTTTTGCAGCCTCC (SEQ ID | NN NG NG NG NG NN HD NI NN |
| | 3 | NO: 81) | HD HD NG HD HD |
| Chr 10 | SHCHR10- | ACAGCTGTGGAACGT (SEQ | NI HD NI NN HD NG NN NG NN NN |
| | 4 | ID NO: 82) | NI NI HD NN NG |
| Chr 10 | SHCHR10- | GGCTCTCTTCCTCCT (SEQ | HD NI NI NN NI HD HD NN NI NN |
| | 5 | ID NO: 83) | HD NI HD NG NN HD NG NN |
| Chr 11 | SHCHR11- | CTATCCCAAAACTCT (SEQ | HD NG NI NG HD HD HD NI NI NI |
| | 1 | ID NO: 84) | NI HD NG HD NG |
| Chr 11 | SHCHR11- | GAAAAACTATGTAT (SEQ ID | NH NI NI NI NI NI HD NG NING NH |
| | 2 | NO: 85) | NG NI NG |
| Chr 11 | SHCHR11- | AGGCAGGCTGGTTGA (SEQ | NI NH NH HD NI NH NH HD NG NH |
| | 3 | ID NO: 86) | NH NG NG NH NI |
| Chr 17 | SHCHR17- | CAATACAACCACGC (SEQ ID | HD NI NI NG NI HD NI NI HD HD NI |
| | 1 | NO: 87) | HD NN HD |
| Chr 17 | SHCHR17- | ATGACGGACTCAACT (SEQ | NI NG NN NI HD NN NN NI HD NG |
| | 2 | ID NO: 88) | HD NI NI HD NG |
| Chr 17 | SHCHR17- | CACAACATTTGTAA (SEQ ID | HD NI HD NI NI HD NI NG NG NG |
| | 3 | NO: 89) | NN NG NI NI |
| Chr 17 | SHCHR17- | ATTTCCAGTGCACA (SEQ ID | NI NG NG NG HD HD NI NN NG |
| | 4 | NO: 90) | NN HD NI HD NI |

In embodiments, TALEs for targeting human genomic safe harbor sites using any of the TALE-based targeting elements to the TTAA site in hROSA26 (e.g., hg38 chr3:9,396,133-9,396,305) are shown in TABLE 4B:

| | DNA SEQUENCE | |
|---|---|---|
| NAME | (SEQ ID NO: _) | RVD AMINO ACID CODE |
| R1 | TCGCCCCTCAAATCTTACAG | HD NH HD HD HD HD NG HD NI NI NI NG HD NG NG NI HD NI NH |
| | (584) | |
| R2 | TCAAATCTTACAGCTGCTCA | HD NI NI NI NG HD NG NG NI HD NI NH HD NG NH HD NG HD NI |
| | (585) | |
| R3 | TCTTACAGCTGCTCACTCCC | HD NG NG NI HD NI NH HD NG NH HD NG HD NI HD NG HD HD HD |
| | (586) | |
| R4 | TACAGCTGCTCACTCCCCTG | NI HD NI NH HD NG NH HD NG HD NI HD NG HD HD HD HD NG NH |
| | (587) | |
| R5 | TGCTCACTCCCCTGCAGGGC | NH HD NG HD NI HD NG HD HD HD HD NG NH HD NI NH NH NH HD |
| | (588) | |
| R6 | TCCCCTGCAGGGCAACGCCC | HD HD HD HD NG NH HD NI NH NH NH HD NI NI HD NH HD HD HD |
| | (456) | |
| R7 | TGCAGGGCAACGCCCAGGGA | NH HD NI NH NH NH HD NI NI HD NH HD HD HD NI NH NH NH NI |
| | (589) | |
| R8 | TCTCGATTATGGGCGGGATT | HD NG HD NH NI NG NG NI NG NH NH NH HD NH NH NH NI NG NG |
| | (590) | |
| R9 | TCGCTTCTCGATTATGGGCG | HD NH HD NG NG HD NG HD NH NI NG NG NI NG NH NH NH HD NH |
| | (591) | |
| R10 | TGTCGAGTCGCTTCTCGATT | NH NG HD NH NI NH NG HD NH HD NG NG HD NG HD NH NI NG NG |
| | (592) | |
| R11 | TCCATGTCGAGTCGCTTCTC | HD HD NI NG NH NG HD NH NI NH NG HD NH HD NG NG HD NG HD |
| | (593) | |
| R12 | TCGCCTCCATGTCGAGTCGC | HD NH HD HD NG HD HD NI NG NH NG HD NH NI NH NG HD NH HD |
| | (594) | |
| R13 | TCGTCATCGCCTCCATGTCG | HD NH NG HD NI NG HD NH HD HD NG HD HD NI NG NH NG HD NH |
| | (595) | |
| R14 | TGATCTCGTCATCGCCTCCA | NH NI NG HD NG HD NH NG HD NI NG HD NH HD HD NG HD HD NI |
| | (596) | |

In embodiments, TALEs for targeting human genomic safe harbor sites using any of the TALE-based targeting elements to the AAVS1 (e.g., hg38 chr19:55,112,851-55,113,324) are shown in TABLE 4C:

RVD AMINO ACID CODE

| | DNA SEQUENCE | |
|---|---|---|
| NAME | (SEQ ID NO: _) | RVD AMINO ACID CODE |
| AAV1c | TGGCCGGCCTGACCACTGGG (597) | NH NH HD HD NH NH HD HD NG NH NI HD HD NI HD NG NH NH |
| | | NH |
| AAV2c | TGAAGGCCTGGCCGGCCTGA (598) | NH NI NI NH NH HD HD NG NH NH HD HD NH NH HD HD NG NH |
| | | NI |
| AAV3c | TGAGCACTGAAGGCCTGGCC (599) | NH NI NH HD NI HD NG NH NI NI NH NH HD HD NG NH NH HD |
| | | HD |
| AAV4c | TCCACTGAGCACTGAAGGCC (600) | HD HD NI HD NG NH NI NH HD NI HD NG NH NI NI NH NH HD |
| | | HD |
| AAV5c | TGGTTTCCACTGAGCACTGA (601) | NH NH NG NG NG HD HD NI HD NG NH NI NH HD NI HD NG NH |
| | | NI |
| AAV6 | TGGGGAAAATGACCCAACAG (602) | NH NH NH NH NI NI NI NI NG NH NI HD HD HD NI NI HD NI |
| | | NH |
| AAV7 | TAGGACAGTGGGGAAAATGA (603) | NI NH NH NI HD NI NH NG NH NH NH NH NI NI NI NI NG NH |
| | | NI |
| AAV8 | TCCAGGGACACGGTGCTAGG (604) | HD HD NI NH NH NH NI HD NI HD NH NH NG NH HD NG NI NH |
| | | NH |
| AAV9 | TCAGAGCCAGGAGTCCTGGC (605) | HD NI NH NI NH HD HD NI NH NH NI NH NG HD HD NG NH NH |
| | | HD |
| AAV10 | TCCTTCAGAGCCAGGAGTCC (606) | HD HD NG NG HD NI NH NI NH HD HD NI NH NH NI NH NG HD |
| | | HD |
| AAV11 | TCCTCCTTCAGAGCCAGGAG (607) | HD HD NG HD HD NG NG HD NI NH NI NH HD HD NI NH NH NI |
| | | NH |
| AAV12 | TCCAGCCCCTCCTCCTTCAG (608) | HD HD NI NH HD HD HD HD NG HD HD NG HD HD NG NG HD NI |
| | | NH |
| AAV13c | TCCGAGCTTGACCCTTGGAA (462) | HD HD NH NI NH HD NG NG NH NI HD HD HD NG NG NH NH NI |
| | | NI |
| AAV14c | TGGTTTCCGAGCTTGACCCT (112) | NH NH NG NG NG HD HD NH NI NH HD NG NG NH NI HD HD HD |
| | | NG |
| AAV15c | TGGGGTGGTTTCCGAGCTTG (609) | NH NH NH NH NG NH NH NG NG NG HD HD NH NI NH HD NG NG |
| | | NH |
| AAV16c | TCTGCTGGGGTGGTTTCCGA (610) | HD NG NH HD NG NH NH NH NH NG NH NH NG NG NG HD HD NH |
| | | NI |
| AAV17c | TGCAGAGTATCTGCTGGGGT (611) | NH HD NI NH NI NH NG NI NG HD NG NH HD NG NH NH NH NH |
| | | NG |

In embodiments, TALEs for targeting human genomic safe harbor sites using any of the TALE-based targeting elements to Chromosome 4 (e.g., hg38 chr4:30,793,534-30,875,476 or hg38 chr4:30,793,533-30,793,537 (9677); chr4:30,875,472-30,875,476 (8948)) are shown in TABLE 4D:

| | DNA SEQUENCE | |
|---|---|---|
| NAME | (SEQ ID NO: _) | RVD AMINO ACID CODE |
| TALE4-R001 | TCTTCCTAGTATTAAAGT (612) | HD NG NG HD HD NG NI NH NG NI NG NG NI NI NI |
| | | NH NG |
| TALE4-R002 | TCCTTAATATTACCAGT (613) | HD HD NG NG NI NI NG NI NG NG NI HD HD NI NH |
| | | NG |
| TALE4-F003 | TACCAAGCTGAAATGACACAAAAGT | NI HD HD NI NI NH HD NG NH NI NI NI NG NH NI |
| | (614) | HD NI HD NI NI NI NI NH NG |
| TALE4-F004 | TGGCTGTGTCACATACCAGCAGAAT | NH NH HD NG NH NG NH NG HD NI HD NI NG NI HD |
| | (615) | HD NI NH HD NI NH NI NI NG |
| TALE4-F005 | TGTTAATTTGAATACAATCACT (616) | NH NG NG NI NI NG NG NG NH NI NI NG NI HD NI |
| | | NI NG HD NI HD NG |
| TALE4-F006 | TGTGTCACATACCAGCAGAAT (617) | NH NG NH NG HD NI HD NI NG NI HD HD NI NH HD |
| | | NI NH NI NI NG |
| TALE4-R007 | TGGTAACTACTAATTT (618) | NH NH NG NI NI HD NG NI HD NG NI NI NG NG NG |
| TALE4-F008 | TGTCACATACCAGCAGAAT (619) | NH NG HD NI HD NI NG NI HD HD NI NH HD NI NH |
| | | NI NI NG |
| TALE4-R009 | TGTGACACAGCCATCAACAAT (620) | NH NG NH NI HD NI HD NI NH HD HD NI NG HD NI |
| | | NI HD NI NI NG |
| TALE4-F010 | TCCTTTGATGAACAGT (621) | HD HD NG NG NG NH NI NG NH NI NI HD NI NH NG |
| TALE4-F011 | TGTGTGCAATAGCGTTAAAGGAACTACAT | NH NG NH NG NH HD NI NI NG NI NH HD NH NG NG |
| | (622) | NI NI NI NH NH NI NI HD NG NI HD NI NG |
| TALE4-F012 | TCTTTCAATAGCCCACT (623) | NG NG NG HD NI NI NG NI NH HD HD HD NI HD |
| | | HD NG |
| TALE4-R013 | TCTCAAATGACAAGAGCACAGT (624) | HD NG HD NI NI NI NG NH NI HD NI NI NH NI NH |
| | | HD NI HD NI NH NG |
| TALE4-F014 | TACCAGTTAATTAGCACT (625) | HD NG HD NI NH NG NG NI NI NG NG NI NH HD NI |
| | | NI HD |
| TALE4-F015 | TGTTGTGACCTAAGCCAT (626) | NI NG NG NH NG NH NI HD HD NG NI NI NH HD HD |
| | | NH NG |
| TALE4-R016 | TCTCATGTTTTAAAGTCAAGAAT (627) | HD NG HD NI NG NH NG NG NG NG NI NI NI NH NG |
| | | HD NI NI NH NI NI NG |
| TALE4-F017 | TCCTGAATTCAGAACAGAT (628) | NH NI NG NI NG NG HD NI NH NI NI HD NI |
| | | HD HD NG NH NI |
| TALE4-F018 | TAGCATGATGTTTCATGTTGTGACCT | NI NH HD NI NG NH NI NG NH NG NG NG HD NI NG |
| | (629) | NH NG NG NH NG NH NI HD HD NG |
| TALE4-F019 | TGTTTCATGTTGTGACCTAAGCCAT | NH NG NG NG HD NI NG NH NG NG NH NG NH NI HD |
| | (630) | HD NG NI NI NH HD HD NI NG |
| TALE4-F020 | TACAACAGTCTATTTCAT (631) | NI NG NI NI HD NI NH NG HD NG NI NG NG NG HD |
| | | NI HD |

In embodiments, TALEs for targeting human genomic safe harbor sites using any of the TALE-based targeting elements to Chromosome 22 (e.g., hg38 chr22:35,370,000-35,380,000 or hg38 chr22:35,373,912-35,373,916 (861); chr22:35,377,843-35,377,847 (1153)) are shown in TABLE 4E:

| | DNA SEQUENCE | |
|---|---|---|
| NAME | (SEQ ID NO: _) | RVD AMINO ACID CODE |
| TALE22F- | TCTTCCTAGTCTCTTCTCTACCCAGT (632) | HD NG NG HD HD NG NI NH NG HD NG HD NG NG HD |
| R001 | | NG HD NG NI HD HD HD NI NH NG |
| TALE22- | TACACTCCAGCCTGGGAAACAGAGT (633) | NI HD NI HD NG HD HD NI NH HD HD NG NH NH NH |
| F002 | | NI NI NI HD NI NH NI NH NG |
| TALE22- | TCTTTTCCTTAGGACGGCT (634) | HD NG NG NG NG HD HD NG NG NI NH NH NI HD NH |
| F003 | | NH HD NG |
| TALE22- | TCGCTCAGGCCTGTCAT (635) | NG NH HD NG HD NI NH NH HD HD NG NH NG HD NI |
| F004 | | HD |
| TALE22- | TCCATATGGAAGACTT (636) | HD HD NI NG NI NG NH NH NI NI NH NI HD NG NG |
| F005 | | |
| TALE22- | TACCCAGTTAACCACCCT (637) | HD NG HD HD NI NH NG NG NI NI HD HD NI HD HD |
| F006 | | NI HD |
| TALE22- | TGGCGCATGCCTGTAATCCCAGCTACT | NH NH HD NH HD NI NG NH HD HD NG NH NG NI NI |
| F007 | (638) | NG HD HD HD NI NH HD NG NI HD NG |
| TALE22- | TATACGAGGAGAAAATTAGCATTCCT (639) | NI NG NI HD NH NI NH NH NI NH NI NI NI NI NG |
| F008 | | NG NI NH HD NI NG NG HD HD NG |
| TALE22- | TCTGCCTCCCAGGTTCACGCAAT (640) | HD NG NH HD HD NG HD HD HD NI NH NH NG NG HD |
| R009 | | NI HD NH HD NI NI NG |
| TALE22- | TGCCTTGTCACGTTTTCACAGT (641) | NH HD HD NG NG NH NG HD NI HD NH NG NG NG NG |
| F010 | | HD NI HD NI NH NG |
| TALE22- | TGTCACCTTCTGTATGTGCAACCAT (642) | NH NG HD NI HD HD NG NG HD NG NH NG NI NG NH |
| F001A | | NG NH HD NI NI HD HD NI NG |
| TALE22- | TCTGTATGTGCAACCAT (643) | NG NG NH NG NI NG NH NG NH HD NI NI HD HD NI |
| F002A | | HD |
| TALE22- | TAGTCAAGCAACAGGAT (644) | NG NH NG HD NI NI NH HD NI NI |
| R03A | | NI |
| TALE22- | TCCAAGATAATTCCCCAT (645) | NI NG NI NI NH NI NG NI NI NG NG HD HD HD HD |
| F004A | | HD HD |
| TALE22- | TCTGCAAGATCCTTTT (646) | HD NG NH HD NI NI NH NI NG HD HD NG NG NG NG |
| F005A | | |
| TALE22- | TGCTATGTAAGGTAGCAAAAAGGTAACCT | NH HD NG NI NG NH NG NI NI NH NH NG NI NH HD |
| F006A | (647) | NI NI NI NI NI NH NH NG NI NI HD HD NG |
| TALE22- | TCTCTCTCCTCCTGCT (648) | HD NG HD NG HD NG HD HD NG HD HD NG NH HD NG |
| R007A | | |
| TALE22- | TCCAAATGCTATTCTCTCT (649) | NG HD NG NI NI NG NH HD NG NI NG NG HD NG HD |
| R008A | | HD HD NI |
| TALE22- | TGCTGATTCAGCCTCCT (650) | NG HD NG NH NI NG NG HD NI NH HD HD NG HD HD |
| R009A | | NH |
| TALE22- | TAGAACAGCCCCCCACACAGT (651) | NI NH NI NI HD NI NH HD HD HD HD HD HD NI HD |
| F010A | | NI HD NI NH NG |

In embodiments, TALEs for targeting human genomic safe harbor sites using any of the TALE-based targeting elements to Chromosome X (e.g., hg38 chrX:134,419,661-134,541,172 or hg38 chrX:134,476,304-134,476,307 (85); chrX:134,476,337-134,476,340 (51)) are shown in TABLE 4F:

| | DNA SEQUENCE | |
|---|---|---|
| NAME | (SEQ ID NO: _) | RVD AMINO ACID CODE |
| TALE F002 | TTTAGCAGATGCATCAGC (652) | NG NG NI NH HD NI NH NI NG NH HD NI NG HD NI NH |
| | | HD |
| TALE F003 | TGACCAGGGGCATGTCCTGG (653) | NH NI HD HD NI NH NH NH NH HD NI NG NH NG HD HD |
| | | NGNH NH |
| TALE F004 | TGGTCCACCTACCTGAAAATG (654) | HD NI NI NH NH NI NH NG NG HD NG NH NH HD NG NH |
| | | NH NH NG HD |
| TALE F007 | TGTCCCACAGGTATTACGGGC (655) | NH NG HD HD HD NI HD NI NH NH NG NI NG NG NI HD |
| | | NH NH NH HD |
| TALE F008 | TACGGGCCAACCTGACAATAC (656) | NI HD NH NH NH HD HD NI NI HD HD NG NH NI HD NI |
| | | NI NG NI HD |
| TALE F009 | TGAGCTTTGGGGACTGAAAGA (657) | NH NI NH HD NG NG NG NH NH NH NH NI HD NG NH NI |
| | | NI NI NH NI |
| TALE R002 | CTGGCATAATCTTTTCCCCCA (658) | NH NH NH NH NH NI NI NI NI NH NI NG NG NI NG NH |
| | | HD HD NI NH |
| TALE R003 | CCAGCCTCCTGGCCATGTGCA (659) | NH HD NI HD NI NG NH NH HD HD NI NH NH NI NH NH |
| | | HD NG NH NH |
| TALE R004 | GGCCATGTGCACAGGGGCTGA (660) | HD NI NH HD HD HD HD NG NH NG NH HD NI HD NI NG |
| | | NH NH HD HD |
| TALE R005 | CTGATATGTGAAGGTTTAGCA (661) | NH HD NG NI NI NI HD HD NG NG HD NI HD NI NG NI |
| | | NG HD NI NH |
| TALE R007 | TGACCAGGCGTGGTGGCTCAC (662) | NH NI HD HD NI NH NH HD NH NG NH NH NG NH NH HD |
| | | NG HD NI HD |
| TALE F020* | TATAGACATTTTCACT (663) | NI NG NI NH NI HD NI NG NG NG NG HD NI HD NG |
| TALE F021* | TCTACATTTAACTATCAACCT (664) | HD NG NI HD NI NG NG NG NI NI HD NG NI NG HD NI |
| | | NI HD HD NG |
| TALE F030* | TCGTGCAAACGTTTGAT (665) | HD NH NG NH HD NI NI NI HD NH NG NG NG NH NI NG |
| TALE F031* | TACATCAATCCTGTAGGT* (666) | NI HD NI NG HD NI NI NG HD HD NG NH NG NI NH NH |
| | | NG |
| TALE F034* | TCTATTTTAGTGACCCAAGT (667) | HD NG NI NG NG NG NG NI NH NG NH NI HD HD HD NI |
| | | NI NH NG |
| TALE F036* | TAGAGTCAAAGCATGTACT (668) | NI NH NI NH NG HD NI NI NI NH HD NI NG NH NG NI |
| | | HD NG |
| TALE F037* | TCCTACCCATAAGCTCCT (669) | HD HD NG NI HD HD HD NI NG NI NI NH HD NG HD HD |
| | | NG |
| TALE F040* | TCCCCATCCCCATCAGT (670) | HD HD HD HD NI NG HD HD HD HD NI NG HD NI NH NG |
| TALE R022* | TCTTTAATTCAAGCAAGACTTTAACAAGT | HD NG NG NG NI NI NG NG HD NI NI NH HD NI NI NH |
| | (671) | NI HD NG NG NG NI NI HD NI NI NH NG |
| TALE R033* | TGCAGTCCCCTTTCTT (672) | NH HD NI NH NG HD HD HD HD NG NG NG HD NG NG |
| TALE R035* | TCTGCACAAATCCCCAAAGAT (673) | HD NG NH HD NI HD NI NI NI NG HD HD HD HD NI NI |
| | | NI NH NI NG |
| TALE R038* | TACATGCTTTGACTCT (674) | NI HD NI NG NH HD NG NG NG NH NI HD NG HD NG |
| TALE R039* | TGGCCAGTTATACTGCCAGCAGCTATAAT | NH NH HD HD NI NH NG NG NI NG NI HD NG NH HD HD |
| | (675) | NI NH HD NI NH HD NG NI NG NI NI NG |

In embodiments, the mobile element enzyme is capable of inserting a donor DNA at a TA dinucleotide site. In embodiments, the mobile element enzyme is capable of inserting a donor DNA at a TTAA (SEQ ID NO: 440) tetranucleotide site.

Illustrative DNA binding codes for human genomic safe harbor in areas of open chromatin via ZNFs, encompassed by various embodiments are provided in TABLE 5A-5E. In embodiments, there is provided a variant of the ZNFs, encompassed by various embodiments are provided in TABLE 5A-5E, e.g., having a sequence having at least about 90%, or at least about 93%, or at least about 95%, or at least about 97%, or at least about 98%, or at least about 99% identity to any of the sequences in TABLE 5A-5E.

In embodiments, ZNFs for targeting human genomic safe harbor sites using any of the ZNF-based targeting elements to the TTAA site in hROSA26 (e.g., hg38 chr3:9,396,133-9,396,305) are shown in TABLE 5A:

| | | TARGET | | |
|---|---|---|---|---|
| hROSA26 | | (SEQ ID | | |
| TTAA | NAME | NO: _) | SCORE | ZFP AMINO ACID CODE (SEQ ID NO: _) |
| 5′ | ZnF3a | TGG GAA GAT | 58.64 | LEPGEKPYKCPECGKSFSQNSTLTEHQRTHTGEKPYKCPECGKSFSQRANLRAHQ |
| | | AAA CTA | | RTHTGEKPYKCPECGKSFSTSGNLVRHQRTHTGEKPYKCPECGKSFSQSSNLVRH |
| | | (676) | | QRTHTGEKPYKCPECGKSFSRSDHLTTHQRTHTGKKTS (677) |
| 5′ | ZnF5a | ACT CCC CTG | 56.25 | LEPGEKPYKCPECGKSFSDSGNLRVHQRTHTGEKPYKCPECGKSFSDPGHLVRHQ |
| | | CAG GGC AAC | | RTHTGEKPYKCPECGKSFSRADNLTEHQRTHTGEKPYKCPECGKSFSRNDALTEH |
| | | (678) | | QRTHTGEKPYKCPECGKSFSSKKHLAEHQRTHTGEKPYKCPECGKSFSTHLDLIR |
| | | | | HQRTHTGKKTS (679) |
| 5′ | ZnF5b | CCC CTG CAG | 56.25 | LEPGEKPYKCPECGKSFSDCRDLARHQRTHTGEKPYKCPECGKSFSDSGNLRVHQ |
| | | GGC AAC GCC | | RTHTGEKPYKCPECGKSFSDPGHLVRHQRTHTGEKPYKCPECGKSFSRADNLTEH |
| | | (680) | | QRTHTGEKPYKCPECGKSFSRNDALTEHQRTHTGEKPYKCPECGKSFSSKKHLAE |
| | | | | HQRTHTGKKTS (681) |
| 5′ | ZnF5c | CTG CAG GGC | 60.58 | LEPGEKPYKCPECGKSFSRADNLTEHQRTHTGEKPYKCPECGKSFSDCRDLARHQ |
| | | AAC GCC CAG | | RTHTGEKPYKCPECGKSFSDSGNLRVHQRTHTGEKPYKCPECGKSFSDPGHLVRH |
| | | (682) | | QRTHTGEKPYKCPECGKSFSRADNLTEHQRTHTGEKPYKCPECGKSFSRNDALTE |
| | | | | HQRTHTGKKTS (683) |
| 5′ | ZnF5d | CAG GGC AAC | 58.08 | LEPGEKPYKCPECGKSFSQRAHLERHQRTHTGEKPYKCPECGKSFSRADNLTEHQ |
| | | GCC CAG GGA | | RTHTGEKPYKCPECGKSFSDCRDLARHQRTHTGEKPYKCPECGKSFSDSGNLRVH |
| | | (684) | | QRTHTGEKPYKCPECGKSFSDPGHLVRHQRTHTGEKPYKCPECGKSFSRADNLTE |
| | | | | HQRTHTGKKTS (685) |
| 5′ | ZnF5e | GGC AAC GCC | 57.32 | LEPGEKPYKCPECGKSFSTSHSLTEHQRTHTGEKPYKCPECGKSFSQRAHLERHQ |
| | | CAG GGA CCA | | RTHTGEKPYKCPECGKSFSRADNLTEHQRTHTGEKPYKCPECGKSFSDCRDLARH |
| | | (686) | | QRTHTGEKPYKCPECGKSFSDSGNLRVHQRTHTGEKPYKCPECGKSFSDPGHLVR |
| | | | | HQRTHTGKKTS (687) |
| 5′ | ZnF5f | AAC GCC CAG | 54.99 | LEPGEKPYKCPECGKSFSHRTTLTNHQRTHTGEKPYKCPECGKSFSTSHSLTEHQ |
| | | GGA CCA AGT | | RTHTGEKPYKCPECGKSFSQRAHLERHQRTHTGEKPYKCPECGKSFSRADNLTEH |
| | | (688) | | QRTHTGEKPYKCPECGKSFSDCRDLARHQRTHTGEKPYKCPECGKSFSDSGNLRV |
| | | | | HQRTHTGKKTS (689) |
| 5′ | ZnF5g | GCC CAG GGA | 55.31 | LEPGEKPYKCPECGKSFSREDNLHTHQRTHTGEKPYKCPECGKSFSHRTTLTNHQ |
| | | CCA AGT TAG | | RTHTGEKPYKCPECGKSFSTSHSLTEHQRTHTGEKPYKCPECGKSFSQRAHLERH |
| | | (690) | | QRTHTGEKPYKCPECGKSFSRADNLTEHQRTHTGEKPYKCPECGKSFSDCRDLAR |
| | | | | HQRTHTGKKTS (691) |
| 5′ | ZnF5h | CAG GGA CCA | 50.76 | LEPGEKPYKCPECGKSFSSKKHLAEHQRTHTGEKPYKCPECGKSFSREDNLHTHQ |
| | | AGT TAG CCC | | RTHTGEKPYKCPECGKSFSHRTTLTNHQRTHTGEKPYKCPECGKSFSTSHSLTEH |
| | | (692) | | QRTHTGEKPYKCPECGKSFSQRAHLERHQRTHTGEKPYKCPECGKSFSRADNLTE |
| | | | | HQRTHTGKKTS (693) |
| 3′ | ZnF12a | GCC TAG GCA | 59.09 | LEPGEKPYKCPECGKSFSQSSNLVRHQRTHTGEKPYKCPECGKSFSQRANLRAHQ |
| | | AAA GAA | | RTHTGEKPYKCPECGKSFSQSGDLRRHQRTHTGEKPYKCPECGKSFSREDNLHTH |
| | | (694) | | QRTHTGEKPYKCPECGKSFSDCRDLARHQRTHTGKKTS (695) |
| 3′ | ZnF13a | CGC GAG GAG | 57.19 | LEPGEKPYKCPECGKSFSRSDHLTNHQRTHTGEKPYKCPECGKSFSRSDHLTNHQ |
| | | GAA AGG AGG | | RTHTGEKPYKCPECGKSFSQSSNLVRHQRTHTGEKPYKCPECGKSFSRSDNLVRH |
| | | (696) | | QRTHTGEKPYKCPECGKSFSRSDNLVRHQRTHTGEKPYKCPECGKSFSHTGHLLE |
| | | | | HQRTHTGKKTS (697) |
| 3′ | ZnF13b | GAG GAG GAA | 57.80 | LEPGEKPYKCPECGKSFSRSDNLVRHQRTHTGEKPYKCPECGKSFSRSDHLTNHQ |
| | | AGG AGG GAG | | RTHTGEKPYKCPECGKSFSRSDHLTNHQRTHTGEKPYKCPECGKSFSQSSNLVRH |
| | | (698) | | QRTHTGEKPYKCPECGKSFSRSDNLVRHQRTHTGEKPYKCPECGKSFSRSDNLVR |
| | | | | HQRTHTGKKTS (699) |
| 3′ | ZnF13c | GAG GAA AGG | 57.61 | LEPGEKPYKCPECGKSFSDPGHLVRHQRTHTGEKPYKCPECGKSFSRSDNLVRHQ |
| | | AGG GAG GGC | | RTHTGEKPYKCPECGKSFSRSDHLTNHQRTHTGEKPYKCPECGKSFSRSDHLTNH |
| | | (700) | | QRTHTGEKPYKCPECGKSFSQSSNLVRHQRTHTGEKPYKCPECGKSFSRSDNLVR |
| | | | | HQRTHTGKKTS (701) |

In embodiments, ZNFs for targeting human genomic safe harbor sites using any of the ZNF-based targeting elements to the AAVS1 (e.g., hg38 chr19:55,112,851-55,113,324) are shown in TABLE 5B:

| AAVS1 | | | | |
|---|---|---|---|---|
| TTAA | NAME | TARGET (SEQ ID NO: _) | SCORE | ZFP AMINO ACID CODE (SEQ ID NO: _) |
| 5′ | ZnF11a | TAG GAC AGT GGG GAA AAT | 57.08 | LEPGEKPYKCPECGKSFSDCRDLARHQRTHTGEKPYKCPECG |
| | | GAC CCA ACA GCC (702) | | KSFSSPADLTRHQRTHTGEKPYKCPECGKSFSTSHSLTEHQR |
| | | | | THTGEKPYKCPECGKSFSDPGNLVRHQRTHTGEKPYKCPECG |
| | | | | KSFSTTGNLTVHQRTHTGEKPYKCPECGKSFSQSSNLVRHQR |
| | | | | THTGEKPYKCPECGKSFSRSDKLVRHQRTHTGEKPYKCPECG |
| | | | | KSFSHRTTLTNHQRTHTGEKPYKCPECGKSFSDPGNLVRHQR |
| | | | | THTGEKPYKCPECGKSFSREDNLHTHQRTHTGKKTS (703) |
| 5′ | ZnF10a | AGA GGG AGC CAC GAA AAC | 56.91 | LEPGEKPYKCPECGKSFSQLAHLRAHQRTHTGEKPYKCPECG |
| | | AGA (704) | | KSFSDSGNLRVHQRTHTGEKPYKCPECGKSFSQSSNLVRHQR |
| | | | | THTGEKPYKCPECGKSFSSKKALTEHQRTHTGEKPYKCPECG |
| | | | | KSFSERSHLREHQRTHTGEKPYKCPECGKSFSRSDKLVRHQR |
| | | | | THTGEKPYKCPECGKSFSQLAHLRAHQRTHTGKKTS (705) |
| 3′ | ZnF12b | GCA GAT AGC CAG GAG | 59.97 | LEPGEKPYKCPECGKSFSRSDNLVRHQRTHTGEKPYKCPECG |
| | | (706) | | KSFSRADNLTEHQRTHTGEKPYKCPECGKSFSERSHLREHQR |
| | | | | THTGEKPYKCPECGKSFSTSGNLVRHQRTHTGEKPYKCPECG |
| | | | | KSFSQSGDLRRHQRTHTGKKTS (707) |
| 3′ | ZnF13b | AGA TAG CCA GGA GTC CTT | 56.80 | LEPGEKPYKCPECGKSFSTTGALTEHQRTHTGEKPYKCPECG |
| | | (708) | | KSFSDPGALVRHQRTHTGEKPYKCPECGKSFSQRAHLERHQR |
| | | | | THTGEKPYKCPECGKSFSTSHSLTEHQRTHTGEKPYKCPECG |
| | | | | KSFSREDNLHTHQRTHTGEKPYKCPECGKSFSQLAHLRAHQR |
| | | | | THTGKKTS (709) |
| 5′ | ZnF14a | CCC AGT GGT CAG GCC GGC | 61.78 | LEPGEKPYKCPECGKSFSDCRDLARHQRTHTGEKPYKCPECG |
| | | CAG GCC (710) | | KSFSRADNLTEHQRTHTGEKPYKCPECGKSFSDPGHLVRHQR |
| | | | | THTGEKPYKCPECGKSFSDCRDLARHQRTHTGEKPYKCPECG |
| | | | | KSFSRADNLTEHQRTHTGEKPYKCPECGKSFSTSGHLVRHQR |
| | | | | THTGEKPYKCPECGKSFSHRTTLTNHQRTHTGEKPYKCPECG |
| | | | | KSFSSKKHLAEHQRTHTGKKTS (711) |
| 5′ | ZnF15a | GGC CGG CCA GGC CTT CAG | 58.15 | LEPGEKPYKCPECGKSFSRADNLTEHQRTHTGEKPYKCPECG |
| | | (712) | | KSFSTTGALTEHQRTHTGEKPYKCPECGKSFSDPGHLVRHQR |
| | | | | THTGEKPYKCPECGKSFSTSHSLTEHQRTHTGEKPYKCPECG |
| | | | | KSFSRSDKLTEHQRTHTGEKPYKCPECGKSFSDPGHLVRHQR |
| | | | | THTGKKTS (713) |
| 5′ | ZnF16a | AGT GCT CAG TGG AAA CCA | 58.65 | LEPGEKPYKCPECGKSFSDPGNLVRHQRTHTGEKPYKCPECG |
| | | CGA AAG GAC (714) | | KSFSRKDNLKNHQRTHTGEKPYKCPECGKSFSQSGHLTEHQR |
| | | | | THTGEKPYKCPECGKSFSTSHSLTEHQRTHTGEKPYKCPECG |
| | | | | KSFSQRANLRAHQRTHTGEKPYKCPECGKSFSRSDHLTTHQR |
| | | | | THTGEKPYKCPECGKSFSRADNLTEHQRTHTGEKPYKCPECG |
| | | | | KSFSTSGELVRHQRTHTGEKPYKCPECGKSFSHRTTLTNHQR |
| | | | | THTGKKTS (715) |
| 5′ | ZnF17a | TGG CCC CCA GCC CCT CCT | 60.89 | LEPGEKPYKCPECGKSFSDCRDLARHQRTHTGEKPYKCPECG |
| | | GCC (716) | | KSFSTKNSLTEHQRTHTGEKPYKCPECGKSFSTKNSLTEHQR |
| | | | | THTGEKPYKCPECGKSFSDCRDLARHQRTHTGEKPYKCPECG |
| | | | | KSFSTSHSLTEHQRTHTGEKPYKCPECGKSFSSKKHLAEHQR |
| | | | | THTGEKPYKCPECGKSFSRSDHLTTHQRTHTGKKTS (717) |
| 5′ | ZnF18a | AGA GCC AGG AGT CCT GGC | 57.23 | LEPGEKPYKCPECGKSFSSKKHLAEHQRTHTGEKPYKCPECG |
| | | CCC CAG CCC (718) | | KSFSRADNLTEHQRTHTGEKPYKCPECGKSFSSKKHLAEHQR |
| | | | | THTGEKPYKCPECGKSFSDPGHLVRHQRTHTGEKPYKCPECG |
| | | | | KSFSTKNSLTEHQRTHTGEKPYKCPECGKSFSHRTTLTNHQR |
| | | | | THTGEKPYKCPECGKSFSRSDHLTNHQRTHTGEKPYKCPECG |
| | | | | KSFSDCRDLARHQRTHTGEKPYKCPECGKSFSQLAHLRAHQR |
| | | | | THTGKKTS (719) |
| 3′ | ZnF19a | GCA GGA GGG GCT GGG GGC | 59.93 | LEPGEKPYKCPECGKSFSDPGNLVRHQRTHTGEKPYKCPECG |
| | | CAG GAC (720) | | KSFSRADNLTEHQRTHTGEKPYKCPECGKSFSDPGHLVRHQR |
| | | | | THTGEKPYKCPECGKSFSRSDKLVRHQRTHTGEKPYKCPECG |
| | | | | KSFSTSGELVRHQRTHTGEKPYKCPECGKSFSRSDKLVRHQR |
| | | | | THTGEKPYKCPECGKSFSQRAHLERHQRTHTGEKPYKCPECG |
| | | | | KSFSQSGDLRRHQRTHTGKKTS (721) |
| 3′ | ZnF20b | ATA GCC CTG GGC CCA CGG | 59.53 | LEPGEKPYKCPECGKSFSSRRTCRAHQRTHTGEKPYKCPECG |
| | | CTT CGT (722) | | KSFSTTGALTEHQRTHTGEKPYKCPECGKSFSRSDKLTEHQR |
| | | | | THTGEKPYKCPECGKSFSTSHSLTEHQRTHTGEKPYKCPECG |
| | | | | KSFSDPGHLVRHQRTHTGEKPYKCPECGKSFSRNDALTEHQR |
| | | | | THTGEKPYKCPECGKSFSDCRDLARHQRTHTGEKPYKCPECG |
| | | | | KSFSQKSSLIAHQRTHTGKKT (723) |
| 3′ | ZnF21b | GAA GGA CCT GGC TGG | 55.22 | LEPGEKPYKCPECGKSFSRSDHLTTHQRTHTGEKPYKCPECG |
| | | (724) | | KSFSDPGHLVRHQRTHTGEKPYKCPECGKSFSTKNSLTEHQR |
| | | | | THTGEKPYKCPECGKSFSQRAHLERHQRTHTGEKPYKCPECG |
| | | | | KSFSQSSNLVRHQRTHTGKKTS (725) |
| 5′ | ZnF22a | GCA GGA ACG AAG CCG TGG | 56.47 | LEPGEKPYKCPECGKSFSDPGHLVRHQRTHTGEKPYKCPECG |
| | | GCC CAG GGC (726) | | KSFSRADNLTEHQRTHTGEKPYKCPECGKSFSDCRDLARHQR |
| | | | | THTGEKPYKCPECGKSFSRSDHLTTHQRTHTGEKPYKCPECG |
| | | | | KSFSRNDTLTEHQRTHTGEKPYKCPECGKSFSRKDNLKNHQR |
| | | | | THTGEKPYKCPECGKSFSRTDTLRDHQRTHTGEKPYKCPECG |
| | | | | KSFSQRAHLERHQRTHTGEKPYKCPECGKSFSQSGDLRRHQR |
| | | | | THTGKKTS (727) |
| 5′ | ZnF23a | GGA AAC CAC CCC AGC AGA | 52.63 | LEPGEKPYKCPECGKSFSQLAHLRAHQRTHTGEKPYKCPECG |
| | | (728) | | KSFSERSHLREHQRTHTGEKPYKCPECGKSFSSKKHLAEHQR |
| | | | | THTGEKPYKCPECGKSFSSKKALTEHQRTHTGEKPYKCPECG |
| | | | | KSFSDSGNLRVHQRTHTGEKPYKCPECGKSFSQRAHLERHQR |
| | | | | THTGKKTS (729) |
| 5′ | ZnF24a | AAG GGT CAA GCT CGG AAA | 55.09 | LEPGEKPYKCPECGKSFSQKSSLIAHQRTHTGEKPYKCPECG |
| | | CCA CCC CAG CAG ATA | | KSFSRADNLTEHQRTHTGEKPYKCPECGKSFSRADNLTEHQR |
| | | (730) | | THTGEKPYKCPECGKSFSSKKHLAEHQRTHTGEKPYKCPECG |
| | | | | KSFSTSHSLTEHQRTHTGEKPYKCPECGKSFSQRANLRAHQR |
| | | | | THTGEKPYKCPECGKSFSRSDKLTEHQRTHTGEKPYKCPECG |
| | | | | KSFSTSGELVRHQRTHTGEKPYKCPECGKSFSQSGNLTEHQR |
| | | | | THTGEKPYKCPECGKSFSTSGHLVRHQRTHTGEKPYKCPECG |
| | | | | KSFSRKDNLKNHQRTHTGKKTS (731) |

In embodiments, ZNFs for targeting human genomic safe harbor sites using any of the ZNF-based targeting elements to Chromosome 4 (e.g., hg38 chr4:30,793,534-30,875,476 or hg38 chr4:30,793,533-30,793,537 (9677); chr4:30,875,472-30,875,476 (8948)) are shown in TABLE 5C:

| Chr4 | | | | |
|---|---|---|---|---|
| TTAA | NAME | TARGET (SEQ ID NO: _) | SCORE | ZFP AMINO ACID CODE (SEQ ID NO: _) |
| 5′ | ZnF31F | CTTTGATGAACAGTCACA (732) | 58. 41 | LEPGEKPYKCPECGKSFSSPADLTRHQRTHTGEKPYKCPECG |
| | | | | KSFSDPGALVRHQRTHTGEKPYKCPECGKSFSSPADLTRHQR |
| | | | | THTGEKPYKCPECGKSFSQAGHLASHQRTHTGEKPYKCPECG |
| | | | | KSFSQAGHLASHQRTHTGEKPYKCPECGKSFSTTGALTEHQR |
| | | | | THTGKKTS (733) |
| 5′ | ZnF32F | CTTCCAATTAGTCCTACC (734) | 55.84 | LEPGEKPYKCPECGKSFSDKKDLTRHQRTHTGEKPYKCPECG |
| | | | | KSFSTKNSLTEHQRTHTGEKPYKCPECGKSFSHRTTLTNHQR |
| | | | | THTGEKPYKCPECGKSFSHKNALQNHQRTHTGEKPYKCPECG |
| | | | | KSFSTSHSLTEHQRTHTGEKPYKCPECGKSFSTTGALTEHQR |
| | | | | THTGKKTS (735) |
| 5′ | ZnF33F | ATACTAGGAAGAAATACAATA | 57.27 | LEPGEKPYKCPECGKSFSQKSSLIAHQRTHTGEKPYKCPECG |
| | | (736) | | KSFSSPADLTRHQRTHTGEKPYKCPECGKSFSTTGNLTVHQR |
| | | | | THTGEKPYKCPECGKSFSQLAHLRAHQRTHTGEKPYKCPECG |
| | | | | KSFSQRAHLERHQRTHTGEKPYKCPECGKSFSQNSTLTEHQR |
| | | | | THTGEKPYKCPECGKSFSQKSSLIAHQRTHTGKKTS (737) |
| 5′ | ZnF34F | GCTCTTGTCATTTGAGAT (738) | 57.38 | LEPGEKPYKCPECGKSFSTSGNLVRHQRTHTGEKPYKCPECG |
| | | | | KSFSQAGHLASHQRTHTGEKPYKCPECGKSFSHKNALQNHQR |
| | | | | THTGEKPYKCPECGKSFSDPGALVRHQRTHTGEKPYKCPECG |
| | | | | KSFSTTGALTEHQRTHTGEKPYKCPECGKSFSTSGELVRHQR |
| | | | | THTGKKTS (739) |
| 5′ | ZnF35F | CCAAGCTGAAATGACACAAAAGTT | 58.23 | LEPGEKPYKCPECGKSFSRKDNLKNHQRTHTGEKPYKCPECG |
| | | AAAACAAAG (740) | | KSFSSPADLTRHQRTHTGEKPYKCPECGKSFSQRANLRAHQR |
| | | | | THTGEKPYKCPECGKSFSTSGSLVRHQRTHTGEKPYKCPECG |
| | | | | KSFSQRANLRAHQRTHTGEKPYKCPECGKSFSSPADLTRHQR |
| | | | | THTGEKPYKCPECGKSFSDPGNLVRHQRTHTGEKPYKCPECG |
| | | | | KSFSTTGNLTVHQRTHTGEKPYKCPECGKSFSQAGHLASHQR |
| | | | | THTGEKPYKCPECGKSFSERSHLREHQRTHTGEKPYKCPECG |
| | | | | KSFSTSHSLTEHQRTHTGKKTS (741) |
| 5′ | ZnF36F | CTTATACCAGTTAATTAGCAC | 49.93 | LEPGEKPYKCPECGKSFSSKKALTEHQRTHTGEKPYKCPECG |
| | | (742) | | KSFSREDNLHTHQRTHTGEKPYKCPECGKSFSTTGNLTVHQR |
| | | | | THTGEKPYKCPECGKSFSTSGSLVRHQRTHTGEKPYKCPECG |
| | | | | KSFSTSHSLTEHQRTHTGEKPYKCPECGKSFSQKSSLIAHQR |
| | | | | THTGEKPYKCPECGKSFSTTGALTEHQRTHTGKKTS (743) |
| 3′ | ZnF37R | AACGCTATTGCACACATAGTTACA | 57.67 | LEPGEKPYKCPECGKSFSSPADLTRHQRTHTGEKPYKCPECG |
| | | (744) | | KSFSTSGSLVRHQRTHTGEKPYKCPECGKSFSQKSSLIAHQR |
| | | | | THTGEKPYKCPECGKSFSSKKALTEHQRTHTGEKPYKCPECG |
| | | | | KSFSQSGDLRRHQRTHTGEKPYKCPECGKSFSHKNALQNHQR |
| | | | | THTGEKPYKCPECGKSFSTSGELVRHQRTHTGEKPYKCPECG |
| | | | | KSFSDSGNLRVHQRTHTGKKTS (745) |
| 3′ | ZnF38R | TGAATTCAGGAACAAAGTATA | 53.21 | LEPGEKPYKCPECGKSFSQKSSLIAHQRTHTGEKPYKCPECG |
| | | (746) | | KSFSHRTTLTNHQRTHTGEKPYKCPECGKSFSQSGNLTEHQR |
| | | | | THTGEKPYKCPECGKSFSQSSNLVRHQRTHTGEKPYKCPECG |
| | | | | KSFSRADNLTEHQRTHTGEKPYKCPECGKSFSHKNALQNHQR |
| | | | | THTGEKPYKCPECGKSFSQAGHLASHQRTHTGKKTS (747) |
| 3′ | ZnF39R | GCTGGTATGTGACACAGCCATCA | 50.63 | LEPGEKPYKCPECGKSFSQSGNLTEHQRTHTGEKPYKCPECG |
| | | ACAA (748) | | KSFSQSGNLTEHQRTHTGEKPYKCPECGKSFSTSGNLTEHQR |
| | | | | THTGEKPYKCPECGKSFSERSHLREHQRTHTGEKPYKCPECG |
| | | | | KSFSSKKALTEHQRTHTGEKPYKCPECGKSFSQAGHLASHQR |
| | | | | THTGEKPYKCPECGKSFSRRDELNVHQRTHTGEKPYKCPECG |
| | | | | KSFSTSGHLVRHQRTHTGEKPYKCPECGKSFSTSGELVRHQR |
| | | | | THTGKKTS (749) |

In embodiments, ZNFs for targeting human genomic safe harbor sites using any of the ZNF-based targeting elements to Chromosome 22 (e.g., hg38 chr22:35,370,000-35,380,000 or hg38 chr22:35,373,912-35,373,916 (861); chr22:35,377,843-35,377,847 (1153)) are shown in TABLE 5D:

| Chr22 | | TARGET | | |
|---|---|---|---|---|
| TTAA | NAME | (SEQ ID NO: _) | SCORE | ZFP (SEQ ID NO: _) |
| 5′ | ZnF1a | CTTCCTGAAAGCAAGA | 57.34 | LEPGEKPYKCPECGKSFSTTGNLTVHQRTHTGEKPYKCPECGKSFSQAGHLASH |
| | | GATGAAAT (750) | | QRTHTGEKPYKCPECGKSFSQLAHLRAHQRTHTGEKPYKCPECGKSFSRKDNLK |
| | | | | NHQRTHTGEKPYKCPECGKSFSERSHLREHQRTHTGEKPYKCPECGKSFSQSSN |
| | | | | LVRHQRTHTGEKPYKCPECGKSFSTKNSLTEHQRTHTGEKPYKCPECGKSFSTT |
| | | | | GALTEHQRTHTGKKTS (751) |
| 5′ | ZnF1b | CTGAAAGCAAGAGATG | 58.92 | LEPGEKPYKCPECGKSFSTSHSLTEHQRTHTGEKPYKCPECGKSFSHKNALQNH |
| | | AAATTCCA (752) | | QRTHTGEKPYKCPECGKSFSQSSNLVRHQRTHTGEKPYKCPECGKSFSTSGNLV |
| | | | | RHQRTHTGEKPYKCPECGKSFSQLAHLRAHQRTHTGEKPYKCPECGKSFSQSGD |
| | | | | LRRHQRTHTGEKPYKCPECGKSFSQRANLRAHQRTHTGEKPYKCPECGKSFSRN |
| | | | | DALTEHQRTHTGKKTS (753) |
| 5′ | ZnF2a | ATACGAGGAGAAAATT | 51.25 | LEPGEKPYKCPECGKSFSTSGNLTEHQRTHTGEKPYKCPECGKSFSREDNLHTH |
| | | AGCAT (754) | | QRTHTGEKPYKCPECGKSFSTTGNLTVHQRTHTGEKPYKCPECGKSFSQSSNLV |
| | | | | RHQRTHTGEKPYKCPECGKSFSQRAHLERHQRTHTGEKPYKCPECGKSFSQSGH |
| | | | | LTEHQRTHTGEKPYKCPECGKSFSQKSSLIAHQRTHTGKKTS (755) |
| 5 | ZnF3a | CATCCATGGCAGGAAG | 58.67 | LEPGEKPYKCPECGKSFSRNDALTEHQRTHTGEKPYKCPECGKSFSTTGNLTVH |
| | | TTGAAGCCAAAATAAA | | QRTHTGEKPYKCPECGKSFSQKSSLIAHQRTHTGEKPYKCPECGKSFSQRANLR |
| | | TCTG (756) | | AHQRTHTGEKPYKCPECGKSFSDCRDLARHQRTHTGEKPYKCPECGKSFSQSSN |
| | | | | LVRHQRTHTGEKPYKCPECGKSFSTSGSLVRHQRTHTGEKPYKCPECGKSFSQS |
| | | | | SNLVRHQRTHTGEKPYKCPECGKSFSRADNLTEHQRTHTGEKPYKCPECGKSFS |
| | | | | RSDHLTTHQRTHTGEKPYKCPECGKSFSTSHSLTEHQRTHTGEKPYKCPECGKS |
| | | | | FSTSGNLTEHQRTHTGKKTS (757) |
| 5′ | ZnF3b | ATGGCAGGAAGTTGAA | 54.14 | LEPGEKPYKCPECGKSFSQRANLRAHQRTHTGEKPYKCPECGKSFSTTGNLTVH |
| | | GCCAAAATAAA | | QRTHTGEKPYKCPECGKSFSQSGNLTEHQRTHTGEKPYKCPECGKSFSERSHLR |
| | | (758) | | EHQRTHTGEKPYKCPECGKSFSQAGHLASHQRTHTGEKPYKCPECGKSFSHRTT |
| | | | | LTNHQRTHTGEKPYKCPECGKSFSQRAHLERHQRTHTGEKPYKCPECGKSFSQS |
| | | | | GDLRRHQRTHTGEKPYKCPECGKSFSRRDELNVHQRTHTGKKTS (759) |
| 3′ | ZnF5a | GAAAAGAAGACTCAAG | 55.40 | LEPGEKPYKCPECGKSFSSKKALTEHQRTHTGEKPYKCPECGKSFSQRANLRAH |
| | R | GAAACAGAGCCAAACA | | QRTHTGEKPYKCPECGKSFSDCRDLARHQRTHTGEKPYKCPECGKSFSQLAHLR |
| | | C (760) | | AHQRTHTGEKPYKCPECGKSFSDSGNLRVHQRTHTGEKPYKCPECGKSFSQRAH |
| | | | | LERHQRTHTGEKPYKCPECGKSFSQSGNLTEHQRTHTGEKPYKCPECGKSFSTH |
| | | | | LDLIRHQRTHTGEKPYKCPECGKSFSRKDNLKNHQRTHTGEKPYKCPECGKSFS |
| | | | | RKDNLKNHQRTHTGEKPYKCPECGKSFSQSSNLVRHQRTHTGKKTS (761) |
| 3′ | ZnF5b | AGGAAACAGAGCCAAA | 54.66 | LEPGEKPYKCPECGKSFSSPADLTRHQRTHTGEKPYKCPECGKSFSTTGALTEH |
| | R | CACTTACA (762) | | QRTHTGEKPYKCPECGKSFSSPADLTRHQRTHTGEKPYKCPECGKSFSQSGNLT |
| | | | | EHQRTHTGEKPYKCPECGKSFSERSHLREHQRTHTGEKPYKCPECGKSFSRADN |
| | | | | LTEHQRTHTGEKPYKCPECGKSFSQRANLRAHQRTHTGEKPYKCPECGKSFSRS |
| | | | | DHLTNHQRTHTGKKTS (763) |
| 3′ | ZnF6a | ATGCAGATTTGGACAC | 58.57 | LEPGEKPYKCPECGKSFSRSDKLVRHQRTHTGEKPYKCPECGKSFSSRRTCRAH |
| | R | AGAGTAGTAAACTGTG | | QRTHTGEKPYKCPECGKSFSRSDHLTTHQRTHTGEKPYKCPECGKSFSRKDNLK |
| | | AAAACGTGACAAGGCA | | NHQRTHTGEKPYKCPECGKSFSQSGDLRRHQRTHTGEKPYKCPECGKSFSRKDN |
| | | AAGTGGCGTGGG | | LKNHQRTHTGEKPYKCPECGKSFSDPGNLVRHQRTHTGEKPYKCPECGKSFSSR |
| | | (764) | | RTCRAHQRTHTGEKPYKCPECGKSFSQRANLRAHQRTHTGEKPYKCPECGKSFS |
| | | | | QAGHLASHQRTHTGEKPYKCPECGKSFSRNDALTEHQRTHTGEKPYKCPECGKS |
| | | | | FSQRANLRAHQRTHTGEKPYKCPECGKSFSHRTTLTNHQRTHTGEKPYKCPECG |
| | | | | KSFSHRTTLTNHQRTHTGEKPYKCPECGKSFSRADNLTEHQRTHTGEKPYKCPE |
| | | | | CGKSFSSPADLTRHQRTHTGEKPYKCPECGKSFSRSDHLTTHQRTHTGEKPYKC |
| | | | | PECGKSFSHKNALQNHQRTHTGEKPYKCPECGKSFSRADNLTEHQRTHTGEKPY |
| | | | | KCPECGKSFSRRDELNVHQRTHTGKKTS (765) |
| 3′ | ZnF6b | GGACACAGAGTAGTAA | 55.80 | LEPGEKPYKCPECGKSFSDSGNLRVHQRTHTGEKPYKCPECGKSFSQSSSLVRH |
| | R | AC (766) | | QRTHTGEKPYKCPECGKSFSQSSSLVRHQRTHTGEKPYKCPECGKSFSQLAHLR |
| | | | | AHQRTHTGEKPYKCPECGKSFSSKKALTEHQRTHTGEKPYKCPECGKSFSQRAH |
| | | | | LERHQRTHTGKKTS (767) |
| 5′ | ZnF10 | AAAGCTAGCAGCATGG | 57.55 | LEPGEKPYKCPECGKSFSQSGDLRRHQRTHTGEKPYKCPECGKSFSRRDELNVH |
| | F | CA (768) | | QRTHTGEKPYKCPECGKSFSERSHLREHQRTHTGEKPYKCPECGKSFSERSHLR |
| | | | | EHQRTHTGEKPYKCPECGKSFSTSGELVRHQRTHTGEKPYKCPECGKSFSQRAN |
| | | | | LRAHQRTHTGKKTS (769) |
| 5′ | ZnF11 | CCTCTTATAAGGCCCA | 52.55 | LEPGEKPYKCPECGKSFSQKSSLIAHQRTHTGEKPYKCPECGKSFSRSDHLTNH |
| | F | AGAGGATA (770) | | QRTHTGEKPYKCPECGKSFSRKDNLKNHQRTHTGEKPYKCPECGKSFSSKKHLA |
| | | | | EHQRTHTGEKPYKCPECGKSFSRSDHLTNHQRTHTGEKPYKCPECGKSFSQKSS |
| | | | | LIAHQRTHTGEKPYKCPECGKSFSTTGALTEHQRTHTGEKPYKCPECGKSFSTK |
| | | | | NSLTEHQRTHTGKKTS (771) |
| 5′ | ZnF12 | CAACATCCTTGACTTA | 55.00 | LEPGEKPYKCPECGKSFSSKKALTEHQRTHTGEKPYKCPECGKSFSTTGNLTVH |
| | F | ATCAC (772) | | QRTHTGEKPYKCPECGKSFSTTGALTEHQRTHTGEKPYKCPECGKSFSQAGHLA |
| | | | | SHQRTHTGEKPYKCPECGKSFSTKNSLTEHQRTHTGEKPYKCPECGKSFSTSGN |
| | | | | LTEHQRTHTGEKPYKCPECGKSFSQSGNLTEHQRTHTGKKTS (773) |
| 5 | ZnF13 | GGTAGCAAAAAGGTAA | 46.33 | LEPGEKPYKCPECGKSFSDKKDLTRHQRTHTGEKPYKCPECGKSFSQSSSLVRH |
| | F | CC (774) | | QRTHTGEKPYKCPECGKSFSRKDNLKNHQRTHTGEKPYKCPECGKSFSQRANLR |
| | | | | AHQRTHTGEKPYKCPECGKSFSERSHLREHQRTHTGEKPYKCPECGKSFSTSGH |
| | | | | LVRHQRTHTGKKTS (775) |
| 3′ | ZnF14 | TGGGGTGCAAGAGGCC | 61.28 | LEPGEKPYKCPECGKSFSDPGALVRHQRTHTGEKPYKCPECGKSFSRNDALTEH |
| | R | AGGCCAGAGTTGTTCT | | QRTHTGEKPYKCPECGKSFSTSGSLVRHQRTHTGEKPYKCPECGKSFSTSGSLV |
| | | GGTC (776) | | RHQRTHTGEKPYKCPECGKSFSQLAHLRAHQRTHTGEKPYKCPECGKSFSDCRD |
| | | | | LARHQRTHTGEKPYKCPECGKSFSRADNLTEHQRTHTGEKPYKCPECGKSFSDP |
| | | | | GHLVRHQRTHTGEKPYKCPECGKSFSQLAHLRAHQRTHTGEKPYKCPECGKSFS |
| | | | | QSGDLRRHQRTHTGEKPYKCPECGKSFSTSGHLVRHQRTHTGEKPYKCPECGKS |
| | | | | FSRSDHLTTHQRTHTGKKTS (777) |
| 3′ | ZnF15 | CGCATGCTGATTCAGC | 58.41 | LEPGEKPYKCPECGKSFSDPGNLVRHQRTHTGEKPYKCPECGKSFSTKNSLTEH |
| | R | CTCCTGAC (778) | | QRTHTGEKPYKCPECGKSFSTKNSLTEHQRTHTGEKPYKCPECGKSFSRADNLT |
| | | | | EHQRTHTGEKPYKCPECGKSFSHKNALQNHQRTHTGEKPYKCPECGKSFSRNDA |
| | | | | LTEHQRTHTGEKPYKCPECGKSFSRRDELNVHQRTHTGEKPYKCPECGKSFSHT |
| | | | | GHLLEHQRTHTGKKTS (779) |
| 3′ | ZnF14 | AGTCAAGCAACAGGAT | 50.89 | LEPGEKPYKCPECGKSFSQAGHLASHQRTHTGEKPYKCPECGKSFSQRAHLERH |
| | R | GA (780) | | QRTHTGEKPYKCPECGKSFSSPADLTRHQRTHTGEKPYKCPECGKSFSQSGDLR |
| | | | | RHQRTHTGEKPYKCPECGKSFSQSGNLTEHQRTHTGEKPYKCPECGKSFSHRTT |
| | | | | LTNHQRTHTGKKTS (781) |
| 3′ | ZnF15 | GTCAAGCAACAGGATG | 59.22 | LEPGEKPYKCPECGKSFSHKNALQNHQRTHTGEKPYKCPECGKSFSTSGELVRH |
| | R | ATCCAAATGCTATT | | QRTHTGEKPYKCPECGKSFSTTGNLTVHQRTHTGEKPYKCPECGKSFSTSHSLT |
| | | (782) | | EHQRTHTGEKPYKCPECGKSFSTSGNLVRHQRTHTGEKPYKCPECGKSFSTSGN |
| | | | | LVRHQRTHTGEKPYKCPECGKSFSRADNLTEHQRTHTGEKPYKCPECGKSFSQS |
| | | | | GNLTEHQRTHTGEKPYKCPECGKSFSRKDNLKNHQRTHTGEKPYKCPECGKSFS |
| | | | | DPGALVRHQRTHTGKKTS (783) |

In embodiments, ZNFs for targeting human genomic safe harbor sites using any of the ZNF-based targeting elements to Chromosome X (e.g., hg38 chrX:134,419,661-134,541,172 or hg38 chrX:134,476,304-134,476,307 (85); chrX:134,476,337-134,476,340 (51)) are shown in TABLE 5E:

| ChrX | | | | |
|---|---|---|---|---|
| TTAA | NAME | TARGET (SEQ ID NO: _) | SCORE | ZFP AMINO ACID CODE (SEQ ID NO: _) |
| 5′ | ZnF41F | GTAGAAACTCGCCTTATG (784) | 54.04 | LEPGEKPYKCPECGKSFSRRDELNVHQRTHTGEKPYKCPECG |
| | | | | KSFSTTGALTEHQRTHTGEKPYKCPECGKSFSHIGHLLEHQR |
| | | | | THTGEKPYKCPECGKSFSTHLDLIRHQRTHTGEKPYKCPECG |
| | | | | KSFSQSSNLVRHQRTHTGEKPYKCPECGKSFSQSSSLVRHQR |
| | | | | THTGKKTS (785) |
| 5′ | ZnF42F | TGAATGAGTCCTGTCCATCTT (786) | 55.08 | LEPGEKPYKCPECGKSFSTTGALTEHQRTHTGEKPYKCPECG |
| | | | | KSFSTSGNLTEHQRTHTGEKPYKCPECGKSFSDPGALVRHQR |
| | | | | THTGEKPYKCPECGKSFSTKNSLTEHQRTHTGEKPYKCPECG |
| | | | | KSFSHRTTLTNHQRTHTGEKPYKCPECGKSFSRRDELNVHQR |
| | | | | THTGEKPYKCPECGKSFSQAGHLASHQRTHTGKKTS (787) |
| 5′ | ZnF43F | AAGATTAGAACAAATGTCCAG (788) | 60.20 | LEPGEKPYKCPECGKSFSRADNLTEHQRTHTGEKPYKCPECG |
| | | | | KSFSDPGALVRHQRTHTGEKPYKCPECGKSFSTTGNLTVHQR |
| | | | | THTGEKPYKCPECGKSFSSPADLTRHQRTHTGEKPYKCPECG |
| | | | | KSFSQLAHLRAHQRTHTGEKPYKCPECGKSFSHKNALQNHQR |
| | | | | THTGEKPYKCPECGKSFSRKDNLKNHQRTHTGKKTS (789) |
| 3′ | ZnF44R | ACTCTAAGCAGCAATGTA (790) | 59.94 | LEPGEKPYKCPECGKSFSQSSSLVRHQRTHTGEKPYKCPECG |
| | | | | KSFSTTGNLTVHQRTHTGEKPYKCPECGKSFSERSHLREHQR |
| | | | | THTGEKPYKCPECGKSFSERSHLREHQRTHTGEKPYKCPECG |
| | | | | KSFSQNSTLTEHQRTHTGEKPYKCPECGKSFSTHLDLIRHQR |
| | | | | THTGKKTS (791) |
| 5′ | ZnF45R | TGGGATAGTGAAAATGTC (792) | 57.10 | LEPGEKPYKCPECGKSFSDPGALVRHQRTHTGEKPYKCPECG |
| | | | | KSFSTTGNLTVHQRTHTGEKPYKCPECGKSFSQSSNLVRHQR |
| | | | | THTGEKPYKCPECGKSFSHRTTLTNHQRTHTGEKPYKCPECG |
| | | | | KSFSTSGNLVRHQRTHTGEKPYKCPECGKSFSRSDHLTTHQR |
| | | | | THTGKKTS (793) |
| 5′ | ZnF46R | AAAACTTGGGTCACTAAAATAGATGAT | 61.20 | LEPGEKPYKCPECGKSFSTSGNLVRHQRTHTGEKPYKCPECG |
| | | (794) | | KSFSTSGNLVRHQRTHTGEKPYKCPECGKSFSQKSSLIAHQR |
| | | | | THTGEKPYKCPECGKSFSQRANLRAHQRTHTGEKPYKCPECG |
| | | | | KSFSTHLDLIRHQRTHTGEKPYKCPECGKSFSDPGALVRHQR |
| | | | | THTGEKPYKCPECGKSFSRSDHLTTHQRTHTGEKPYKCPECG |
| | | | | KSFSTHLDLIRHQRTHTGEKPYKCPECGKSFSQRANLRAHQR |
| | | | | THTGKKTS (795) |
| 5′ | ZnF47R | AAACATGGAAAAGGTCAAAAACTTGGG | 43.59 | LEPGEKPYKCPECGKSFSRSDKLVRHQRTHTGEKPYKCPECG |
| | | (796) | | KSFSTTGALTEHQRTHTGEKPYKCPECGKSFSQRANLRAHQR |
| | | | | THTGEKPYKCPECGKSFSQSGNLTEHQRTHTGEKPYKCPECG |
| | | | | KSFSTSGHLVRHQRTHTGEKPYKCPECGKSFSQRANLRAHQR |
| | | | | THTGEKPYKCPECGKSFSQRAHLERHQRTHTGEKPYKCPECG |
| | | | | KSFSTSGNLTEHQRTHTGEKPYKCPECGKSFSQRANLRAHQR |
| | | | | THTGKKTS (797) |
| 3′ | ZnF48R | AATGACTAGAATGAAGTCCTACTG | 59.44 | LEPGEKPYKCPECGKSFSRNDALTEHQRTHTGEKPYKCPECG |
| | | (798) | | KSFSQNSTLTEHQRTHTGEKPYKCPECGKSFSDPGALVRHQR |
| | | | | THTGEKPYKCPECGKSFSQSSNLVRHQRTHTGEKPYKCPECG |
| | | | | KSFSTTGNLTVHQRTHTGEKPYKCPECGKSFSREDNLHTHQR |
| | | | | THTGEKPYKCPECGKSFSDPGNLVRHQRTHTGEKPYKCPECG |
| | | | | KSFSTTGNLTVHQRTHTGKKTS (799) |

In embodiments, the mobile element enzyme is capable of inserting a donor DNA at a TA dinucleotide site. In embodiments, the mobile element enzyme is capable of inserting a donor DNA at a TTAA (SEQ ID NO: 440) tetranucleotide site.

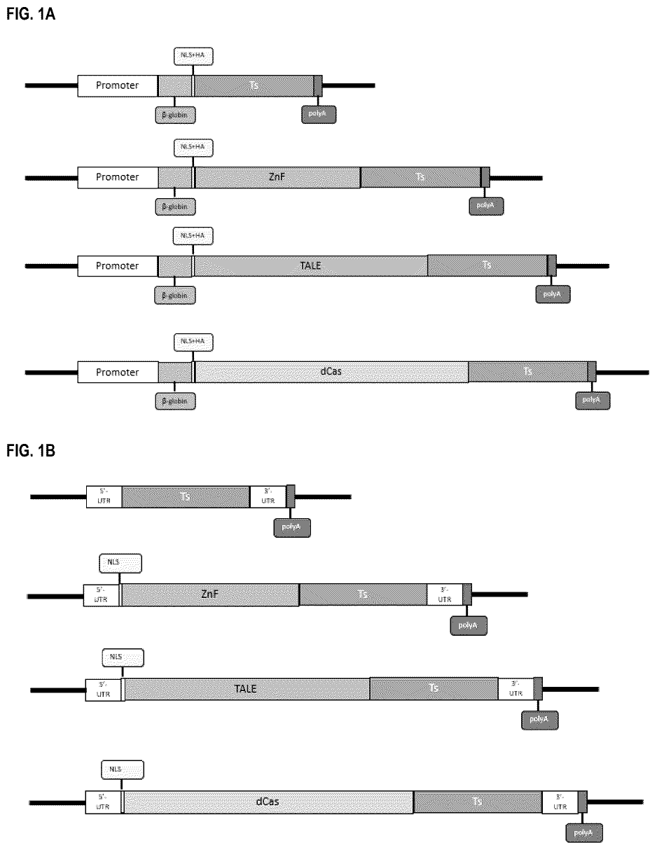

In embodiments, the present disclosure relates to a system having nucleic acids encoding the enzyme and the donor DNA, respectively. FIGS. 1A-1D show examples of a system in accordance with embodiments of the present disclosure.

### Linkers

In embodiments, the targeting element comprises a nucleic acid binding component of the gene-editing system. In embodiments, the enzyme capable of performing targeted genomic integration (e.g., without limitation, a chimeric mobile element enzyme) and the targeting element, e.g., nucleic acid binding component of the gene-editing system are fused or linked to one another. For example, in embodiments, the mobile element enzyme and the targeting element, e.g., nucleic acid binding component of the gene-editing system are fused or linked to one another. In embodiments, the mobile element enzyme and the targeting element, e.g., nucleic acid binding component of the gene-editing system are connected via a linker.

In embodiments, the linker is a flexible linker. In embodiments, the flexible linker is substantially comprised of glycine and serine residues, optionally wherein the flexible linker comprises (Gly₄Ser)ₙ, where n is from about 1 to about 12. In embodiments, the flexible linker is of about 20, or about 30, or about 40, or about 50, or about 60 amino acid residues.

In embodiments, the flexible linker is about 50, or about 100, or about 150, or about 200 amino acid residues in length.

In embodiments, the flexible linker comprises at least about 150 nucleotides (nt), or at least about 200 nt, or at least about 250 nt, or at least about 300 nt, or at least about 350 nt, or at least about 400 nt, or at least about 450 nt, or at least about 500 nt, or at least about 500 nt, or at least about 600 nt. In embodiments, the flexible linker comprises from about 450 nt to about 500 nt.

In embodiments, the mobile element enzyme and the targeting element, e.g., nucleic acid binding component of the gene-editing system are encoded on a single polypeptide.

### Inteins

Inteins (INTervening protEINS) are mobile genetic elements that are protein domains, found in nature, with the capability to carry out the process of protein splicing. See Sarmiento & Camarero (2019) Current Protein & Peptide Science, 20(5), 408-424, which is incorporated by reference herein in its entirety. Protein spicing is a post-translation biochemical modification which results in the cleavage and formation of peptide bonds between precursor polypeptide segments flanking the intein. Id. Inteins apply standard enzymatic strategies to excise themselves post-translationally from a precursor protein via protein splicing. Nanda et al., Microorganisms vol. 8,12 2004. 16 Dec. 2020, doi:10.3390/microorganisms8122004. An intein can splice its flanking N- and C-terminal domains to become a mature protein and excise itself from a sequence. For example, split inteins have been used to control the delivery of heterologous genes into transgenic organisms. See Wood & Camarero (2014) J Biol Chem. 289(21):14512-14519. This approach relies on splitting the target protein into two segments, which are then post-translationally reconstituted in vivo by protein trans-splicing (PTS). See Aboye & Camarero (2012) J. Biol. Chem. 287, 27026-27032. More recently, an intein-mediated split-Cas9 system has been developed to incorporate Cas9 into cells and reconstitute nuclease activity efficiently. Truong et al., Nucleic Acids Res. 2015, 43 (13), 6450-6458. The protein splicing excises the internal region of the precursor protein, which is then followed by the ligation of the N-extein and C-extein fragments, resulting in two polypeptides—the excised intein and the new polypeptide produced by joining the C- and N-exteins. Sarmiento & Camarero (2019).

In embodiments, intein-mediated incorporation of DNA binders such as, without limitation, dCas9, dCas12j, or TALEs, allows creation of a split-enzyme system such as, without limitation, split-MLT mobile element enzyme system, that permits reconstitution of the full-length enzyme, e.g., MLT mobile element enzyme, from two smaller fragments. This allows avoiding the need to express DNA binders at the N- or C-terminus of an enzyme, e.g., MLT mobile element enzyme. In this approach, the two portions of an enzyme, e.g., MLT mobile element enzyme, are fused to the intein and, after co-expression, the intein allows producing a full-length enzyme, e.g., MLT mobile element enzyme, by post-translation modification. Thus, in embodiments, a nucleic acid encoding the enzyme capable of performing targeted genomic integration comprises an intein. In embodiments, the nucleic acid encodes the enzyme in the form of first and second portions with the intein encoded between the first and second portions, such that the first and second portions are fused into a functional enzyme upon post-translational excision of the intein from the enzyme.

In embodiments, an intein is a suitable ligand-dependent intein, for example, an intein selected from those described in U.S. Pat. No. 9,200,045; Mootz et al., J. Am. Chem. Soc. 2002; 124, 9044-9045; Mootz et al., J. Am. Chem. Soc. 2003; 125, 10561-10569; Buskirk et al., Proc. Natl. Acad. Sci. USA. 2004; 101, 10505-10510; Skretas & Wood. Protein Sci. 2005; 14, 523-532; Schwartz, et al., Nat. Chem. Biol. 2007; 3, 50-54; Peck et al., Chem. Biol. 2011; 18 (5), 619-630; the entire contents of each of which are hereby incorporated by reference herein.

In embodiments the intein is NpuN (Intein-N) (SEQ ID NO: 423) and/or NpuC (Intein-C) (SEQ ID NO: 424), or a variant thereof, e.g., a sequence having at least about 90%, or at least about 93%, or at least about 95%, or at least about 97%, or at least about 98%, or at least about 99% identity thereto.

SEQ ID NO: 423: nucleotide sequence of NpuN (Intein-N)
GGCGGATCTGGCGGTAGTGCTGAGTATTGTCTGAGTTACGAAACGGAAATACTCACGGTTGAGTATGGGCTTCTTCC
AATTGGCAAAATCGTTGAAAAGCGCATAGAGTGTACGGTGTATTCCGTCGATAACAACGGTAATATCTACACCCAGC
CGGTAGCTCAGTGGCACGACCGAGGCGAACAGGAAGTGTTCGAGTATTGCTTGGAAGATGGCTCCCTTATCCGCGCC
ACTAAAGACCATAAGTTTATGACGGTTGACGGGCAGATGCTGCCTATAGACGAAATATTTGAGAGAGAGCTGGACTT
GATGAGAGTCGATAATCTGCCAAAT
SEQ ID NO: 424: nucleotide sequence of NpuC (Intein-C)
GGCGGATCTGGCGGTAGTGGGGGTTCCGGATCCATAAAGATAGCTACTAGGAAATATCTTGGCAAACAAAACGTCTA
TGACATAGGAGTTGAGCGAGATCACAATTTTGCTTTGAAGAATGGGTTCATCGCGTCTAATTGCTTCAACGCTAGCG
GCGGGTCAGGAGGCTCTGGTGGAAGC

### Nucleic Acids of the Disclosure

In embodiments, a nucleic acid encoding the enzyme is RNA. In embodiments, a nucleic acid encoding the donor DNA is DNA.

In embodiments, the donor DNA comprises a gene encoding a complete polypeptide. In embodiments, the donor DNA comprises a gene which is defective or substantially absent in a disease state.

In embodiments, the enzyme (e.g., without limitation, the mobile element enzyme) is encoded by a recombinant or synthetic nucleic acid. In embodiments, the nucleic acid is RNA, optionally a helper RNA. In embodiments, the nucleic acid is RNA that has a 5′-m7G cap (cap0, or cap1, or cap2), optionally with pseudouridine substitution (e.g., without limitation n-methyl-pseudouridine) and/or a 5-methoxy substitution (e.g., without limitation, 5-methoxy-U) and optionally a poly-A tail of about 30, or about 34, or about 50, or about 55, or about 70, or about 80, or about 100, of about 150 nucleotides in length. In embodiments, the poly-A tail is of about 30 nucleotides in length, optionally 34 nucleotides in length. In embodiments, a nuclear localization signal is placed before the enzyme start codon at the N-terminus, optionally at the C-terminus.

In embodiments, the first nucleic acid RNA comprises one or more uridine modifications. In embodiments, the first nucleic acid RNA comprises N1-methyl-psuedo U and/or 5-methoxy-U.

In embodiments, the first nucleic acid RNA comprises a poly-A tail of about 80 nucleotides in length.

In embodiments, the nucleic acid that is RNA has a 5′-m7G cap (cap 0, or cap 1, or cap 2).

In embodiments, the nucleic acid comprises a 5′ cap structure, a 5′-UTR comprising a Kozak consensus sequence, a 5′-UTR comprising a sequence that increases RNA stability in vivo, a 3′-UTR comprising a sequence that increases RNA stability in vivo, and/or a 3′ poly(A) tail.

In embodiments, the enzyme (e.g., without limitation, a mobile element enzyme) is incorporated into a vector or a vector-like particle. In embodiments, the vector is a non-viral vector.

In embodiments, a nucleic acid encoding the enzyme in accordance with embodiments of the present disclosure, is DNA.

In various embodiments, a construct comprising a donor DNA is any suitable genetic construct, such as a nucleic acid construct, a plasmid, or a vector. In various embodiments, the construct is DNA, which is referred to herein as a donor DNA. In embodiments, sequences of a nucleic acid encoding the donor DNA is codon optimized to provide improved mRNA stability and protein expression in mammalian systems.

In embodiments, the enzyme and the donor DNA are included in different vectors. In embodiments, the enzyme and the donor DNA are included in the same vector.

In various embodiments, a nucleic acid encoding the enzyme capable of performing targeted genomic integration (e.g., without limitation, a mobile element enzyme which is a chimeric mobile element enzyme) is RNA (e.g., helper RNA), and a nucleic acid encoding a donor DNA is DNA.

As would be appreciated in the art, a donor DNA often includes an open reading frame that encodes a transgene at the middle of donor DNA and terminal repeat sequences at the 5′ and 3′ end of the donor DNA. The translated mobile element enzyme binds to the 5′ and 3′ sequence of the donor DNA and carries out the transposition function.

In embodiments, mobile elements, which are used to refer to polynucleotides capable of inserting copies of themselves into other polynucleotides. The term, mobile element is well known to those skilled in the art and includes classes of mobile element that can be distinguished on the basis of sequence organization, for example inverted terminal sequences at each end, and/or directly repeated long terminal repeats (LTRs) at the ends. In embodiments, the mobile element as described herein may be described as a piggyBac like element, e.g., a mobile element that is characterized by its traceless excision, which recognizes TTAA (SEQ ID NO: 440) sequence and restores the sequence at the insert site back to the original TTAA (SEQ ID NO: 440) sequence.

In embodiments, donor DNA or transgene are used interchangeably with mobile elements.

In embodiments, the donor DNA is flanked by one or more end sequences or terminal ends. In embodiments, the donor DNA is or comprises a gene encoding a complete polypeptide. In embodiments, the donor DNA is or comprises a gene which is defective or substantially absent in a disease state.

In embodiments, the mobile element includes a MLT mobile element enzyme (e.g., without limitation, a MLT mobile element enzyme having at least about 90% identity to the amino acid sequence of SEQ ID NO: 1, SEQ ID NO: 10, or SEQ ID NO: 11). For example, the mobile element enzyme can act on a left terminal end having a nucleotide sequence of SEQ ID NO: 431 or a sequence having at least about 90%, or at least about 93%, or at least about 95%, or at least about 97%, or at least about 98%, or at least about 99% identity thereto. In embodiments, the enzyme can act on a right terminal end having a nucleotide sequence of SEQ ID NO: 432 or a sequence having at least about 90%, or at least about 93%, or at least about 95%, or at least about 97%, or at least about 98%, or at least about 99% identity thereto. In embodiments, the enzyme acts on both MLT left end and MLT right end, having nucleotide sequences of SEQ ID NO: 431 and of SEQ ID NO: 432 respectively, or a sequence having at least about 90%, or at least about 93%, or at least about 95%, or at least about 97%, or at least about 98%, or at least about 99% identity thereto.

(SEQ ID NO: 431)
TTAACACTTGGATTGCGGGAAACGAGITAAGTCGGCTCGCGTGAATTGCGCGTACTCCGCGGGAGCCGTC
TTAACTCGGITCATATAGATTTGCGGTGGAGTGCGGGAAACGTGTAAACTCGGGCCGATTGTAACTGCGT
ATTACCAAATATTTGTT

In embodiments, a MLT left end (5′ to 3′) is as follows

(SEQ ID NO: 432)
AATTATTTATGTACTGAATAGATAAAAAAATGTCTGTGATTGAATAAATTTTCATTTTTTACACAAGAAA
CCGAAAATTTCATTTCAATCGAACCCATACTTCAAAAGATATAGGCATTTTAAACTAACTCTGATTTTGC
GCGGGAAACCTAAATAATTGCCCGCGCCATCITATATTTTGGCGGGAAATTCACCCGACACCGTGGTGTT
AA

In embodiments, a transgene of the donor DNA, e.g., chimeric CAR or chimeric CAAR, is associated with various regulatory elements that are selected to ensure stable expression of a construct with the transgene. Thus, in embodiments, a transgene is encoded by a non-viral vector (e.g., without limitation, a DNA plasmid) that can comprise one or more insulator sequences that prevent or mitigate activation or inactivation of nearby genes. The insulators flank the donor DNA (transgene cassette) to reduce transcriptional silencing and position effects imparted by chromosomal sequences. As an additional effect, the insulators can eliminate functional interactions of the transgene enhancer and promoter sequences with neighboring chromosomal sequences. In embodiments, the one or more insulator sequences comprise an HS4 insulator (1.2-kb 5′-HS4 chicken β-globin (cHS4) insulator element) and an D4Z4 insulator (tandem macrosatellite repeats linked to Facio-Scapulo-Humeral Dystrophy (FSHD). In embodiments, the sequences of the HS4 insulator and the D4Z4 insulator are as described in Rival-Gervier et al. Mol Ther. 2013 August; 21(8):1536-50, which is incorporated herein by reference in its entirety.

In embodiments, the donor DNA, e.g., chimeric CAR or chimeric CAAR is inserted into a GSHS location in a host genome. GSHSs is defined as loci well-suited for gene transfer, as integrations within these sites are not associated with adverse effects such as proto-oncogene activation, tumor suppressor inactivation, or insertional mutagenesis. GSHSs can defined by the following criteria: (1) distance of at least 50 kb from the 5′ end of any gene, (2) distance of at least 300 kb from any cancer-related gene, (3) distance of at least 300 kb from any microRNA (miRNA), (4) location outside a transcription unit, and (5) location outside ultra-conserved regions (UCRs) of the human genome. See Papapetrou et al. Nat Biotechnol 2011; 29:73-8; Bejerano et al. Science 2004; 304:1321-5.

Furthermore, the use of GSHS locations can allow stable transgene expression across multiple cell types. One such site, chemokine C—C motif receptor 5 (CCR5) has been identified and used for integrative gene transfer. CCR5 is a member of the beta chemokine receptor family and is required for the entry of R5 tropic viral strains involved in primary infections. A homozygous 32 bp deletion in the CCR5 gene confers resistance to HIV-1 virus infections in humans. Disrupted CCR5 expression, naturally occurring in about 1% of the Caucasian population, does not appear to result in any reduction in immunity. Lobritz at al., Viruses 2010; 2:1069-105. A clinical trial has demonstrated safety and efficacy of disrupting CCR5 via targetable nucleases. Tebas at al., HIV. N Engl J Med 2014; 370:901-10.

In embodiments, the donor DNA is under control of a tissue-specific promoter. The tissue-specific promoter is, e.g., without limitation, a liver-specific promoter. In embodiments, the liver-specific promoter is an LP1 promoter that, in embodiments, is a human LP1 promoter. The LP1 promoter is described, e.g., in Nathwani et al. Blood vol. 2006; 107(7):2653-61, and it is constructed, without limitation, as described in Nathawani et al.

It should be appreciated however that a variety of promoters can be used, including other tissue-specific promoters, inducible promoters, constitutive promoters, etc.

In embodiments, the present nucleic acids include polymeric form of nucleotides of any length, either ribonucleotides or deoxyribonucleotides, or analogs or derivatives thereof. In embodiments, there is provided double- and single-stranded DNA, as well as double- and single-stranded RNA, and RNA-DNA hybrids. In embodiments, transcriptionally-activated polynucleotides such as methylated or capped polynucleotides are provided. In embodiments, the present compositions are mRNA or DNA.

In embodiments, the present non-viral vectors are linear or circular DNA molecules that comprise a polynucleotide encoding a polypeptide and is operably linked to control sequences, wherein the control sequences provide for expression of the polynucleotide encoding the polypeptide. In embodiments, the non-viral vector comprises a promoter sequence, and transcriptional and translational stop signal sequences. Such vectors may include, among others, chromosomal and episomal vectors, e.g., vectors bacterial plasmids, from donor DNAs, from yeast episomes, from insertion elements, from yeast chromosomal elements, and vectors from combinations thereof. The present constructs may contain control regions that regulate as well as engender expression.

In embodiments, the construct comprising the enzyme and/or donor DNA is codon optimized. Donor DNA codon optimization is used to optimize therapeutic potential of the donor DNA and its expression in the host organism. Codon optimization is performed to match the codon usage in the donor DNA with the abundance of transfer RNA (tRNA) for each codon in a host organism or cell. Codon optimization methods are known in the art and described in, for example, WO 2007/142954, which is incorporated by reference herein in its entirety. Optimization strategies can include, for example, the modification of translation initiation regions, alteration of mRNA structural elements, and the use of different codon biases.

In embodiments, the construct comprising the enzyme and/or donor DNA includes several other regulatory elements that are selected to ensure stable expression of the construct. Thus, in embodiments, the non-viral vector is a DNA plasmid that can comprise one or more insulator sequences that prevent or mitigate activation or inactivation of nearby genes. In embodiments, the one or more insulator sequences comprise an HS4 insulator (1.2-kb 5′-HS4 chicken β-globin (cHS4) insulator element) and an D4Z4 insulator (tandem macrosatellite repeats linked to Facio-Scapulo-Humeral Dystrophy (FSHD). In embodiments, the sequences of the HS4 insulator and the D4Z4 insulator are as described in Rival-Gervier et al. Mol Ther. 2013 August; 21(8):1536-50, which is incorporated herein by reference in its entirety. In embodiments, the gene of the construct comprising the enzyme and/or donor DNA is capable of transposition in the presence of a mobile element enzyme. In embodiments, the non-viral vector in accordance with embodiments of the present disclosure comprises a nucleic acid construct encoding a mobile element enzyme. The mobile element enzyme is an RNA mobile element enzyme plasmid. In embodiments, the non-viral vector further comprises a nucleic acid construct encoding a DNA mobile element enzyme plasmid. In embodiments, the mobile element enzyme is an in vitro-transcribed mRNA mobile element enzyme. The mobile element enzyme is capable of excising and/or transposing the gene from the construct comprising the enzyme and/or donor DNA to site- or locus-specific genomic regions.

In embodiments, the enzyme and the donor DNA are included in the same vector.

In embodiments, the enzyme is disposed on the same (cis) or different vector (trans) than a donor DNA with a transgene, e.g., chimeric CAR or chimeric CAAR. Accordingly, in embodiments, the enzyme and the donor DNA encompassing a transgene, e.g., chimeric CAR or chimeric CAAR are in cis configuration such that they are included in the same vector.

In embodiments, the enzyme and the donor DNA encompassing a transgene, e.g., chimeric CAR or chimeric CAAR are in trans configuration such that they are included in different vectors. The vector is any non-viral vector in accordance with the present disclosure.

In some aspects, a nucleic acid encoding the enzyme capable of performing targeted genomic integration (e.g., a mobile element enzyme or a chimeric mobile element enzyme) in accordance with embodiments of the present disclosure is provided. The nucleic acid is or comprises DNA or RNA. In embodiments, the nucleic acid encoding the enzyme is DNA. In embodiments, the nucleic acid encoding the enzyme capable of performing targeted genomic integration (e.g., a chimeric mobile element enzyme) is RNA such as, e.g., helper RNA. In embodiments, the chimeric mobile element enzyme is incorporated into a vector. In embodiments, the vector is a non-viral vector.

In embodiments, a nucleic acid encoding the transgene, e.g., chimeric CAR or chimeric CAAR, in accordance with embodiments of the present disclosure is provided. The nucleic acid is or comprises DNA or RNA. In embodiments, the nucleic acid encoding the t transgene, e.g., chimeric CAR or chimeric CAAR, is DNA. In embodiments, the nucleic acid encoding the transgene, e.g., chimeric CAR or chimeric CAAR, is RNA such as, e.g., helper RNA. In embodiments, the transgene is incorporated into a vector. In embodiments, the vector is a non-viral vector.

In embodiments, the present enzyme can be in the form or an RNA or DNA and have one or two N-terminus nuclear localization signal (NLS) to shuttle the protein more efficiently into the nucleus. For example, in embodiments, the present enzyme further comprises one, two, three, four, five, or more NLSs. Examples of NLS are provided in Kosugi et al. (J. Biol. Chem. (2009) 284:478-485; incorporated by reference herein). In a particular embodiment, the NLS comprises the consensus sequence K(K/R)X(K/R) (SEQ ID NO: 348). In an embodiment, the NLS comprises the consensus sequence (K/R)(K/R)X₁₀₋₁₂(K/R)_{3/5}(SEQ ID NO: 349), where (K/R)_{3/5} represents at least three of the five amino acids is either lysine or arginine. In an embodiment, the NLS comprises the c-myc NLS. In a particular embodiment, the c-myc NLS comprises the sequence PAAKRVKLD (SEQ ID NO: 350). In a particular embodiment, the NLS is the nucleoplasmin NLS. In embodiments, the nucleoplasmin NLS comprises the sequence KRPAATKKAGQAKKKK (SEQ ID NO: 351). In embodiments, the NLS comprises the SV40 Large T-antigen NLS. In embodiments, the SV40 Large T-antigen NLS comprises the sequence PKKKRKV (SEQ ID NO: 352). In a particular embodiment, the NLS comprises three SV40 Large T-antigen NLSs (e.g., DPKKKRKVDPKKKRKVDPKKKRKV (SEQ ID NO: 353). In embodiments, the NLS may comprise mutations/variations in the above sequences such that they contain 1 or more substitutions, additions or deletions (e.g., about 1, or about 2, or about 3, or about 4, or about 5, or about 10 substitutions, additions, or deletions).

In some aspects, a host cell comprising the nucleic acid in accordance with embodiments of the present disclosure is provided.

### Lipids

In embodiments, at least one of the first nucleic acid and the second nucleic acid is in the form of a lipid nanoparticle (LNP). In embodiments, a composition comprising the first and second nucleic acids is in the form of an LNP.

In embodiments, a nucleic acid encoding the enzyme and a nucleic acid encoding the transgene, e.g., chimeric CAR or chimeric CAAR, are contained within the same lipid nanoparticle (LNP). In embodiments, the nucleic acid encoding the enzyme and the nucleic acid encoding the donor DNA are a mixture incorporated into or associated with the same LNP. In embodiments, the nucleic acid encoding the enzyme and the nucleic acid encoding the donor DNA are in the form of a co-formulation incorporated into or associated with the same LNP.

In embodiments, the LNP is selected from 1,2-dioleoyl-3-trimethylammonium propane (DOTAP), a cationic cholesterol derivative mixed with dimethylaminoethane-carbamoyl (DC-Chol), phosphatidylcholine (PC), triolein (glyceryl trioleate), and 1,2-distearoyl-sn-glycero-3-phosphoethanolamine-N-[carboxy(polyethylene glycol)-2000](DSPE-PEG), 1,2-dimyristoyl-rac-glycero-3-methoxypolyethyleneglycol—2000 (DMG-PEG 2K), and 1,2 distearol-sn-glycerol-3phosphocholine (DSPC) and/or comprising of one or more molecules selected from polyethylenimine (PEI) and poly(lactic-co-glycolic acid) (PLGA), and N-Acetylgalactosamine (GalNAc).

In embodiments, an LNP is as described, e.g., in Patel et al., J Control Release 2019; 303:91-100. The LNP can comprise one or more of a structural lipid (e.g., DSPC), a PEG-conjugated lipid (CDM-PEG), a cationic lipid (MC3), cholesterol, and a targeting ligand (e.g., GalNAc).

In embodiments, a nanoparticle is a particle having a diameter of less than about 1000 nm. In embodiments, nanoparticles of the present disclosure have a greatest dimension (e.g., diameter) of about 500 nm or less, or about 400 nm or less, or about 300 nm or less, or about 200 nm or less, or about 100 nm or less. In embodiments, nanoparticles of the present invention have a greatest dimension ranging between about 50 nm and about 150 nm, or between about 70 nm and about 130 nm, or between about 80 nm and about 120 nm, or between about 90 nm and about 110 nm. In embodiments, the nanoparticles of the present disclosure have a greatest dimension (e.g., a diameter) of about 100 nm.

In some aspects, the cell in accordance with the present disclosure is prepared via an in vivo genetic modification method. In embodiments, a genetic modification in accordance with the present disclosure is performed via an ex vivo method.

In some aspects, the cell in accordance with the present disclosure is prepared by contacting a cell with an enzyme capable of performing targeted genomic integration (e.g., without limitation, a mammalian mobile element enzyme) in vivo. In embodiments, the cell is contacted with the enzyme ex vivo.

In embodiments, the present method provides reduced insertional mutagenesis or oncogenesis as compared to a method with a non-chimeric mobile element enzyme.

### Therapeutic Applications

In embodiments, the disclosure provides a CAR-immune cell or CAAR-immune cell generated by a method described herein.

In embodiments, the disclosure provides a method of delivering a CAR-immune cell or CAAR-immune cell, comprising administering to a patient in need thereof the CAR-immune cell generated by a method described herein.

In embodiments, the disclosure provides a method of treating a disease or condition using a CAR-immune cell or CAAR-immune cell therapy, comprising administering to a patient in need thereof the CAR-immune cell generated by a method described herein.

In embodiments, the disease or condition is or comprises cancer. In embodiments, the cancer is or comprises an adrenal cancer, a biliary track cancer, a bladder cancer, a bone/bone marrow cancer, a brain cancer, a breast cancer, a cervical cancer, a colorectal cancer, a cancer of the esophagus, a gastric cancer, a head/neck cancer, a hepatobiliary cancer, a kidney cancer, a liver cancer, a lung cancer, an ovarian cancer, a pancreatic cancer, a pelvis cancer, a pleura cancer, a prostate cancer, a renal cancer, a skin cancer, a stomach cancer, a testis cancer, a thymus cancer, a thyroid cancer, a uterine cancer, a lymphoma, a melanoma, a multiple myeloma, or a leukemia.

In embodiments, the cancer is selected from one or more of the basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer; glioblastoma; hepatic carcinoma; hepatoma; intra-epithelial neoplasm; kidney or renal cancer; larynx cancer; leukemia; liver cancer; lung cancer; melanoma; myeloma; neuroblastoma; oral cavity cancer; ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; Hodgkin's lymphoma; non-Hodgkin's lymphoma; B-cell lymphoma; small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's Macroglobulinemia; chronic lymphocytic leukemia (CLL); acute lymphoblastic leukemia (ALL); and Hairy cell leukemia.

In embodiments, the cancer is selected from one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulvar cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), and Meigs syndrome.

In embodiments, the disease or condition is or comprises cancer, optionally selected from acute lymphoblastic leukemia, chronic lymphocytic leukemia, non-Hodgkin lymphoma (NHL), and/or multiple myeloma. In embodiments, the cancer is relapsed or refractory acute lymphoblastic leukemia (ALL), a chronic lymphocytic leukemia (CLL), a chronic myelogenous leukemia (CML), a multiple myeloma (MM), an acute myeloid leukemia (AML), diffuse large B-cell lymphoma, primary mediastinal B-cell lymphoma, high grade B-cell lymphoma, transformed follicular lymphoma, and/or Mantle cell lymphoma. In embodiments, the disease or condition is or comprises cancer, optionally a solid tumor, optionally selected from a small cell lung cancer (SCLC), large cell neuroendocrine carcinoma (LCNEC), a gastric cancer, a colon cancer, a renal cell carcinoma, a hepatocellular carcinoma, a bladder urothelial carcinoma, a metastatic melanoma, a breast cancer, an ovarian cancer, a cervical cancer, a head and neck cancer, a pancreatic cancer, a glioma, and/or a glioblastoma.

In embodiments, the disease or condition is or comprises an autoimmune disease or disorder. In embodiments, the autoimmune disease is or comprises multiple sclerosis, diabetes mellitus, lupus, celiac disease, Crohn's disease, ulcerative colitis, Guillain-Barre syndrome, sclerodermas, Goodpasture's syndrome, Wegener's granulomatosis, autoimmune epilepsy, Rasmussen's encephalitis, Primary biliary sclerosis, Sclerosing cholangitis, Autoimmune hepatitis, Addison's disease, Hashimoto's thyroiditis, Fibromyalgia, Meniere's syndrome; transplantation rejection (e.g., prevention of allograft rejection) pernicious anemia, rheumatoid arthritis, systemic lupus erythematosus, dermatomyositis, Sjogren's syndrome, lupus erythematosus, multiple sclerosis, myasthenia gravis, Reiter's syndrome, Grave's disease, and other autoimmune diseases.

In embodiments, the disease or condition is pemphigus vulgaris, paraneoplastic pemphigus, myasthenia gravis, and pemphigus foliaceus.

In embodiments, the cell expresses the CAAR and has high affinity to autoantibodies expressed on B cells.

In embodiments, the cell expresses the CAAR and induces targeted killing of B cells expressing autoantibodies.

In embodiments, the cell expresses the CAAR and has low affinity to antibodies bound to a Fc receptor.

In embodiments, the method of delivering a gene therapy is non-immunogenic, optionally free of cytokine release syndrome. In embodiments, the method of delivering a gene therapy reduces or avoids off-target effects.

In embodiments, the method of comprises delivery via two or more doses. In embodiments, the method has reversible, inducible- or kill-switch capabilities.

In embodiments, the CAR-immune cell is administered by injection or infusion.

In embodiments, the method of delivering a CAR-immune cell comprises delivery via two or more doses.

In embodiments, the method of delivering a CAR-immune cell comprises creating a high copy number of the CAR-immune cell in a subject.

In embodiments, the method requires a single administration. In embodiments, the method requires a plurality of administrations.

### Isolated Cell

In some aspects of the present disclosure, an isolated CAR-immune cell is provided.

In some aspects of the present disclosure, an isolated CAR-immune cell is provided that comprises the transfected cell in accordance with embodiments of the present disclosure.

In some aspects, the present disclosure provides an ex vivo gene therapy approach. Accordingly, in embodiments, the method that is used to treat an inherited or acquired disease in a patient in need thereof comprises (a) contacting a cell obtained from a patient (autologous) or another individual (allogeneic) with a transfected cell in accordance with embodiments of the present disclosure; and (b) administering the cell to a patient in need thereof.

One of the advantages of ex vivo gene therapy is the ability to “sample” the transduced cells before patient administration. This facilitates efficacy and allows performing safety checks before introducing the cell(s) to the patient.

For example, the transduction efficiency and/or the clonality of integration can be assessed before infusion of the product. The present disclosure provides transfected cells and methods that can be effectively used for ex vivo gene modification.

In embodiments, a composition comprising transfected cells in accordance with the present disclosure comprises a pharmaceutically acceptable carrier, excipient or diluent.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile, and the fluid should be easy to draw up by a syringe. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition.

Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Therapeutic compounds can be prepared with carriers that will protect the therapeutic compounds against rapid elimination from the body, such as a controlled release formulation, including implants and microencapsulated delivery systems. Biodegradable, biocompatible polymers can be used, such as collagen, ethylene vinyl acetate, polyanhydrides (e.g., poly[1,3-bis(carboxyphenoxy)propane-co-sebacic-acid](PCPP-SA) matrix, fatty acid dimer-sebacic acid (FAD-SA) copolymer, poly(lactide-co-glycolide)), polyglycolic acid, collagen, polyorthoesters, polyethyleneglycol-coated liposomes, and polylactic acid. Such formulations can be prepared using standard techniques, or obtained commercially, e.g., from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811. Semisolid, gelling, soft-gel, or other formulations (including controlled release) can be used, e.g., when administration to a surgical site is desired. Methods of making such formulations are known in the art and can include the use of biodegradable, biocompatible polymers. See, e.g., Sawyer et al., Yale J Biol Med. 2006; 79(3-4): 141-152.

In embodiments, there is provided a transgenic organism that may comprise cells which have been transformed by the methods of the present disclosure. In embodiments, the organism may be a mammal or an insect. When the organism is a mammal, the organism may include, but is not limited to, a mouse, a rat, a monkey, a dog, a rabbit, a panda and the like. When the organism is an insect, the organism may include, but is not limited to, a fruit fly, a mosquito, a bollworm and the like.

In embodiments, the cells produced in accordance with embodiments of the present disclosure, and/or components for generating cells, is included in a container, kit, pack, or dispenser together with instructions for administration.

Also provided herein are kits comprising: one or more genetic constructs encoding the present enzyme and donor DNA and instructions and/or reagents for the use of the same.

Also provided herein are kits comprising: i) a transfected cell in accordance with embodiments of the present disclosure, ii) instructions for the use of the transfected cell.

Furthermore, in embodiments, a kit is provided for creating a CAR-immune cell or a CAAR-immune cell, and instructions for creating the same, and. optionally, reagents for the same (e.g., media, factors, and the like).

In embodiments, a kit is provided that comprises an enzyme (e.g., without limitation, a recombinant mammalian mobile element enzyme) or a nucleic acid in accordance with embodiments of the present disclosure, and instructions for introducing DNA and/or RNA into a cell using the enzyme.

### Definitions

The following definitions are used in connection with the disclosure disclosed herein. Unless defined otherwise, all technical and scientific terms used herein have the same meaning as commonly understood to one of skill in the art to which this invention belongs.

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50” covers the range of 45 to 55.

An “effective amount,” when used in connection with medical uses is an amount that is effective for providing a measurable treatment, prevention, or reduction in the rate of pathogenesis of a disease of interest.

The term “in vivo” refers to an event that takes place in a subject's body.

The term “ex vivo” refers to an event which involves treating or performing a procedure on a cell, tissue and/or organ which has been removed from a subject's body. Aptly, the cell, tissue and/or organ may be returned to the subject's body in a method of treatment or surgery.

As used herein, the term “variant” encompasses but is not limited to nucleic acids or proteins which comprise a nucleic acid or amino acid sequence which differs from the nucleic acid or amino acid sequence of a reference by way of one or more substitutions, deletions and/or additions at certain positions. The variant may comprise one or more conservative substitutions. Conservative substitutions may involve, e.g., the substitution of similarly charged or uncharged amino acids.

“Carrier” or “vehicle” as used herein refer to carrier materials suitable for drug administration. Carriers and vehicles useful herein include any such materials known in the art, e.g., any liquid, gel, solvent, liquid diluent, solubilizer, surfactant, lipid or the like, which is nontoxic and which does not interact with other components of the composition in a deleterious manner.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs, can also be incorporated into the described compositions and methods.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

The amount of compositions described herein needed for achieving a therapeutic effect may be determined empirically in accordance with conventional procedures for the particular purpose. Generally, for administering therapeutic agents for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures or experimental animals, e.g., for determining the LD₅₀ (the dose lethal to about 50% of the population) and the ED₅₀ (the dose therapeutically effective in about 50% of the population). The dosage can vary depending upon the dosage form employed and the route of administration utilized. The dose ratio between toxic and therapeutic effects is the therapeutic index and can be expressed as the ratio LD₅₀/ED₅₀. In embodiments, compositions and methods that exhibit large therapeutic indices are preferred. A therapeutically effective dose can be estimated initially from in vitro assays, including, for example, cell culture assays. Also, a dose can be formulated in animal models to achieve a circulating plasma concentration range that includes the IC₅₀ as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

As used herein, “methods of treatment” are equally applicable to use of a composition for treating the diseases or disorders described herein and/or compositions for use and/or uses in the manufacture of a medicaments for treating the diseases or disorders described herein.

### Selected Sequences

In embodiments, the present disclosure provides for any of the sequence provided herein, including the below, and a variant sequence having at least about 90%, or at least about 93%, or at least about 95%, or at least about 97%, or at least about 98%, or at least about 99% identity thereto, or at least about 10 mutations, or at least about 9 mutations, or at least about 8 mutations, or at least about 7 mutations, or at least about 6 mutations, or at least about 5 mutations, or at least about 4 mutations, or at least about 3 mutations, or at least about 2 mutations, or at least about 1 mutation.

MLT mobile element enzyme protein
MAQHSDYSDDEFCADKLSNYSCDSDLENASTSDEDSSDDEVMVRPRTLRRRRISSSSSDSESDIEGGREEWSHV
DNPPVLEDFLGHQGLNTDAVINNIEDAVKLFIGDDFFEFLVEESNRYYNQNRNNFKLSKKSLKWKDITPQEMKK
FLGLIVLMGQVRKDRRDDYWITTEPWTETPYFGKIMTRDRFRQIWKAWHFNNNADIVNESDRLCKVRPVLDYFVP
KFINIYKPHQQLSLDEGIVPWRGRLFFRVYNAGKIVKYGILVRLLCESDTGYICNMEIYCGEGKRLLETIQTVV
SPYTDSWYHIYMDNYYNSVANCEALMKNKFRICGTIRKNRGIPKDFQTISLKKGETKFIRKNDILLQVWQSKKP
VYLISSIHSAEMEESQNIDRISKKKIVKPNALIDYNKHMKGVDRADQYLSYYSILRRTVKWTKRLAMYMINCAL
FNSYAVYKSVRQRKMGFKMFLKQTAIHWLTDDIPEDMDIVPDLQPVPSTSGMRAKPPTSDPPCRLSMDMRKHTL
QAIVGSGKKKNILRRCRVCSVHKLRSETRYMCKFCNIPLHKGACFEKYHTLKNY (SEQ ID NO: 1)
MLT codon-optimized Mobile element enzyme DNA
ATGGCCCAGCACAGCGACTACAGCGACGACGAGTTCTGTGCCGATAAGCTGAGTAACTACAGCTGCGACAGCGA
CCTGGAAAACGCCAGCACATCCGACGAGGACAGCTCTGACGACGAGGTGATGGTGCGGCCCAGAACCCTGAGAC
GGAGAAGAATCAGCAGCTCTAGCAGCGACTCTGAATCCGACATCGAGGGCGGCCGGGAAGAGTGGAGCCACGTG
GACAACCCTCCTGTTCTGGAAGATTTTCTGGGCCATCAGGGCCTGAACACCGACGCCGTGATCAACAACATCGA
GGATGCCGTGAAGCTGTTCATAGGAGATGATTTCTTTGAGTTCCTGGTCGAGGAATCCAACCGCTATTACAACC
AGAATAGAAACAACTTCAAGCTGAGCAAGAAAAGCCTGAAGTGGAAGGACATCACCCCTCAGGAGATGAAAAAG
TTCCTGGGACTGATCGTTCTGATGGGACAGGTGCGGAAGGACAGAAGGGATGATTACTGGACAACCGAACCTTG
GACCGAGACCCCTTACTTTGGCAAGACCATGACCAGAGACAGATTCAGACAGATCTGGAAAGCCTGGCACTTCA
ACAACAATGCTGATATCGTGAACGAGTCTGATAGACTGTGTAAAGTGCGGCCAGTGTTGGATTACTTCGTGCCT
AAGTTCATCAACATCTATAAGCCTCACCAGCAGCTGAGCCTGGATGAAGGCATCGTGCCCTGGCGGGGCAGACT
GTTCTTCAGAGTGTACAATGCTGGCAAGATCGTCAAATACGGCATCCTGGTGCGCCTTCTGTGCGAGAGCGATA
CAGGCTACATCTGTAATATGGAAATCTACTGCGGCGAGGGCAAAAGACTGCTGGAAACCATCCAGACCGTCGTT
TCCCCTTATACCGACAGCTGGTACCACATCTACATGGACAACTACTACAATTCTGTGGCCAACTGCGAGGCCCT
GATGAAGAACAAGTTTAGAATCTGCGGCACAATCAGAAAAAACAGAGGCATCCCTAAGGACTTCCAGACCATCT
CTCTGAAGAAGGGCGAAACCAAGTTCATCAGAAAGAACGACATCCTGCTCCAAGTGTGGCAGTCCAAGAAACCC
GTGTACCTGATCAGCAGCATCCATAGCGCCGAGATGGAAGAAAGCCAGAACATCGACAGAACAAGCAAGAAGAA
GATCGTGAAGCCCAATGCTCTGATCGACTACAACAAGCACATGAAAGGCGTGGACCGGGCCGACCAGTACCTGT
CTTATTACTCTATCCTGAGAAGAACAGTGAAATGGACCAAGAGACTGGCCATGTACATGATCAATTGCGCCCTG
TTCAACAGCTACGCCGTGTACAAGTCCGTGCGACAAAGAAAAATGGGATTCAAGATGTTCCTGAAGCAGACAGC
CATCCACTGGCTGACAGACGACATTCCTGAGGACATGGACATTGTGCCAGATCTGCAACCTGTGCCCAGCACCT
CTGGTATGAGAGCTAAGCCTCCCACCAGCGATCCTCCATGTAGACTGAGCATGGACATGCGGAAGCACACCCTG
CAGGCCATCGTCGGCAGCGGCAAGAAGAAGAACATCCTTAGACGGTGCAGGGTGTGCAGCGTGCACAAGCTGCG
GAGCGAGACTCGGTACATGTGCAAGTTTTGCAACATTCCCCTGCACAAGGGAGCCTGCTTCGAGAAGTACCACA
CCCTGAAGAATTACTAG (SEQ ID NO: 2)
PGBD4 Amino Acid Sequence (585 Amino Acids). (SEQ ID NO: 3)

| | |
|---|---|
| MSNPRKRSIP MRDSNTGLEQ LLAEDSFDES DFSEIDDSDN FSDSALEADK | 50 |
| IRPLSHLESD GKSSTSSDSG RSMKWSARAM IPRQRYDFTG TPGRKVDVSD | 100 |
| ITDPLQYFEL FFTEELVSKI TRETNAQAAL LASKPPGPKG FSRMDKWKDT | 150 |
| DNDELKVFFA VMLLQGIVQK PELEMFWSTR PLLDTPYLRQ IMTGERFLLL | 200 |
| FRCLHFVNNS SISAGQSKAQ ISLQKIKPVF DFLVNKFSTV YTPNRNIAVD | 250 |
| ESLMLFKGPL AMKQYLPTKR VRFGLKLYVL CESQSGYVWN ALVHTGPGMN | 300 |
| LKDSADGLKS SRIVLTLVND LLGQGYCVFL DNFNISPMLF RELHQNRTDA | 350 |
| VGTARLNRKQ IPNDLKKRIA KGTTVARFCG ELMALKWCDG KEVTMLSTFH | 400 |
| NDTVIEVNNR NGKKTKRPRV IVDYNENMGA VDSADQMLTS YPSERKRHKV | 450 |
| WYKKFFHHLL HITVLNSYIL FKKDNPEHTM SHINFRLALI ERMLEKHHKP | 500 |
| GQQHLRGRPC SDDVTPLRLS GRHFPKSIPA TSGKQNPTGR CKICCSQYDK | 550 |
| DGKKIRKETR YFCAECDVPL CVVPCFEIYH TKKNY | 585 |

PGBD4 Hyperactive Mutant (S8P, G17R, K134K) Amino Acid Sequence (585 Amino Acids).
(SEQ ID NO: 4)

| | |
|---|---|
| MSNPRKRPIP MRDSNTRLEQ LLAEDSFDES DFSEIDDSDN FSDSALEADK | 50 |
| IRPLSHLESD GKSSTSSDSG RSMKWSARAM IPRQRYDFTG TPGRKVDVSD | 100 |
| ITDPLQYFEL FFTEELVSKI TRETNAQAAL LASKPPGPKG FSRMDKWKDT | 150 |
| DNDELKVFFA VMLLQGIVQK PELEMFWSTR PLLDTPYLRQ IMTGERFLLL | 200 |
| FRCLHFVNNS SISAGQSKAQ ISLQKIKPVF DFLVNKFSTV YTPNRNIAVD | 250 |
| ESLMLFKGPL AMKQYLPTKR VRFGLKLYVL CESQSGYVWN ALVHTGPGMN | 300 |
| LKDSADGLKS SRIVLTLVND LLGQGYCVFL DNFNISPMLF RELHQNRTDA | 350 |
| VGTARLNRKQ IPNDLKKRIA KGTTVARFCG ELMALKWCDG KEVTMLSTFH | 400 |
| NDTVIEVNNR NGKKTKRPRV IVDYNENMGA VDSADQMLTS YPSERKRHKV | 450 |
| WYKKFFHHLL HITVLNSYIL FKKDNPEHTM SHINFRLALI ERMLEKHHKP | 500 |
| GQQHLRGRPC SDDVTPLRLS GRHFPKSIPA TSGKQNPTGR CKICCSQYDK | 550 |
| DGKKIRKETR YFCAECDVPL CVVPCFEIYH TKKNY | 585 |

PGBD4 Hyperactive Mutant (S8P, G17R, K134K) Nucleotide Sequence (1758 bp).
(SEQ ID NO: 5)

| | |
|---|---|
| ATGTCAAATC CTAGAAAACG TCCCATTCCT ATGCGTGATA GTAATACCCG TCTCGAACAG | 60 |
| TTGTTGGCTG AAGATTCATT TGATGAATCT GATTTTTCGG AAATAGATGA TTCTGATAAT | 120 |
| TTTTCGGATA GTGCTTTAGA AGCCGATAAG ATCAGGCCTC TGTCCCATTT AGAATCTGAT | 180 |
| GGAAAGAGCT CTACATCAAG TGACTCAGGG CGCTCCATGA AATGGTCAGC TCGTGCTATG | 240 |
| ATTCCACGTC AAAGGTATGA CTTTACCGGC ACACCTGGCA GAAAAGTCGA TGTCAGTGAT | 300 |
| ATCACTGACC CATTGCAGTA TTTTGAACTG TTCTTTACTG AGGAATTAGT TTCAAAAATT | 360 |
| ACTAGAGAAA CAAATGCCCA AGCTGCCTTG TTGGCTTCAA AGCCACCGGG TCCGAAAGGA | 420 |
| TTTTCGCGAA TGGATAAATG GAAAGACACT GACAATGACG AGCTCAAAGT CTTTTTTGCA | 480 |
| GTAATGTTAC TGCAAGGTAT TGTGCAGAAA CCTGAGCTGG AGATGTTTTG GTCAACAAGG | 540 |
| CCTCTTTTGG ATACACCTTA TCTCAGGCAA ATTATGACTG GTGAAAGATT TTTACTTTTG | 600 |
| TTTCGGTGCC TGCATTTTGT CAACAATTCT TCTATATCTG CTGGTCAATC AAAGGCCCAG | 660 |
| ATTTCATTGC AGAAGATCAA ACCTGTGTTC GACTTTCTTG TAAATAAATT TTCCACTGTA | 720 |
| TATACTCCAA ACAGAAACAT TGCAGTTGAT GAATCACTGA TGCTGTTCAA GGGGCCATTA | 780 |
| GCTATGAAGC AGTACCTCCC GACAAAACGA GTACGATTTG GTCTGAAGCT ATATGTACTT | 840 |
| TGTGAAAGTC AGTCTGGTTA TGTGTGGAAT GCGCTTGTTC ACACAGGGCC TGGCATGAAT | 900 |
| TTGAAAGATT CAGCGGATGG CCTGAAATCA TCACGCATTG TTCTTACCTT GGTCAATGAC | 960 |
| CTTCTTGGCC AAGGGTATTG TGTCTTCCTC GATAACTTTA ATATATCTCC CATGCTTTTC | 1020 |
| AGAGAATTAC ATCAAAATAG GACTGATGCA GTTGGGACAG CTCGTTTGAA CAGAAAACAG | 1080 |
| ATTCCAAATG ATCTGAAAAA AAGGATTGCA AAGGGGACGA CTGTAGCCAG ATTCTGTGGT | 1140 |
| GAACTTATGG CACTGAAATG GTGTGACGGC AAGGAGGTGA CAATGTTGTC AACATTCCAC | 1200 |
| AATGATACTG TGATTGAAGT AAACAATAGA AATGGAAAGA AAACTAAAAG GCCACGTGTC | 1260 |
| ATTGTGGATT ATAACGAGAA TATGGGAGCA GTGGACTCGG CTGATCAAAT GCTTACTTCT | 1320 |
| TATCCATCTG AGCGCAAAAG ACACAAGGTT TGGTATAAGA AATTCTTTCA CCATCTTCTA | 1380 |
| CACATTACAG TGCTGAACTC CTACATCCTG TTCAAGAAGG ATAATCCTGA GCACACGATG | 1440 |
| AGCCATATAA ACTTCAGACT GGCATTGATT GAAAGAATGC TGGAAAAGCA TCACAAGCCA | 1500 |
| GGGCAGCAAC ATCTTCGAGG TCGTCCTTGC TCCGATGATG TCACACCTCT TCGTCTGTCT | 1560 |
| GGAAGACATT TCCCCAAGAG CATACCAGCA ACGTCCGGGA AACAGAATCC AACTGGTCGC | 1620 |
| TGCAAAATTT GCTGCTCCCA ATACGACAAG GATGGCAAGA AGATCCGGAA AGAAACGCGC | 1680 |
| TATTTTTGTG CCGAATGTGA TGTTCCGCTT TGTGTTGTTC CGTGCTTTGA AATTTACCAC | 1740 |
| ACGAAAAAAA ATTATTAA | 1758 |

PGBD1 Amino Acid Sequence (809 Amino Acids). (SEQ ID NO: 6)

| | |
|---|---|
| MYEALPGPAP ENEDGLVKVK EEDPTWEQVC NSQEGSSHTQ EICRLRFRHF CYQEAHGPQE | 60 |
| ALAQLRELCH QWLRPEMHTK EQIMELLVLE QFLTILPKEL QPCVKTYPLE SGEEAVTVLE | 120 |
| NLETGSGDTG QQASVYIQGQ DMHPMVAEYQ GVSLECQSLQ LLPGITTLKC EPPQRPQGNP | 180 |
| QEVSGPVPHG SAHLQEKNPR DKAVVPVENP VRSQTLVKTE EETAQAVAAE KWSHLSLTRR | 240 |
| NLCGNSAQET VMSLSPMTEE IVTKDRLFKA KQETSEEMEQ SGEASGKPNR ECAPQIPCST | 300 |
| PIATERTVAH LNTLKDRHPG DLWARMHISS LEYAAGDITR KGRKKDKARV SELLQGLSFS | 360 |
| GDSDVEKDNE PEIQPAQKKL KVSCFPEKSW TKRDIKPNFP SWSALDSGLL NLKSEKLNPV | 420 |
| ELFELFFDDE TFNLIVNETN NYASQKNVSL EVTVQEMRCV FGVLLLSGFM RHPRREMYWE | 480 |
| VSDTDQNLVR DAIRRDRFEL IFSNLHFADN GHLDQKDKFT KLRPLIKQMN KNFLLYAPLE | 540 |
| EYYCFDKSMC ECFDSDQFLN GKPIRIGYKI WCGTTTQGYL VWFEPYQEES TMKVDEDPDL | 600 |
| GLGGNLVMNF ADVLLERGQY PYHLCFDSFF TSVKLLSALK KKGVRATGTI RENRTEKCPL | 660 |
| MNVEHMKKMK RGYFDFRIEE NNEIILCRWY GDGIISLCSN AVGIEPVNEV SCCDADNEEI | 720 |
| PQISQPSIVK VYDECKEGVA KMDQIISKYR VRIRSKKWYS ILVSYMIDVA MNNAWQLHRA | 780 |
| CNPGASLDPL DERRFVAHFY LEHNAHLSD | 809 |

PGBD2 Amino Acid Sequence (592 Amino Acids). (SEQ ID NO: 7)

| | |
|---|---|
| MASTSRDVIA GRGIHSKVKS AKLLEVLNAM EEEESNNNRE EIFIAPPDNA AGEFTDEDSG | 60 |
| DEDSQRGAHL PGSVLHASVL CEDSGTGEDN DDLELQPAKK RQKAVVKPQR IWTKRDIRPD | 120 |
| FGSWTASDPH IEDLKSQELS PVGLFELFFD EGTINFIVNE TNRYAWQKNV NLSLTAQELK | 180 |
| CVLGILILSG YISYPRRRMF WETSPDSHHH LVADAIRRDR FELIFSYLHF ADNNELDASD | 240 |
| RFAKVRPLII RMNCNFQKHA PLEEFYSFGE SMCEYFGHRG SKQLHRGKPV RLGYKIWCGT | 300 |
| TSRGYLVWFE PSQGTLFTKP DRSLDLGGSM VIKFVDALQE RGFLPYHIFF DKVFTSVKLM | 360 |
| SILRKKGVKA TGTVREYRTE RCPLKDPKEL KKMKRGSFDY KVDESEEIIV CRWHDSSVVN | 420 |
| ICSNAVGIEP VRLTSRHSGA AKTRTQVHQP SLVKLYQEKV GGVGRMDQNI AKYKVKIRGM | 480 |
| KWYSSFIGYV IDAALNNAWQ LHRICCQDAQ VDLLAFRRYI ACVYLESNAD TTSQGRRSRR | 540 |
| LETESREDMI GHWITHQDKR TRCALCHSQT NTRCEKCQKG VHAKCFREYH IR | 592 |

PGBD3 Amino Acid Sequence (593 Amino Acids). (SEQ ID NO: 8)

| | |
|---|---|
| MPRTLSLHEI TDLLETDDSI EASAIVIQPP ENATAPVSDE ESGDEEGGTI NNLPGSLLHT | 60 |
| AAYLIQDGSD AESDSDDPSY APKDDSPDEV PSTFTVQQPP PSRRRKMTKI LCKWKKADLT | 120 |
| VQPVAGRVTA PPNDFFTVMR TPTEILELFL DDEVIELIVK YSNLYACSKG VHLGLISSEF | 180 |
| KCFLGIIFLS GYVSVPRRRM FWEQRTDVHN VLVSAAMRRD RFETIFSNLH VADNANLDPV | 240 |
| DKFSKLRPLI SKLNERCMKF VPNETYFSFD EFMVPYFGRH GCKQFIRGKP IRFGYKFWCG | 300 |
| ATCLGYICWF QPYQGKNPNT KHEEYGVGAS LVLQFSEALT EAHPGQYHFV FNNEFTSIAL | 360 |
| LDKLSSMGHQ ATGTVRKDHI DRVPLESDVA LKKKERGTFD YRIDGKGNIV CRWNDNSVVT | 420 |
| VASSGAGIHP LCLVSRYSQK LKKKIQVQQP NMIKVYNQFM GGVDRADENI DKYRASIRGK | 480 |
| KWYSSPLLFC FELVLQNAWQ LHKTYDEKPV DFLEFRRRVV CHYLETHGHP PEPGQKGRPQ | 540 |
| KRNIDSRYDG INHVIVKQGK QTRCAECHKN TTFRCEKCDV ALHVKCSVEY HTE | 593 |

PGBD5 Amino Acid Sequence (524 Amino Acids). (SEQ ID NO: 9)

| | |
|---|---|
| MAEGGGGARR RAPALLEAAR ARYESLHISD DVFGESGPDS GGNPFYSTSA ASRSSSAASS | 60 |
| DDEREPPGPP GAAPPPPRAP DAQEPEEDEA GAGWSAALRD RPPPRFEDTG GPTRKMPPSA | 120 |
| SAVDFFQLFV PDNVLKNMVV QTNMYAKKFQ ERFGSDGAWV EVTLTEMKAF LGYMISTSIS | 180 |
| HCESVLSIWS GGFYSNRSLA LVMSQARFEK ILKYFHVVAF RSSQTTHGLY KVQPFLDSLQ | 240 |
| NSFDSAFRPS QTQVLHEPLI DEDPVFIATC TERELRKRKK RKFSLWVRQC SSTGFIIQIY | 300 |
| VHLKEGGGPD GLDALKNKPQ LHSMVARSLC RNAAGKNYII FTGPSITSLT LFEEFEKQGI | 360 |
| YCCGLLRARK SDCTGLPLSM LINPATPPAR GQYQIKMKGN MSLICWYNKG HFRFLTNAYS | 420 |
| PVQQGVIIKR KSGEIPCPLA VEAFAAHLSY ICRYDDKYSK YFISHKPNKT WQQVFWFAIS | 480 |
| IAINNAYILY KMSDAYHVKR YSRAQFGERL VRELLGLEDA SPTH | 524 |

Myotis lucifugus (Wild-type) Amino Acid Sequence with Hyperactive Mutations
(S8P, C13R, N125K) 572 Amino Acids. (SEQ ID NO: 10)

| | |
|---|---|
| MSQHSDYPDD EFRADKLSNY SCDSDLENAS TSDEDSSDDE VMVRPRTLRR RRISSSSSDS | 60 |
| ESDIEGGREE WSHVDNPPVL EDFLGHQGLN TDAVINNIED AVKLFIGDDF FEFLVEESNR | 120 |
| YYNQKRNNFK LSKKSLKWKD ITPQEMKKFL GLIVLMGQVR KDRRDDYWTT EPWTETPYFG | 180 |
| KTMTRDRFRQ IWKAWHENNN ADIVNESDRL CKVRPVLDYF VPKFINIYKP HQQLSLDEGI | 240 |
| VPWRGRLFFR VYNAGKIVKY GILVRLLCES DTGYICNMEI YCGEGKRLLE TIQTVVSPYT | 300 |
| DSWYHIYMDN YYNSVANCEA LMKNKFRICG TIRKNRGIPK DFQTISLKKG ETKFIRKNDI | 360 |
| LLQVWQSKKP VYLISSIHSA EMEESQNIDR TSKKKIVKPN ALIDYNKHMK GVDRADQYLS | 420 |
| YYSILRRTVK WTKRLAMYMI NCALFNSYAV YKSVRQRKMG FKMELKQTAI HWLTDDIPED | 480 |
| MDIVPDLQPV PSTSGMRAKP PTSDPPCRLS MDMRKHTLQA IVGSGKKKNI LRRCRVCSVH | 540 |
| KLRSETRYMC KFCNIPLHKG ACFEKYHTLK NY | 572 |

Myotis lucifugus Corrected Amino Acid Sequence with Hyperactive Mutations
(S8P, C13R) 571 Amino Acids. (SEQ ID NO: 11)
MAQHSDYPDDEFRADKLSNYSCDSDLENASTSDEDSSDDEVMVRPRTLRRRRISSSSSDSESDIEGGREEWSHVDNPPVLEDFLGH
QGLNTDAVINNIEDAVKLFIGDDFFEFLVEESNRYYNQNRNNFKLSKKSLKWKDITPQEMKKFLGLIVLMGQVRKDRRDDYWTTEP
WTETPYFGKTMTRDRFRQIWKAWHENNNADIVNESDRLCKVRPVLDYFVPKFINIYKPHQQLSLDEGIVPWRGRLFFRVYNAGKIV
KYGILVRLLCESDTGYICNMEIYCGEGKRLLETIQTVVSPYTDSWYHIYMDNYYNSVANCEALMKNKFRICGTIRKNRGIPKDFQT
ISLKKGETKFIRKNDILLQVWQSKKPVYLISSIHSAEMEESQNIDRTSKKKIVKPNALIDYNKHMKGVDRADQYLSYYSILRRTVK
WTKRLAMYMINCALFNSYAVYKSVRQRKMGFKMFLKQTAIHWLTDDIPEDMDIVPDLQPVPSTSGMRAKPPTSDPPCRLSMDMRKH
TLQAIVGSGKKKNILRRCRVCSVHKLRSETRYMCKFCNIPLHKGACFEKYHTLKNY
Pteropus vampyrus Left End Sequence
Sequence 381 bp. (SEQ ID NO: 12)

| | |
|---|---|
| TTAACCCATT TCCTGTTTGC CCCGAGAATA CTCACCAGCG GCACTTGCAG CTGCAGCGTT | 60 |
| TACCCCGAGA TAACTCGTCG ATTACAGTCC TAACCTTACC CCCAAAGTTT GCCATGAAAT | 120 |
| ATCTCGCTTT TATTATTATT TTCGCATCGC TCTAGTATAT CGATAGTCTT TGGAAACAAA | 180 |
| TGACATCATT CTATTTACAG CATTCTGTTT TTAGTAGTGG TATTTCCATT TACAAAATAT | 240 |
| AGTAATTTTC TATCGCTGAA AATGTCAAAT CCTAGAAAAC GTAGCATTCC TACATGTGAT | 300 |
| GTTAACTTCG TTCTCGAACA GTTGTTAGCC GAAGATTCAT TTGATGAATC CGATTTTTCC | 360 |
| GAAATAGACG ATTCTGATGA T | 381 |

PGBD4 Left End Nucleotide Sequence
Sequence 373 bp. (SEQ ID NO: 13)

| | |
|---|---|
| TTAACTCATT TCTCCTTAGC CCCGAGATTA CGCGCTGCTG TGCCTGCGAC TGCAGCGTTT | 60 |
| ACGCCGAGAT AACTCGTGGA TTACAGTGCC AACCTTACTC CCAAAGTTTG CCACGAAATA | 120 |
| TCTCGCTTCT GTTATTTTCG CATGGTTCTG GTATATTGAC TTTTGAAACA AAAGACATCA | 180 |
| TTCTGTTTAT AGCATTCTGT TTTTAGTAGT GGGATTTCCA TCTACAAAAT ATAGTAATTC | 240 |
| TCGATCGCTG AAATGTCAAA TCCTAGAAAA CGTAGCATTC CTATGCGTGA TAGTAATACC | 300 |
| GGTCTCGAAC AGTTGTTGGC TGAAGATTCA TTTGATGAAT CTGATTTTTC GGAAATAGAT | 360 |
| GATTCTGATA ATT | 373 |

MER75 Left End Nucleotide Sequence
Sequence 344 bp. (SEQ ID NO: 14)

| | |
|---|---|
| TTAACCCTTT TCCCGTTTGC CCCGAGAATA CTCGCCGGCG GCGCTTGCGG CTGCAGCGTT | 60 |
| TACCCCGAGA TAACTTTGCC ACGAAATATC TCGCTTTTAT TATTATTTTC GCATCGCTCT | 120 |
| AGTATATCGA CTTTGGAAAC AAAAGACATC ATTCTATTTA TAGCATTCTG TTTTTAGTAG | 180 |
| TGGTATTTCC ATTTACAAAA TATAGTAATT CTCGATCGCT GAAAATGTCA AATCCTAGAA | 240 |
| AACGTAGCAT TCCTACGCGT GATGTTAACA TCGTTCTCGA ACAGTTGTTG GCCGAAGATT | 300 |
| CATTTGATGA ATCCGATTTT TCCGAAATAG ACGATTCTGA TGAT | 344 |

MER75B Left End Nucleotide Sequence
Sequence 91 bp. (SEQ ID NO: 15)

| | |
|---|---|
| TTAACCCATT TCCCGTTTGC CCCGAGAATA CTCTTGTCTC TAATCCTAAT GTAACATCAT | 60 |
| ATACATTTCT GTTACATTAG GATTAGAGAC A | 91 |

MER75A Left End Nucleotide Sequence
Sequence 32 bp. (SEQ ID NO: 16)

| | |
|---|---|
| TTAACCCATT TCCCGTTTGC CCCGAGAATA CT | 32 |

Pteropus vampyrus Right End Sequence
Sequence 171 bp. (SEQ ID NO: 17)

| | |
|---|---|
| TAGGATTAGA GACAAGTTCT GTTTAGAAAT AACTCCAAGA ACAGTTTTTA TATTTTATTT | 60 |
| TCACATTGAA AACCAGTCAG ATTTGCTTCA GCCTCAAAGA GCATGTTTAT GTAAAATTAA | 120 |
| ATTAACGCTG GCAGCGAGCT GCACTTTTTT TCTAAACGGG AAATGGGTTA A | 171 |

PGBD4 Right End Nucleotide Sequences
Sequence 176 bp. (SEQ ID NO: 18)

| | |
|---|---|
| CCTGGGATTA TAGGCATGAG CCACTGCGCC TAGCACCAAG AACAGTTTTT ATATTTTATT | 60 |
| TTCACATTGA AAATCAGTCA GATTTGCTTC AGCCTCAAAG AGGGTGTTTA TGTAAAACTA | 120 |
| AATGAGTGCA GGCAGCGAGC TACACTTTTT TTTTTCCTAA ATGGAAAATG GGTTAA | 176 |

MER75 Right End Nucleotide Sequences
Sequence 178 bp. (SEQ ID NO: 19)

| | |
|---|---|
| TCAGACGATT CTGATGTTAG TTCTGTTTAG AAATAACTCC AAGAACAGTT TTTATATTTT | 60 |
| ATTTTCACAT TGAAAATCAG TCAGATTTGC TTCAGCCTCA AAGAGCGTGT TTATGTAAAA | 120 |
| TTAAATGAGC GCTGGCAGCG AGCTGCACTT TTTTTTTTCT AAACGGGAAA AGGGTTAA | 178 |

MER75B Right End Nucleotide Sequences
Sequence 160 bp. (SEQ ID NO: 20)

| | |
|---|---|
| AGTTCTGTTT AGAAATAACT CCAAGAACAG TTTTTATATT TTATTTTCAC ATTGAAAATC | 60 |
| AGTCAGATTT GCTTCAGCCT CAAAGAGCGT GTTTATGTAA AATTAAATGA GCGCTGGCAG | 120 |
| CGAGCTGCAC TTTTTTTTTT CTAAACGGGA AAAGGGTTAA | 160 |

MER75A Right End Nucleotide Sequences
Sequence 46 bp. (SEQ ID NO: 441)

| | |
|---|---|
| CGCTGGCAGC GAGCTGCACT TTTTTTCTAA ACGGGAAATG GGTTAA | 46 |

Extended Pteropus vampyrus Nucleotide Sequence* 2210 BP (SEQ ID NO: 429)

| | |
|---|---|
| CCCATTTCCT GTTTGCCCCG AGAATACTCA CCAGCGGCAC TTGCAGCTGC AGCGTTTACC | 60 |
| CCGAGATAAC TCGYCGATTA CAGTCCTAAC CTTACCCCCA AAGTTTGCCA TGAAATATCT | 120 |
| CGCTTTTATT ATTATTTTCG CATCGCTCTA GTATATCGAT AGTCTTTGGA AACAAATGAC | 180 |
| ATCATTNTAT TTACAGCATT CTGTTTTTAN TAGTGGTATT TCCATTTACA AAATATAGTA | 240 |
| ATTTTCTATC GCTGAAAATG TCAAATCCTA GAAAACGTAG CATTCCTACA TGTGATGTTA | 300 |
| ACTTCGTTCT CGAACAGTTG TTAGCCGAAG ATTCATTTGA TGAATCCGAT TTTTCCGAAA | 360 |
| TAGACGATTC TGATGATTTT TCGGATAGTG CTTCGGAAGA CTATACGGTC AGGCCTCCGT | 420 |
| CCGATTCGGA ATCTGATGGA AATAGCCCTA CATCAGCTGA CTCGGGTCGC GCTCTGAAAT | 480 |
| GGTCAACTCG TGTTATGATT CCACGTCAAA GGTATGACTT TACCGGCACA CCTGGCAGAA | 540 |
| AAGTTGATGT CAGTGATACC ACTGACCCAC TGCAGTATTT TGAACTGTTC TTTACTGAGG | 600 |
| AATTAGTTTC AAAAATTACC AGTGAAATGA ATGCCCAAGC TGCCTTGTTG GCTTCAAAGC | 660 |
| CACCTGGTCC GAAAGGATTT TCGCGAATGG ATAAATGGAA AGACACTGAC AATGATGAAC | 720 |
| TGAAAGTCTT TTTTGCAGTA ATGTTACTGC AAGGTATTGT GCAGAAACCT GAGCTGGAGA | 780 |
| TGTTTTGGTC GACAAGGCCT CTTTTGGATA TACCTTATCT CAGGCAAATT ATGACTGGTG | 840 |
| AAAGATTTTT ACTTTTGCTT CGGTGCCTGC ATTTTGTCAA CAATTCTTCC ATATCCGCTG | 900 |
| GTCAATCAAA GGCCCAGATT TCATTGCAGA AGATCAAACC TGTGTTCGAC TTTCTTGTAA | 960 |
| ATAAGTTTTC AACTGTATAT ACTCCAAACA GAAACATTGC AGTCGATGAA TCACTGATGC | 1020 |
| TGTTCAAGGG GCGGTTAGCT ATGAAGCAGT ACATCCCGAC GAAATGtGCA CGATTTGGTC | 1080 |
| TCAAGCTNTA TGTACTTTGT GAAAGTCAAT CTGGTTACGT GTGGAATGCG CTTGTTCACA | 1140 |
| CAGGGCCCAG TATGAATTTG AAAGATTCAG CTGATGGTCT GAAATCGTCA TGCATTGTTC | 1200 |
| TTACCTTGGT CAATGACCTT CTTGGCCAAG GATATTGTGT CTTCCTCAAT AACTTTTATA | 1260 |
| CATCTCCCAT GCTTTTCAGA GAATTACATC AAAACAGGAC TGATGCAGTT GGGACAGCTC | 1320 |
| GTTTGAACAG AAAACAGATG CCAAATGATC TGAAAAAAAG GATTGCAAAG GGGACGACTG | 1380 |
| TAGCCAGATT CTGTGGTGAA CTTATGGCAC TGAAATGGTG TGACAAGAAG GAGGTGACAA | 1440 |
| TGTTGTCAAC ATTCCACAAT GATACTGTGA TTGAAGTAGA CAACAGAAAT GGAAAGAAAA | 1500 |
| CTAAGAAGCC ATGTGTCATT GTGGATTATA ACGAGAATAT GGGAGCAGTG GACTCGGCTG | 1560 |
| ATCAGATGCT CACTTCTTAT CCAACTGAGC GCAAAAGGCA CAAGTTTTGG TATAAGAAAT | 1620 |
| TCTTTCGCCA CCTTCTAAAC ATTACAGTGC TGAACTCCTA CATCCTGTTC AAGAAGGACA | 1680 |
| ATCCTGAGCA CACGATCAGC CATGTAAACT TCAGACTGAC GTTGATTGAA AGAATGCTGG | 1740 |
| AAAAGCATCA CAAGCCAGGG CAGCAACGTC TTCGAGGTCG TCCGTGCTCT GATGATGTCA | 1800 |
| CACCTCTTCG CCTGTCTGGA AGACATTTCC CCAAGAGCAT ACCACCAACA TCAGGGAAAC | 1860 |
| AGAATCCAAC TGGTCGCTGC AAAGTTTGCT GCTCGCACGA CAAGGATGGC AAGAAGATCC | 1920 |
| GGAGAGAAAC GTtATATTTT TGTGCGGAAT GTGATGTTCC GCTTTGTGTT GTTCCGTGCT | 1980 |
| TTGAAATTTA CCACACGAAA AAAAATTATT AAATACTGAT CATCATATAC ATTTCTGTTA | 2040 |
| CATTAGGATT AGAGACAAGT TCTGTTTAGA AATAACTCCA AGAACAGTTT TTATATTTTA | 2100 |
| TTTTCACATT GAAAACCAGT CAGATTTGCT TCAGCCTCAA AGAGCATGTT TATGTAAAAT | 2160 |
| TAAATTAACG CTGGCAGCGA GCTGCACTTN TTTTCTAAAC GGGAAATGGG | 2210 |

Extended Pteropus vampyrus Amino Acid Sequence 584 Amino Acids. (SEQ ID NO: 430)

| | |
|---|---|
| MSNPRKRSIP TCDVNFVLEQ LLAEDSFDES DFSEIDDSDD FSDSASEDYT VRPPSDSESD | 60 |
| GNSPTSADSG RALKWSTRVM IPRQRYDFTG TPGRKVDVSD TTDPLQYFEL FFTEELVSKI | 120 |
| TSEMNAQAAL LASKPPGPKG FSRMDKWKDT DNDELKVFFA VMLLQGIVQK PELEMFWSTR | 180 |
| PLLDIPYLRQ IMTGERFLLL LRCLHFVNNS SISAGQSKAQ ISLQKIKPVF DFLVNKFSTV | 240 |
| YTPNRNIAVD ESLMLFKGRL AMKQYIPTKC ARFGLKLYVL CESQSGYVWN ALVHTGPSMN | 300 |
| LKDSADGLKS SCIVLTLVND LLGQGYCVFL NNFYTSPMLF RELHQNRTDA VGTARLNRKQ | 360 |
| MPNDLKKRIA KGTTVARFCG ELMALKWCDK KEVTMLSTFH NDTVIEVDNR NGKKTKKPCV | 420 |
| IVDYNENMGA VDSADQMLTS YPTERKRHKF WYKKFFRHLL NITVLNSYIL FKKDNPEHTI | 480 |
| SHVNFRLTLI ERMLEKHHKP GQQRLRGRPC SDDVTPLRLS GRHFPKSIPP TSGKQNPTGR | 540 |
| CKVCCSHDKD GKKIRRETLY FCAECDVPLC VVPCFEIYHT KKNY | |

A MLT left end (5′ to 3′) is as follows
TTAACACTTGGATTGCGGGAAACGAGTTAAGTCGGCTCGCGTGAATTGCGCGTACTCCGCGGGAGCCGTCTTAACTC
GGTTCATATAGATTTGCGGTGGAGTGCGGGAAACGTGTAAACTCGGGCCGATTGTAACTGCGTATTACCAAATATTT
GTT (SEQ ID NO: 431)
A MLT right end (5′ to 3′) is as follows
AATTATTTATGTACTGAATAGATAAAAAAATGTCTGTGATTGAATAAATTTTCATTTTTTACACAAGAAACCGAAAA
TTTCATTTCAATCGAACCCATACTTCAAAAGATATAGGCATTTTAAACTAACTCTGATTTTGCGCGGGAAACCTAAA
TAATTGCCCGCGCCATCTTATATTTTGGCGGGAAATTCACCCGACACCGTGGTGTTAA (SEQ ID NO: 432)
Trichnoplusia ni

| | |
|---|---|
| 1 | MGSSLDDEHI LSALLQSDDE LVGEDSDSEI SDHVSEDDVQ SDTEEAFIDE VHEVQPTSSG |
| 61 | SEILDEQNVI EQPGSSLASN KILTLPQRTI RGKNKHCWST SKSTRRSRVS ALNIVRSQRG |
| 121 | PTRMCRNIYD PLLCFKLFFT DEIISEIVKW TNAEISLKRR ESMTGATERD TNEDEIYAFF |
| 181 | GILVMTAVRK DNHMSTDDLF DRSLSMVYVS VMSRDREDFL IRCLRMDDKS IRPTLRENDV |
| 241 | FTPVRKIWDL FIHQCIQNYT PGAHLTIDEQ LLGFRGRCPF RMYIPNKPSK YGIKILMMCD |
| 301 | SGTKYMINGM PYLGRGTQTN GVPLGEYYVK ELSKPVRGSC RNITCDNWFT SIPLAKNLLQ |
| 361 | EPYKLTIVGT VRSNKREIPE VLKNSRSRPV GTSMFCFDGP LTLVSYKPKP AKMVYLLSSC |
| 421 | DEDASINEST GKPQMVMYYN QTKGGVDILD QMCSVMTCSR KINRWPMALL YGMINIACIN |
| 481 | SFIIYSHNVS SKGEKVQSRK KFMRNLYMSL TSSFMRKRLE APTLKRYLRD NISNILPNEV |
| 541 | PGTSDDSTEE PVTKKRTYCT YCPSKIRRKA NASCKKCKKV ICREHNIDMC QSCF |
| | (SEQ ID NO: 433) |

Pteropus vampyrus

| | |
|---|---|
| 1 | MSNPRKRSIP TCDVNFVLEQ LLAEDSFDES DESEIDDSDD FSDSASEDYT VRPPSDSESD |
| 61 | GNSPTSADSG RALKWSTRVM IPRQRYDETG TPGRKVDVSD TTDPLQYFEL FFTEELVSKI |
| 121 | TSEMNAQAAL LASKPPGPKG FSRMDKWKDT DNDELKVFFA VMLLQGIVQK PELEMFWSTR |
| 181 | PLLDIPYLRQ IMTGERFLLL LRCLHFVNNS SISAGQSKAQ ISLQKIKPVF DFLVNKFSTV |
| 241 | YTPNRNIAVD ESLMLFKGRL AMKQYIPTKM NLKDSADGLK |
| | (SEQ ID NQ: 434) |

Myotis myotis (″2a″)

| | |
|---|---|
| 1 | MDLRCQHTVL SIRESRGLLP NLKMKTSRMK KGDIIFSRKG DILLLAWKDK RVVRMISIHD |
| 61 | TSVSTTGKKN RKTGENIVKP ACIKEYNAHM KGVDRADQFL SCCSILRKMM KWTKKVVLYL |
| 121 | INCGLENSFR VYNVLNPQAK MKYKQFLLSV ARDWIMDDNN EGSPEPETNL SSPSPGGARR |
| 181 | APRKDPPKRL SGDMKQHEPT CIPASGKKKF PTRACRVCAH GKRSESRYLC KFCLVPLHRG |
| 241 | KCFTQYHTLK KY (SEQ ID NQ: 435) |

Myotis myotis (″1″)

| | |
|---|---|
| 1 | MKAFLGVILN MGVLNHPNLQ SYWSMDFESH IPFFRSVFKR ERFLQIFWML HLKNDQKSSK |
| 61 | DLRTRTEKVN CFLSYLEMKF RERFCPGREI AVDEAVVGFK GKIHFITYNP KKPTKWGIRL |
| 121 | YVLSDSKCGY VHSFVPYYGG ITSETLVRPD LPFTSRIVLE LHERLKNSVP GSQGYHFFTD |
| 181 | RYYTSVTLAK ELFKEKTHLT GTIMPNRKDN PPVIKHQKLK KGEIVAFRDE NVMLLAWKDK |
| 241 | RIVTLSTWDS ETESVERRVG GGKEIVLKPK VVTNYTKFMG GVDIADYTST YCFMRKTLKW |
| 301 | WRTLFFWGLE VSVVNSYILY KECQKRKNEK PITHVKFIRK LVHDLVGEFR DGTLTSRGRL |
| 361 | LSTNLEQRLD GKLHIITPHP NKKHKDCVVC SNRKIKGGRR ETIYICETCE CKPGLHVGEC |
| 421 | FKKYHTMKNY RD (SEQ ID NO: 436) |

Myotis lucifugus (″2″)

| | |
|---|---|
| 1 | MPSLRKRKET NETDTLPEVF NDNLSDIPSE IEDADDCFDD SGDDSTDSTD SEIIRPVRKR |
| 61 | KVAVLSSDSD TDEATDNCWS EIDTPPRLQM FEGHAGVTTF PSQCDSVPSV TNLFFGDELF |
| 121 | EMLCKELSNY HDQTAMKRKT PSRTLKWSPV TQKDIKKELG LIILMGQTRK DSLKDYWSTD |
| 181 | PLICTPIFPQ TMSRHRFEQI WTFWHENDNA KMDSRSGRLF KIQPVLDYFL HKFRTIYKPK |
| 241 | QQLSLDEGMI PWRGRFKERT YNPAKITKYG LLVRMVCESD TGYICSMEIY TAEGRKLQET |
| 301 | VLSVLGPYLG IWHHIYQDNY YNATSTAELL LQNKTRVCGT IRESRGLPPN LEMKTSRMKK |
| 361 | GDIIFSRKGD ILLLAWKDKR VVRMISTIHD TSVSTTGKKN RKTGENIVKP TCIKEYNAHM |
| 421 | KGVDRADQFL SCCSILRKTM KWTKKVVLYL INCGLENSFR VYNVLNPQAK MKYKQFLLSV |
| 481 | ARDWITDDNN EGSPEPETNL SSPSPGGARR APRKDPPKRL SGDMKQHEPT CIPASGKKKE |
| 541 | PTRACRVCAA HGKRSESRYL CKFCLVPLHR GKCFTQYHTL KKYMDLRCQH TVLSTVGRGY |
| 601 | SVLARFKPRT NERTGSSHCH VQVPAGGQGP PSTIIANGCG CKLEPMVRTR SPTCLVIEFG |
| 661 | CM (SEQ ID NO: 437) |

Myotis myotis (″2″)

| | |
|---|---|
| 1 | MPSLRKRKET NETDTLPEVF NDNLSDIPSE IEDADDCEDD SGDDSIDSTE SEIIRPVRKR |
| 61 | KVAVLSSDSN TDEATDNCWS EIDTPPRLQM FEGHAGVTTF PSQCDSVPSV TNLFFGDELF |
| 121 | EMLCKELSNY HDQTAMKRKT PSRTLKWSPV TQKDIKKFLG LIILMGQTRK DSWKDYWSTD |
| 181 | PLICTPIFPQ TMSRHRFEQI WTFWHENDNA KMDSCSGRLF KIQPVLDYFL HKFRTIYKPK |
| 241 | QQLSLDEGMI PWRGRLKFTY NPAITKYGLL VRMVCESDTG YICNMEIYTA ERKKLQETVL |
| 301 | SVLGPYLGIW HHIYQDNYYN ATSTAELLLQ NKTRVCGTIR ESRGLPPNLK MKTSRMKKGD |
| 361 | IIFSRKGDIL LLAWKDKRVV RMISTIHDTS VSTTGKKNRK TGENIVKPTC IKEYNAHMKG |
| 421 | VDRADQFLSC CSILRKTTKW TKKVVLYLIN CGLENSERVY NILNPQAKMK YKQFLLSVAR |
| 481 | DWITDDNNEG SPEPETNLSS PSSGGARRAP RKDQPKRLSG DMKQHEPTCI PASGKKKFPT |
| 541 | ACRVCAAHGK RSESRYLRKF CFVPLRGKCF MYHTLKKYSE LFSLIVVSKI QNVIIYKITK |
| 601 | VYMRYVMRSH CPLSFLVFAP SVKDRSRVFS FFTRHLLWTL DVNTLSCPHR MKRSHWWKPC |
| 661 | RSIYEKLYNC TNP (SEQ ID NO: 438) |

Myotis myotis (″2b″)

| | |
|---|---|
| 1 | MDLRCQHTVL SIRESRGLPP NLKMKTSRMK KGDIIFSRKG DILLLAWKDK RVVRMISTIH |
| 61 | DTSVSTTGKK NRKTGENIVK PACIKEYNAH MKGVDRADQF LSCCSILRKT MKWTKKVVLY |
| 121 | LINCGLFNSF RVYNVLNPQA KMKYKQFLLS VARDWITDDN NEGSPEPETN LSSPSPGGAR |
| 181 | RAPRKDPPKR LSGDMKQHEP TCIPASGKKK FPTRACRVCA AHGKRSESRY LCKFCLVPLH |
| 241 | RGKCFTQYHT LKKY (SEQ ID NO: 439) |

TTAA (SEQ ID NO: 440)

This invention is further illustrated by the following non-limiting examples.

### EXAMPLES

Hereinafter, the present disclosure will be described in further detail with reference to examples. These examples are illustrative purposes only and are not to be construed to limit the scope of the present invention. In addition, various modifications and variations can be made without departing from the technical scope of the present invention.

### Example 1: Generation of CAR-Immune Cells

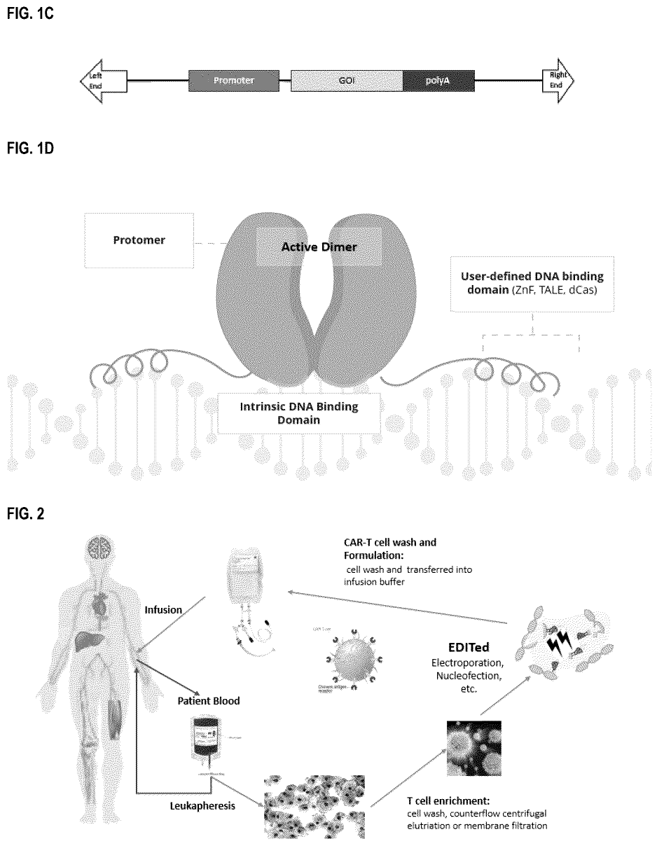

FIGS. 1A-D and FIG. 2 depict schematic diagrams of the process to produce CAR-immune (e.g., CAR-T) cells.

In an illustrative method, a sample (e.g., blood) is obtained from patient (donor) and peripheral blood mononuclear cells (PBMCs) are separated, e.g., via leukapheresis and the reminder of the blood is optionally returned to the patient (<1 day). PBMCs are washed and concentrated using, for instance, centrifugation of membrane separation (<1 day). Cells can be sorted at this point as well. The present enzyme (e.g., the enzyme of SEQ ID NO: 2, or a variant thereof) and the present donor DNA are introduced into donor cells, e.g., by electroporation, nucleofection, or fluid dynamics. Cells are washed and formulated into infusion buffer (1 days) and then infused into the patient without the need for further expansion.

In illustrative methods, processing 3×10⁹ PBMCs generates 1.5×10⁸ CAR-immune cells, equivalent to 2.1×10⁶ CAR-immune cells/kg for a 70 kg patient. In the illustrative methods, production time is approximately 1-2 days.

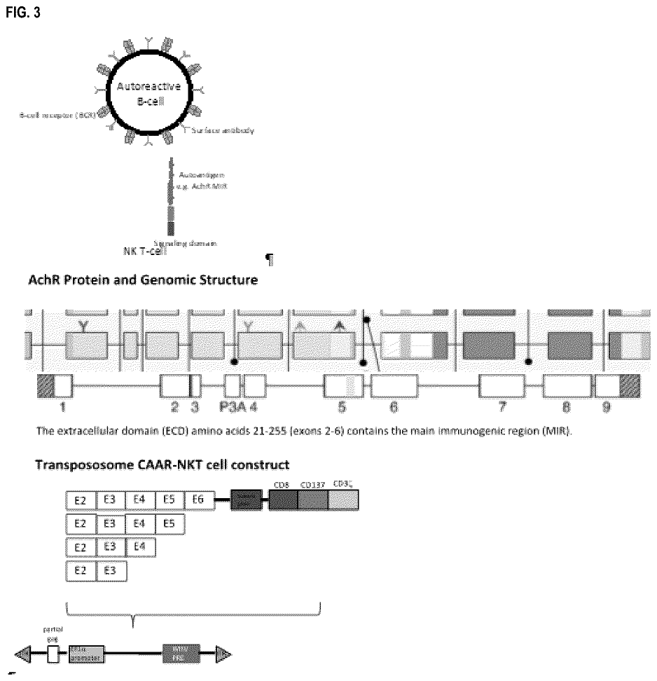

An illustrative process for making CAAR-T cells is shown in FIG. 3.

### Example 2: Engineering of Human Primary T Cells with Donor DNA and MLT Transposase Enzyme

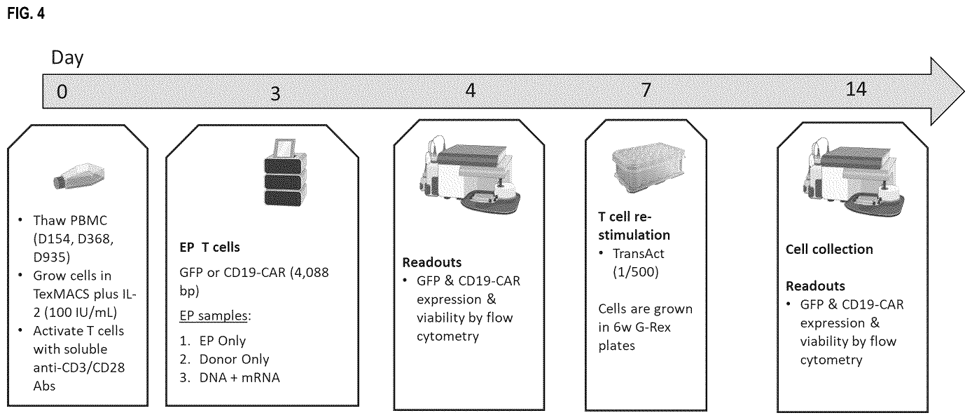

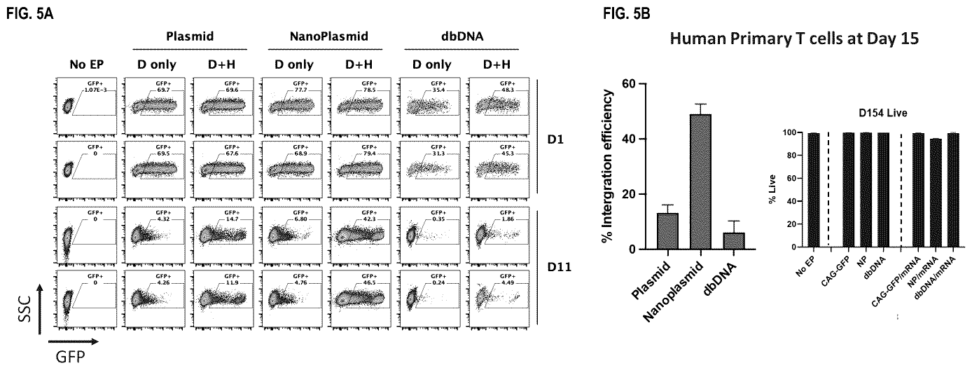

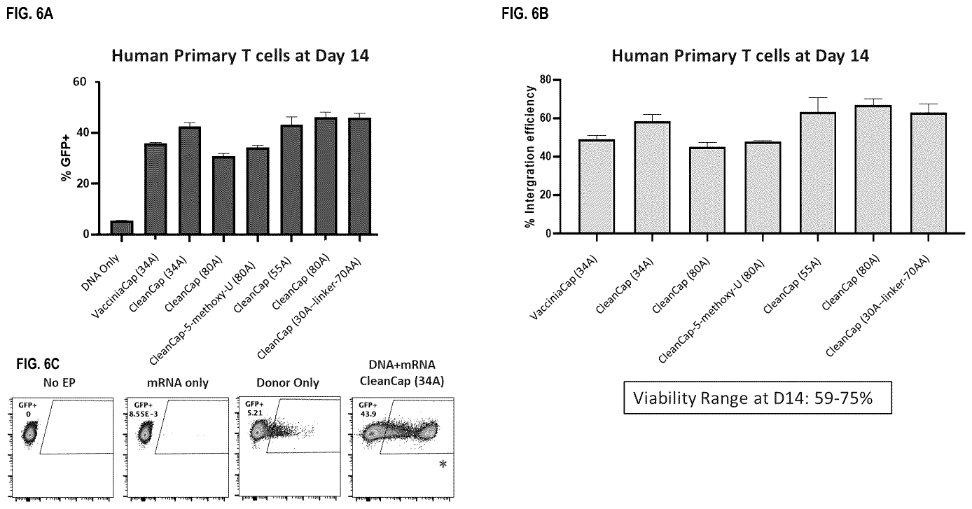

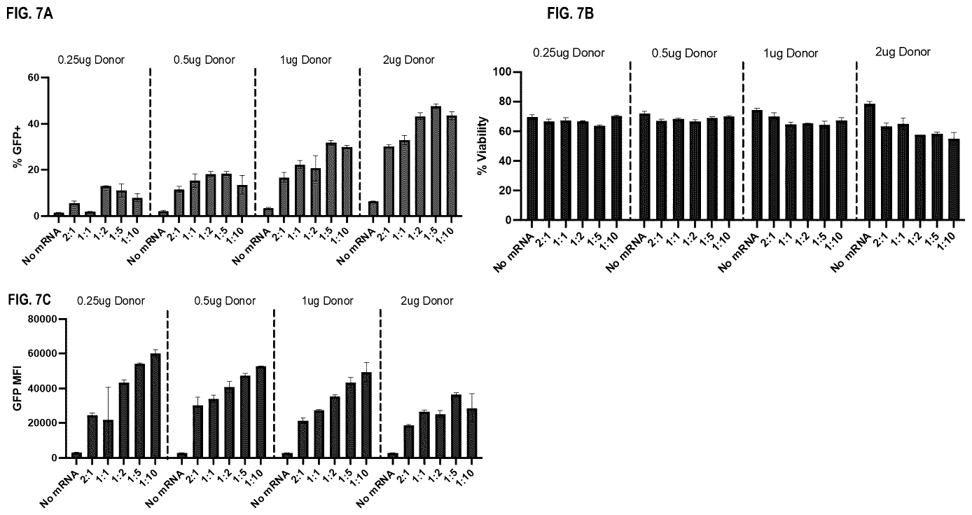

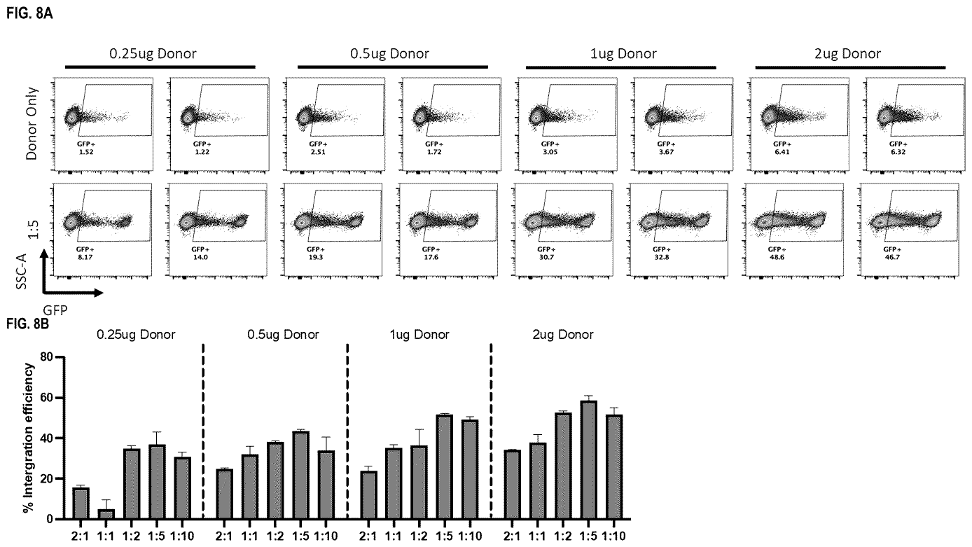

### Methods for FIG. 4, FIGS. 5A-B, FIGS. 6A-C, FIGS. 7A-C, and FIGS. 8A-B.

Peripheral Blood Mononuclear Cells (PBMCs) were purified from Leukopaks obtained from healthy donors (HemaCare) using standard ficoll gradient purification. To generate activated T cells PBMC cells were grown at 37° C. in TexMACS medium (Miltenyi, cat #130-097-196) supplemented with 5% Human AB serum (Valley Biomedical, cat #HP1022HI) plus 100 IU/mL of IL-2 (Miltenyi, cat #130-097-748) and 50 ng/mL of soluble anti-CD3 (Miltenyi, cat #130-093-387) and anti-CD28 antibodies (Miltenyi, cat #130-093-375) for 3 days. After activation, T cells were collected and electroporated using Lonza's 4D-nucleofector device following manufacturer's protocol. Briefly, 10⁶ activated T cells were electroporated using 16w strips (Lonza, cat #V4XP-3032) with 2 μg of donor DNA and 3 μg of MLT transposase helper mRNA. As controls, cells were either not electroporated or electroporated with donor DNA only. After electroporation, cells were placed back in culture in TexMACS medium supplemented with 5% Human AB serum plus 100 IU/mL of IL-2 and allowed to recover for 4 days. At day 7 of culture (4 days post-electroporation), cells were restimulated with TransAct (Miltenyi, cat #130-111-160) and expanded in 24w G-Rex (WilsonWolf, cat #80192M) for 7 additional days. Cell viability and transgene expression were evaluated by flow cytometry using a CytoFLEX flow cytometer (Beckman).

When mentioned, integration efficiency was calculated as: [(% GFP expression Sample at D14)−(% GFP expression Donor only at D14)/(% GFP expression Sample at D1 post-EP)]×100.

### Example 3: Evaluation of Different Donor DNA Designs for Effective Engineering of Human Primary T Cells Using MLT Transposase

Three different versions of a donor DNA encoding the reporter gene GFP in which its expression was driven by the CAG promoter (CAG-GFP Donor) were evaluated: standard plasmid DNA (VectorBuilder), Nanoplasmid (Nature Technology Corporation, NTC), and doggybone (Touchlight). Activated T cells were engineered as described above and cell viability and GFP transgene expression were determined at 24 hours post-electroporation and at the end of culture by flow cytometry. Delivery of circular CAG-GFP plasmid or nanoplasmid into primary activated T cells by electroporation was very efficient as shown by high GFP expression (>60% GFP positive cells) at 24 hours post-electroporation in samples transfected with Donor only or Donor plus Helper mRNA (FIGS. 5A-B). Of note, electroporation efficiency of a linear dsDNA (doggybone) was lower than that of plasmid or nanoplasmid (35-40% GFP expression for dbDNA, FIGS. 5A-B). Upon T cell expansion, GFP expression was lost in samples that were transfected with donor only, since in the absence of MLT transposase enzyme there is no stable integration of the donor DNA into the genome of the T cells, and GFP expression is coming from episomal DNA molecules which are diluted out as the T cells divide over time. Samples that were transfected with Donor plus Helper show sustained GFP expression over time with the nanoplasmid donor showing the highest level of expression at D14 (>40% GFP positive, FIGS. 5A-B). Consistent with this result, the CAG-GFP nanoplasmid donor yielded the highest integration efficiency (approx. 50%) compared to standard plasmid or dbDNA (<20%) (FIGS. 5A-B). Of note, at D14 of culture all samples showed high viability (>90%, FIGS. 5A-B).

### Example 4: Evaluation of Different MLT Transposase Helper mRNA Designs for Effective Engineering of Human Primary T Cells

To determine an effective mRNA design for engineering T cells using MLT transposase, seven different MLT transposase-encoding mRNAs containing different CAP, nucleotide modifications, and poly(A) tail length were evaluated (TABLE 6). Nucleofection of activated T cells with a CAG-GFP nanoplasmid donor DNA plus each of the seven different MLT transposase-encoding mRNAs resulted in high GFP expression (>60%) at 24 hr post-electroporation with no significant differences between the different mRNA designs or donor only control sample (data not shown). However, GFP expression and integration efficiency at D14 of culture were significantly lower for Vaccinia capped MLT transposase mRNA and CleanCap MLT transposase mRNA either unmodified or with 5-Methoxy-U modified nucleotides. Importantly, CleanCap MLT transposase mRNA with N1-Methyl-Pseudo-U modified nucleotides provided the highest GFP expression and integration efficiency at D14, with those of longer poly(A) tail providing the best results (FIGS. 6A-C).

TABLE 6
mRNA designs

| Gene | Vendor | CAP | Poly(A) length (nt) | Nucleotide modifications |
|---|---|---|---|---|
| Hyper-MLT transposase | TriLink | CleanCap | 80 | Unmodified |
| Hyper-MLT transposase | TriLink | CleanCap | 80 | 5-Methoxy-U |
| Hyper-MLT transposase | TriLink | CleanCap | 55 | N1-Methyl-Pseudo-U |
| Hyper-MLT transposase | TriLink | CleanCap | 80 | N1-Methyl-Pseudo-U |
| Hyper-MLT transposase | TriLink | CleanCap | 30A, Linker, 70A | N1-Methyl-Pseudo-U |
| Hyper-MLT transposase | TriLink | CleanCap | 34A | N1-Methyl-Pseudo-U |
| Hyper-MLT transposase | Aldevron | Vaccinia | 34A | N1-Methyl-Pseudo-U |

### Example 5: Determining an Effective Donor DNA: Helper mRNA Ratio for Efficient Engineering of Human Primary T Cells Using MLT Transposase

To determine an effective Donor DNA:Helper mRNA ratio for engineering human primary T cells, activated T cells were electroporated with different amounts of a CAG-GFP Donor nanoplasmid (Nature Technology Corporation) DNA and MLT transposase mRNA (TriLink) covering a wide range of concentrations and ratios (TABLE 7). Viability at the end of culture, day 14, was high (>60%) across all samples (FIGS. 7A-C). GFP expression and MFI (Mean Fluorescence Intensity) at day 14 increased with the amount of Donor DNA transfected and at higher Donor to Helper ratios (FIGS. 7A-C). The results show that Donor to Helper ratios of 1:2 and 1:5 provide the highest percentage of GFP positive cells at D14 of culture when transfecting activated T cells with 2 μg of CAG-GFP Donor DNA (FIGS. 7A-C). Furthermore, when transfecting 1 or 2 μg of Donor DNA the Donor:Helper ratio of 1:5 provided the highest integration efficiency (FIGS. 7A-C and FIGS. 8A-B).

TABLE 7
Matrix of donor DNA amounts and donor:helper
ratios used in experiment

| D:H ratio | Donor DNA (ug) | | | | |
|---|---|---|---|---|---|
| matrix | 0.25 | 0.5 | 1 | 2 | Ratio |
| Helper | 0 | 0 | 0 | 0 | No mRNA |
| enzyme | 0.125 | 0.25 | 0.5 | 1 | 2:1 |
| SalioB | 0.25 | 0.5 | 1 | 2 | 1:1 |
| mRNA | 0.5 | 1 | 2 | 4 | 1:2 |
| (ug) | 1.25 | 2.5 | 5 | 10 | 1:5 |
| | 2.5 | 5 | 10 | 20 | 1:10 |

### Example 6: Methods for Showing Efficient Engineering of T Cells by MLT Transposase

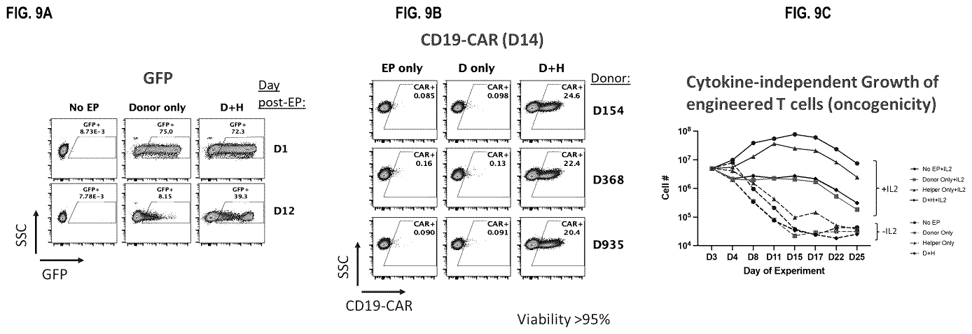

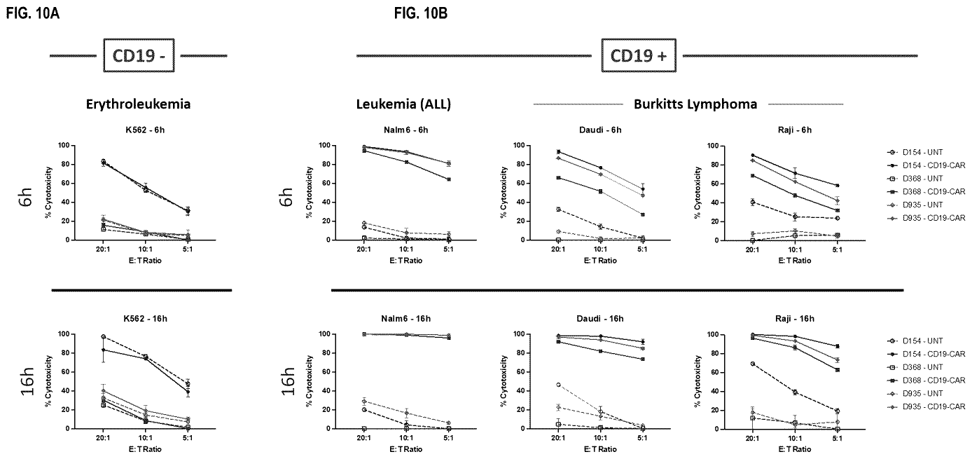

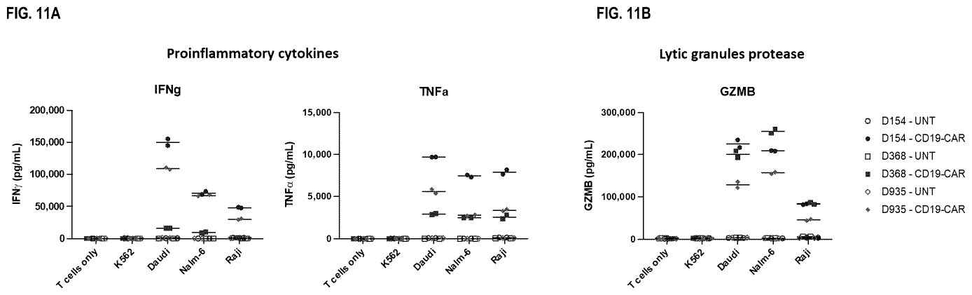

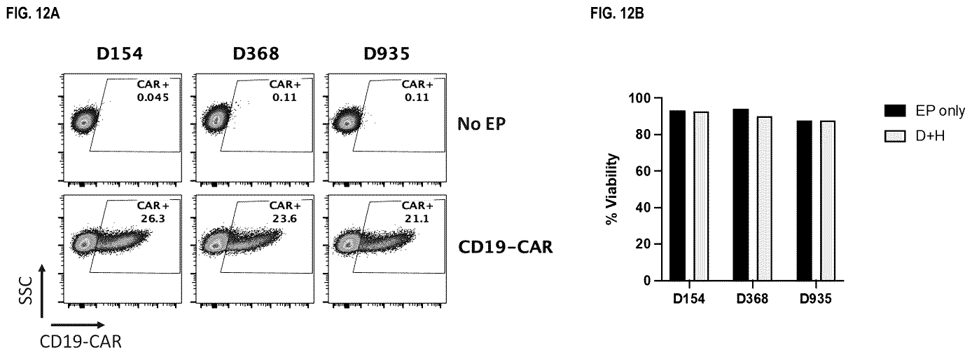

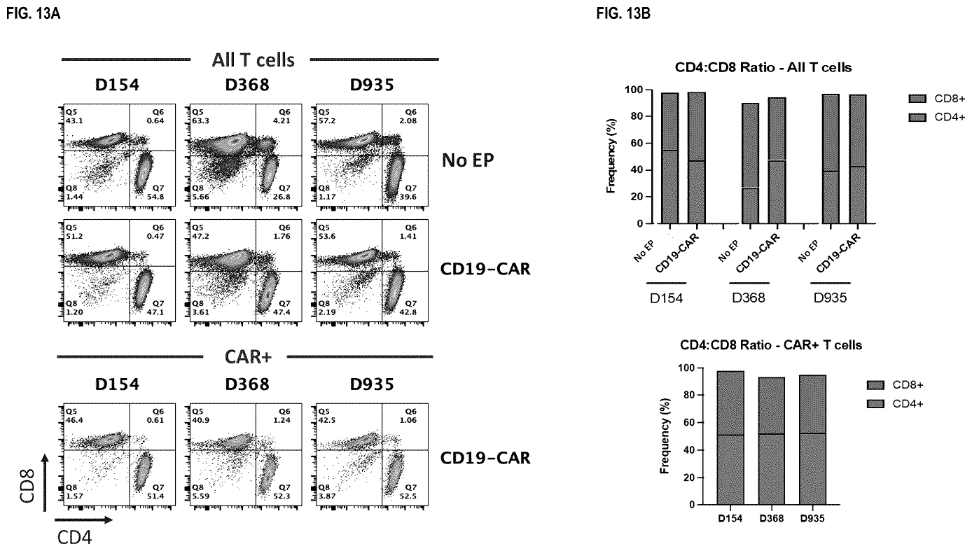

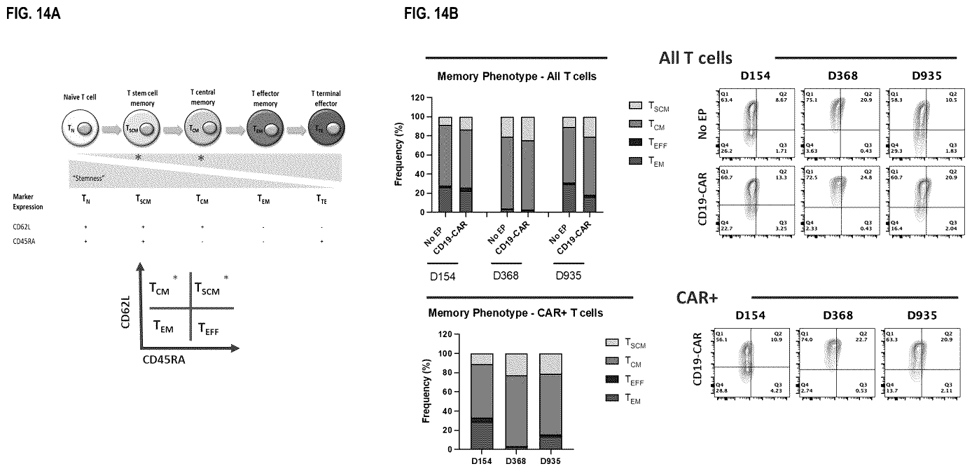

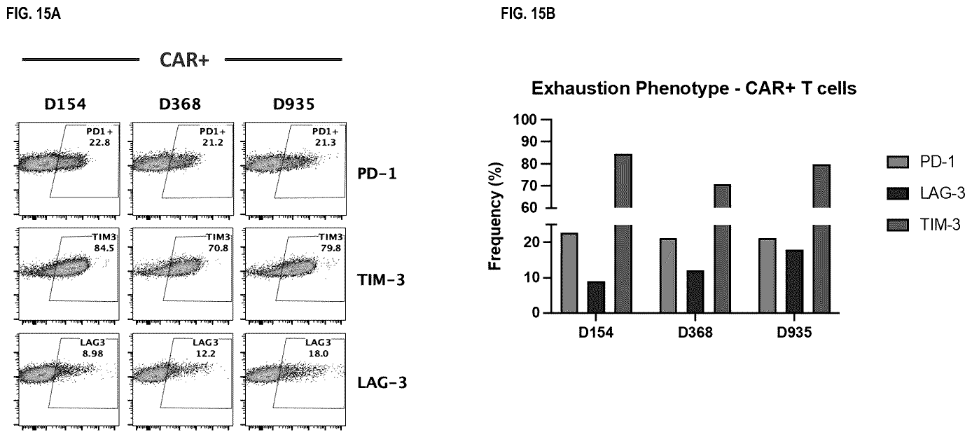

### Methods for FIGS. 9A-C, FIGS. 10A-B, FIGS. 11A-B, FIGS. 12A-B, FIGS. 13A-B, FIGS. 14A-C, and FIGS. 15A-B.

### Cytokine Independent Growth Assay

Activated T cells were generated from healthy donor PBMC cells grown at 37° C. in TexMACS medium (Miltenyi, cat #130-097-196) supplemented with 5% Human AB serum (Valley Biomedical, cat #HP1022HI) plus 100 IU/mL of IL-2 and 50 ng/mL of soluble anti-CD3 (Miltenyi, cat #130-093-387) and anti-CD28 antibodies (Miltenyi, cat #130-093-375) for 3 days. After activation, T cells were collected and electroporated using Lonza's 4D-nucleofector device following manufacturer's protocol. Briefly, 10⁶ activated T cells were electroporated using 16w strips (Lonza, cat #V4XP-3032) with 2 μg of Donor DNA, 3 μg of MLT transposase Helper mRNA or both. As control, cells were electroporated without adding nucleic acids. After electroporation cells were split into two and put back in culture in TexMACS medium in the absence or presence of 100 IU/mL of IL-2. Cell proliferation was evaluated counting the number of cells by flow cytometry using CountBright beads (ThermoFisher, cat #C36995) every 2-3 days, for a total of at least 20 days.

### Generation of CD19-CAR T Cells

Peripheral Blood Mononuclear Cells (PBMCs) were purified from Leukopaks obtained from healthy donors (HemaCare) using standard ficoll gradient purification. To generate activated T cells, PBMC cells were grown at 37° C. in TexMACS medium (Miltenyi, cat #130-097-196) supplemented with 5% Human AB serum serum (Valley Biomedical, cat #HP1022HI) plus 100 IU/mL of IL-2 (Miltenyi, cat #130-097-748) and 50 ng/mL of soluble anti-CD3 (Miltenyi, cat #130-093-387) and anti-CD28 antibodies (Miltenyi, cat #130-093-375) for 3 days. After activation, T cells were collected and electroporated using Lonza's 4D-nucleofector device following manufacturer's protocol. Briefly, 5e⁶ activated T cells were electroporated using 100 μL cuvettes (Lonza, cat #V4XP-3024) with 4 μg of CD19-CAR Donor DNA nanoplasmid (Nature Technology Corporation) plus 6 μg of MLT transposase Helper mRNA (TriLink). As controls, cells were either not electroporated or electroporated with Donor DNA only or no nucleic acid. After electroporation, cells were placed back in culture in TexMACS medium supplemented with 5% Human AB serum plus 100 IU/mL of IL-2 and let recover for 4 days. At day 7 of culture (4 days post-electroporation) cells were restimulated with TransAct (Miltenyi, cat #130-111-160) and expanded in 6w G-Rex (WilsonWolf, cat #80240M) for 7 additional days. At day 14 of culture cells were collected and cryopreserved using CryoStor CS10 cell preservation medium (StemCellTechnologies, cat #CS10).

### Luciferase Based Cytotoxicity Assays

The killing ability of CD19-CAR T cells was evaluated using a bioluminescence-based assay with luciferase-expressing tumor targets (TABLE 8). Co-cultures of CD19-CAR T cells and luciferase-expressing targets were set up in flat-bottom 96 well plates (Corning, cat #353296). Briefly, 100 μL of luciferase expressing targets at a concentration of 1e⁵ cells/mL were added per well in triplicates. Effector cells were added in a volume of 100 μL at 10:1, 5:1 and 2.5:1 effector-to-target ratios. Target cells incubated without effector cells or with 1% Triton X-100 (Fisher Scientific, cat #ICN19485450) were used as a measure of spontaneous and maximum lysis, respectively. Plates were incubated at 37° C. for 6 or 16 hours, and luciferase activity was determined by adding 100 μL of Bright-Glo™ Luciferase Assay Substrate (Promega, cat #E2620) and measuring the luminescence signal in an Infinite® 200 PRO plate reader (Tecan).

Cytotoxicity was calculated according to the formula: % Cytotoxicity=100−[(test counts−T×100 counts)/(No T cells counts−T×100 counts))×100].

TABLE 8
List of tumor targets used in cytotoxicity assays

| Cell line | Disease | Vendor | Catalog # |
|---|---|---|---|
| Raji-Fluc-EmGFP | Burkitt's Lymphoma | Imanis Life | CL 159 |
| | | Sciences | |
| Nalm6-Fluc- | Acute lymphoblastic | Imanis Life | CL 150 |
| Neo/eGFP-Puro | leukemia (ALL) | Sciences | |
| Daudi-Fluc-eGFP | Burkitt's Lymphoma | Imanis Life | CL 158 |
| | | Sciences | |
| K-562-luc2 | Erythroleukemia | ATCC | CCL-243- |
| | | | LUC2 |

### Cytokine Release Assay

CD19-CAR T cells or control untransfected T cells were co-culture with tumor targets at a 1:1 ratio in flat-bottom 96w plates, 5e⁵ cells/well in triplicates, for 24 hours and the amount of proinflammatory cytokines IFNγ and TNFα, and lytic granule protease granzyme B released into the supernatant was measured by ELISA (R&D systems) following manufacturer's instructions.

### Phenotypic Analysis of CAR T Cells

CD19-CAR expression was evaluated by flow cytometry using human recombinant CD19 protein conjugated to atto647N (R&D Systems, cat #ATM9269). CD4/CD8 ratio, and expression of memory and exhaustion markers were evaluated by flow cytometry analysis with one or several of the antibodies shown in TABLE 9. Stained samples were acquired in a CytoFLEX flow cytometer (Beckman Coulter).

TABLE 9
Antibodies used for immunophenotype of CAR T cells

| Antibody | Clone | Fluorophore | Vendor | Cat # |
|---|---|---|---|---|
| CD3 | SK7 | FITC | BioLegend | 344804 |
| CD4 | SK3/OKT4 | BV711 | BioLegend | 344648 |
| CD8 | SK1 | BV510 | BioLegend | 344732 |
| CD62L | DREG-56 | PE | BioLegend | 304806 |
| CCR7 | G043H7 | PE-Cy7 | BioLegend | 353226 |
| CD45RA | HI100 | PerCP | BioLegend | 304156 |
| PD-1 | EH12.2H7 | PE | BioLegend | 329906 |
| TIM-3 | F38-2E2 | PE-Cy7 | BioLegend | 345014 |
| LAG-3 | C9B7W | PE-Dazzle 594 | BioLegend | 125224 |

### Example 7: Efficient Engineering of T Cells by MLT Transposase Shows Stable GFP and CAR Expression and Good Safety Profile

Primary activated T cells were electroporated with a donor DNA encoding a reporter GFP or a CD19-CAR receptor plus or minus MLT transposase-expressing helper mRNA. Engineered T cells were expanded in vitro and sustained GFP or CAR expression was evaluated by flow cytometry at day 12 or 14 post-electroporation, respectively. As shown in FIGS. 9A-C, transgene expression was lost in cells transfected with donor DNA only, while sustained GFP (approximately 40%) or CD19-CAR (20-25%) expression was observed in cells transfected with donor plus MLT transposase mRNA helper, consistent with stable MLT transposase-mediated genomic integration of the DNA donor.

To evaluate the safety of MLT transposase-engineered T cells, primary activated T cells were electroporated with Donor DNA only, Helper mRNA only or Donor DNA plus Helper mRNA, and their tumorigenic potential was evaluated assessing their growth in the presence or absence of IL-2 (FIGS. 9A-C). As control, non-engineered T cells (No EP) were tested in parallel. As shown in FIGS. 9A-C, non-engineered T cells, in the presence of IL-2, can proliferate during the first 10 days, reach a plateau at D15 and then their growth declines steadily until D25. T cells engineered with Helper mRNA only follow a very similar pattern, while the growth of T cells engineered with Donor only or Donor plus Helper plateaus earlier due to the higher toxicity of the DNA during electroporation. Importantly, in the absence of IL-2 the growth of engineered and non-engineered T cells declines steadily shortly after electroporation with most of the cells dying off by D16 (FIGS. 9A-C). The lack of abnormal uncontrolled growth of MLT transposase-engineered T cells suggests that human primary T cells can be safely engineered with MLT transposase.

FIGS. 12A-B depict CD19-CAR expression of 21-26% at Day 14 (without enrichment) and >95% viability. FIG. 12A depicts CAR expression by flow cytometry of untransfected and CD19-CAR T cells at time of collection (D14). FIG. 12B depicts viability of CD19-CAR T cells at time of collection (D14).

### Example 8: Ex Vivo Efficacy—CD19-CAR-T Cells from 3 Healthy Donors Generated by MLT Transposase Efficiently Kill CD19-Expressing Tumor Target Cells

The cytotoxicity potential of MLT transposase-engineered CD19-CAR T cells was evaluated in co-cultures with CD19-positive leukemia (Nalm6) or lymphoma cells (Daudi, Raji) ex vivo. CD19-negative erythroleukemia K562 cells were used as control. As shown in FIGS. 10A-B, CD19-CAR T cells from all three donors kill very efficiently CD19-positive tumor targets, at both time points evaluated 6 and 16 hours, compared to non-engineered T cells. Importantly, cytotoxicity is highly specific since CD19-negative K562 cells are not killed by CD19-CAR T cells. Of note, donor D154 seems to react against K562 cells. This reactivity is likely due to an alloresponse since both, non-engineered and CD19-CAR T cells, kill equally well K562 cells indicating that the cytotoxic response is CD19 independent. In all, MLT transposase-engineered CD19-CAR T cells are highly cytotoxic, and their cytotoxicity is highly specific of the expression of the CD19 tumor antigen by the target cells.

### Example 9: Ex Vivo Efficacy—High Levels of Proinflammatory Cytokine Release by CD19 CAR-T Cells Upon Recognition of CD19 Expressing Tumor Targets

The ability of MLT transposase engineered CD19-CAR T cells to release cytokines upon recognition of CD19-expressing tumor targets was evaluated in tumor co-cultures, ex vivo, with CD19-positive or negative tumor targets. As shown in FIGS. 11A-B, after overnight incubation with CD19-positive B-cell leukemia or lymphoma targets, CD19-CAR T cells from all three donors released high levels of proinflammatory cytokines (i.e., INFγ, TNFα) and lytic granule protease granzyme B. Of note, higher levels of proinflammatory cytokines and granzyme B were observed for Daudi and Nalm-6 tumor targets. Importantly, very low or undetectable levels of cytokines were observed in the absence of tumor targets or in co-cultures with CD19-negative K562 cells. This indicates that MLT transposase engineered CD19-CAR T are highly specific and do not display tonic signaling.

### Example 10: Immunophenotype of MLT Transposase Engineered CD19-CAR T Cells

To determine the cellular immunophenotype of MLT transposase engineered CD19-CAR T cells, the expression of different cell surface markers was evaluated by flow cytometry (TABLE 9). CD19-CAR T cells from all donors had a balanced CD4:CD8 ratio (FIGS. 13A-B), and their ratio was similar to that of non-engineered T cells. Importantly, the CD19-CAR T cells had a high percentage of cells with a central memory or effector memory phenotype, and a low percentage of fully differentiated effector cells (FIGS. 14A-C). This is consistent with a favorable memory phenotype since it has been shown that CAR-T products enriched in less differentiated T cells are associated with better clinical performance and persist longer in vivo. Last, the expression of the exhaustion markers PD-1, LAG-3, and TIM-3 was evaluated. The results of this analysis show that MLT transposase engineered CD19-CAR T cells have a balance expression of these exhaustion markers, with low expression of PD-1 and LAG-3 and higher expression of TIM-3 (FIGS. 15A-B).

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features herein set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

1. A method of making a chimeric antigen receptor (CAR)-immune cell, the method comprising:
obtaining an immune cell from a sample obtained from a subject, the sample comprising immune cells or immune cell progenitors; and
transfecting the immune cell with a first nucleic acid encoding an enzyme capable of performing targeted genomic integration, and
a second, non-viral nucleic acid encoding a donor DNA comprising a chimeric CAR and flanked by ends recognized by the enzyme capable of performing targeted genomic integration,
to thereby create a transfected CAR-immune cell,
wherein the immune cell is selected from a T cell, a macrophage, and a natural killer (NK) cell.

2. A method of making a chimeric antigen receptor (CAR)-immune cell, the method comprising administering to a subject:
a first nucleic acid encoding an enzyme capable of performing targeted genomic integration, wherein the first nucleic acid is RNA, and
a second, non-viral nucleic acid encoding a donor DNA comprising a chimeric CAR and flanked by ends recognized by the enzyme capable of performing targeted genomic integration,
wherein the administration uses lipid nanoparticles (LNPs) capable of directing the first and second nucleic acids to an immune cell, to thereby create a CAR-immune cell,
wherein the immune cell is selected from a T cell, a macrophage, and a natural killer (NK) cell.

3. The method of claim 1, wherein the immune cell is a T cell.

4-8. (canceled)

9. A method of making a chimeric autoantibody receptor (CAAR)-immune cell, the method comprising:
obtaining a cell from a sample obtained from a subject, the sample comprising T cells or T cell progenitors; and
transfecting the cell with
a first nucleic acid encoding an enzyme capable of performing targeted genomic integration, wherein the first nucleic acid is RNA, and
a second, non-viral nucleic acid encoding a donor DNA comprising a chimeric CAAR comprising an autoantigen or a fragment thereof, and flanked by ends recognized by the enzyme capable of performing targeted genomic integration, to thereby create a transfected CAAR-immune cell,
wherein the immune cell is selected from a T cell, a macrophage, and a natural killer (NK) cell.

10. A method of making a chimeric autoantibody receptor (CAAR)-immune cell, the method comprising administering to a skin cell in a subject:
a first nucleic acid encoding an enzyme capable of performing targeted genomic integration, wherein the first nucleic acid is RNA, and
a second, non-viral nucleic acid encoding a donor DNA comprising a chimeric CAAR comprising an autoantigen or a fragment thereof, and flanked by ends recognized by the enzyme capable of performing targeted genomic integration,
wherein the administration is performed via intradermal or subcutaneous delivery and using lipid nanoparticles (LNPs) capable of directing the first and second nucleic acids to an immune cell, to thereby create a CAR-immune cell,
wherein the immune cell is selected from a T cell, a macrophage, and a natural killer (NK) cell.

11-48. (canceled)

49. The method of claim 1, wherein the CAR comprises:
a) an extracellular domain that binds an antigen selected from alpha folate receptor, 5T4, αvβ6 integrin, BCMA, B7-H3, B7-H6, CAIX, CD19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, GD2, GD3, Glypican-3 (GPC3), HLA-A1+MAGE1, HLA-A2+MAGE1, HLA-A3+MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, and VEGFR2;
b) a transmembrane domain derived from a polypeptide selected from CD8α, CD4, CD28, CD45, PD-1, and CD152;
c) one or more intracellular costimulatory signaling domains selected from CD28, CD54 (ICAM), CD134 (OX40), CD137 (41BB), IL-2Rβ, CD152 (CTLA4), CD273 (PD-L2), CD274 (PD-L1), and CD278 (ICOS); and
d) a CD3ζ signaling domain.

50-51. (canceled)

52. The method of claim 1, wherein the donor DNA is flanked by one or more inverted terminal ends.

53-60. (canceled)

61. The method of claim 1, wherein the enzyme capable of performing targeted genomic integration is a recombinase.

62.-63 (canceled)

64. The method of claim 61, wherein the enzyme is derived from Bombyx mori, Xenopus tropicalis, Trichoplusia ni, Myotis lucifugus, Rhinolophus ferrumequinum, Rousettus aegyptiacus, Phyllostomus discolor, Myotis myotis, Pteropus vampyrus, Pipistrellus kuhlii, Molossus molossus, Pan troglodytes, or Homo sapiens.

65. (canceled)

66. The method of claim 1, wherein the enzyme is from one or more of the Tn1, Tn2, Tn3, Tn5, Tn7, Tn9, Tn10, Tn552, Tn903, Tn1000/Gamma-delta, Tn/O, tnsA, tnsB, tnsC, tniQ, IS10, ISS, IS911, Minos, Sleeping beauty, piggyBac, Tol2, Mos1, Himar1, Hermes, Tol2, Minos, Tel, P-element, MuA, Ty1, Chapaev, transib, Tc1/mariner, or Tc3 donor DNA system, or biologically active fragments variants thereof, inclusive of hyperactive variants.

67. The method of claim 1, wherein the enzyme has an amino acid sequence having at least about 80% identity to SEQ ID NO: 1, SEQ ID NO: 3, SEQ ID NO: 4, SEQ ID NO: 6, SEQ ID NO: 7, SEQ ID NO: 8, SEQ ID NO: 9, SEQ ID NO: 10, SEQ ID NO: 11, or SEQ ID NO: 430.

68. The method of claim 67, wherein the enzyme has an amino acid sequence having at least about 80% identity to SEQ ID NO: 1 and comprises an amino acid other than serine at the position corresponding to position 2 of SEQ ID NO: 1.

69. The method of claim 68, wherein the amino acid is selected from G, A, V, L, I, and P.

70-71. (canceled)

72. The method of claim 1, wherein the enzyme has one or more amino acid substitutions selected from S8X1, C13X2, and/or N125X3, at positions corresponding to SEQ ID NO: 1, wherein X1 is selected from G, A, V, L, I and P; X2 is selected from K, R, and H; and X3 is selected from K, R, and H.

73-81. (canceled)

82. The method of claim 1, wherein the enzyme is capable of inserting the donor DNA at a TA dinucleotide site or at a TTAA (SEQ ID NO: 440) tetranucleotide site.

83.-88. (canceled)

89. The method of claim 1, wherein;
the enzyme has an amino acid sequence having S8P and G17R mutations relative to the amino acid sequence of SEQ ID NO: 3 or SEQ ID NO: 4, or a functional equivalent thereof;
the enzyme has an amino acid sequence having I83P and/or V118R mutations relative to the amino acid sequence of SEQ ID NO: 6, or a functional equivalent thereof;
the enzyme has an amino acid sequence having S20P and/or A29R mutations relative to the amino acid sequence of SEQ ID NO: 7, or a functional equivalent thereof;
the enzyme has an amino acid sequence having A12P, I28R, and/or R152K mutations relative to the amino acid sequence of SEQ ID NO: 9, or a functional equivalent thereof; or
the enzyme has an amino acid sequence having T4P and/or L13R mutations relative to the amino acid sequence of SEQ ID NO: 8, or a functional equivalent thereof.

90-95. (canceled)

96. The method of claim 94, wherein the end sequences are selected from nucleotide sequences having at least about 90% identity to SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 441, and SEQ ID NO: 22.

97-152. (canceled)

153. The method of claim 1, wherein the enzyme is encoded by a recombinant or synthetic RNA.

154-163. (canceled)

164. A genetically modified immune cell, generated by a method of claim 1.

165. A method of treating a disease or condition using a cell therapy and/or delivering a therapy to treat cancer, comprising administering to a patient in need thereof a cell generated by the method of claim 1.

166-179. (canceled)


SEQUENCE LISTING (815 sequences)

SEQ ID NO: 1 - protein, 572 aa, synthetic construct
  REGION 1..572: note = Synthetic Sequence
MAQHSDYSDDEFCADKLSNYSCDSDLENASTSDEDSSDDEVMVRPRTLRRRRISSSSSDSESDIEGGREEWSHVDNPPVLEDFLGHQGLNTDAVINNIEDAVKLFIGDDFFEFLVEESNRYYNQNRNNFKLSKKSLKWKDITPQEMKKFLGLIVLMGQVRKDRRDDYWTTEPWTETPYFGKTMTRDRFRQIWKAWHFNNNADIVNESDRLCKVRPVLDYFVPKFINIYKPHQQLSLDEGIVPWRGRLFFRVYNAGKIVKYGILVRLLCESDTGYICNMEIYCGEGKRLLETIQTVVSPYTDSWYHIYMDNYYNSVANCEALMKNKFRICGTIRKNRGIPKDFQTISLKKGETKFIRKNDILLQVWQSKKPVYLISSIHSAEMEESQNIDRTSKKKIVKPNALIDYNKHMKGVDRADQYLSYYSILRRTVKWTKRLAMYMINCALFNSYAVYKSVRQRKMGFKMFLKQTAIHWLTDDIPEDMDIVPDLQPVPSTSGMRAKPPTSDPPCRLSMDMRKHTLQAIVGSGKKKNILRRCRVCSVHKLRSETRYMCKFCNIPLHKGACFEKYHTLKNY

SEQ ID NO: 2 - DNA, 1719 bp, synthetic construct
  misc_feature 1..1719: note = Synthetic Sequence
  source 1..1719: mol_type = other DNA
atggcccagcacagcgactacagcgacgacgagttctgtgccgataagctgagtaactacagctgcgacagcgacctggaaaacgccagcacatccgacgaggacagctctgacgacgaggtgatggtgcggcccagaaccctgagacggagaagaatcagcagctctagcagcgactctgaatccgacatcgagggcggccgggaagagtggagccacgtggacaaccctcctgttctggaagattttctgggccatcagggcctgaacaccgacgccgtgatcaacaacatcgaggatgccgtgaagctgttcataggagatgatttctttgagttcctggtcgaggaatccaaccgctattacaaccagaatagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcagaaaaaacagaggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccgaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaacatccttagacggtgcagggtgtgcagcgtgcacaagctgcggagcgagactcggtacatgtgcaagttttgcaacattcccctgcacaagggagcctgcttcgagaagtaccacaccctgaagaattactag

SEQ ID NO: 3 - protein, 585 aa, synthetic construct
  REGION 1..585: note = Synthetic Sequence
MSNPRKRSIPMRDSNTGLEQLLAEDSFDESDFSEIDDSDNFSDSALEADKIRPLSHLESDGKSSTSSDSGRSMKWSARAMIPRQRYDFTGTPGRKVDVSDITDPLQYFELFFTEELVSKITRETNAQAALLASKPPGPKGFSRMDKWKDTDNDELKVFFAVMLLQGIVQKPELEMFWSTRPLLDTPYLRQIMTGERFLLLFRCLHFVNNSSISAGQSKAQISLQKIKPVFDFLVNKFSTVYTPNRNIAVDESLMLFKGPLAMKQYLPTKRVRFGLKLYVLCESQSGYVWNALVHTGPGMNLKDSADGLKSSRIVLTLVNDLLGQGYCVFLDNFNISPMLFRELHQNRTDAVGTARLNRKQIPNDLKKRIAKGTTVARFCGELMALKWCDGKEVTMLSTFHNDTVIEVNNRNGKKTKRPRVIVDYNENMGAVDSADQMLTSYPSERKRHKVWYKKFFHHLLHITVLNSYILFKKDNPEHTMSHINFRLALIERMLEKHHKPGQQHLRGRPCSDDVTPLRLSGRHFPKSIPATSGKQNPTGRCKICCSQYDKDGKKIRKETRYFCAECDVPLCVVPCFEIYHTKKNY

SEQ ID NO: 4 - protein, 585 aa, synthetic construct
  REGION 1..585: note = Synthetic Sequence
MSNPRKRPIPMRDSNTRLEQLLAEDSFDESDFSEIDDSDNFSDSALEADKIRPLSHLESDGKSSTSSDSGRSMKWSARAMIPRQRYDFTGTPGRKVDVSDITDPLQYFELFFTEELVSKITRETNAQAALLASKPPGPKGFSRMDKWKDTDNDELKVFFAVMLLQGIVQKPELEMFWSTRPLLDTPYLRQIMTGERFLLLFRCLHFVNNSSISAGQSKAQISLQKIKPVFDFLVNKFSTVYTPNRNIAVDESLMLFKGPLAMKQYLPTKRVRFGLKLYVLCESQSGYVWNALVHTGPGMNLKDSADGLKSSRIVLTLVNDLLGQGYCVFLDNFNISPMLFRELHQNRTDAVGTARLNRKQIPNDLKKRIAKGTTVARFCGELMALKWCDGKEVTMLSTFHNDTVIEVNNRNGKKTKRPRVIVDYNENMGAVDSADQMLTSYPSERKRHKVWYKKFFHHLLHITVLNSYILFKKDNPEHTMSHINFRLALIERMLEKHHKPGQQHLRGRPCSDDVTPLRLSGRHFPKSIPATSGKQNPTGRCKICCSQYDKDGKKIRKETRYFCAECDVPLCVVPCFEIYHTKKNY

SEQ ID NO: 5 - DNA, 1758 bp, synthetic construct
  misc_feature 1..1758: note = Synthetic Sequence
  source 1..1758: mol_type = other DNA
atgtcaaatcctagaaaacgtcccattcctatgcgtgatagtaatacccgtctcgaacagttgttggctgaagattcatttgatgaatctgatttttcggaaatagatgattctgataatttttcggatagtgctttagaagccgataagatcaggcctctgtcccatttagaatctgatggaaagagctctacatcaagtgactcagggcgctccatgaaatggtcagctcgtgctatgattccacgtcaaaggtatgactttaccggcacacctggcagaaaagtcgatgtcagtgatatcactgacccattgcagtattttgaactgttctttactgaggaattagtttcaaaaattactagagaaacaaatgcccaagctgccttgttggcttcaaagccaccgggtccgaaaggattttcgcgaatggataaatggaaagacactgacaatgacgagctcaaagtcttttttgcagtaatgttactgcaaggtattgtgcagaaacctgagctggagatgttttggtcaacaaggcctcttttggatacaccttatctcaggcaaattatgactggtgaaagatttttacttttgtttcggtgcctgcattttgtcaacaattcttctatatctgctggtcaatcaaaggcccagatttcattgcagaagatcaaacctgtgttcgactttcttgtaaataaattttccactgtatatactccaaacagaaacattgcagttgatgaatcactgatgctgttcaaggggccattagctatgaagcagtacctcccgacaaaacgagtacgatttggtctgaagctatatgtactttgtgaaagtcagtctggttatgtgtggaatgcgcttgttcacacagggcctggcatgaatttgaaagattcagcggatggcctgaaatcatcacgcattgttcttaccttggtcaatgaccttcttggccaagggtattgtgtcttcctcgataactttaatatatctcccatgcttttcagagaattacatcaaaataggactgatgcagttgggacagctcgtttgaacagaaaacagattccaaatgatctgaaaaaaaggattgcaaaggggacgactgtagccagattctgtggtgaacttatggcactgaaatggtgtgacggcaaggaggtgacaatgttgtcaacattccacaatgatactgtgattgaagtaaacaatagaaatggaaagaaaactaaaaggccacgtgtcattgtggattataacgagaatatgggagcagtggactcggctgatcaaatgcttacttcttatccatctgagcgcaaaagacacaaggtttggtataagaaattctttcaccatcttctacacattacagtgctgaactcctacatcctgttcaagaaggataatcctgagcacacgatgagccatataaacttcagactggcattgattgaaagaatgctggaaaagcatcacaagccagggcagcaacatcttcgaggtcgtccttgctccgatgatgtcacacctcttcgtctgtctggaagacatttccccaagagcataccagcaacgtccgggaaacagaatccaactggtcgctgcaaaatttgctgctcccaatacgacaaggatggcaagaagatccggaaagaaacgcgctatttttgtgccgaatgtgatgttccgctttgtgttgttccgtgctttgaaatttaccacacgaaaaaaaattattaa

SEQ ID NO: 6 - protein, 809 aa, synthetic construct
  REGION 1..809: note = Synthetic Sequence
MYEALPGPAPENEDGLVKVKEEDPTWEQVCNSQEGSSHTQEICRLRFRHFCYQEAHGPQEALAQLRELCHQWLRPEMHTKEQIMELLVLEQFLTILPKELQPCVKTYPLESGEEAVTVLENLETGSGDTGQQASVYIQGQDMHPMVAEYQGVSLECQSLQLLPGITTLKCEPPQRPQGNPQEVSGPVPHGSAHLQEKNPRDKAVVPVFNPVRSQTLVKTEEETAQAVAAEKWSHLSLTRRNLCGNSAQETVMSLSPMTEEIVTKDRLFKAKQETSEEMEQSGEASGKPNRECAPQIPCSTPIATERTVAHLNTLKDRHPGDLWARMHISSLEYAAGDITRKGRKKDKARVSELLQGLSFSGDSDVEKDNEPEIQPAQKKLKVSCFPEKSWTKRDIKPNFPSWSALDSGLLNLKSEKLNPVELFELFFDDETFNLIVNETNNYASQKNVSLEVTVQEMRCVFGVLLLSGFMRHPRREMYWEVSDTDQNLVRDAIRRDRFELIFSNLHFADNGHLDQKDKFTKLRPLIKQMNKNFLLYAPLEEYYCFDKSMCECFDSDQFLNGKPIRIGYKIWCGTTTQGYLVWFEPYQEESTMKVDEDPDLGLGGNLVMNFADVLLERGQYPYHLCFDSFFTSVKLLSALKKKGVRATGTIRENRTEKCPLMNVEHMKKMKRGYFDFRIEENNEIILCRWYGDGIISLCSNAVGIEPVNEVSCCDADNEEIPQISQPSIVKVYDECKEGVAKMDQIISKYRVRIRSKKWYSILVSYMIDVAMNNAWQLHRACNPGASLDPLDFRRFVAHFYLEHNAHLSD

SEQ ID NO: 7 - protein, 592 aa, synthetic construct
  REGION 1..592: note = Synthetic Sequence
MASTSRDVIAGRGIHSKVKSAKLLEVLNAMEEEESNNNREEIFIAPPDNAAGEFTDEDSGDEDSQRGAHLPGSVLHASVLCEDSGTGEDNDDLELQPAKKRQKAVVKPQRIWTKRDIRPDFGSWTASDPHIEDLKSQELSPVGLFELFFDEGTINFIVNETNRYAWQKNVNLSLTAQELKCVLGILILSGYISYPRRRMFWETSPDSHHHLVADAIRRDRFELIFSYLHFADNNELDASDRFAKVRPLIIRMNCNFQKHAPLEEFYSFGESMCEYFGHRGSKQLHRGKPVRLGYKIWCGTTSRGYLVWFEPSQGTLFTKPDRSLDLGGSMVIKFVDALQERGFLPYHIFFDKVFTSVKLMSILRKKGVKATGTVREYRTERCPLKDPKELKKMKRGSFDYKVDESEEIIVCRWHDSSVVNICSNAVGIEPVRLTSRHSGAAKTRTQVHQPSLVKLYQEKVGGVGRMDQNIAKYKVKIRGMKWYSSFIGYVIDAALNNAWQLHRICCQDAQVDLLAFRRYIACVYLESNADTTSQGRRSRRLETESRFDMIGHWIIHQDKRTRCALCHSQTNTRCEKCQKGVHAKCFREYHIR

SEQ ID NO: 8 - protein, 593 aa, synthetic construct
  REGION 1..593: note = Synthetic Sequence
MPRTLSLHEITDLLETDDSIEASAIVIQPPENATAPVSDEESGDEEGGTINNLPGSLLHTAAYLIQDGSDAESDSDDPSYAPKDDSPDEVPSTFTVQQPPPSRRRKMTKILCKWKKADLTVQPVAGRVTAPPNDFFTVMRTPTEILELFLDDEVIELIVKYSNLYACSKGVHLGLTSSEFKCFLGIIFLSGYVSVPRRRMFWEQRTDVHNVLVSAAMRRDRFETIFSNLHVADNANLDPVDKFSKLRPLISKLNERCMKFVPNETYFSFDEFMVPYFGRHGCKQFIRGKPIRFGYKFWCGATCLGYICWFQPYQGKNPNTKHEEYGVGASLVLQFSEALTEAHPGQYHFVFNNFFTSIALLDKLSSMGHQATGTVRKDHIDRVPLESDVALKKKERGTFDYRIDGKGNIVCRWNDNSVVTVASSGAGIHPLCLVSRYSQKLKKKIQVQQPNMIKVYNQFMGGVDRADENIDKYRASIRGKKWYSSPLLFCFELVLQNAWQLHKTYDEKPVDFLEFRRRVVCHYLETHGHPPEPGQKGRPQKRNIDSRYDGINHVIVKQGKQTRCAECHKNTTFRCEKCDVALHVKCSVEYHTE

SEQ ID NO: 9 - protein, 524 aa, synthetic construct
  REGION 1..524: note = Synthetic Sequence
MAEGGGGARRRAPALLEAARARYESLHISDDVFGESGPDSGGNPFYSTSAASRSSSAASSDDEREPPGPPGAAPPPPRAPDAQEPEEDEAGAGWSAALRDRPPPRFEDTGGPTRKMPPSASAVDFFQLFVPDNVLKNMVVQTNMYAKKFQERFGSDGAWVEVTLTEMKAFLGYMISTSISHCESVLSIWSGGFYSNRSLALVMSQARFEKILKYFHVVAFRSSQTTHGLYKVQPFLDSLQNSFDSAFRPSQTQVLHEPLIDEDPVFIATCTERELRKRKKRKFSLWVRQCSSTGFIIQIYVHLKEGGGPDGLDALKNKPQLHSMVARSLCRNAAGKNYIIFTGPSITSLTLFEEFEKQGIYCCGLLRARKSDCTGLPLSMLTNPATPPARGQYQIKMKGNMSLICWYNKGHFRFLTNAYSPVQQGVIIKRKSGEIPCPLAVEAFAAHLSYICRYDDKYSKYFISHKPNKTWQQVFWFAISIAINNAYILYKMSDAYHVKRYSRAQFGERLVRELLGLEDASPTH

SEQ ID NO: 10 - protein, 572 aa, synthetic construct
  REGION 1..572: note = Synthetic Sequence
MSQHSDYPDDEFRADKLSNYSCDSDLENASTSDEDSSDDEVMVRPRTLRRRRISSSSSDSESDIEGGREEWSHVDNPPVLEDFLGHQGLNTDAVINNIEDAVKLFIGDDFFEFLVEESNRYYNQKRNNFKLSKKSLKWKDITPQEMKKFLGLIVLMGQVRKDRRDDYWTTEPWTETPYFGKTMTRDRFRQIWKAWHFNNNADIVNESDRLCKVRPVLDYFVPKFINIYKPHQQLSLDEGIVPWRGRLFFRVYNAGKIVKYGILVRLLCESDTGYICNMEIYCGEGKRLLETIQTVVSPYTDSWYHIYMDNYYNSVANCEALMKNKFRICGTIRKNRGIPKDFQTISLKKGETKFIRKNDILLQVWQSKKPVYLISSIHSAEMEESQNIDRTSKKKIVKPNALIDYNKHMKGVDRADQYLSYYSILRRTVKWTKRLAMYMINCALFNSYAVYKSVRQRKMGFKMFLKQTAIHWLTDDIPEDMDIVPDLQPVPSTSGMRAKPPTSDPPCRLSMDMRKHTLQAIVGSGKKKNILRRCRVCSVHKLRSETRYMCKFCNIPLHKGACFEKYHTLKNY

SEQ ID NO: 11 - protein, 572 aa, synthetic construct
  REGION 1..572: note = Synthetic Sequence
MAQHSDYPDDEFRADKLSNYSCDSDLENASTSDEDSSDDEVMVRPRTLRRRRISSSSSDSESDIEGGREEWSHVDNPPVLEDFLGHQGLNTDAVINNIEDAVKLFIGDDFFEFLVEESNRYYNQNRNNFKLSKKSLKWKDITPQEMKKFLGLIVLMGQVRKDRRDDYWTTEPWTETPYFGKTMTRDRFRQIWKAWHFNNNADIVNESDRLCKVRPVLDYFVPKFINIYKPHQQLSLDEGIVPWRGRLFFRVYNAGKIVKYGILVRLLCESDTGYICNMEIYCGEGKRLLETIQTVVSPYTDSWYHIYMDNYYNSVANCEALMKNKFRICGTIRKNRGIPKDFQTISLKKGETKFIRKNDILLQVWQSKKPVYLISSIHSAEMEESQNIDRTSKKKIVKPNALIDYNKHMKGVDRADQYLSYYSILRRTVKWTKRLAMYMINCALFNSYAVYKSVRQRKMGFKMFLKQTAIHWLTDDIPEDMDIVPDLQPVPSTSGMRAKPPTSDPPCRLSMDMRKHTLQAIVGSGKKKNILRRCRVCSVHKLRSETRYMCKFCNIPLHKGACFEKYHTLKNY

SEQ ID NO: 12 - DNA, 381 bp, synthetic construct
  misc_feature 1..381: note = Synthetic Sequence
  source 1..381: mol_type = other DNA
ttaacccatttcctgtttgccccgagaatactcaccagcggcacttgcagctgcagcgtttaccccgagataactcgtcgattacagtcctaaccttacccccaaagtttgccatgaaatatctcgcttttattattattttcgcatcgctctagtatatcgatagtctttggaaacaaatgacatcattctatttacagcattctgtttttagtagtggtatttccatttacaaaatatagtaattttctatcgctgaaaatgtcaaatcctagaaaacgtagcattcctacatgtgatgttaacttcgttctcgaacagttgttagccgaagattcatttgatgaatccgatttttccgaaatagacgattctgatgat

SEQ ID NO: 13 - DNA, 373 bp, synthetic construct
  misc_feature 1..373: note = Synthetic Sequence
  source 1..373: mol_type = other DNA
ttaactcatttctccttagccccgagattacgcgctgctgtgcctgcgactgcagcgtttacgccgagataactcgtggattacagtgccaaccttactcccaaagtttgccacgaaatatctcgcttctgttattttcgcatggttctggtatattgacttttgaaacaaaagacatcattctgtttatagcattctgtttttagtagtgggatttccatctacaaaatatagtaattctcgatcgctgaaatgtcaaatcctagaaaacgtagcattcctatgcgtgatagtaataccggtctcgaacagttgttggctgaagattcatttgatgaatctgatttttcggaaatagatgattctgataatt

SEQ ID NO: 14 - DNA, 344 bp, synthetic construct
  misc_feature 1..344: note = Synthetic Sequence
  source 1..344: mol_type = other DNA
ttaacccttttcccgtttgccccgagaatactcgccggcggcgcttgcggctgcagcgtttaccccgagataactttgccacgaaatatctcgcttttattattattttcgcatcgctctagtatatcgactttggaaacaaaagacatcattctatttatagcattctgtttttagtagtggtatttccatttacaaaatatagtaattctcgatcgctgaaaatgtcaaatcctagaaaacgtagcattcctacgcgtgatgttaacatcgttctcgaacagttgttggccgaagattcatttgatgaatccgatttttccgaaatagacgattctgatgat

SEQ ID NO: 15 - DNA, 91 bp, synthetic construct
  misc_feature 1..91: note = Synthetic Sequence
  source 1..91: mol_type = other DNA
ttaacccatttcccgtttgccccgagaatactcttgtctctaatcctaatgtaacatcatatacatttctgttacattaggattagagaca

SEQ ID NO: 16 - DNA, 32 bp, synthetic construct
  misc_feature 1..32: note = Synthetic Sequence
  source 1..32: mol_type = other DNA
ttaacccatttcccgtttgccccgagaatact

SEQ ID NO: 17 - DNA, 171 bp, synthetic construct
  misc_feature 1..171: note = Synthetic Sequence
  source 1..171: mol_type = other DNA
taggattagagacaagttctgtttagaaataactccaagaacagtttttatattttattttcacattgaaaaccagtcagatttgcttcagcctcaaagagcatgtttatgtaaaattaaattaacgctggcagcgagctgcactttttttctaaacgggaaatgggttaa

SEQ ID NO: 18 - DNA, 176 bp, synthetic construct
  misc_feature 1..176: note = Synthetic Sequence
  source 1..176: mol_type = other DNA
cctgggattataggcatgagccactgcgcctagcaccaagaacagtttttatattttattttcacattgaaaatcagtcagatttgcttcagcctcaaagagggtgtttatgtaaaactaaatgagtgcaggcagcgagctacacttttttttttcctaaatggaaaatgggttaa

SEQ ID NO: 19 - DNA, 178 bp, synthetic construct
  misc_feature 1..178: note = Synthetic Sequence
  source 1..178: mol_type = other DNA
tcagacgattctgatgttagttctgtttagaaataactccaagaacagtttttatattttattttcacattgaaaatcagtcagatttgcttcagcctcaaagagcgtgtttatgtaaaattaaatgagcgctggcagcgagctgcacttttttttttctaaacgggaaaagggttaa

SEQ ID NO: 20 - DNA, 160 bp, synthetic construct
  misc_feature 1..160: note = Synthetic Sequence
  source 1..160: mol_type = other DNA
agttctgtttagaaataactccaagaacagtttttatattttattttcacattgaaaatcagtcagatttgcttcagcctcaaagagcgtgtttatgtaaaattaaatgagcgctggcagcgagctgcacttttttttttctaaacgggaaaagggttaa

SEQ ID NO: 21 - DNA, 157 bp, synthetic construct
  misc_feature 1..157: note = Synthetic Sequence
  source 1..157: mol_type = other DNA
ttaacacttggattgcgggaaacgagttaagtcggctcgcgtgaattgcgcgtactccgcgggagccgtcttaactcggttcatatagatttgcggtggagtgcgggaaacgtgtaaactcgggccgattgtaactgcgtattaccaaatatttgtt

SEQ ID NO: 22 - DNA, 212 bp, synthetic construct
  misc_feature 1..212: note = Synthetic Sequence
  source 1..212: mol_type = other DNA
aattatttatgtactgaatagataaaaaaatgtctgtgattgaataaattttcattttttacacaagaaaccgaaaatttcatttcaatcgaacccatacttcaaaagatataggcattttaaactaactctgattttgcgcgggaaacctaaataattgcccgcgccatcttatattttggcgggaaattcacccgacaccgtagtgttaa

SEQ ID NO: 23 - DNA, 19 bp, synthetic construct
  misc_feature 1..19: note = Synthetic Sequence
  source 1..19: mol_type = other DNA
tggccggcctgaccactgg

SEQ ID NO: 24 - DNA, 19 bp, synthetic construct
  misc_feature 1..19: note = Synthetic Sequence
  source 1..19: mol_type = other DNA
tgaaggcctggccggcctg

SEQ ID NO: 25 - DNA, 19 bp, synthetic construct
  misc_feature 1..19: note = Synthetic Sequence
  source 1..19: mol_type = other DNA
tgagcactgaaggcctggc

SEQ ID NO: 26 - DNA, 19 bp, synthetic construct
  misc_feature 1..19: note = Synthetic Sequence
  source 1..19: mol_type = other DNA
tccactgagcactgaaggc

SEQ ID NO: 27 - DNA, 19 bp, synthetic construct
  misc_feature 1..19: note = Synthetic Sequence
  source 1..19: mol_type = other DNA
tggtttccactgagcactg

SEQ ID NO: 28 - DNA, 19 bp, synthetic construct
  misc_feature 1..19: note = Synthetic Sequence
  source 1..19: mol_type = other DNA
tggggaaaatgacccaaca

SEQ ID NO: 29 - DNA, 19 bp, synthetic construct
  misc_feature 1..19: note = Synthetic Sequence
  source 1..19: mol_type = other DNA
taggacagtggggaaaatg

SEQ ID NO: 30 - DNA, 19 bp, synthetic construct
  misc_feature 1..19: note = Synthetic Sequence
  source 1..19: mol_type = other DNA
tccagggacacggtgctag

SEQ ID NO: 31 - DNA, 19 bp, synthetic construct
  misc_feature 1..19: note = Synthetic Sequence
  source 1..19: mol_type = other DNA
tcagagccaggagtcctgg

SEQ ID NO: 32 - DNA, 19 bp, synthetic construct
  misc_feature 1..19: note = Synthetic Sequence
  source 1..19: mol_type = other DNA
tccttcagagccaggagtc

SEQ ID NO: 33 - DNA, 19 bp, synthetic construct
  misc_feature 1..19: note = Synthetic Sequence
  source 1..19: mol_type = other DNA
tcctccttcagagccagga

SEQ ID NO: 34 - DNA, 19 bp, synthetic construct
  misc_feature 1..19: note = Synthetic Sequence
  source 1..19: mol_type = other DNA
tccagcccctcctccttca

SEQ ID NO: 35 - DNA, 19 bp, synthetic construct
  misc_feature 1..19: note = Synthetic Sequence
  source 1..19: mol_type = other DNA
tccgagcttgacccttgga

SEQ ID NO: 36 - DNA, 19 bp, synthetic construct
  misc_feature 1..19: note = Synthetic Sequence
  source 1..19: mol_type = other DNA
tggtttccgagcttgaccc

SEQ ID NO: 37 - DNA, 19 bp, synthetic construct
  misc_feature 1..19: note = Synthetic Sequence
  source 1..19: mol_type = other DNA
tggggtggtttccgagctt

SEQ ID NO: 38 - DNA, 19 bp, synthetic construct
  misc_feature 1..19: note = Synthetic Sequence
  source 1..19: mol_type = other DNA
tctgctggggtggtttccg

SEQ ID NO: 39 - DNA, 19 bp, synthetic construct
  misc_feature 1..19: note = Synthetic Sequence
  source 1..19: mol_type = other DNA
tgcagagtatctgctgggg

SEQ ID NO: 40 - DNA, 14 bp, synthetic construct
  misc_feature 1..14: note = Synthetic Sequence
  source 1..14: mol_type = other DNA
ccaatcccctcagt

SEQ ID NO: 41 - DNA, 15 bp, synthetic construct
  misc_feature 1..15: note = Synthetic Sequence
  source 1..15: mol_type = other DNA
cagtgctcagtggaa

SEQ ID NO: 42 - DNA, 18 bp, synthetic construct
  misc_feature 1..18: note = Synthetic Sequence
  source 1..18: mol_type = other DNA
gaaacatccggcgactca

SEQ ID NO: 43 - DNA, 19 bp, synthetic construct
  misc_feature 1..19: note = Synthetic Sequence
  source 1..19: mol_type = other DNA
tcgcccctcaaatcttaca

SEQ ID NO: 44 - DNA, 19 bp, synthetic construct
  misc_feature 1..19: note = Synthetic Sequence
  source 1..19: mol_type = other DNA
tcaaatcttacagctgctc

SEQ ID NO: 45 - DNA, 19 bp, synthetic construct
  misc_feature 1..19: note = Synthetic Sequence
  source 1..19: mol_type = other DNA
tcttacagctgctcactcc

SEQ ID NO: 46 - DNA, 19 bp, synthetic construct
  misc_feature 1..19: note = Synthetic Sequence
  source 1..19: mol_type = other DNA
tacagctgctcactcccct

SEQ ID NO: 47 - DNA, 19 bp, synthetic construct
  misc_feature 1..19: note = Synthetic Sequence
  source 1..19: mol_type = other DNA
tgctcactcccctgcaggg

SEQ ID NO: 48 - DNA, 19 bp, synthetic construct
  misc_feature 1..19: note = Synthetic Sequence
  source 1..19: mol_type = other DNA
tcccctgcagggcaacgcc

SEQ ID NO: 49 - DNA, 19 bp, synthetic construct
  misc_feature 1..19: note = Synthetic Sequence
  source 1..19: mol_type = other DNA
tgcagggcaacgcccaggg

SEQ ID NO: 50 - DNA, 19 bp, synthetic construct
  misc_feature 1..19: note = Synthetic Sequence
  source 1..19: mol_type = other DNA
tctcgattatgggcgggat

SEQ ID NO: 51 - DNA, 19 bp, synthetic construct
  misc_feature 1..19: note = Synthetic Sequence
  source 1..19: mol_type = other DNA
tcgcttctcgattatgggc

SEQ ID NO: 52 - DNA, 19 bp, synthetic construct
  misc_feature 1..19: note = Synthetic Sequence
  source 1..19: mol_type = other DNA
tgtcgagtcgcttctcgat

SEQ ID NO: 53 - DNA, 19 bp, synthetic construct
  misc_feature 1..19: note = Synthetic Sequence
  source 1..19: mol_type = other DNA
tccatgtcgagtcgcttct

SEQ ID NO: 54 - DNA, 19 bp, synthetic construct
  misc_feature 1..19: note = Synthetic Sequence
  source 1..19: mol_type = other DNA
tcgcctccatgtcgagtcg

SEQ ID NO: 55 - DNA, 19 bp, synthetic construct
  misc_feature 1..19: note = Synthetic Sequence
  source 1..19: mol_type = other DNA
tcgtcatcgcctccatgtc

SEQ ID NO: 56 - DNA, 19 bp, synthetic construct
  misc_feature 1..19: note = Synthetic Sequence
  source 1..19: mol_type = other DNA
tgatctcgtcatcgcctcc

SEQ ID NO: 57 - DNA, 14 bp, synthetic construct
  misc_feature 1..14: note = Synthetic Sequence
  source 1..14: mol_type = other DNA
gcttcagcttccta

SEQ ID NO: 58 - DNA, 14 bp, synthetic construct
  misc_feature 1..14: note = Synthetic Sequence
  source 1..14: mol_type = other DNA
ctgtgatcatgcca

SEQ ID NO: 59 - DNA, 16 bp, synthetic construct
  misc_feature 1..16: note = Synthetic Sequence
  source 1..16: mol_type = other DNA
acagtggtacacacct

SEQ ID NO: 60 - DNA, 15 bp, synthetic construct
  misc_feature 1..15: note = Synthetic Sequence
  source 1..15: mol_type = other DNA
ccaccccccactaag

SEQ ID NO: 61 - DNA, 14 bp, synthetic construct
  misc_feature 1..14: note = Synthetic Sequence
  source 1..14: mol_type = other DNA
cattggccgggcac

SEQ ID NO: 62 - DNA, 16 bp, synthetic construct
  misc_feature 1..16: note = Synthetic Sequence
  source 1..16: mol_type = other DNA
gcttgaacccaggaga

SEQ ID NO: 63 - DNA, 17 bp, synthetic construct
  misc_feature 1..17: note = Synthetic Sequence
  source 1..17: mol_type = other DNA
acacccgatccactggg

SEQ ID NO: 64 - DNA, 14 bp, synthetic construct
  misc_feature 1..14: note = Synthetic Sequence
  source 1..14: mol_type = other DNA
gctgcatcaacccc

SEQ ID NO: 65 - DNA, 16 bp, synthetic construct
  misc_feature 1..16: note = Synthetic Sequence
  source 1..16: mol_type = other DNA
gccacaaacagaaata

SEQ ID NO: 66 - DNA, 15 bp, synthetic construct
  misc_feature 1..15: note = Synthetic Sequence
  source 1..15: mol_type = other DNA
ggtggctcatgcctg

SEQ ID NO: 67 - DNA, 16 bp, synthetic construct
  misc_feature 1..16: note = Synthetic Sequence
  source 1..16: mol_type = other DNA
gatttgcacagctcat

SEQ ID NO: 68 - DNA, 15 bp, synthetic construct
  misc_feature 1..15: note = Synthetic Sequence
  source 1..15: mol_type = other DNA
aagctctgaggagca

SEQ ID NO: 69 - DNA, 13 bp, synthetic construct
  misc_feature 1..13: note = Synthetic Sequence
  source 1..13: mol_type = other DNA
ccctagctgtccc

SEQ ID NO: 70 - DNA, 14 bp, synthetic construct
  misc_feature 1..14: note = Synthetic Sequence
  source 1..14: mol_type = other DNA
gcctagcatgctag

SEQ ID NO: 71 - DNA, 15 bp, synthetic construct
  misc_feature 1..15: note = Synthetic Sequence
  source 1..15: mol_type = other DNA
atgggcttcacggat

SEQ ID NO: 72 - DNA, 14 bp, synthetic construct
  misc_feature 1..14: note = Synthetic Sequence
  source 1..14: mol_type = other DNA
gaaactatgcctgc

SEQ ID NO: 73 - DNA, 14 bp, synthetic construct
  misc_feature 1..14: note = Synthetic Sequence
  source 1..14: mol_type = other DNA
gcaccattgctccc

SEQ ID NO: 74 - DNA, 14 bp, synthetic construct
  misc_feature 1..14: note = Synthetic Sequence
  source 1..14: mol_type = other DNA
gacatgcaactcag

SEQ ID NO: 75 - DNA, 14 bp, synthetic construct
  misc_feature 1..14: note = Synthetic Sequence
  source 1..14: mol_type = other DNA
acaccactaggggt

SEQ ID NO: 76 - DNA, 14 bp, synthetic construct
  misc_feature 1..14: note = Synthetic Sequence
  source 1..14: mol_type = other DNA
gtctgctagacagg

SEQ ID NO: 77 - DNA, 15 bp, synthetic construct
  misc_feature 1..15: note = Synthetic Sequence
  source 1..15: mol_type = other DNA
ggcctagacaggctg

SEQ ID NO: 78 - DNA, 15 bp, synthetic construct
  misc_feature 1..15: note = Synthetic Sequence
  source 1..15: mol_type = other DNA
gaggcattcttatcg

SEQ ID NO: 79 - DNA, 15 bp, synthetic construct
  misc_feature 1..15: note = Synthetic Sequence
  source 1..15: mol_type = other DNA
gcctggaaacgttcc

SEQ ID NO: 80 - DNA, 14 bp, synthetic construct
  misc_feature 1..14: note = Synthetic Sequence
  source 1..14: mol_type = other DNA
gtgctctgacaata

SEQ ID NO: 81 - DNA, 14 bp, synthetic construct
  misc_feature 1..14: note = Synthetic Sequence
  source 1..14: mol_type = other DNA
gttttgcagcctcc

SEQ ID NO: 82 - DNA, 15 bp, synthetic construct
  misc_feature 1..15: note = Synthetic Sequence
  source 1..15: mol_type = other DNA
acagctgtggaacgt

SEQ ID NO: 83 - DNA, 15 bp, synthetic construct
  misc_feature 1..15: note = Synthetic Sequence
  source 1..15: mol_type = other DNA
ggctctcttcctcct

SEQ ID NO: 84 - DNA, 15 bp, synthetic construct
  misc_feature 1..15: note = Synthetic Sequence
  source 1..15: mol_type = other DNA
ctatcccaaaactct

SEQ ID NO: 85 - DNA, 14 bp, synthetic construct
  misc_feature 1..14: note = Synthetic Sequence
  source 1..14: mol_type = other DNA
gaaaaactatgtat

SEQ ID NO: 86 - DNA, 15 bp, synthetic construct
  misc_feature 1..15: note = Synthetic Sequence
  source 1..15: mol_type = other DNA
aggcaggctggttga

SEQ ID NO: 87 - DNA, 14 bp, synthetic construct
  misc_feature 1..14: note = Synthetic Sequence
  source 1..14: mol_type = other DNA
caatacaaccacgc

SEQ ID NO: 88 - DNA, 15 bp, synthetic construct
  misc_feature 1..15: note = Synthetic Sequence
  source 1..15: mol_type = other DNA
atgacggactcaact

SEQ ID NO: 89 - DNA, 14 bp, synthetic construct
  misc_feature 1..14: note = Synthetic Sequence
  source 1..14: mol_type = other DNA
cacaacatttgtaa

SEQ ID NO: 90 - DNA, 14 bp, synthetic construct
  misc_feature 1..14: note = Synthetic Sequence
  source 1..14: mol_type = other DNA
atttccagtgcaca

SEQ ID NO: 91 - DNA, 20 bp, synthetic construct
  misc_feature 1..20: note = Synthetic Sequence
  source 1..20: mol_type = other DNA
gtttagctcacccgtgagcc

SEQ ID NO: 92 - DNA, 20 bp, synthetic construct
  misc_feature 1..20: note = Synthetic Sequence
  source 1..20: mol_type = other DNA
cccaatattattgttctctg

SEQ ID NO: 93 - DNA, 20 bp, synthetic construct
  misc_feature 1..20: note = Synthetic Sequence
  source 1..20: mol_type = other DNA
ggggtgggataggggatacg

SEQ ID NO: 94 - DNA, 20 bp, synthetic construct
  misc_feature 1..20: note = Synthetic Sequence
  source 1..20: mol_type = other DNA
ggatccccctctacatttaa

SEQ ID NO: 95 - DNA, 20 bp, synthetic construct
  misc_feature 1..20: note = Synthetic Sequence
  source 1..20: mol_type = other DNA
gtgatcttgtacaaatcatt

SEQ ID NO: 96 - DNA, 20 bp, synthetic construct
  misc_feature 1..20: note = Synthetic Sequence
  source 1..20: mol_type = other DNA
ctacacagaatctgttagaa

SEQ ID NO: 97 - DNA, 20 bp, synthetic construct
  misc_feature 1..20: note = Synthetic Sequence
  source 1..20: mol_type = other DNA
taagctagagaatagatctc

SEQ ID NO: 98 - DNA, 20 bp, synthetic construct
  misc_feature 1..20: note = Synthetic Sequence
  source 1..20: mol_type = other DNA
tcaatacacttaatgattta

SEQ ID NO: 99 - DNA, 25 bp, synthetic construct
  misc_feature 1..25: note = Synthetic Sequence
  source 1..25: mol_type = other DNA
caccgggagccacgaaaacagatcc

SEQ ID NO: 100 - DNA, 25 bp, synthetic construct
  misc_feature 1..25: note = Synthetic Sequence
  source 1..25: mol_type = other DNA
caccgcgaaaacagatccagggaca

SEQ ID NO: 101 - DNA, 25 bp, synthetic construct
  misc_feature 1..25: note = Synthetic Sequence
  source 1..25: mol_type = other DNA
caccgagatccagggacacggtgct

SEQ ID NO: 102 - DNA, 25 bp, synthetic construct
  misc_feature 1..25: note = Synthetic Sequence
  source 1..25: mol_type = other DNA
caccggacacggtgctaggacagtg

SEQ ID NO: 103 - DNA, 25 bp, synthetic construct
  misc_feature 1..25: note = Synthetic Sequence
  source 1..25: mol_type = other DNA
caccggaaaatgacccaacagcctc

SEQ ID NO: 104 - DNA, 25 bp, synthetic construct
  misc_feature 1..25: note = Synthetic Sequence
  source 1..25: mol_type = other DNA
caccggcctggccggcctgaccact

SEQ ID NO: 105 - DNA, 25 bp, synthetic construct
  misc_feature 1..25: note = Synthetic Sequence
  source 1..25: mol_type = other DNA
caccgctgagcactgaaggcctggc

SEQ ID NO: 106 - DNA, 25 bp, synthetic construct
  misc_feature 1..25: note = Synthetic Sequence
  source 1..25: mol_type = other DNA
caccgtggtttccactgagcactga

SEQ ID NO: 107 - DNA, 25 bp, synthetic construct
  misc_feature 1..25: note = Synthetic Sequence
  source 1..25: mol_type = other DNA
caccggatagccaggagtcctttcg

SEQ ID NO: 108 - DNA, 25 bp, synthetic construct
  misc_feature 1..25: note = Synthetic Sequence
  source 1..25: mol_type = other DNA
caccggcgcttccagtgctcagact

SEQ ID NO: 109 - DNA, 25 bp, synthetic construct
  misc_feature 1..25: note = Synthetic Sequence
  source 1..25: mol_type = other DNA
caccgcagtgctcagactagggaag

SEQ ID NO: 110 - DNA, 25 bp, synthetic construct
  misc_feature 1..25: note = Synthetic Sequence
  source 1..25: mol_type = other DNA
caccggcccctcctccttcagagcc

SEQ ID NO: 111 - DNA, 25 bp, synthetic construct
  misc_feature 1..25: note = Synthetic Sequence
  source 1..25: mol_type = other DNA
caccgtccttcagagccaggagtcc

SEQ ID NO: 112 - DNA, 25 bp, synthetic construct
  misc_feature 1..25: note = Synthetic Sequence
  source 1..25: mol_type = other DNA
caccgtggtttccgagcttgaccct

SEQ ID NO: 113 - DNA, 25 bp, synthetic construct
  misc_feature 1..25: note = Synthetic Sequence
  source 1..25: mol_type = other DNA
caccgctgcagagtatctgctgggg

SEQ ID NO: 114 - DNA, 25 bp, synthetic construct
  misc_feature 1..25: note = Synthetic Sequence
  source 1..25: mol_type = other DNA
caccgcgttcctgcagagtatctgc

SEQ ID NO: 115-129 - skipped (15 entries)

SEQ ID NO: 130 - DNA, 25 bp, synthetic construct
  misc_feature 1..25: note = Synthetic Sequence
  source 1..25: mol_type = other DNA
aaacgcagatactctgcaggaacgc

SEQ ID NO: 131 - DNA, 20 bp, synthetic construct
  misc_feature 1..20: note = Synthetic Sequence
  source 1..20: mol_type = other DNA
tcccctcccagaaagacctg

SEQ ID NO: 132 - DNA, 20 bp, synthetic construct
  misc_feature 1..20: note = Synthetic Sequence
  source 1..20: mol_type = other DNA
tgggctccaagcaatcctgg

SEQ ID NO: 133 - DNA, 20 bp, synthetic construct
  misc_feature 1..20: note = Synthetic Sequence
  source 1..20: mol_type = other DNA
gtggctcaggaggtacctgg

SEQ ID NO: 134 - DNA, 20 bp, synthetic construct
  misc_feature 1..20: note = Synthetic Sequence
  source 1..20: mol_type = other DNA
gagccacgaaaacagatcca

SEQ ID NO: 135 - DNA, 20 bp, synthetic construct
  misc_feature 1..20: note = Synthetic Sequence
  source 1..20: mol_type = other DNA
aagtgaacggggaagggagg

SEQ ID NO: 136 - DNA, 20 bp, synthetic construct
  misc_feature 1..20: note = Synthetic Sequence
  source 1..20: mol_type = other DNA
gacaaaagccgaagtccagg

SEQ ID NO: 137 - DNA, 20 bp, synthetic construct
  misc_feature 1..20: note = Synthetic Sequence
  source 1..20: mol_type = other DNA
gtggttgataaacccacgtg

SEQ ID NO: 138 - DNA, 20 bp, synthetic construct
  misc_feature 1..20: note = Synthetic Sequence
  source 1..20: mol_type = other DNA
tgggaacagccacagcaggg

SEQ ID NO: 139 - DNA, 20 bp, synthetic construct
  misc_feature 1..20: note = Synthetic Sequence
  source 1..20: mol_type = other DNA
gcaggggaacggggatgcag

SEQ ID NO: 140 - DNA, 20 bp, synthetic construct
  misc_feature 1..20: note = Synthetic Sequence
  source 1..20: mol_type = other DNA
gagatggtggacgaggaagg

SEQ ID NO: 141 - DNA, 20 bp, synthetic construct
  misc_feature 1..20: note = Synthetic Sequence
  source 1..20: mol_type = other DNA
gagatggctccaggaaatgg

SEQ ID NO: 142 - DNA, 20 bp, synthetic construct
  misc_feature 1..20: note = Synthetic Sequence
  source 1..20: mol_type = other DNA
taaggaatctgcctaacagg

SEQ ID NO: 143 - DNA, 20 bp, synthetic construct
  misc_feature 1..20: note = Synthetic Sequence
  source 1..20: mol_type = other DNA
tcaggagactaggaaggagg

SEQ ID NO: 144 - DNA, 20 bp, synthetic construct
  misc_feature 1..20: note = Synthetic Sequence
  source 1..20: mol_type = other DNA
tataaggtggtcccagctcg

SEQ ID NO: 145 - DNA, 20 bp, synthetic construct
  misc_feature 1..20: note = Synthetic Sequence
  source 1..20: mol_type = other DNA
ctggaagatgccatgacagg

SEQ ID NO: 146 - DNA, 20 bp, synthetic construct
  misc_feature 1..20: note = Synthetic Sequence
  source 1..20: mol_type = other DNA
gcacagactagagaggtaag

SEQ ID NO: 147 - DNA, 20 bp, synthetic construct
  misc_feature 1..20: note = Synthetic Sequence
  source 1..20: mol_type = other DNA
acagactagagaggtaaggg

SEQ ID NO: 148 - DNA, 20 bp, synthetic construct
  misc_feature 1..20: note = Synthetic Sequence
  source 1..20: mol_type = other DNA
gagaggtgacccgaatccac

SEQ ID NO: 149 - DNA, 20 bp, synthetic construct
  misc_feature 1..20: note = Synthetic Sequence
  source 1..20: mol_type = other DNA
gcacaggccccagaaggaga

SEQ ID NO: 150 - DNA, 20 bp, synthetic construct
  misc_feature 1..20: note = Synthetic Sequence
  source 1..20: mol_type = other DNA
ccggagaggacccagacacg

SEQ ID NO: 151 - DNA, 20 bp, synthetic construct
  misc_feature 1..20: note = Synthetic Sequence
  source 1..20: mol_type = other DNA
gagaggacccagacacgggg

SEQ ID NO: 152 - DNA, 20 bp, synthetic construct
  misc_feature 1..20: note = Synthetic Sequence
  source 1..20: mol_type = other DNA
gcaacacagcagagagcaag

SEQ ID NO: 153 - DNA, 20 bp, synthetic construct
  misc_feature 1..20: note = Synthetic Sequence
  source 1..20: mol_type = other DNA
gaagagggagtggaggaaga

SEQ ID NO: 154 - DNA, 20 bp, synthetic construct
  misc_feature 1..20: note = Synthetic Sequence
  source 1..20: mol_type = other DNA
aagacggaacctgaaggagg

SEQ ID NO: 155 - DNA, 20 bp, synthetic construct
  misc_feature 1..20: note = Synthetic Sequence
  source 1..20: mol_type = other DNA
agaaagcggcacaggcccag

SEQ ID NO: 156 - DNA, 20 bp, synthetic construct
  misc_feature 1..20: note = Synthetic Sequence
  source 1..20: mol_type = other DNA
gggaaacagtgggccagagg

SEQ ID NO: 157 - DNA, 20 bp, synthetic construct
  misc_feature 1..20: note = Synthetic Sequence
  source 1..20: mol_type = other DNA
gtccggactcaggagagaga

SEQ ID NO: 158 - DNA, 20 bp, synthetic construct
  misc_feature 1..20: note = Synthetic Sequence
  source 1..20: mol_type = other DNA
ggcacagcaagggcactcgg

SEQ ID NO: 159 - DNA, 20 bp, synthetic construct
  misc_feature 1..20: note = Synthetic Sequence
  source 1..20: mol_type = other DNA
gaagaggggaagtcgaggga

SEQ ID NO: 160 - DNA, 20 bp, synthetic construct
  misc_feature 1..20: note = Synthetic Sequence
  source 1..20: mol_type = other DNA
gggaatggtaaggaggcctg

SEQ ID NO: 161 - DNA, 20 bp, synthetic construct
  misc_feature 1..20: note = Synthetic Sequence
  source 1..20: mol_type = other DNA
gcagagtggtcagcacagag

SEQ ID NO: 162 - DNA, 20 bp, synthetic construct
  misc_feature 1..20: note = Synthetic Sequence
  source 1..20: mol_type = other DNA
gcacagagtggctaagccca

SEQ ID NO: 163 - DNA, 20 bp, synthetic construct
  misc_feature 1..20: note = Synthetic Sequence
  source 1..20: mol_type = other DNA
gacggggtgtcagcataggg

SEQ ID NO: 164 - DNA, 20 bp, synthetic construct
  misc_feature 1..20: note = Synthetic Sequence
  source 1..20: mol_type = other DNA
gcccagggccaggaacgacg

SEQ ID NO: 165 - DNA, 20 bp, synthetic construct
  misc_feature 1..20: note = Synthetic Sequence
  source 1..20: mol_type = other DNA
ggtggagtccagcacggcgc

SEQ ID NO: 166 - DNA, 20 bp, synthetic construct
  misc_feature 1..20: note = Synthetic Sequence
  source 1..20: mol_type = other DNA
acaggccgccaggaactcgg

SEQ ID NO: 167 - DNA, 20 bp, synthetic construct
  misc_feature 1..20: note = Synthetic Sequence
  source 1..20: mol_type = other DNA
actaggaagtgtgtagcacc

SEQ ID NO: 168 - DNA, 20 bp, synthetic construct
  misc_feature 1..20: note = Synthetic Sequence
  source 1..20: mol_type = other DNA
atgaatagcagactgccccg

SEQ ID NO: 169 - DNA, 20 bp, synthetic construct
  misc_feature 1..20: note = Synthetic Sequence
  source 1..20: mol_type = other DNA
acacccctaaaagcacagtg

SEQ ID NO: 170 - DNA, 20 bp, synthetic construct
  misc_feature 1..20: note = Synthetic Sequence
  source 1..20: mol_type = other DNA
caaggagttccagcaggtgg

SEQ ID NO: 171 - DNA, 20 bp, synthetic construct
  misc_feature 1..20: note = Synthetic Sequence
  source 1..20: mol_type = other DNA
aaggagttccagcaggtggg

SEQ ID NO: 172 - DNA, 20 bp, synthetic construct
  misc_feature 1..20: note = Synthetic Sequence
  source 1..20: mol_type = other DNA
tggaaagaggagggaagagg

SEQ ID NO: 173 - DNA, 20 bp, synthetic construct
  misc_feature 1..20: note = Synthetic Sequence
  source 1..20: mol_type = other DNA
tcgaattcctaactgccccg

SEQ ID NO: 174 - DNA, 20 bp, synthetic construct
  misc_feature 1..20: note = Synthetic Sequence
  source 1..20: mol_type = other DNA
gacctgcccagcacaccctg

SEQ ID NO: 175 - DNA, 20 bp, synthetic construct
  misc_feature 1..20: note = Synthetic Sequence
  source 1..20: mol_type = other DNA
ggagcagctgcggcagtggg

SEQ ID NO: 176 - DNA, 20 bp, synthetic construct
  misc_feature 1..20: note = Synthetic Sequence
  source 1..20: mol_type = other DNA
gggagggagagcttggcagg

SEQ ID NO: 177 - DNA, 20 bp, synthetic construct
  misc_feature 1..20: note = Synthetic Sequence
  source 1..20: mol_type = other DNA
gttacgtggccaagaagcag

SEQ ID NO: 178 - DNA, 20 bp, synthetic construct
  misc_feature 1..20: note = Synthetic Sequence
  source 1..20: mol_type = other DNA
gctgaacagagaagagctgg

SEQ ID NO: 179 - DNA, 20 bp, synthetic construct
  misc_feature 1..20: note = Synthetic Sequence
  source 1..20: mol_type = other DNA
tctgagggtggagggactgg

SEQ ID NO: 180 - DNA, 20 bp, synthetic construct
  misc_feature 1..20: note = Synthetic Sequence
  source 1..20: mol_type = other DNA
ggagaggtgagggacttggg

SEQ ID NO: 181 - DNA, 20 bp, synthetic construct
  misc_feature 1..20: note = Synthetic Sequence
  source 1..20: mol_type = other DNA
gtgaaccaggcagacaacga

SEQ ID NO: 182 - DNA, 20 bp, synthetic construct
  misc_feature 1..20: note = Synthetic Sequence
  source 1..20: mol_type = other DNA
caggtacctcctgagccacg

SEQ ID NO: 183 - DNA, 20 bp, synthetic construct
  misc_feature 1..20: note = Synthetic Sequence
  source 1..20: mol_type = other DNA
gggggagtaggggcatgcag

SEQ ID NO: 184 - DNA, 20 bp, synthetic construct
  misc_feature 1..20: note = Synthetic Sequence
  source 1..20: mol_type = other DNA
gcaaatggccagcaagggtg

SEQ ID NO: 185 - DNA, 25 bp, synthetic construct
  misc_feature 1..25: note = Synthetic Sequence
  source 1..25: mol_type = other DNA
caccgaatcgagaagcgactcgaca

SEQ ID NO: 186 - DNA, 25 bp, synthetic construct
  misc_feature 1..25: note = Synthetic Sequence
  source 1..25: mol_type = other DNA
caccggtccctgggcgttgccctgc

SEQ ID NO: 187 - DNA, 25 bp, synthetic construct
  misc_feature 1..25: note = Synthetic Sequence
  source 1..25: mol_type = other DNA
caccgccctgggcgttgccctgcag

SEQ ID NO: 188 - DNA, 25 bp, synthetic construct
  misc_feature 1..25: note = Synthetic Sequence
  source 1..25: mol_type = other DNA
caccgccgtgggaagataaactaat

SEQ ID NO: 189 - DNA, 25 bp, synthetic construct
  misc_feature 1..25: note = Synthetic Sequence
  source 1..25: mol_type = other DNA
caccgtcccctgcagggcaacgccc

SEQ ID NO: 190 - DNA, 25 bp, synthetic construct
  misc_feature 1..25: note = Synthetic Sequence
  source 1..25: mol_type = other DNA
caccggtcgagtcgcttctcgatta

SEQ ID NO: 191 - DNA, 25 bp, synthetic construct
  misc_feature 1..25: note = Synthetic Sequence
  source 1..25: mol_type = other DNA
caccgctgctgcctcccgtcttgta

SEQ ID NO: 192 - DNA, 25 bp, synthetic construct
  misc_feature 1..25: note = Synthetic Sequence
  source 1..25: mol_type = other DNA
caccggagtgccgcaatacctttat

SEQ ID NO: 193 - DNA, 25 bp, synthetic construct
  misc_feature 1..25: note = Synthetic Sequence
  source 1..25: mol_type = other DNA
caccgacactttggtggtgcagcaa

SEQ ID NO: 194 - DNA, 25 bp, synthetic construct
  misc_feature 1..25: note = Synthetic Sequence
  source 1..25: mol_type = other DNA
caccgtctcaaatggtataaaactc

SEQ ID NO: 195 - DNA, 25 bp, synthetic construct
  misc_feature 1..25: note = Synthetic Sequence
  source 1..25: mol_type = other DNA
caccgaatcccgcccataatcgaga

SEQ ID NO: 196 - DNA, 25 bp, synthetic construct
  misc_feature 1..25: note = Synthetic Sequence
  source 1..25: mol_type = other DNA
caccgtcccgcccataatcgagaag

SEQ ID NO: 197 - DNA, 25 bp, synthetic construct
  misc_feature 1..25: note = Synthetic Sequence
  source 1..25: mol_type = other DNA
caccgcccataatcgagaagcgact

SEQ ID NO: 198 - DNA, 25 bp, synthetic construct
  misc_feature 1..25: note = Synthetic Sequence
  source 1..25: mol_type = other DNA
caccggagaagcgactcgacatgga

SEQ ID NO: 199 - DNA, 25 bp, synthetic construct
  misc_feature 1..25: note = Synthetic Sequence
  source 1..25: mol_type = other DNA
caccggaagcgactcgacatggagg

SEQ ID NO: 200 - DNA, 25 bp, synthetic construct
  misc_feature 1..25: note = Synthetic Sequence
  source 1..25: mol_type = other DNA
caccggcgactcgacatggaggcga

SEQ ID NO: 201 - DNA, 25 bp, synthetic construct
  misc_feature 1..25: note = Synthetic Sequence
  source 1..25: mol_type = other DNA
aaactgtcgagtcgcttctcgattc

SEQ ID NO: 202 - DNA, 25 bp, synthetic construct
  misc_feature 1..25: note = Synthetic Sequence
  source 1..25: mol_type = other DNA
aaacgcagggcaacgcccagggacc

SEQ ID NO: 203 - DNA, 25 bp, synthetic construct
  misc_feature 1..25: note = Synthetic Sequence
  source 1..25: mol_type = other DNA
aaacctgcagggcaacgcccagggc

SEQ ID NO: 204 - DNA, 25 bp, synthetic construct
  misc_feature 1..25: note = Synthetic Sequence
  source 1..25: mol_type = other DNA
aaacattagtttatcttcccacggc

SEQ ID NO: 205 - DNA, 25 bp, synthetic construct
  misc_feature 1..25: note = Synthetic Sequence
  source 1..25: mol_type = other DNA
aaacgggcgttgccctgcaggggac

SEQ ID NO: 206 - DNA, 25 bp, synthetic construct
  misc_feature 1..25: note = Synthetic Sequence
  source 1..25: mol_type = other DNA
aaactaatcgagaagcgactcgacc

SEQ ID NO: 207 - DNA, 25 bp, synthetic construct
  misc_feature 1..25: note = Synthetic Sequence
  source 1..25: mol_type = other DNA
aaactacaagacgggaggcagcagc

SEQ ID NO: 208 - DNA, 25 bp, synthetic construct
  misc_feature 1..25: note = Synthetic Sequence
  source 1..25: mol_type = other DNA
aaacataaaggtattgcggcactcc

SEQ ID NO: 209 - DNA, 25 bp, synthetic construct
  misc_feature 1..25: note = Synthetic Sequence
  source 1..25: mol_type = other DNA
aaacttgctgcaccaccaaagtgtc

SEQ ID NO: 210 - DNA, 25 bp, synthetic construct
  misc_feature 1..25: note = Synthetic Sequence
  source 1..25: mol_type = other DNA
aaacgagttttataccatttgagac

SEQ ID NO: 211 - DNA, 25 bp, synthetic construct
  misc_feature 1..25: note = Synthetic Sequence
  source 1..25: mol_type = other DNA
aaactctcgattatgggcgggattc

SEQ ID NO: 212 - DNA, 25 bp, synthetic construct
  misc_feature 1..25: note = Synthetic Sequence
  source 1..25: mol_type = other DNA
aaaccttctcgattatgggcgggac

SEQ ID NO: 213 - DNA, 25 bp, synthetic construct
  misc_feature 1..25: note = Synthetic Sequence
  source 1..25: mol_type = other DNA
aaacagtcgcttctcgattatgggc

SEQ ID NO: 214 - DNA, 25 bp, synthetic construct
  misc_feature 1..25: note = Synthetic Sequence
  source 1..25: mol_type = other DNA
aaactccatgtcgagtcgcttctcc

SEQ ID NO: 215 - DNA, 25 bp, synthetic construct
  misc_feature 1..25: note = Synthetic Sequence
  source 1..25: mol_type = other DNA
aaaccctccatgtcgagtcgcttcc

SEQ ID NO: 216 - DNA, 25 bp, synthetic construct
  misc_feature 1..25: note = Synthetic Sequence
  source 1..25: mol_type = other DNA
aaactcgcctccatgtcgagtcgcc

SEQ ID NO: 217 - DNA, 25 bp, synthetic construct
  misc_feature 1..25: note = Synthetic Sequence
  source 1..25: mol_type = other DNA
caccgacagggttaatgtgaagtcc

SEQ ID NO: 218 - DNA, 25 bp, synthetic construct
  misc_feature 1..25: note = Synthetic Sequence
  source 1..25: mol_type = other DNA
caccgtccccctctacatttaaagt

SEQ ID NO: 219 - DNA, 25 bp, synthetic construct
  misc_feature 1..25: note = Synthetic Sequence
  source 1..25: mol_type = other DNA
caccgcatttaaagttggtttaagt

SEQ ID NO: 220 - DNA, 25 bp, synthetic construct
  misc_feature 1..25: note = Synthetic Sequence
  source 1..25: mol_type = other DNA
caccgttagaaaatataaagaataa

SEQ ID NO: 221 - DNA, 25 bp, synthetic construct
  misc_feature 1..25: note = Synthetic Sequence
  source 1..25: mol_type = other DNA
caccgtaaatgcttactggtttgaa

SEQ ID NO: 222 - DNA, 25 bp, synthetic construct
  misc_feature 1..25: note = Synthetic Sequence
  source 1..25: mol_type = other DNA
caccgtcctgggtccagaaaaagat

SEQ ID NO: 223 - DNA, 25 bp, synthetic construct
  misc_feature 1..25: note = Synthetic Sequence
  source 1..25: mol_type = other DNA
caccgttgggtggtgagcatctgtg

SEQ ID NO: 224 - DNA, 25 bp, synthetic construct
  misc_feature 1..25: note = Synthetic Sequence
  source 1..25: mol_type = other DNA
caccgcggggagagtggagaaaaag

SEQ ID NO: 225 - DNA, 25 bp, synthetic construct
  misc_feature 1..25: note = Synthetic Sequence
  source 1..25: mol_type = other DNA
caccggttaaaactctttagacaac

SEQ ID NO: 226 - DNA, 25 bp, synthetic construct
  misc_feature 1..25: note = Synthetic Sequence
  source 1..25: mol_type = other DNA
caccggaaaatccccactaagatcc

SEQ ID NO: 227 - DNA, 25 bp, synthetic construct
  misc_feature 1..25: note = Synthetic Sequence
  source 1..25: mol_type = other DNA
aaacggacttcacattaaccctgtc

SEQ ID NO: 228 - DNA, 25 bp, synthetic construct
  misc_feature 1..25: note = Synthetic Sequence
  source 1..25: mol_type = other DNA
aaacactttaaatgtagagggggac

SEQ ID NO: 229 - DNA, 25 bp, synthetic construct
  misc_feature 1..25: note = Synthetic Sequence
  source 1..25: mol_type = other DNA
aaacacttaaaccaactttaaatgc

SEQ ID NO: 230 - DNA, 25 bp, synthetic construct
  misc_feature 1..25: note = Synthetic Sequence
  source 1..25: mol_type = other DNA
aaacttattctttatattttctaac

SEQ ID NO: 231 - DNA, 25 bp, synthetic construct
  misc_feature 1..25: note = Synthetic Sequence
  source 1..25: mol_type = other DNA
aaacttcaaaccagtaagcatttac

SEQ ID NO: 232 - DNA, 25 bp, synthetic construct
  misc_feature 1..25: note = Synthetic Sequence
  source 1..25: mol_type = other DNA
aaacatctttttctggacccaggac

SEQ ID NO: 233 - DNA, 25 bp, synthetic construct
  misc_feature 1..25: note = Synthetic Sequence
  source 1..25: mol_type = other DNA
aaaccacagatgctcaccacccaac

SEQ ID NO: 234 - DNA, 25 bp, synthetic construct
  misc_feature 1..25: note = Synthetic Sequence
  source 1..25: mol_type = other DNA
aaacctttttctccactctccccgc

SEQ ID NO: 235 - DNA, 25 bp, synthetic construct
  misc_feature 1..25: note = Synthetic Sequence
  source 1..25: mol_type = other DNA
aaacgttgtctaaagagttttaacc

SEQ ID NO: 236 - DNA, 25 bp, synthetic construct
  misc_feature 1..25: note = Synthetic Sequence
  source 1..25: mol_type = other DNA
aaacggatcttagtggggattttcc

SEQ ID NO: 237 - DNA, 20 bp, synthetic construct
  misc_feature 1..20: note = Synthetic Sequence
  source 1..20: mol_type = other DNA
agtagcagtaatgaagctgg

SEQ ID NO: 238 - DNA, 20 bp, synthetic construct
  misc_feature 1..20: note = Synthetic Sequence
  source 1..20: mol_type = other DNA
atacccagacgagaaagctg

SEQ ID NO: 239 - DNA, 20 bp, synthetic construct
  misc_feature 1..20: note = Synthetic Sequence
  source 1..20: mol_type = other DNA
tacccagacgagaaagctga

SEQ ID NO: 240 - DNA, 20 bp, synthetic construct
  misc_feature 1..20: note = Synthetic Sequence
  source 1..20: mol_type = other DNA
ggtggtgagcatctgtgtgg

SEQ ID NO: 241 - DNA, 20 bp, synthetic construct
  misc_feature 1..20: note = Synthetic Sequence
  source 1..20: mol_type = other DNA
aaatgagaagaagaggcaca

SEQ ID NO: 242 - DNA, 20 bp, synthetic construct
  misc_feature 1..20: note = Synthetic Sequence
  source 1..20: mol_type = other DNA
cttgtggcctgggagagctg

SEQ ID NO: 243 - DNA, 19 bp, synthetic construct
  misc_feature 1..19: note = Synthetic Sequence
  source 1..19: mol_type = other DNA
gctgtagaaggagacagag

SEQ ID NO: 244 - DNA, 20 bp, synthetic construct
  misc_feature 1..20: note = Synthetic Sequence
  source 1..20: mol_type = other DNA
gagctggttgggaagacatg

SEQ ID NO: 245 - DNA, 20 bp, synthetic construct
  misc_feature 1..20: note = Synthetic Sequence
  source 1..20: mol_type = other DNA
ctggttgggaagacatgggg

SEQ ID NO: 246 - DNA, 20 bp, synthetic construct
  misc_feature 1..20: note = Synthetic Sequence
  source 1..20: mol_type = other DNA
cgtgaggatgggaaggaggg

SEQ ID NO: 247 - DNA, 20 bp, synthetic construct
  misc_feature 1..20: note = Synthetic Sequence
  source 1..20: mol_type = other DNA
atgcagagtcagcagaactg

SEQ ID NO: 248 - DNA, 20 bp, synthetic construct
  misc_feature 1..20: note = Synthetic Sequence
  source 1..20: mol_type = other DNA
aagacatcaagcacagaagg

SEQ ID NO: 249 - DNA, 20 bp, synthetic construct
  misc_feature 1..20: note = Synthetic Sequence
  source 1..20: mol_type = other DNA
tcaagcacagaaggaggagg

SEQ ID NO: 250 - DNA, 20 bp, synthetic construct
  misc_feature 1..20: note = Synthetic Sequence
  source 1..20: mol_type = other DNA
aaccgtcaataggcaaaggg

SEQ ID NO: 251 - DNA, 20 bp, synthetic construct
  misc_feature 1..20: note = Synthetic Sequence
  source 1..20: mol_type = other DNA
ccgtatttcagactgaatgg

SEQ ID NO: 252 - DNA, 20 bp, synthetic construct
  misc_feature 1..20: note = Synthetic Sequence
  source 1..20: mol_type = other DNA
gagaggacaggtgctacagg

SEQ ID NO: 253 - DNA, 20 bp, synthetic construct
  misc_feature 1..20: note = Synthetic Sequence
  source 1..20: mol_type = other DNA
aaccaaggaagggcaggagg

SEQ ID NO: 254 - DNA, 20 bp, synthetic construct
  misc_feature 1..20: note = Synthetic Sequence
  source 1..20: mol_type = other DNA
gacctctgggtggagacaga

SEQ ID NO: 255 - DNA, 20 bp, synthetic construct
  misc_feature 1..20: note = Synthetic Sequence
  source 1..20: mol_type = other DNA
cagatgaccatgacaagcag

SEQ ID NO: 256 - DNA, 20 bp, synthetic construct
  misc_feature 1..20: note = Synthetic Sequence
  source 1..20: mol_type = other DNA
aacaccagtgagtagagcgg

SEQ ID NO: 257 - DNA, 20 bp, synthetic construct
  misc_feature 1..20: note = Synthetic Sequence
  source 1..20: mol_type = other DNA
aggaccttgaagcacagaga

SEQ ID NO: 258 - DNA, 20 bp, synthetic construct
  misc_feature 1..20: note = Synthetic Sequence
  source 1..20: mol_type = other DNA
tacagaggcagactaaccca

SEQ ID NO: 259 - DNA, 20 bp, synthetic construct
  misc_feature 1..20: note = Synthetic Sequence
  source 1..20: mol_type = other DNA
acagaggcagactaacccag

SEQ ID NO: 260 - DNA, 20 bp, synthetic construct
  misc_feature 1..20: note = Synthetic Sequence
  source 1..20: mol_type = other DNA
taaatgacgtgctagacctg

SEQ ID NO: 261 - DNA, 20 bp, synthetic construct
  misc_feature 1..20: note = Synthetic Sequence
  source 1..20: mol_type = other DNA
agtaaccactcaggacaggg

SEQ ID NO: 262 - DNA, 20 bp, synthetic construct
  misc_feature 1..20: note = Synthetic Sequence
  source 1..20: mol_type = other DNA
accacaaaacagaaacacca

SEQ ID NO: 263 - DNA, 20 bp, synthetic construct
  misc_feature 1..20: note = Synthetic Sequence
  source 1..20: mol_type = other DNA
gtttgaagacaagcctgagg

SEQ ID NO: 264 - DNA, 20 bp, synthetic construct
  misc_feature 1..20: note = Synthetic Sequence
  source 1..20: mol_type = other DNA
gctgaaccccaaaagacagg

SEQ ID NO: 265 - DNA, 20 bp, synthetic construct
  misc_feature 1..20: note = Synthetic Sequence
  source 1..20: mol_type = other DNA
gcagctgagacacacaccag

SEQ ID NO: 266 - DNA, 20 bp, synthetic construct
  misc_feature 1..20: note = Synthetic Sequence
  source 1..20: mol_type = other DNA
aggacaccccaaagaagctg

SEQ ID NO: 267 - DNA, 20 bp, synthetic construct
  misc_feature 1..20: note = Synthetic Sequence
  source 1..20: mol_type = other DNA
ggacaccccaaagaagctga

SEQ ID NO: 268 - DNA, 20 bp, synthetic construct
  misc_feature 1..20: note = Synthetic Sequence
  source 1..20: mol_type = other DNA
ccagtgcaatggacagaaga

SEQ ID NO: 269 - DNA, 20 bp, synthetic construct
  misc_feature 1..20: note = Synthetic Sequence
  source 1..20: mol_type = other DNA
agaagagggagcctgcaagt

SEQ ID NO: 270 - DNA, 20 bp, synthetic construct
  misc_feature 1..20: note = Synthetic Sequence
  source 1..20: mol_type = other DNA
gtgtttgggccctagagcga

SEQ ID NO: 271 - DNA, 20 bp, synthetic construct
  misc_feature 1..20: note = Synthetic Sequence
  source 1..20: mol_type = other DNA
catgtgcctggtgcaatgca

SEQ ID NO: 272 - DNA, 20 bp, synthetic construct
  misc_feature 1..20: note = Synthetic Sequence
  source 1..20: mol_type = other DNA
tacaaagaggaagataagtg

SEQ ID NO: 273 - DNA, 20 bp, synthetic construct
  misc_feature 1..20: note = Synthetic Sequence
  source 1..20: mol_type = other DNA
gtcacagaatacaccactag

SEQ ID NO: 274 - DNA, 20 bp, synthetic construct
  misc_feature 1..20: note = Synthetic Sequence
  source 1..20: mol_type = other DNA
gggttaccctggacatggaa

SEQ ID NO: 275 - DNA, 20 bp, synthetic construct
  misc_feature 1..20: note = Synthetic Sequence
  source 1..20: mol_type = other DNA
catggaagggtattcactcg

SEQ ID NO: 276 - DNA, 20 bp, synthetic construct
  misc_feature 1..20: note = Synthetic Sequence
  source 1..20: mol_type = other DNA
agagtggcctagacaggctg

SEQ ID NO: 277 - DNA, 20 bp, synthetic construct
  misc_feature 1..20: note = Synthetic Sequence
  source 1..20: mol_type = other DNA
catgctggacagctcggcag

SEQ ID NO: 278 - DNA, 20 bp, synthetic construct
  misc_feature 1..20: note = Synthetic Sequence
  source 1..20: mol_type = other DNA
agtgaaagaagagaaaattc

SEQ ID NO: 279 - DNA, 20 bp, synthetic construct
  misc_feature 1..20: note = Synthetic Sequence
  source 1..20: mol_type = other DNA
tggtaagtctaagaaaccta

SEQ ID NO: 280 - DNA, 20 bp, synthetic construct
  misc_feature 1..20: note = Synthetic Sequence
  source 1..20: mol_type = other DNA
cccacagcctaaccacccta

SEQ ID NO: 281 - DNA, 20 bp, synthetic construct
  misc_feature 1..20: note = Synthetic Sequence
  source 1..20: mol_type = other DNA
aatatttcaaagccctaggg

SEQ ID NO: 282 - DNA, 20 bp, synthetic construct
  misc_feature 1..20: note = Synthetic Sequence
  source 1..20: mol_type = other DNA
gcactcggaacagggtctgg

SEQ ID NO: 283 - DNA, 20 bp, synthetic construct
  misc_feature 1..20: note = Synthetic Sequence
  source 1..20: mol_type = other DNA
agataggagctccaacagtg

SEQ ID NO: 284 - DNA, 20 bp, synthetic construct
  misc_feature 1..20: note = Synthetic Sequence
  source 1..20: mol_type = other DNA
aagttagagcagccaggaaa

SEQ ID NO: 285 - DNA, 20 bp, synthetic construct
  misc_feature 1..20: note = Synthetic Sequence
  source 1..20: mol_type = other DNA
tagagcagccaggaaaggga

SEQ ID NO: 286 - DNA, 20 bp, synthetic construct
  misc_feature 1..20: note = Synthetic Sequence
  source 1..20: mol_type = other DNA
tgaatacccttccatgtcca

SEQ ID NO: 287 - DNA, 20 bp, synthetic construct
  misc_feature 1..20: note = Synthetic Sequence
  source 1..20: mol_type = other DNA
cctgcattgcaccaggcaca

SEQ ID NO: 288 - DNA, 20 bp, synthetic construct
  misc_feature 1..20: note = Synthetic Sequence
  source 1..20: mol_type = other DNA
tctagggcccaaacacacct

SEQ ID NO: 289 - DNA, 20 bp, synthetic construct
  misc_feature 1..20: note = Synthetic Sequence
  source 1..20: mol_type = other DNA
tccctccatctatcaaaagg

SEQ ID NO: 290 - DNA, 20 bp, synthetic construct
  misc_feature 1..20: note = Synthetic Sequence
  source 1..20: mol_type = other DNA
agccctgagacagaagcagg

SEQ ID NO: 291 - DNA, 20 bp, synthetic construct
  misc_feature 1..20: note = Synthetic Sequence
  source 1..20: mol_type = other DNA
gccctgagacagaagcaggt

SEQ ID NO: 292 - DNA, 20 bp, synthetic construct
  misc_feature 1..20: note = Synthetic Sequence
  source 1..20: mol_type = other DNA
aggagatgcagtgatacgca

SEQ ID NO: 293 - DNA, 20 bp, synthetic construct
  misc_feature 1..20: note = Synthetic Sequence
  source 1..20: mol_type = other DNA
acaataccaagggtatccgg

SEQ ID NO: 294 - DNA, 20 bp, synthetic construct
  misc_feature 1..20: note = Synthetic Sequence
  source 1..20: mol_type = other DNA
tgataaagaaaacaaagtga

SEQ ID NO: 295 - DNA, 20 bp, synthetic construct
  misc_feature 1..20: note = Synthetic Sequence
  source 1..20: mol_type = other DNA
aaagaaaacaaagtgaggga

SEQ ID NO: 296 - DNA, 20 bp, synthetic construct
  misc_feature 1..20: note = Synthetic Sequence
  source 1..20: mol_type = other DNA
gtggcaagtggagaaattga

SEQ ID NO: 297 - DNA, 20 bp, synthetic construct
  misc_feature 1..20: note = Synthetic Sequence
  source 1..20: mol_type = other DNA
caagtggagaaattgaggga

SEQ ID NO: 298 - DNA, 20 bp, synthetic construct
  misc_feature 1..20: note = Synthetic Sequence
  source 1..20: mol_type = other DNA
gtggtgatgattgcagctgg

SEQ ID NO: 299 - DNA, 20 bp, synthetic construct
  misc_feature 1..20: note = Synthetic Sequence
  source 1..20: mol_type = other DNA
ctatgtgcctgacacacagg

SEQ ID NO: 300 - DNA, 20 bp, synthetic construct
  misc_feature 1..20: note = Synthetic Sequence
  source 1..20: mol_type = other DNA
gggttggaccaggaaagagg

SEQ ID NO: 301 - DNA, 20 bp, synthetic construct
  misc_feature 1..20: note = Synthetic Sequence
  source 1..20: mol_type = other DNA
gatgcctggaaaaggaaaga

SEQ ID NO: 302 - DNA, 20 bp, synthetic construct
  misc_feature 1..20: note = Synthetic Sequence
  source 1..20: mol_type = other DNA
tagtatgcacctgcaagagg

SEQ ID NO: 303 - DNA, 20 bp, synthetic construct
  misc_feature 1..20: note = Synthetic Sequence
  source 1..20: mol_type = other DNA
tatgcacctgcaagaggcgg

SEQ ID NO: 304 - DNA, 20 bp, synthetic construct
  misc_feature 1..20: note = Synthetic Sequence
  source 1..20: mol_type = other DNA
aggggaagaagagaagcaga

SEQ ID NO: 305 - DNA, 20 bp, synthetic construct
  misc_feature 1..20: note = Synthetic Sequence
  source 1..20: mol_type = other DNA
gctgaatcaagagacaagcg

SEQ ID NO: 306 - DNA, 20 bp, synthetic construct
  misc_feature 1..20: note = Synthetic Sequence
  source 1..20: mol_type = other DNA
aagcaaataaatctcctggg

SEQ ID NO: 307 - DNA, 20 bp, synthetic construct
  misc_feature 1..20: note = Synthetic Sequence
  source 1..20: mol_type = other DNA
agatgagtgctagagactgg

SEQ ID NO: 308 - DNA, 20 bp, synthetic construct
  misc_feature 1..20: note = Synthetic Sequence
  source 1..20: mol_type = other DNA
ctgatggttgagcacagcag

SEQ ID NO: 309 - DNA, 20 bp, synthetic construct
  misc_feature 1..20: note = Synthetic Sequence
  source 1..20: mol_type = other DNA
caaatggccagcaagggtgg

SEQ ID NO: 310 - DNA, 20 bp, synthetic construct
  misc_feature 1..20: note = Synthetic Sequence
  source 1..20: mol_type = other DNA
gcagaacctgaggatatgga

SEQ ID NO: 311 - DNA, 20 bp, synthetic construct
  misc_feature 1..20: note = Synthetic Sequence
  source 1..20: mol_type = other DNA
aatacacagaatgaaaatag

SEQ ID NO: 312 - DNA, 20 bp, synthetic construct
  misc_feature 1..20: note = Synthetic Sequence
  source 1..20: mol_type = other DNA
ctggtgactagaataggcag

SEQ ID NO: 313 - DNA, 20 bp, synthetic construct
  misc_feature 1..20: note = Synthetic Sequence
  source 1..20: mol_type = other DNA
tggtgactagaataggcagt

SEQ ID NO: 314 - DNA, 20 bp, synthetic construct
  misc_feature 1..20: note = Synthetic Sequence
  source 1..20: mol_type = other DNA
taaaagaatgtgaaaagatg

SEQ ID NO: 315 - DNA, 20 bp, synthetic construct
  misc_feature 1..20: note = Synthetic Sequence
  source 1..20: mol_type = other DNA
tcaggagttcaagaccaccc

SEQ ID NO: 316 - DNA, 20 bp, synthetic construct
  misc_feature 1..20: note = Synthetic Sequence
  source 1..20: mol_type = other DNA
tgtagtcccagttatgcagg

SEQ ID NO: 317 - DNA, 20 bp, synthetic construct
  misc_feature 1..20: note = Synthetic Sequence
  source 1..20: mol_type = other DNA
gggttcacaccacaaatgca

SEQ ID NO: 318 - DNA, 20 bp, synthetic construct
  misc_feature 1..20: note = Synthetic Sequence
  source 1..20: mol_type = other DNA
ggcaaatggccagcaagggt

SEQ ID NO: 319 - DNA, 20 bp, synthetic construct
  misc_feature 1..20: note = Synthetic Sequence
  source 1..20: mol_type = other DNA
agaaaccaatcccaaagcaa

SEQ ID NO: 320 - DNA, 20 bp, synthetic construct
  misc_feature 1..20: note = Synthetic Sequence
  source 1..20: mol_type = other DNA
gccaaggacaccaaaaccca

SEQ ID NO: 321 - DNA, 20 bp, synthetic construct
  misc_feature 1..20: note = Synthetic Sequence
  source 1..20: mol_type = other DNA
agtggtgataaggcaacagt

SEQ ID NO: 322 - DNA, 20 bp, synthetic construct
  misc_feature 1..20: note = Synthetic Sequence
  source 1..20: mol_type = other DNA
cctgagacagaagtattaag

SEQ ID NO: 323 - DNA, 20 bp, synthetic construct
  misc_feature 1..20: note = Synthetic Sequence
  source 1..20: mol_type = other DNA
aaggtcacacaatgaatagg

SEQ ID NO: 324 - DNA, 20 bp, synthetic construct
  misc_feature 1..20: note = Synthetic Sequence
  source 1..20: mol_type = other DNA
caccatactagggaagaaga

SEQ ID NO: 325 - DNA, 20 bp, synthetic construct
  misc_feature 1..20: note = Synthetic Sequence
  source 1..20: mol_type = other DNA
aataccctgcccttagtggg

SEQ ID NO: 326 - DNA, 20 bp, synthetic construct
  misc_feature 1..20: note = Synthetic Sequence
  source 1..20: mol_type = other DNA
ttagtggggggtggagtggg

SEQ ID NO: 327 - DNA, 20 bp, synthetic construct
  misc_feature 1..20: note = Synthetic Sequence
  source 1..20: mol_type = other DNA
caataccctgcccttagtgg

SEQ ID NO: 328 - DNA, 20 bp, synthetic construct
  misc_feature 1..20: note = Synthetic Sequence
  source 1..20: mol_type = other DNA
gtggggggtggagtgggggg

SEQ ID NO: 329 - DNA, 20 bp, synthetic construct
  misc_feature 1..20: note = Synthetic Sequence
  source 1..20: mol_type = other DNA
ggggggtggagtggggggtg

SEQ ID NO: 330 - DNA, 20 bp, synthetic construct
  misc_feature 1..20: note = Synthetic Sequence
  source 1..20: mol_type = other DNA
ggggtggagtggggggtggg

SEQ ID NO: 331 - DNA, 20 bp, synthetic construct
  misc_feature 1..20: note = Synthetic Sequence
  source 1..20: mol_type = other DNA
gggtggagtggggggtgggg

SEQ ID NO: 332 - DNA, 20 bp, synthetic construct
  misc_feature 1..20: note = Synthetic Sequence
  source 1..20: mol_type = other DNA
gggggtggggaaagacatcg

SEQ ID NO: 333 - DNA, 20 bp, synthetic construct
  misc_feature 1..20: note = Synthetic Sequence
  source 1..20: mol_type = other DNA
gcagctgtgaattctgatag

SEQ ID NO: 334 - DNA, 20 bp, synthetic construct
  misc_feature 1..20: note = Synthetic Sequence
  source 1..20: mol_type = other DNA
gagatcagagaaaccagatg

SEQ ID NO: 335 - DNA, 20 bp, synthetic construct
  misc_feature 1..20: note = Synthetic Sequence
  source 1..20: mol_type = other DNA
tctatactgattgcagccag

SEQ ID NO: 336-347 - skipped (12 entries)

SEQ ID NO: 348 - protein, 18 aa, synthetic construct
  REGION 1..18: note = Synthetic Sequence
  REGION 1..6: note = MISC_FEATURE - Xaa can be any naturally occurring amino acid; SEQ ID NO: 348corresponds to the sequence K(K/R)X(K/R)
  SITE 10: note = misc_feature - Xaa can be any naturally occurring amino acid
LYSLYSARGXAALYSARG

SEQ ID NO: 349 - protein, 21 aa, synthetic construct
  REGION 1..21: note = Synthetic Sequence
  REGION 1..7: note = MISC_FEATURE - Xaa can be any naturally occurring amino acid; SEQ ID NO: 349corresponds to the sequence (K/R)(K/R)X10-12(K/R)3/5
  SITE 13: note = misc_feature - Xaa can be any naturally occurring amino acid
LYSARGLYSARGXAALYSARG

SEQ ID NO: 350 - protein, 9 aa, synthetic construct
  REGION 1..9: note = Synthetic Sequence
PAAKRVKLD

SEQ ID NO: 351 - protein, 16 aa, synthetic construct
  REGION 1..16: note = Synthetic Sequence
KRPAATKKAGQAKKKK

SEQ ID NO: 352 - protein, 7 aa, synthetic construct
  REGION 1..7: note = Synthetic Sequence
PKKKRKV

SEQ ID NO: 353 - protein, 24 aa, synthetic construct
  REGION 1..24: note = Synthetic Sequence
DPKKKRKVDPKKKRKVDPKKKRKV

SEQ ID NO: 354-422 - skipped (69 entries)

SEQ ID NO: 423 - DNA, 333 bp, synthetic construct
  misc_feature 1..333: note = Synthetic Sequence
  source 1..333: mol_type = other DNA
ggcggatctggcggtagtgctgagtattgtctgagttacgaaacggaaatactcacggttgagtatgggcttcttccaattggcaaaatcgttgaaaagcgcatagagtgtacggtgtattccgtcgataacaacggtaatatctacacccagccggtagctcagtggcacgaccgaggcgaacaggaagtgttcgagtattgcttggaagatggctcccttatccgcgccactaaagaccataagtttatgacggttgacgggcagatgctgcctatagacgaaatatttgagagagagctggacttgatgagagtcgataatctgccaaat

SEQ ID NO: 424 - DNA, 180 bp, synthetic construct
  misc_feature 1..180: note = Synthetic Sequence
  source 1..180: mol_type = other DNA
ggcggatctggcggtagtgggggttccggatccataaagatagctactaggaaatatcttggcaaacaaaacgtctatgacataggagttgagcgagatcacaattttgctttgaagaatgggttcatcgcgtctaattgcttcaacgctagcggcgggtcaggaggctctggtggaagc

SEQ ID NO: 425 - DNA, 20 bp, synthetic construct
  misc_feature 1..20: note = Synthetic Sequence
  source 1..20: mol_type = other DNA
aatcgagaagcgactcgaca

SEQ ID NO: 426 - DNA, 20 bp, synthetic construct
  misc_feature 1..20: note = Synthetic Sequence
  source 1..20: mol_type = other DNA
tgccctgcaggggagtgagc

SEQ ID NO: 427 - DNA, 20 bp, synthetic construct
  misc_feature 1..20: note = Synthetic Sequence
  source 1..20: mol_type = other DNA
gaagcgactcgacatggagg

SEQ ID NO: 428 - DNA, 20 bp, synthetic construct
  misc_feature 1..20: note = Synthetic Sequence
  source 1..20: mol_type = other DNA
cctgcaggggagtgagcagc

SEQ ID NO: 429 - DNA, 2210 bp, synthetic construct
  misc_feature 1..2210: note = Synthetic Sequence
  misc_feature 187: note = n is a, c, g, or t
  misc_feature 210: note = n is a, c, g, or t
  misc_feature 1088: note = n is a, c, g, or t
  misc_feature 2190: note = n is a, c, g, or t
  source 1..2210: mol_type = other DNA
cccatttcctgtttgccccgagaatactcaccagcggcacttgcagctgcagcgtttaccccgagataactcgycgattacagtcctaaccttacccccaaagtttgccatgaaatatctcgcttttattattattttcgcatcgctctagtatatcgatagtctttggaaacaaatgacatcattntatttacagcattctgtttttantagtggtatttccatttacaaaatatagtaattttctatcgctgaaaatgtcaaatcctagaaaacgtagcattcctacatgtgatgttaacttcgttctcgaacagttgttagccgaagattcatttgatgaatccgatttttccgaaatagacgattctgatgatttttcggatagtgcttcggaagactatacggtcaggcctccgtccgattcggaatctgatggaaatagccctacatcagctgactcgggtcgcgctctgaaatggtcaactcgtgttatgattccacgtcaaaggtatgactttaccggcacacctggcagaaaagttgatgtcagtgataccactgacccactgcagtattttgaactgttctttactgaggaattagtttcaaaaattaccagtgaaatgaatgcccaagctgccttgttggcttcaaagccacctggtccgaaaggattttcgcgaatggataaatggaaagacactgacaatgatgaactgaaagtcttttttgcagtaatgttactgcaaggtattgtgcagaaacctgagctggagatgttttggtcgacaaggcctcttttggatataccttatctcaggcaaattatgactggtgaaagatttttacttttgcttcggtgcctgcattttgtcaacaattcttccatatccgctggtcaatcaaaggcccagatttcattgcagaagatcaaacctgtgttcgactttcttgtaaataagttttcaactgtatatactccaaacagaaacattgcagtcgatgaatcactgatgctgttcaaggggcggttagctatgaagcagtacatcccgacgaaatgtgcacgatttggtctcaagctntatgtactttgtgaaagtcaatctggttacgtgtggaatgcgcttgttcacacagggcccagtatgaatttgaaagattcagctgatggtctgaaatcgtcatgcattgttcttaccttggtcaatgaccttcttggccaaggatattgtgtcttcctcaataacttttatacatctcccatgcttttcagagaattacatcaaaacaggactgatgcagttgggacagctcgtttgaacagaaaacagatgccaaatgatctgaaaaaaaggattgcaaaggggacgactgtagccagattctgtggtgaacttatggcactgaaatggtgtgacaagaaggaggtgacaatgttgtcaacattccacaatgatactgtgattgaagtagacaacagaaatggaaagaaaactaagaagccatgtgtcattgtggattataacgagaatatgggagcagtggactcggctgatcagatgctcacttcttatccaactgagcgcaaaaggcacaagttttggtataagaaattctttcgccaccttctaaacattacagtgctgaactcctacatcctgttcaagaaggacaatcctgagcacacgatcagccatgtaaacttcagactgacgttgattgaaagaatgctggaaaagcatcacaagccagggcagcaacgtcttcgaggtcgtccgtgctctgatgatgtcacacctcttcgcctgtctggaagacatttccccaagagcataccaccaacatcagggaaacagaatccaactggtcgctgcaaagtttgctgctcgcacgacaaggatggcaagaagatccggagagaaacgttatatttttgtgcggaatgtgatgttccgctttgtgttgttccgtgctttgaaatttaccacacgaaaaaaaattattaaatactgatcatcatatacatttctgttacattaggattagagacaagttctgtttagaaataactccaagaacagtttttatattttattttcacattgaaaaccagtcagatttgcttcagcctcaaagagcatgtttatgtaaaattaaattaacgctggcagcgagctgcacttnttttctaaacgggaaatggg

SEQ ID NO: 430 - protein, 584 aa, synthetic construct
  REGION 1..584: note = Synthetic Sequence
MSNPRKRSIPTCDVNFVLEQLLAEDSFDESDFSEIDDSDDFSDSASEDYTVRPPSDSESDGNSPTSADSGRALKWSTRVMIPRQRYDFTGTPGRKVDVSDTTDPLQYFELFFTEELVSKITSEMNAQAALLASKPPGPKGFSRMDKWKDTDNDELKVFFAVMLLQGIVQKPELEMFWSTRPLLDIPYLRQIMTGERFLLLLRCLHFVNNSSISAGQSKAQISLQKIKPVFDFLVNKFSTVYTPNRNIAVDESLMLFKGRLAMKQYIPTKCARFGLKLYVLCESQSGYVWNALVHTGPSMNLKDSADGLKSSCIVLTLVNDLLGQGYCVFLNNFYTSPMLFRELHQNRTDAVGTARLNRKQMPNDLKKRIAKGTTVARFCGELMALKWCDKKEVTMLSTFHNDTVIEVDNRNGKKTKKPCVIVDYNENMGAVDSADQMLTSYPTERKRHKFWYKKFFRHLLNITVLNSYILFKKDNPEHTISHVNFRLTLIERMLEKHHKPGQQRLRGRPCSDDVTPLRLSGRHFPKSIPPTSGKQNPTGRCKVCCSHDKDGKKIRRETLYFCAECDVPLCVVPCFEIYHTKKNY

SEQ ID NO: 431 - DNA, 157 bp, synthetic construct; identical to SEQ ID NO: 21
  misc_feature 1..157: note = Synthetic Sequence
  source 1..157: mol_type = other DNA

SEQ ID NO: 432 - DNA, 212 bp, synthetic construct
  misc_feature 1..212: note = Synthetic Sequence
  source 1..212: mol_type = other DNA
aattatttatgtactgaatagataaaaaaatgtctgtgattgaataaattttcattttttacacaagaaaccgaaaatttcatttcaatcgaacccatacttcaaaagatataggcattttaaactaactctgattttgcgcgggaaacctaaataattgcccgcgccatcttatattttggcgggaaattcacccgacaccgtggtgttaa

SEQ ID NO: 433 - protein, 594 aa, synthetic construct
  REGION 1..594: note = Synthetic Sequence
MGSSLDDEHILSALLQSDDELVGEDSDSEISDHVSEDDVQSDTEEAFIDEVHEVQPTSSGSEILDEQNVIEQPGSSLASNKILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVRGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVTKKRTYCTYCPSKIRRKANASCKKCKKVICREHNIDMCQSCF

SEQ ID NO: 434 - protein, 280 aa, synthetic construct
  REGION 1..280: note = Synthetic Sequence
MSNPRKRSIPTCDVNFVLEQLLAEDSFDESDFSEIDDSDDFSDSASEDYTVRPPSDSESDGNSPTSADSGRALKWSTRVMIPRQRYDFTGTPGRKVDVSDTTDPLQYFELFFTEELVSKITSEMNAQAALLASKPPGPKGFSRMDKWKDTDNDELKVFFAVMLLQGIVQKPELEMFWSTRPLLDIPYLRQIMTGERFLLLLRCLHFVNNSSISAGQSKAQISLQKIKPVFDFLVNKFSTVYTPNRNIAVDESLMLFKGRLAMKQYIPTKMNLKDSADGLK

SEQ ID NO: 435 - protein, 252 aa, synthetic construct
  REGION 1..252: note = Synthetic Sequence
MDLRCQHTVLSIRESRGLLPNLKMKTSRMKKGDIIFSRKGDILLLAWKDKRVVRMISIHDTSVSTTGKKNRKTGENIVKPACIKEYNAHMKGVDRADQFLSCCSILRKMMKWTKKVVLYLINCGLFNSFRVYNVLNPQAKMKYKQFLLSVARDWIMDDNNEGSPEPETNLSSPSPGGARRAPRKDPPKRLSGDMKQHEPTCIPASGKKKFPTRACRVCAHGKRSESRYLCKFCLVPLHRGKCFTQYHTLKKY

SEQ ID NO: 436 - protein, 432 aa, synthetic construct
  REGION 1..432: note = Synthetic Sequence
MKAFLGVILNMGVLNHPNLQSYWSMDFESHIPFFRSVFKRERFLQIFWMLHLKNDQKSSKDLRTRTEKVNCFLSYLEMKFRERFCPGREIAVDEAVVGFKGKIHFITYNPKKPTKWGIRLYVLSDSKCGYVHSFVPYYGGITSETLVRPDLPFTSRIVLELHERLKNSVPGSQGYHFFTDRYYTSVTLAKELFKEKTHLTGTIMPNRKDNPPVIKHQKLKKGEIVAFRDENVMLLAWKDKRIVTLSTWDSETESVERRVGGGKEIVLKPKVVTNYTKFMGGVDIADYTSTYCFMRKTLKWWRTLFFWGLEVSVVNSYILYKECQKRKNEKPITHVKFIRKLVHDLVGEFRDGTLTSRGRLLSTNLEQRLDGKLHIITPHPNKKHKDCVVCSNRKIKGGRRETIYICETCECKPGLHVGECFKKYHTMKNYRD

SEQ ID NO: 437 - protein, 662 aa, synthetic construct
  REGION 1..662: note = Synthetic Sequence
MPSLRKRKETNETDTLPEVFNDNLSDIPSEIEDADDCFDDSGDDSTDSTDSEIIRPVRKRKVAVLSSDSDTDEATDNCWSEIDTPPRLQMFEGHAGVTTFPSQCDSVPSVTNLFFGDELFEMLCKELSNYHDQTAMKRKTPSRTLKWSPVTQKDIKKFLGLIILMGQTRKDSLKDYWSTDPLICTPIFPQTMSRHRFEQIWTFWHFNDNAKMDSRSGRLFKIQPVLDYFLHKFRTIYKPKQQLSLDEGMIPWRGRFKFRTYNPAKITKYGLLVRMVCESDTGYICSMEIYTAEGRKLQETVLSVLGPYLGIWHHIYQDNYYNATSTAELLLQNKTRVCGTIRESRGLPPNLEMKTSRMKKGDIIFSRKGDILLLAWKDKRVVRMISTIHDTSVSTTGKKNRKTGENIVKPTCIKEYNAHMKGVDRADQFLSCCSILRKTMKWTKKVVLYLINCGLFNSFRVYNVLNPQAKMKYKQFLLSVARDWITDDNNEGSPEPETNLSSPSPGGARRAPRKDPPKRLSGDMKQHEPTCIPASGKKKFPTRACRVCAAHGKRSESRYLCKFCLVPLHRGKCFTQYHTLKKYMDLRCQHTVLSTVGRGYSVLARFKPRTNERTGSSHCHVQVPAGGQGPPSTIIANGCGCKLEPMVRTRSPTCLVIEFGCM

SEQ ID NO: 438 - protein, 673 aa, synthetic construct
  REGION 1..673: note = Synthetic Sequence
MPSLRKRKETNETDTLPEVFNDNLSDIPSEIEDADDCFDDSGDDSTDSTESEIIRPVRKRKVAVLSSDSNTDEATDNCWSEIDTPPRLQMFEGHAGVTTFPSQCDSVPSVTNLFFGDELFEMLCKELSNYHDQTAMKRKTPSRTLKWSPVTQKDIKKFLGLIILMGQTRKDSWKDYWSTDPLICTPIFPQTMSRHRFEQIWTFWHFNDNAKMDSCSGRLFKIQPVLDYFLHKFRTIYKPKQQLSLDEGMIPWRGRLKFTYNPAITKYGLLVRMVCESDTGYICNMEIYTAERKKLQETVLSVLGPYLGIWHHIYQDNYYNATSTAELLLQNKTRVCGTIRESRGLPPNLKMKTSRMKKGDIIFSRKGDILLLAWKDKRVVRMISTIHDTSVSTTGKKNRKTGENIVKPTCIKEYNAHMKGVDRADQFLSCCSILRKTTKWTKKVVLYLINCGLFNSFRVYNILNPQAKMKYKQFLLSVARDWITDDNNEGSPEPETNLSSPSSGGARRAPRKDQPKRLSGDMKQHEPTCIPASGKKKFPTACRVCAAHGKRSESRYLRKFCFVPLRGKCFMYHTLKKYSELFSLIVVSKIQNVIIYKTTKVYMRYVMRSHCPLSFLVFAPSVKDRSRVFSFFTRHLLWTLDVNTLSCPHRMKRSHWWKPCRSIYEKLYNCTNP

SEQ ID NO: 439 - protein, 254 aa, synthetic construct
  REGION 1..254: note = Synthetic Sequence
MDLRCQHTVLSIRESRGLPPNLKMKTSRMKKGDIIFSRKGDILLLAWKDKRVVRMISTIHDTSVSTTGKKNRKTGENIVKPACIKEYNAHMKGVDRADQFLSCCSILRKTMKWTKKVVLYLINCGLFNSFRVYNVLNPQAKMKYKQFLLSVARDWITDDNNEGSPEPETNLSSPSPGGARRAPRKDPPKRLSGDMKQHEPTCIPASGKKKFPTRACRVCAAHGKRSESRYLCKFCLVPLHRGKCFTQYHTLKKY

SEQ ID NO: 440 - skipped

SEQ ID NO: 441 - DNA, 46 bp, synthetic construct
  misc_feature 1..46: note = Synthetic Sequence
  source 1..46: mol_type = other DNA
cgctggcagcgagctgcactttttttctaaacgggaaatgggttaa

SEQ ID NO: 442 - DNA, 20 bp, synthetic construct
  misc_feature 1..20: note = Synthetic Sequence
  source 1..20: mol_type = other DNA
gtccctgggcgttgccctgc

SEQ ID NO: 443 - DNA, 20 bp, synthetic construct
  misc_feature 1..20: note = Synthetic Sequence
  source 1..20: mol_type = other DNA
ccctgggcgttgccctgcag

SEQ ID NO: 444 - DNA, 20 bp, synthetic construct
  misc_feature 1..20: note = Synthetic Sequence
  source 1..20: mol_type = other DNA
gagtgagcagctgtaagatt

SEQ ID NO: 445 - DNA, 20 bp, synthetic construct
  misc_feature 1..20: note = Synthetic Sequence
  source 1..20: mol_type = other DNA
caggggagtgagcagctgta

SEQ ID NO: 446 - DNA, 20 bp, synthetic construct
  misc_feature 1..20: note = Synthetic Sequence
  source 1..20: mol_type = other DNA
cgttgccctgcaggggagtg

SEQ ID NO: 447 - DNA, 20 bp, synthetic construct
  misc_feature 1..20: note = Synthetic Sequence
  source 1..20: mol_type = other DNA
tgggcgttgccctgcagggg

SEQ ID NO: 448 - DNA, 20 bp, synthetic construct
  misc_feature 1..20: note = Synthetic Sequence
  source 1..20: mol_type = other DNA
ttggtccctgggcgttgccc

SEQ ID NO: 449 - DNA, 20 bp, synthetic construct
  misc_feature 1..20: note = Synthetic Sequence
  source 1..20: mol_type = other DNA
aagaatcccgcccataatcg

SEQ ID NO: 450 - DNA, 20 bp, synthetic construct
  misc_feature 1..20: note = Synthetic Sequence
  source 1..20: mol_type = other DNA
aatcccgcccataatcgaga

SEQ ID NO: 451 - DNA, 20 bp, synthetic construct
  misc_feature 1..20: note = Synthetic Sequence
  source 1..20: mol_type = other DNA
tcccgcccataatcgagaag

SEQ ID NO: 452 - DNA, 20 bp, synthetic construct
  misc_feature 1..20: note = Synthetic Sequence
  source 1..20: mol_type = other DNA
cccataatcgagaagcgact

SEQ ID NO: 453 - DNA, 20 bp, synthetic construct
  misc_feature 1..20: note = Synthetic Sequence
  source 1..20: mol_type = other DNA
gagaagcgactcgacatgga

SEQ ID NO: 454 - DNA, 20 bp, synthetic construct
  misc_feature 1..20: note = Synthetic Sequence
  source 1..20: mol_type = other DNA
gcgactcgacatggaggcga

SEQ ID NO: 455 - DNA, 20 bp, synthetic construct
  misc_feature 1..20: note = Synthetic Sequence
  source 1..20: mol_type = other DNA
ccgtgggaagataaactaat

SEQ ID NO: 456 - DNA, 20 bp, synthetic construct
  misc_feature 1..20: note = Synthetic Sequence
  source 1..20: mol_type = other DNA
tcccctgcagggcaacgccc

SEQ ID NO: 457 - DNA, 20 bp, synthetic construct
  misc_feature 1..20: note = Synthetic Sequence
  source 1..20: mol_type = other DNA
gtcgagtcgcttctcgatta

SEQ ID NO: 458 - DNA, 20 bp, synthetic construct
  misc_feature 1..20: note = Synthetic Sequence
  source 1..20: mol_type = other DNA
cgacaccaactctagtccgt

SEQ ID NO: 459 - DNA, 20 bp, synthetic construct
  misc_feature 1..20: note = Synthetic Sequence
  source 1..20: mol_type = other DNA
cagctgctcactcccctgca

SEQ ID NO: 460 - DNA, 20 bp, synthetic construct
  misc_feature 1..20: note = Synthetic Sequence
  source 1..20: mol_type = other DNA
agtcgcttctcgattatggg

SEQ ID NO: 461 - DNA, 23 bp, synthetic construct
  misc_feature 1..23: note = Synthetic Sequence
  source 1..23: mol_type = other DNA
acccttggaaggacctggctggg

SEQ ID NO: 462 - DNA, 20 bp, synthetic construct
  misc_feature 1..20: note = Synthetic Sequence
  source 1..20: mol_type = other DNA
tccgagcttgacccttggaa

SEQ ID NO: 463 - DNA, 23 bp, synthetic construct
  misc_feature 1..23: note = Synthetic Sequence
  source 1..23: mol_type = other DNA
ggagccacgaaaacagatccagg

SEQ ID NO: 464 - DNA, 23 bp, synthetic construct
  misc_feature 1..23: note = Synthetic Sequence
  source 1..23: mol_type = other DNA
agatccagggacacggtgctagg

SEQ ID NO: 465 - DNA, 23 bp, synthetic construct
  misc_feature 1..23: note = Synthetic Sequence
  source 1..23: mol_type = other DNA
gacacggtgctaggacagtgggg

SEQ ID NO: 466 - DNA, 23 bp, synthetic construct
  misc_feature 1..23: note = Synthetic Sequence
  source 1..23: mol_type = other DNA
gaaaatgacccaacagcctctgg

SEQ ID NO: 467 - DNA, 23 bp, synthetic construct
  misc_feature 1..23: note = Synthetic Sequence
  source 1..23: mol_type = other DNA
gcctggccggcctgaccactggg

SEQ ID NO: 468 - DNA, 23 bp, synthetic construct
  misc_feature 1..23: note = Synthetic Sequence
  source 1..23: mol_type = other DNA
ctgagcactgaaggcctggccgg

SEQ ID NO: 469 - DNA, 23 bp, synthetic construct
  misc_feature 1..23: note = Synthetic Sequence
  source 1..23: mol_type = other DNA
tggtttccactgagcactgaagg

SEQ ID NO: 470 - DNA, 23 bp, synthetic construct
  misc_feature 1..23: note = Synthetic Sequence
  source 1..23: mol_type = other DNA
ggtgctttcctgaggaccgatag

SEQ ID NO: 471 - DNA, 23 bp, synthetic construct
  misc_feature 1..23: note = Synthetic Sequence
  source 1..23: mol_type = other DNA
gcgcttccagtgctcagactagg

SEQ ID NO: 472 - DNA, 23 bp, synthetic construct
  misc_feature 1..23: note = Synthetic Sequence
  source 1..23: mol_type = other DNA
cagtgctcagactagggaagagg

SEQ ID NO: 473 - DNA, 23 bp, synthetic construct
  misc_feature 1..23: note = Synthetic Sequence
  source 1..23: mol_type = other DNA
gcccctcctccttcagagccagg

SEQ ID NO: 474 - DNA, 23 bp, synthetic construct
  misc_feature 1..23: note = Synthetic Sequence
  source 1..23: mol_type = other DNA
tccttcagagccaggagtcctgg

SEQ ID NO: 475 - DNA, 23 bp, synthetic construct
  misc_feature 1..23: note = Synthetic Sequence
  source 1..23: mol_type = other DNA
ccaagggtcaagctcggaaacca

SEQ ID NO: 476 - DNA, 23 bp, synthetic construct
  misc_feature 1..23: note = Synthetic Sequence
  source 1..23: mol_type = other DNA
ctgcagagtatctgctggggtgg

SEQ ID NO: 477 - DNA, 23 bp, synthetic construct
  misc_feature 1..23: note = Synthetic Sequence
  source 1..23: mol_type = other DNA
cgttcctgcagagtatctgctgg

SEQ ID NO: 478 - DNA, 20 bp, synthetic construct
  misc_feature 1..20: note = Synthetic Sequence
  source 1..20: mol_type = other DNA
gtggggaaaatgacccaaca

SEQ ID NO: 479 - DNA, 20 bp, synthetic construct
  misc_feature 1..20: note = Synthetic Sequence
  source 1..20: mol_type = other DNA
gaaggcctggccggcctgac

SEQ ID NO: 480 - DNA, 20 bp, synthetic construct
  misc_feature 1..20: note = Synthetic Sequence
  source 1..20: mol_type = other DNA
actcctggctctgaaggagg

SEQ ID NO: 481 - DNA, 20 bp, synthetic construct
  misc_feature 1..20: note = Synthetic Sequence
  source 1..20: mol_type = other DNA
gggctgggggccaggactcc

SEQ ID NO: 482 - DNA, 20 bp, synthetic construct
  misc_feature 1..20: note = Synthetic Sequence
  source 1..20: mol_type = other DNA
gtccttccaagggtcaagct

SEQ ID NO: 483 - DNA, 20 bp, synthetic construct
  misc_feature 1..20: note = Synthetic Sequence
  source 1..20: mol_type = other DNA
tcaagctcggaaaccacccc

SEQ ID NO: 484 - DNA, 23 bp, synthetic construct
  misc_feature 1..23: note = Synthetic Sequence
  source 1..23: mol_type = other DNA
attgtcttcactaaacccgttgg

SEQ ID NO: 485 - DNA, 23 bp, synthetic construct
  misc_feature 1..23: note = Synthetic Sequence
  source 1..23: mol_type = other DNA
taaacccgttgggaatacaatgg

SEQ ID NO: 486 - DNA, 23 bp, synthetic construct
  misc_feature 1..23: note = Synthetic Sequence
  source 1..23: mol_type = other DNA
ttgtcttcactaaacccgttggg

SEQ ID NO: 487 - DNA, 23 bp, synthetic construct
  misc_feature 1..23: note = Synthetic Sequence
  source 1..23: mol_type = other DNA
tgattcataggagtctattaagg

SEQ ID NO: 488 - DNA, 23 bp, synthetic construct
  misc_feature 1..23: note = Synthetic Sequence
  source 1..23: mol_type = other DNA
ttacatatgcttcgagtttgtgg

SEQ ID NO: 489 - DNA, 23 bp, synthetic construct
  misc_feature 1..23: note = Synthetic Sequence
  source 1..23: mol_type = other DNA
actcttaaggtaggactaattgg

SEQ ID NO: 490 - DNA, 23 bp, synthetic construct
  misc_feature 1..23: note = Synthetic Sequence
  source 1..23: mol_type = other DNA
tatgtgtgcaatagcgttaaagg

SEQ ID NO: 491 - DNA, 23 bp, synthetic construct
  misc_feature 1..23: note = Synthetic Sequence
  source 1..23: mol_type = other DNA
cgttgggaatacaatggcttagg

SEQ ID NO: 492 - DNA, 23 bp, synthetic construct
  misc_feature 1..23: note = Synthetic Sequence
  source 1..23: mol_type = other DNA
tcacaatggaactctgcctttgg

SEQ ID NO: 493 - DNA, 23 bp, synthetic construct
  misc_feature 1..23: note = Synthetic Sequence
  source 1..23: mol_type = other DNA
gaccacaaatcaatgcccaaagg

SEQ ID NO: 494 - DNA, 23 bp, synthetic construct
  misc_feature 1..23: note = Synthetic Sequence
  source 1..23: mol_type = other DNA
ctaagccattgtattcccaacgg

SEQ ID NO: 495 - DNA, 23 bp, synthetic construct
  misc_feature 1..23: note = Synthetic Sequence
  source 1..23: mol_type = other DNA
agcattctggagtgtcacaatgg

SEQ ID NO: 496 - DNA, 23 bp, synthetic construct
  misc_feature 1..23: note = Synthetic Sequence
  source 1..23: mol_type = other DNA
caatagcccactttaatactagg

SEQ ID NO: 497 - DNA, 23 bp, synthetic construct
  misc_feature 1..23: note = Synthetic Sequence
  source 1..23: mol_type = other DNA
ctttatccaagtgaatcctttgg

SEQ ID NO: 498 - DNA, 23 bp, synthetic construct
  misc_feature 1..23: note = Synthetic Sequence
  source 1..23: mol_type = other DNA
ggcattgatttgtggtcatttgg

SEQ ID NO: 499 - DNA, 23 bp, synthetic construct
  misc_feature 1..23: note = Synthetic Sequence
  source 1..23: mol_type = other DNA
taagccattgtattcccaacggg

SEQ ID NO: 500 - DNA, 23 bp, synthetic construct
  misc_feature 1..23: note = Synthetic Sequence
  source 1..23: mol_type = other DNA
aatacaatcactcttaaggtagg

SEQ ID NO: 501 - DNA, 23 bp, synthetic construct
  misc_feature 1..23: note = Synthetic Sequence
  source 1..23: mol_type = other DNA
gaagtacctttcactattttggg

SEQ ID NO: 502 - DNA, 23 bp, synthetic construct
  misc_feature 1..23: note = Synthetic Sequence
  source 1..23: mol_type = other DNA
caagcaacaaatgacttctaagg

SEQ ID NO: 503 - DNA, 23 bp, synthetic construct
  misc_feature 1..23: note = Synthetic Sequence
  source 1..23: mol_type = other DNA
tttgaatacaatcactcttaagg

SEQ ID NO: 504 - DNA, 23 bp, synthetic construct
  misc_feature 1..23: note = Synthetic Sequence
  source 1..23: mol_type = other DNA
acaaacggactacgtaaacttgg

SEQ ID NO: 505 - DNA, 23 bp, synthetic construct
  misc_feature 1..23: note = Synthetic Sequence
  source 1..23: mol_type = other DNA
acaagatgtgaacacgacgatgg

SEQ ID NO: 506 - DNA, 23 bp, synthetic construct
  misc_feature 1..23: note = Synthetic Sequence
  source 1..23: mol_type = other DNA
gttgcaccgttgattccttcagg

SEQ ID NO: 507 - DNA, 23 bp, synthetic construct
  misc_feature 1..23: note = Synthetic Sequence
  source 1..23: mol_type = other DNA
agtaatattgaattagggcgtgg

SEQ ID NO: 508 - DNA, 23 bp, synthetic construct
  misc_feature 1..23: note = Synthetic Sequence
  source 1..23: mol_type = other DNA
cctgatgttggctcgacattagg

SEQ ID NO: 509 - DNA, 23 bp, synthetic construct
  misc_feature 1..23: note = Synthetic Sequence
  source 1..23: mol_type = other DNA
ctttgttgggtcttagcttaagg

SEQ ID NO: 510 - DNA, 23 bp, synthetic construct
  misc_feature 1..23: note = Synthetic Sequence
  source 1..23: mol_type = other DNA
tcggaacagctccttcctgaagg

SEQ ID NO: 511 - DNA, 23 bp, synthetic construct
  misc_feature 1..23: note = Synthetic Sequence
  source 1..23: mol_type = other DNA
agtagtttctgaggtcatgttgg

SEQ ID NO: 512 - DNA, 23 bp, synthetic construct
  misc_feature 1..23: note = Synthetic Sequence
  source 1..23: mol_type = other DNA
cttgaaaatacgatgatgtgagg

SEQ ID NO: 513 - DNA, 23 bp, synthetic construct
  misc_feature 1..23: note = Synthetic Sequence
  source 1..23: mol_type = other DNA
gcattaatctagagagagggagg

SEQ ID NO: 514 - DNA, 23 bp, synthetic construct
  misc_feature 1..23: note = Synthetic Sequence
  source 1..23: mol_type = other DNA
gggtcatgttagaattcatgtgg

SEQ ID NO: 515 - DNA, 23 bp, synthetic construct
  misc_feature 1..23: note = Synthetic Sequence
  source 1..23: mol_type = other DNA
tgatgcattaatctagagagagg

SEQ ID NO: 516 - DNA, 23 bp, synthetic construct
  misc_feature 1..23: note = Synthetic Sequence
  source 1..23: mol_type = other DNA
acatcatcgtattttcaagttgg

SEQ ID NO: 517 - DNA, 23 bp, synthetic construct
  misc_feature 1..23: note = Synthetic Sequence
  source 1..23: mol_type = other DNA
ctagctgacaaacatgtgagtgg

SEQ ID NO: 518 - DNA, 23 bp, synthetic construct
  misc_feature 1..23: note = Synthetic Sequence
  source 1..23: mol_type = other DNA
aacatgacccaagtgagtccagg

SEQ ID NO: 519 - DNA, 23 bp, synthetic construct
  misc_feature 1..23: note = Synthetic Sequence
  source 1..23: mol_type = other DNA
gattccgtatttgctttgttggg

SEQ ID NO: 520 - DNA, 23 bp, synthetic construct
  misc_feature 1..23: note = Synthetic Sequence
  source 1..23: mol_type = other DNA
tacgatgatgtgaggaaataagg

SEQ ID NO: 521 - DNA, 23 bp, synthetic construct
  misc_feature 1..23: note = Synthetic Sequence
  source 1..23: mol_type = other DNA
gtaatatgtctaagtactgatgg

SEQ ID NO: 522 - DNA, 23 bp, synthetic construct
  misc_feature 1..23: note = Synthetic Sequence
  source 1..23: mol_type = other DNA
gtaaagtgagctggttcattagg

SEQ ID NO: 523 - DNA, 23 bp, synthetic construct
  misc_feature 1..23: note = Synthetic Sequence
  source 1..23: mol_type = other DNA
actagagtccttaagaagggggg

SEQ ID NO: 524 - DNA, 23 bp, synthetic construct
  misc_feature 1..23: note = Synthetic Sequence
  source 1..23: mol_type = other DNA
ataacacgtgagccgtcctaagg

SEQ ID NO: 525 - DNA, 23 bp, synthetic construct
  misc_feature 1..23: note = Synthetic Sequence
  source 1..23: mol_type = other DNA
ggaagacttttctctatacgagg

SEQ ID NO: 526 - DNA, 23 bp, synthetic construct
  misc_feature 1..23: note = Synthetic Sequence
  source 1..23: mol_type = other DNA
gcattcctttcatccatggcagg

SEQ ID NO: 527 - DNA, 23 bp, synthetic construct
  misc_feature 1..23: note = Synthetic Sequence
  source 1..23: mol_type = other DNA
gacatatggttataaaaatcagg

SEQ ID NO: 528 - DNA, 23 bp, synthetic construct
  misc_feature 1..23: note = Synthetic Sequence
  source 1..23: mol_type = other DNA
ggagtgcagtccctgacatatgg

SEQ ID NO: 529 - DNA, 23 bp, synthetic construct
  misc_feature 1..23: note = Synthetic Sequence
  source 1..23: mol_type = other DNA
gtgggttagggtggttaactggg

SEQ ID NO: 530 - DNA, 23 bp, synthetic construct
  misc_feature 1..23: note = Synthetic Sequence
  source 1..23: mol_type = other DNA
aggtgcaaaaaggttgctgtggg

SEQ ID NO: 531 - DNA, 23 bp, synthetic construct
  misc_feature 1..23: note = Synthetic Sequence
  source 1..23: mol_type = other DNA
cgtgacaaggcaaagtggcgtgg

SEQ ID NO: 532 - DNA, 23 bp, synthetic construct
  misc_feature 1..23: note = Synthetic Sequence
  source 1..23: mol_type = other DNA
gaaggactgcccctgacgtcagg

SEQ ID NO: 533 - DNA, 23 bp, synthetic construct
  misc_feature 1..23: note = Synthetic Sequence
  source 1..23: mol_type = other DNA
ctgcccctgacgtcaggagttgg

SEQ ID NO: 534 - DNA, 23 bp, synthetic construct
  misc_feature 1..23: note = Synthetic Sequence
  source 1..23: mol_type = other DNA
tgtgggttagggtggttaactgg

SEQ ID NO: 535 - DNA, 23 bp, synthetic construct
  misc_feature 1..23: note = Synthetic Sequence
  source 1..23: mol_type = other DNA
acccttttagagttttctgctgg

SEQ ID NO: 536 - DNA, 23 bp, synthetic construct
  misc_feature 1..23: note = Synthetic Sequence
  source 1..23: mol_type = other DNA
aacttcctgccatggatgaaagg

SEQ ID NO: 537 - DNA, 23 bp, synthetic construct
  misc_feature 1..23: note = Synthetic Sequence
  source 1..23: mol_type = other DNA
gcaaaaaggttgctgtgggttgg

SEQ ID NO: 538 - DNA, 23 bp, synthetic construct
  misc_feature 1..23: note = Synthetic Sequence
  source 1..23: mol_type = other DNA
aatttgggggtagataggcatgg

SEQ ID NO: 539 - DNA, 23 bp, synthetic construct
  misc_feature 1..23: note = Synthetic Sequence
  source 1..23: mol_type = other DNA
agaaaactctaaaagggtatagg

SEQ ID NO: 540 - DNA, 23 bp, synthetic construct
  misc_feature 1..23: note = Synthetic Sequence
  source 1..23: mol_type = other DNA
attagcattcctttcatccatgg

SEQ ID NO: 541 - DNA, 23 bp, synthetic construct
  misc_feature 1..23: note = Synthetic Sequence
  source 1..23: mol_type = other DNA
cccagcagaaaactctaaaaggg

SEQ ID NO: 542 - DNA, 23 bp, synthetic construct
  misc_feature 1..23: note = Synthetic Sequence
  source 1..23: mol_type = other DNA
caggtgcaaaaaggttgctgtgg

SEQ ID NO: 543 - DNA, 23 bp, synthetic construct
  misc_feature 1..23: note = Synthetic Sequence
  source 1..23: mol_type = other DNA
gcaagagatgaaattccatatgg

SEQ ID NO: 544 - DNA, 23 bp, synthetic construct
  misc_feature 1..23: note = Synthetic Sequence
  source 1..23: mol_type = other DNA
gggctgttctaacgaagtctggg

SEQ ID NO: 545 - DNA, 23 bp, synthetic construct
  misc_feature 1..23: note = Synthetic Sequence
  source 1..23: mol_type = other DNA
tgtccattcagcgaccctagagg

SEQ ID NO: 546 - DNA, 23 bp, synthetic construct
  misc_feature 1..23: note = Synthetic Sequence
  source 1..23: mol_type = other DNA
ggctgttctaacgaagtctgggg

SEQ ID NO: 547 - DNA, 23 bp, synthetic construct
  misc_feature 1..23: note = Synthetic Sequence
  source 1..23: mol_type = other DNA
gtccattcagcgaccctagaggg

SEQ ID NO: 548 - DNA, 23 bp, synthetic construct
  misc_feature 1..23: note = Synthetic Sequence
  source 1..23: mol_type = other DNA
ggggctgttctaacgaagtctgg

SEQ ID NO: 549 - DNA, 23 bp, synthetic construct
  misc_feature 1..23: note = Synthetic Sequence
  source 1..23: mol_type = other DNA
ggctgaatcagcatgcgaaaggg

SEQ ID NO: 550 - DNA, 23 bp, synthetic construct
  misc_feature 1..23: note = Synthetic Sequence
  source 1..23: mol_type = other DNA
ttccaatggggggcatagcctgg

SEQ ID NO: 551 - DNA, 23 bp, synthetic construct
  misc_feature 1..23: note = Synthetic Sequence
  source 1..23: mol_type = other DNA
taccctctagggtcgctgaatgg

SEQ ID NO: 552 - DNA, 23 bp, synthetic construct
  misc_feature 1..23: note = Synthetic Sequence
  source 1..23: mol_type = other DNA
atcctcttgggccttataagagg

SEQ ID NO: 553 - DNA, 23 bp, synthetic construct
  misc_feature 1..23: note = Synthetic Sequence
  source 1..23: mol_type = other DNA
ggccaggctatgccccccattgg

SEQ ID NO: 554 - DNA, 23 bp, synthetic construct
  misc_feature 1..23: note = Synthetic Sequence
  source 1..23: mol_type = other DNA
ctagaggaccagaacaactctgg

SEQ ID NO: 555 - DNA, 23 bp, synthetic construct
  misc_feature 1..23: note = Synthetic Sequence
  source 1..23: mol_type = other DNA
ctagaggaccagaacaactctgg

SEQ ID NO: 556 - DNA, 23 bp, synthetic construct
  misc_feature 1..23: note = Synthetic Sequence
  source 1..23: mol_type = other DNA
aggctgaatcagcatgcgaaagg

SEQ ID NO: 557 - DNA, 23 bp, synthetic construct
  misc_feature 1..23: note = Synthetic Sequence
  source 1..23: mol_type = other DNA
ggaccagaacaactctggcctgg

SEQ ID NO: 558 - DNA, 23 bp, synthetic construct
  misc_feature 1..23: note = Synthetic Sequence
  source 1..23: mol_type = other DNA
gggcttttatttggcccagcagg

SEQ ID NO: 559 - DNA, 23 bp, synthetic construct
  misc_feature 1..23: note = Synthetic Sequence
  source 1..23: mol_type = other DNA
gtcgctgaatggacagactctgg

SEQ ID NO: 560 - DNA, 23 bp, synthetic construct
  misc_feature 1..23: note = Synthetic Sequence
  source 1..23: mol_type = other DNA
ctcatgagttttaccctctaggg

SEQ ID NO: 561 - DNA, 23 bp, synthetic construct
  misc_feature 1..23: note = Synthetic Sequence
  source 1..23: mol_type = other DNA
tcctcttgggccttataagaggg

SEQ ID NO: 562 - DNA, 23 bp, synthetic construct
  misc_feature 1..23: note = Synthetic Sequence
  source 1..23: mol_type = other DNA
tcttgggccttataagagggagg

SEQ ID NO: 563 - DNA, 23 bp, synthetic construct
  misc_feature 1..23: note = Synthetic Sequence
  source 1..23: mol_type = other DNA
tagaacagccccccacacagtgg

SEQ ID NO: 564 - DNA, 23 bp, synthetic construct
  misc_feature 1..23: note = Synthetic Sequence
  source 1..23: mol_type = other DNA
gttacgttatgactaatctttgg

SEQ ID NO: 565 - DNA, 23 bp, synthetic construct
  misc_feature 1..23: note = Synthetic Sequence
  source 1..23: mol_type = other DNA
tacgttatgactaatctttgggg

SEQ ID NO: 566 - DNA, 23 bp, synthetic construct
  misc_feature 1..23: note = Synthetic Sequence
  source 1..23: mol_type = other DNA
ggaagtagtgttatgatgtatgg

SEQ ID NO: 567 - DNA, 23 bp, synthetic construct
  misc_feature 1..23: note = Synthetic Sequence
  source 1..23: mol_type = other DNA
gttatgatgtatgggcataaagg

SEQ ID NO: 568 - DNA, 23 bp, synthetic construct
  misc_feature 1..23: note = Synthetic Sequence
  source 1..23: mol_type = other DNA
gaagtagtgttatgatgtatggg

SEQ ID NO: 569 - DNA, 23 bp, synthetic construct
  misc_feature 1..23: note = Synthetic Sequence
  source 1..23: mol_type = other DNA
atagctgctggcagtataactgg

SEQ ID NO: 570 - DNA, 23 bp, synthetic construct
  misc_feature 1..23: note = Synthetic Sequence
  source 1..23: mol_type = other DNA
gcatcacaacattgacactgtgg

SEQ ID NO: 571 - DNA, 23 bp, synthetic construct
  misc_feature 1..23: note = Synthetic Sequence
  source 1..23: mol_type = other DNA
aaggcgagtttctacaaagatgg

SEQ ID NO: 572 - DNA, 23 bp, synthetic construct
  misc_feature 1..23: note = Synthetic Sequence
  source 1..23: mol_type = other DNA
ttacgttatgactaatctttggg

SEQ ID NO: 573 - DNA, 23 bp, synthetic construct
  misc_feature 1..23: note = Synthetic Sequence
  source 1..23: mol_type = other DNA
caagactgattaagactgatggg

SEQ ID NO: 574 - DNA, 23 bp, synthetic construct
  misc_feature 1..23: note = Synthetic Sequence
  source 1..23: mol_type = other DNA
agcagcaatgtattaaaggctgg

SEQ ID NO: 575 - DNA, 23 bp, synthetic construct
  misc_feature 1..23: note = Synthetic Sequence
  source 1..23: mol_type = other DNA
ctacaggattgatgtaaacatgg

SEQ ID NO: 576 - DNA, 23 bp, synthetic construct
  misc_feature 1..23: note = Synthetic Sequence
  source 1..23: mol_type = other DNA
tgggcataaagggttttaatggg

SEQ ID NO: 577 - DNA, 23 bp, synthetic construct
  misc_feature 1..23: note = Synthetic Sequence
  source 1..23: mol_type = other DNA
acatcaatcctgtaggtgattgg

SEQ ID NO: 578 - DNA, 23 bp, synthetic construct
  misc_feature 1..23: note = Synthetic Sequence
  source 1..23: mol_type = other DNA
attctagtcattatagctgctgg

SEQ ID NO: 579 - DNA, 23 bp, synthetic construct
  misc_feature 1..23: note = Synthetic Sequence
  source 1..23: mol_type = other DNA
catcaatcctgtaggtgattggg

SEQ ID NO: 580 - DNA, 23 bp, synthetic construct
  misc_feature 1..23: note = Synthetic Sequence
  source 1..23: mol_type = other DNA
gttataagatcaattctgagtgg

SEQ ID NO: 581 - DNA, 23 bp, synthetic construct
  misc_feature 1..23: note = Synthetic Sequence
  source 1..23: mol_type = other DNA
ggcagactgtggatcaaaagtgg

SEQ ID NO: 582 - DNA, 23 bp, synthetic construct
  misc_feature 1..23: note = Synthetic Sequence
  source 1..23: mol_type = other DNA
atggctgcccaatcacctacagg

SEQ ID NO: 583 - DNA, 23 bp, synthetic construct
  misc_feature 1..23: note = Synthetic Sequence
  source 1..23: mol_type = other DNA
tcaaagcatgtacttagagttgg

SEQ ID NO: 584 - DNA, 20 bp, synthetic construct
  misc_feature 1..20: note = Synthetic Sequence
  source 1..20: mol_type = other DNA
tcgcccctcaaatcttacag

SEQ ID NO: 585 - DNA, 20 bp, synthetic construct
  misc_feature 1..20: note = Synthetic Sequence
  source 1..20: mol_type = other DNA
tcaaatcttacagctgctca

SEQ ID NO: 586 - DNA, 20 bp, synthetic construct
  misc_feature 1..20: note = Synthetic Sequence
  source 1..20: mol_type = other DNA
tcttacagctgctcactccc

SEQ ID NO: 587 - DNA, 20 bp, synthetic construct
  misc_feature 1..20: note = Synthetic Sequence
  source 1..20: mol_type = other DNA
tacagctgctcactcccctg

SEQ ID NO: 588 - DNA, 20 bp, synthetic construct
  misc_feature 1..20: note = Synthetic Sequence
  source 1..20: mol_type = other DNA
tgctcactcccctgcagggc

SEQ ID NO: 589 - DNA, 20 bp, synthetic construct
  misc_feature 1..20: note = Synthetic Sequence
  source 1..20: mol_type = other DNA
tgcagggcaacgcccaggga

SEQ ID NO: 590 - DNA, 20 bp, synthetic construct
  misc_feature 1..20: note = Synthetic Sequence
  source 1..20: mol_type = other DNA
tctcgattatgggcgggatt

SEQ ID NO: 591 - DNA, 20 bp, synthetic construct
  misc_feature 1..20: note = Synthetic Sequence
  source 1..20: mol_type = other DNA
tcgcttctcgattatgggcg

SEQ ID NO: 592 - DNA, 20 bp, synthetic construct
  misc_feature 1..20: note = Synthetic Sequence
  source 1..20: mol_type = other DNA
tgtcgagtcgcttctcgatt

SEQ ID NO: 593 - DNA, 20 bp, synthetic construct
  misc_feature 1..20: note = Synthetic Sequence
  source 1..20: mol_type = other DNA
tccatgtcgagtcgcttctc

SEQ ID NO: 594 - DNA, 20 bp, synthetic construct
  misc_feature 1..20: note = Synthetic Sequence
  source 1..20: mol_type = other DNA
tcgcctccatgtcgagtcgc

SEQ ID NO: 595 - DNA, 20 bp, synthetic construct
  misc_feature 1..20: note = Synthetic Sequence
  source 1..20: mol_type = other DNA
tcgtcatcgcctccatgtcg

SEQ ID NO: 596 - DNA, 20 bp, synthetic construct
  misc_feature 1..20: note = Synthetic Sequence
  source 1..20: mol_type = other DNA
tgatctcgtcatcgcctcca

SEQ ID NO: 597 - DNA, 20 bp, synthetic construct
  misc_feature 1..20: note = Synthetic Sequence
  source 1..20: mol_type = other DNA
tggccggcctgaccactggg

SEQ ID NO: 598 - DNA, 20 bp, synthetic construct
  misc_feature 1..20: note = Synthetic Sequence
  source 1..20: mol_type = other DNA
tgaaggcctggccggcctga

SEQ ID NO: 599 - DNA, 20 bp, synthetic construct
  misc_feature 1..20: note = Synthetic Sequence
  source 1..20: mol_type = other DNA
tgagcactgaaggcctggcc

SEQ ID NO: 600 - DNA, 20 bp, synthetic construct
  misc_feature 1..20: note = Synthetic Sequence
  source 1..20: mol_type = other DNA
tccactgagcactgaaggcc

SEQ ID NO: 601 - DNA, 20 bp, synthetic construct
  misc_feature 1..20: note = Synthetic Sequence
  source 1..20: mol_type = other DNA
tggtttccactgagcactga

SEQ ID NO: 602 - DNA, 20 bp, synthetic construct
  misc_feature 1..20: note = Synthetic Sequence
  source 1..20: mol_type = other DNA
tggggaaaatgacccaacag

SEQ ID NO: 603 - DNA, 20 bp, synthetic construct
  misc_feature 1..20: note = Synthetic Sequence
  source 1..20: mol_type = other DNA
taggacagtggggaaaatga

SEQ ID NO: 604 - DNA, 20 bp, synthetic construct
  misc_feature 1..20: note = Synthetic Sequence
  source 1..20: mol_type = other DNA
tccagggacacggtgctagg

SEQ ID NO: 605 - DNA, 20 bp, synthetic construct
  misc_feature 1..20: note = Synthetic Sequence
  source 1..20: mol_type = other DNA
tcagagccaggagtcctggc

SEQ ID NO: 606 - DNA, 20 bp, synthetic construct
  misc_feature 1..20: note = Synthetic Sequence
  source 1..20: mol_type = other DNA
tccttcagagccaggagtcc

SEQ ID NO: 607 - DNA, 20 bp, synthetic construct
  misc_feature 1..20: note = Synthetic Sequence
  source 1..20: mol_type = other DNA
tcctccttcagagccaggag

SEQ ID NO: 608 - DNA, 20 bp, synthetic construct
  misc_feature 1..20: note = Synthetic Sequence
  source 1..20: mol_type = other DNA
tccagcccctcctccttcag

SEQ ID NO: 609 - DNA, 20 bp, synthetic construct
  misc_feature 1..20: note = Synthetic Sequence
  source 1..20: mol_type = other DNA
tggggtggtttccgagcttg

SEQ ID NO: 610 - DNA, 20 bp, synthetic construct
  misc_feature 1..20: note = Synthetic Sequence
  source 1..20: mol_type = other DNA
tctgctggggtggtttccga

SEQ ID NO: 611 - DNA, 20 bp, synthetic construct
  misc_feature 1..20: note = Synthetic Sequence
  source 1..20: mol_type = other DNA
tgcagagtatctgctggggt

SEQ ID NO: 612 - DNA, 18 bp, synthetic construct
  misc_feature 1..18: note = Synthetic Sequence
  source 1..18: mol_type = other DNA
tcttcctagtattaaagt

SEQ ID NO: 613 - DNA, 17 bp, synthetic construct
  misc_feature 1..17: note = Synthetic Sequence
  source 1..17: mol_type = other DNA
tccttaatattaccagt

SEQ ID NO: 614 - DNA, 25 bp, synthetic construct
  misc_feature 1..25: note = Synthetic Sequence
  source 1..25: mol_type = other DNA
taccaagctgaaatgacacaaaagt

SEQ ID NO: 615 - DNA, 25 bp, synthetic construct
  misc_feature 1..25: note = Synthetic Sequence
  source 1..25: mol_type = other DNA
tggctgtgtcacataccagcagaat

SEQ ID NO: 616 - DNA, 22 bp, synthetic construct
  misc_feature 1..22: note = Synthetic Sequence
  source 1..22: mol_type = other DNA
tgttaatttgaatacaatcact

SEQ ID NO: 617 - DNA, 21 bp, synthetic construct
  misc_feature 1..21: note = Synthetic Sequence
  source 1..21: mol_type = other DNA
tgtgtcacataccagcagaat

SEQ ID NO: 618 - DNA, 16 bp, synthetic construct
  misc_feature 1..16: note = Synthetic Sequence
  source 1..16: mol_type = other DNA
tggtaactactaattt

SEQ ID NO: 619 - DNA, 19 bp, synthetic construct
  misc_feature 1..19: note = Synthetic Sequence
  source 1..19: mol_type = other DNA
tgtcacataccagcagaat

SEQ ID NO: 620 - DNA, 21 bp, synthetic construct
  misc_feature 1..21: note = Synthetic Sequence
  source 1..21: mol_type = other DNA
tgtgacacagccatcaacaat

SEQ ID NO: 621 - DNA, 16 bp, synthetic construct
  misc_feature 1..16: note = Synthetic Sequence
  source 1..16: mol_type = other DNA
tcctttgatgaacagt

SEQ ID NO: 622 - DNA, 29 bp, synthetic construct
  misc_feature 1..29: note = Synthetic Sequence
  source 1..29: mol_type = other DNA
tgtgtgcaatagcgttaaaggaactacat

SEQ ID NO: 623 - DNA, 17 bp, synthetic construct
  misc_feature 1..17: note = Synthetic Sequence
  source 1..17: mol_type = other DNA
tctttcaatagcccact

SEQ ID NO: 624 - DNA, 22 bp, synthetic construct
  misc_feature 1..22: note = Synthetic Sequence
  source 1..22: mol_type = other DNA
tctcaaatgacaagagcacagt

SEQ ID NO: 625 - DNA, 18 bp, synthetic construct
  misc_feature 1..18: note = Synthetic Sequence
  source 1..18: mol_type = other DNA
taccagttaattagcact

SEQ ID NO: 626 - DNA, 18 bp, synthetic construct
  misc_feature 1..18: note = Synthetic Sequence
  source 1..18: mol_type = other DNA
tgttgtgacctaagccat

SEQ ID NO: 627 - DNA, 23 bp, synthetic construct
  misc_feature 1..23: note = Synthetic Sequence
  source 1..23: mol_type = other DNA
tctcatgttttaaagtcaagaat

SEQ ID NO: 628 - DNA, 19 bp, synthetic construct
  misc_feature 1..19: note = Synthetic Sequence
  source 1..19: mol_type = other DNA
tcctgaattcagaacagat

SEQ ID NO: 629 - DNA, 26 bp, synthetic construct
  misc_feature 1..26: note = Synthetic Sequence
  source 1..26: mol_type = other DNA
tagcatgatgtttcatgttgtgacct

SEQ ID NO: 630 - DNA, 25 bp, synthetic construct
  misc_feature 1..25: note = Synthetic Sequence
  source 1..25: mol_type = other DNA
tgtttcatgttgtgacctaagccat

SEQ ID NO: 631 - DNA, 18 bp, synthetic construct
  misc_feature 1..18: note = Synthetic Sequence
  source 1..18: mol_type = other DNA
tacaacagtctatttcat

SEQ ID NO: 632 - DNA, 26 bp, synthetic construct
  misc_feature 1..26: note = Synthetic Sequence
  source 1..26: mol_type = other DNA
tcttcctagtctcttctctacccagt

SEQ ID NO: 633 - DNA, 25 bp, synthetic construct
  misc_feature 1..25: note = Synthetic Sequence
  source 1..25: mol_type = other DNA
tacactccagcctgggaaacagagt

SEQ ID NO: 634 - DNA, 19 bp, synthetic construct
  misc_feature 1..19: note = Synthetic Sequence
  source 1..19: mol_type = other DNA
tcttttccttaggacggct

SEQ ID NO: 635 - DNA, 17 bp, synthetic construct
  misc_feature 1..17: note = Synthetic Sequence
  source 1..17: mol_type = other DNA
tcgctcaggcctgtcat

SEQ ID NO: 636 - DNA, 16 bp, synthetic construct
  misc_feature 1..16: note = Synthetic Sequence
  source 1..16: mol_type = other DNA
tccatatggaagactt

SEQ ID NO: 637 - DNA, 18 bp, synthetic construct
  misc_feature 1..18: note = Synthetic Sequence
  source 1..18: mol_type = other DNA
tacccagttaaccaccct

SEQ ID NO: 638 - DNA, 27 bp, synthetic construct
  misc_feature 1..27: note = Synthetic Sequence
  source 1..27: mol_type = other DNA
tggcgcatgcctgtaatcccagctact

SEQ ID NO: 639 - DNA, 26 bp, synthetic construct
  misc_feature 1..26: note = Synthetic Sequence
  source 1..26: mol_type = other DNA
tatacgaggagaaaattagcattcct

SEQ ID NO: 640 - DNA, 23 bp, synthetic construct
  misc_feature 1..23: note = Synthetic Sequence
  source 1..23: mol_type = other DNA
tctgcctcccaggttcacgcaat

SEQ ID NO: 641 - DNA, 22 bp, synthetic construct
  misc_feature 1..22: note = Synthetic Sequence
  source 1..22: mol_type = other DNA
tgccttgtcacgttttcacagt

SEQ ID NO: 642 - DNA, 25 bp, synthetic construct
  misc_feature 1..25: note = Synthetic Sequence
  source 1..25: mol_type = other DNA
tgtcaccttctgtatgtgcaaccat

SEQ ID NO: 643 - DNA, 17 bp, synthetic construct
  misc_feature 1..17: note = Synthetic Sequence
  source 1..17: mol_type = other DNA
tctgtatgtgcaaccat

SEQ ID NO: 644 - DNA, 17 bp, synthetic construct
  misc_feature 1..17: note = Synthetic Sequence
  source 1..17: mol_type = other DNA
tagtcaagcaacaggat

SEQ ID NO: 645 - DNA, 18 bp, synthetic construct
  misc_feature 1..18: note = Synthetic Sequence
  source 1..18: mol_type = other DNA
tccaagataattccccat

SEQ ID NO: 646 - DNA, 16 bp, synthetic construct
  misc_feature 1..16: note = Synthetic Sequence
  source 1..16: mol_type = other DNA
tctgcaagatcctttt

SEQ ID NO: 647 - DNA, 29 bp, synthetic construct
  misc_feature 1..29: note = Synthetic Sequence
  source 1..29: mol_type = other DNA
tgctatgtaaggtagcaaaaaggtaacct

SEQ ID NO: 648 - DNA, 16 bp, synthetic construct
  misc_feature 1..16: note = Synthetic Sequence
  source 1..16: mol_type = other DNA
tctctctcctcctgct

SEQ ID NO: 649 - DNA, 19 bp, synthetic construct
  misc_feature 1..19: note = Synthetic Sequence
  source 1..19: mol_type = other DNA
tccaaatgctattctctct

SEQ ID NO: 650 - DNA, 17 bp, synthetic construct
  misc_feature 1..17: note = Synthetic Sequence
  source 1..17: mol_type = other DNA
tgctgattcagcctcct

SEQ ID NO: 651 - DNA, 21 bp, synthetic construct
  misc_feature 1..21: note = Synthetic Sequence
  source 1..21: mol_type = other DNA
tagaacagccccccacacagt

SEQ ID NO: 652 - DNA, 18 bp, synthetic construct
  misc_feature 1..18: note = Synthetic Sequence
  source 1..18: mol_type = other DNA
tttagcagatgcatcagc

SEQ ID NO: 653 - DNA, 20 bp, synthetic construct
  misc_feature 1..20: note = Synthetic Sequence
  source 1..20: mol_type = other DNA
tgaccaggggcatgtcctgg

SEQ ID NO: 654 - DNA, 21 bp, synthetic construct
  misc_feature 1..21: note = Synthetic Sequence
  source 1..21: mol_type = other DNA
tctacatttaactatcaacct

SEQ ID NO: 655 - DNA, 21 bp, synthetic construct
  misc_feature 1..21: note = Synthetic Sequence
  source 1..21: mol_type = other DNA
tgtcccacaggtattacgggc

SEQ ID NO: 656 - DNA, 21 bp, synthetic construct
  misc_feature 1..21: note = Synthetic Sequence
  source 1..21: mol_type = other DNA
tacgggccaacctgacaatac

SEQ ID NO: 657 - DNA, 21 bp, synthetic construct
  misc_feature 1..21: note = Synthetic Sequence
  source 1..21: mol_type = other DNA
tgagctttggggactgaaaga

SEQ ID NO: 658 - DNA, 21 bp, synthetic construct
  misc_feature 1..21: note = Synthetic Sequence
  source 1..21: mol_type = other DNA
ctggcataatcttttccccca

SEQ ID NO: 659 - DNA, 21 bp, synthetic construct
  misc_feature 1..21: note = Synthetic Sequence
  source 1..21: mol_type = other DNA
ccagcctcctggccatgtgca

SEQ ID NO: 660 - DNA, 21 bp, synthetic construct
  misc_feature 1..21: note = Synthetic Sequence
  source 1..21: mol_type = other DNA
ggccatgtgcacaggggctga

SEQ ID NO: 661 - DNA, 21 bp, synthetic construct
  misc_feature 1..21: note = Synthetic Sequence
  source 1..21: mol_type = other DNA
ctgatatgtgaaggtttagca

SEQ ID NO: 662 - DNA, 21 bp, synthetic construct
  misc_feature 1..21: note = Synthetic Sequence
  source 1..21: mol_type = other DNA
tgaccaggcgtggtggctcac

SEQ ID NO: 663 - DNA, 16 bp, synthetic construct
  misc_feature 1..16: note = Synthetic Sequence
  source 1..16: mol_type = other DNA
tatagacattttcact

SEQ ID NO: 664 - DNA, 21 bp, synthetic construct
  misc_feature 1..21: note = Synthetic Sequence
  source 1..21: mol_type = other DNA
tggtccacctacctgaaaatg

SEQ ID NO: 665 - DNA, 17 bp, synthetic construct
  misc_feature 1..17: note = Synthetic Sequence
  source 1..17: mol_type = other DNA
tcgtgcaaacgtttgat

SEQ ID NO: 666 - DNA, 18 bp, synthetic construct
  misc_feature 1..18: note = Synthetic Sequence
  source 1..18: mol_type = other DNA
tacatcaatcctgtaggt

SEQ ID NO: 667 - DNA, 20 bp, synthetic construct
  misc_feature 1..20: note = Synthetic Sequence
  source 1..20: mol_type = other DNA
tctattttagtgacccaagt

SEQ ID NO: 668 - DNA, 19 bp, synthetic construct
  misc_feature 1..19: note = Synthetic Sequence
  source 1..19: mol_type = other DNA
tagagtcaaagcatgtact

SEQ ID NO: 669 - DNA, 18 bp, synthetic construct
  misc_feature 1..18: note = Synthetic Sequence
  source 1..18: mol_type = other DNA
tcctacccataagctcct

SEQ ID NO: 670 - DNA, 17 bp, synthetic construct
  misc_feature 1..17: note = Synthetic Sequence
  source 1..17: mol_type = other DNA
tccccatccccatcagt

SEQ ID NO: 671 - DNA, 29 bp, synthetic construct
  misc_feature 1..29: note = Synthetic Sequence
  source 1..29: mol_type = other DNA
tctttaattcaagcaagactttaacaagt

SEQ ID NO: 672 - DNA, 16 bp, synthetic construct
  misc_feature 1..16: note = Synthetic Sequence
  source 1..16: mol_type = other DNA
tgcagtcccctttctt

SEQ ID NO: 673 - DNA, 21 bp, synthetic construct
  misc_feature 1..21: note = Synthetic Sequence
  source 1..21: mol_type = other DNA
tctgcacaaatccccaaagat

SEQ ID NO: 674 - DNA, 16 bp, synthetic construct
  misc_feature 1..16: note = Synthetic Sequence
  source 1..16: mol_type = other DNA
tacatgctttgactct

SEQ ID NO: 675 - DNA, 29 bp, synthetic construct
  misc_feature 1..29: note = Synthetic Sequence
  source 1..29: mol_type = other DNA
tggccagttatactgccagcagctataat

SEQ ID NO: 676 - DNA, 15 bp, synthetic construct
  misc_feature 1..15: note = Synthetic Sequence
  source 1..15: mol_type = other DNA
tgggaagataaacta

SEQ ID NO: 677 - protein, 148 aa, synthetic construct
  REGION 1..148: note = Synthetic Sequence
LEPGEKPYKCPECGKSFSQNSTLTEHQRTHTGEKPYKCPECGKSFSQRANLRAHQRTHTGEKPYKCPECGKSFSTSGNLVRHQRTHTGEKPYKCPECGKSFSQSSNLVRHQRTHTGEKPYKCPECGKSFSRSDHLTTHQRTHTGKKTS

SEQ ID NO: 678 - DNA, 18 bp, synthetic construct
  misc_feature 1..18: note = Synthetic Sequence
  source 1..18: mol_type = other DNA
actcccctgcagggcaac

SEQ ID NO: 679 - protein, 176 aa, synthetic construct
  REGION 1..176: note = Synthetic Sequence
LEPGEKPYKCPECGKSFSDSGNLRVHQRTHTGEKPYKCPECGKSFSDPGHLVRHQRTHTGEKPYKCPECGKSFSRADNLTEHQRTHTGEKPYKCPECGKSFSRNDALTEHQRTHTGEKPYKCPECGKSFSSKKHLAEHQRTHTGEKPYKCPECGKSFSTHLDLIRHQRTHTGKKTS

SEQ ID NO: 680 - DNA, 18 bp, synthetic construct
  misc_feature 1..18: note = Synthetic Sequence
  source 1..18: mol_type = other DNA
cccctgcagggcaacgcc

SEQ ID NO: 681 - protein, 176 aa, synthetic construct
  REGION 1..176: note = Synthetic Sequence
LEPGEKPYKCPECGKSFSDCRDLARHQRTHTGEKPYKCPECGKSFSDSGNLRVHQRTHTGEKPYKCPECGKSFSDPGHLVRHQRTHTGEKPYKCPECGKSFSRADNLTEHQRTHTGEKPYKCPECGKSFSRNDALTEHQRTHTGEKPYKCPECGKSFSSKKHLAEHQRTHTGKKTS

SEQ ID NO: 682 - DNA, 18 bp, synthetic construct
  misc_feature 1..18: note = Synthetic Sequence
  source 1..18: mol_type = other DNA
ctgcagggcaacgcccag

SEQ ID NO: 683 - protein, 176 aa, synthetic construct
  REGION 1..176: note = Synthetic Sequence
LEPGEKPYKCPECGKSFSRADNLTEHQRTHTGEKPYKCPECGKSFSDCRDLARHQRTHTGEKPYKCPECGKSFSDSGNLRVHQRTHTGEKPYKCPECGKSFSDPGHLVRHQRTHTGEKPYKCPECGKSFSRADNLTEHQRTHTGEKPYKCPECGKSFSRNDALTEHQRTHTGKKTS

SEQ ID NO: 684 - DNA, 18 bp, synthetic construct
  misc_feature 1..18: note = Synthetic Sequence
  source 1..18: mol_type = other DNA
cagggcaacgcccaggga

SEQ ID NO: 685 - protein, 176 aa, synthetic construct
  REGION 1..176: note = Synthetic Sequence
LEPGEKPYKCPECGKSFSQRAHLERHQRTHTGEKPYKCPECGKSFSRADNLTEHQRTHTGEKPYKCPECGKSFSDCRDLARHQRTHTGEKPYKCPECGKSFSDSGNLRVHQRTHTGEKPYKCPECGKSFSDPGHLVRHQRTHTGEKPYKCPECGKSFSRADNLTEHQRTHTGKKTS

SEQ ID NO: 686 - DNA, 18 bp, synthetic construct
  misc_feature 1..18: note = Synthetic Sequence
  source 1..18: mol_type = other DNA
ggcaacgcccagggacca

SEQ ID NO: 687 - protein, 176 aa, synthetic construct
  REGION 1..176: note = Synthetic Sequence
LEPGEKPYKCPECGKSFSTSHSLTEHQRTHTGEKPYKCPECGKSFSQRAHLERHQRTHTGEKPYKCPECGKSFSRADNLTEHQRTHTGEKPYKCPECGKSFSDCRDLARHQRTHTGEKPYKCPECGKSFSDSGNLRVHQRTHTGEKPYKCPECGKSFSDPGHLVRHQRTHTGKKTS

SEQ ID NO: 688 - DNA, 18 bp, synthetic construct
  misc_feature 1..18: note = Synthetic Sequence
  source 1..18: mol_type = other DNA
aacgcccagggaccaagt

SEQ ID NO: 689 - protein, 176 aa, synthetic construct
  REGION 1..176: note = Synthetic Sequence
LEPGEKPYKCPECGKSFSHRTTLTNHQRTHTGEKPYKCPECGKSFSTSHSLTEHQRTHTGEKPYKCPECGKSFSQRAHLERHQRTHTGEKPYKCPECGKSFSRADNLTEHQRTHTGEKPYKCPECGKSFSDCRDLARHQRTHTGEKPYKCPECGKSFSDSGNLRVHQRTHTGKKTS

SEQ ID NO: 690 - DNA, 18 bp, synthetic construct
  misc_feature 1..18: note = Synthetic Sequence
  source 1..18: mol_type = other DNA
gcccagggaccaagttag

SEQ ID NO: 691 - protein, 176 aa, synthetic construct
  REGION 1..176: note = Synthetic Sequence
LEPGEKPYKCPECGKSFSREDNLHTHQRTHTGEKPYKCPECGKSFSHRTTLTNHQRTHTGEKPYKCPECGKSFSTSHSLTEHQRTHTGEKPYKCPECGKSFSQRAHLERHQRTHTGEKPYKCPECGKSFSRADNLTEHQRTHTGEKPYKCPECGKSFSDCRDLARHQRTHTGKKTS

SEQ ID NO: 692 - DNA, 18 bp, synthetic construct
  misc_feature 1..18: note = Synthetic Sequence
  source 1..18: mol_type = other DNA
cagggaccaagttagccc

SEQ ID NO: 693 - protein, 176 aa, synthetic construct
  REGION 1..176: note = Synthetic Sequence
LEPGEKPYKCPECGKSFSSKKHLAEHQRTHTGEKPYKCPECGKSFSREDNLHTHQRTHTGEKPYKCPECGKSFSHRTTLTNHQRTHTGEKPYKCPECGKSFSTSHSLTEHQRTHTGEKPYKCPECGKSFSQRAHLERHQRTHTGEKPYKCPECGKSFSRADNLTEHQRTHTGKKTS

SEQ ID NO: 694 - DNA, 15 bp, synthetic construct
  misc_feature 1..15: note = Synthetic Sequence
  source 1..15: mol_type = other DNA
gcctaggcaaaagaa

SEQ ID NO: 695 - protein, 148 aa, synthetic construct
  REGION 1..148: note = Synthetic Sequence
LEPGEKPYKCPECGKSFSQSSNLVRHQRTHTGEKPYKCPECGKSFSQRANLRAHQRTHTGEKPYKCPECGKSFSQSGDLRRHQRTHTGEKPYKCPECGKSFSREDNLHTHQRTHTGEKPYKCPECGKSFSDCRDLARHQRTHTGKKTS

SEQ ID NO: 696 - DNA, 18 bp, synthetic construct
  misc_feature 1..18: note = Synthetic Sequence
  source 1..18: mol_type = other DNA
cgcgaggaggaaaggagg

SEQ ID NO: 697 - protein, 176 aa, synthetic construct
  REGION 1..176: note = Synthetic Sequence
LEPGEKPYKCPECGKSFSRSDHLTNHQRTHTGEKPYKCPECGKSFSRSDHLTNHQRTHTGEKPYKCPECGKSFSQSSNLVRHQRTHTGEKPYKCPECGKSFSRSDNLVRHQRTHTGEKPYKCPECGKSFSRSDNLVRHQRTHTGEKPYKCPECGKSFSHTGHLLEHQRTHTGKKTS

SEQ ID NO: 698 - DNA, 18 bp, synthetic construct
  misc_feature 1..18: note = Synthetic Sequence
  source 1..18: mol_type = other DNA
gaggaggaaaggagggag

SEQ ID NO: 699 - protein, 176 aa, synthetic construct
  REGION 1..176: note = Synthetic Sequence
LEPGEKPYKCPECGKSFSRSDNLVRHQRTHTGEKPYKCPECGKSFSRSDHLTNHQRTHTGEKPYKCPECGKSFSRSDHLTNHQRTHTGEKPYKCPECGKSFSQSSNLVRHQRTHTGEKPYKCPECGKSFSRSDNLVRHQRTHTGEKPYKCPECGKSFSRSDNLVRHQRTHTGKKTS

SEQ ID NO: 700 - DNA, 18 bp, synthetic construct
  misc_feature 1..18: note = Synthetic Sequence
  source 1..18: mol_type = other DNA
gaggaaaggagggagggc

SEQ ID NO: 701 - protein, 176 aa, synthetic construct
  REGION 1..176: note = Synthetic Sequence
LEPGEKPYKCPECGKSFSDPGHLVRHQRTHTGEKPYKCPECGKSFSRSDNLVRHQRTHTGEKPYKCPECGKSFSRSDHLTNHQRTHTGEKPYKCPECGKSFSRSDHLTNHQRTHTGEKPYKCPECGKSFSQSSNLVRHQRTHTGEKPYKCPECGKSFSRSDNLVRHQRTHTGKKTS

SEQ ID NO: 702 - DNA, 30 bp, synthetic construct
  misc_feature 1..30: note = Synthetic Sequence
  source 1..30: mol_type = other DNA
taggacagtggggaaaatgacccaacagcc

SEQ ID NO: 703 - protein, 288 aa, synthetic construct
  REGION 1..288: note = Synthetic Sequence
LEPGEKPYKCPECGKSFSDCRDLARHQRTHTGEKPYKCPECGKSFSSPADLTRHQRTHTGEKPYKCPECGKSFSTSHSLTEHQRTHTGEKPYKCPECGKSFSDPGNLVRHQRTHTGEKPYKCPECGKSFSTTGNLTVHQRTHTGEKPYKCPECGKSFSQSSNLVRHQRTHTGEKPYKCPECGKSFSRSDKLVRHQRTHTGEKPYKCPECGKSFSHRTTLTNHQRTHTGEKPYKCPECGKSFSDPGNLVRHQRTHTGEKPYKCPECGKSFSREDNLHTHQRTHTGKKTS

SEQ ID NO: 704 - DNA, 21 bp, synthetic construct
  misc_feature 1..21: note = Synthetic Sequence
  source 1..21: mol_type = other DNA
agagggagccacgaaaacaga

SEQ ID NO: 705 - protein, 204 aa, synthetic construct
  REGION 1..204: note = Synthetic Sequence
LEPGEKPYKCPECGKSFSQLAHLRAHQRTHTGEKPYKCPECGKSFSDSGNLRVHQRTHTGEKPYKCPECGKSFSQSSNLVRHQRTHTGEKPYKCPECGKSFSSKKALTEHQRTHTGEKPYKCPECGKSFSERSHLREHQRTHTGEKPYKCPECGKSFSRSDKLVRHQRTHTGEKPYKCPECGKSFSQLAHLRAHQRTHTGKKTS

SEQ ID NO: 706 - DNA, 15 bp, synthetic construct
  misc_feature 1..15: note = Synthetic Sequence
  source 1..15: mol_type = other DNA
gcagatagccaggag

SEQ ID NO: 707 - protein, 148 aa, synthetic construct
  REGION 1..148: note = Synthetic Sequence
LEPGEKPYKCPECGKSFSRSDNLVRHQRTHTGEKPYKCPECGKSFSRADNLTEHQRTHTGEKPYKCPECGKSFSERSHLREHQRTHTGEKPYKCPECGKSFSTSGNLVRHQRTHTGEKPYKCPECGKSFSQSGDLRRHQRTHTGKKTS

SEQ ID NO: 708 - DNA, 18 bp, synthetic construct
  misc_feature 1..18: note = Synthetic Sequence
  source 1..18: mol_type = other DNA
agatagccaggagtcctt

SEQ ID NO: 709 - protein, 176 aa, synthetic construct
  REGION 1..176: note = Synthetic Sequence
LEPGEKPYKCPECGKSFSTTGALTEHQRTHTGEKPYKCPECGKSFSDPGALVRHQRTHTGEKPYKCPECGKSFSQRAHLERHQRTHTGEKPYKCPECGKSFSTSHSLTEHQRTHTGEKPYKCPECGKSFSREDNLHTHQRTHTGEKPYKCPECGKSFSQLAHLRAHQRTHTGKKTS

SEQ ID NO: 710 - DNA, 24 bp, synthetic construct
  misc_feature 1..24: note = Synthetic Sequence
  source 1..24: mol_type = other DNA
cccagtggtcaggccggccaggcc

SEQ ID NO: 711 - protein, 232 aa, synthetic construct
  REGION 1..232: note = Synthetic Sequence
LEPGEKPYKCPECGKSFSDCRDLARHQRTHTGEKPYKCPECGKSFSRADNLTEHQRTHTGEKPYKCPECGKSFSDPGHLVRHQRTHTGEKPYKCPECGKSFSDCRDLARHQRTHTGEKPYKCPECGKSFSRADNLTEHQRTHTGEKPYKCPECGKSFSTSGHLVRHQRTHTGEKPYKCPECGKSFSHRTTLTNHQRTHTGEKPYKCPECGKSFSSKKHLAEHQRTHTGKKTS

SEQ ID NO: 712 - DNA, 18 bp, synthetic construct
  misc_feature 1..18: note = Synthetic Sequence
  source 1..18: mol_type = other DNA
ggccggccaggccttcag

SEQ ID NO: 713 - protein, 176 aa, synthetic construct
  REGION 1..176: note = Synthetic Sequence
LEPGEKPYKCPECGKSFSRADNLTEHQRTHTGEKPYKCPECGKSFSTTGALTEHQRTHTGEKPYKCPECGKSFSDPGHLVRHQRTHTGEKPYKCPECGKSFSTSHSLTEHQRTHTGEKPYKCPECGKSFSRSDKLTEHQRTHTGEKPYKCPECGKSFSDPGHLVRHQRTHTGKKTS

SEQ ID NO: 714 - DNA, 27 bp, synthetic construct
  misc_feature 1..27: note = Synthetic Sequence
  source 1..27: mol_type = other DNA
agtgctcagtggaaaccacgaaaggac

SEQ ID NO: 715 - protein, 260 aa, synthetic construct
  REGION 1..260: note = Synthetic Sequence
LEPGEKPYKCPECGKSFSDPGNLVRHQRTHTGEKPYKCPECGKSFSRKDNLKNHQRTHTGEKPYKCPECGKSFSQSGHLTEHQRTHTGEKPYKCPECGKSFSTSHSLTEHQRTHTGEKPYKCPECGKSFSQRANLRAHQRTHTGEKPYKCPECGKSFSRSDHLTTHQRTHTGEKPYKCPECGKSFSRADNLTEHQRTHTGEKPYKCPECGKSFSTSGELVRHQRTHTGEKPYKCPECGKSFSHRTTLTNHQRTHTGKKTS

SEQ ID NO: 716 - DNA, 21 bp, synthetic construct
  misc_feature 1..21: note = Synthetic Sequence
  source 1..21: mol_type = other DNA
tggcccccagcccctcctgcc

SEQ ID NO: 717 - protein, 204 aa, synthetic construct
  REGION 1..204: note = Synthetic Sequence
LEPGEKPYKCPECGKSFSDCRDLARHQRTHTGEKPYKCPECGKSFSTKNSLTEHQRTHTGEKPYKCPECGKSFSTKNSLTEHQRTHTGEKPYKCPECGKSFSDCRDLARHQRTHTGEKPYKCPECGKSFSTSHSLTEHQRTHTGEKPYKCPECGKSFSSKKHLAEHQRTHTGEKPYKCPECGKSFSRSDHLTTHQRTHTGKKTS

SEQ ID NO: 718 - DNA, 27 bp, synthetic construct
  misc_feature 1..27: note = Synthetic Sequence
  source 1..27: mol_type = other DNA
agagccaggagtcctggcccccagccc

SEQ ID NO: 719 - protein, 260 aa, synthetic construct
  REGION 1..260: note = Synthetic Sequence
LEPGEKPYKCPECGKSFSSKKHLAEHQRTHTGEKPYKCPECGKSFSRADNLTEHQRTHTGEKPYKCPECGKSFSSKKHLAEHQRTHTGEKPYKCPECGKSFSDPGHLVRHQRTHTGEKPYKCPECGKSFSTKNSLTEHQRTHTGEKPYKCPECGKSFSHRTTLTNHQRTHTGEKPYKCPECGKSFSRSDHLTNHQRTHTGEKPYKCPECGKSFSDCRDLARHQRTHTGEKPYKCPECGKSFSQLAHLRAHQRTHTGKKTS

SEQ ID NO: 720 - DNA, 24 bp, synthetic construct
  misc_feature 1..24: note = Synthetic Sequence
  source 1..24: mol_type = other DNA
gcaggaggggctgggggccaggac

SEQ ID NO: 721 - protein, 232 aa, synthetic construct
  REGION 1..232: note = Synthetic Sequence
LEPGEKPYKCPECGKSFSDPGNLVRHQRTHTGEKPYKCPECGKSFSRADNLTEHQRTHTGEKPYKCPECGKSFSDPGHLVRHQRTHTGEKPYKCPECGKSFSRSDKLVRHQRTHTGEKPYKCPECGKSFSTSGELVRHQRTHTGEKPYKCPECGKSFSRSDKLVRHQRTHTGEKPYKCPECGKSFSQRAHLERHQRTHTGEKPYKCPECGKSFSQSGDLRRHQRTHTGKKTS

SEQ ID NO: 722 - DNA, 24 bp, synthetic construct
  misc_feature 1..24: note = Synthetic Sequence
  source 1..24: mol_type = other DNA
atagccctgggcccacggcttcgt

SEQ ID NO: 723 - protein, 231 aa, synthetic construct
  REGION 1..231: note = Synthetic Sequence
LEPGEKPYKCPECGKSFSSRRTCRAHQRTHTGEKPYKCPECGKSFSTTGALTEHQRTHTGEKPYKCPECGKSFSRSDKLTEHQRTHTGEKPYKCPECGKSFSTSHSLTEHQRTHTGEKPYKCPECGKSFSDPGHLVRHQRTHTGEKPYKCPECGKSFSRNDALTEHQRTHTGEKPYKCPECGKSFSDCRDLARHQRTHTGEKPYKCPECGKSFSQKSSLIAHQRTHTGKKT

SEQ ID NO: 724 - DNA, 15 bp, synthetic construct
  misc_feature 1..15: note = Synthetic Sequence
  source 1..15: mol_type = other DNA
gaaggacctggctgg

SEQ ID NO: 725 - protein, 148 aa, synthetic construct
  REGION 1..148: note = Synthetic Sequence
LEPGEKPYKCPECGKSFSRSDHLTTHQRTHTGEKPYKCPECGKSFSDPGHLVRHQRTHTGEKPYKCPECGKSFSTKNSLTEHQRTHTGEKPYKCPECGKSFSQRAHLERHQRTHTGEKPYKCPECGKSFSQSSNLVRHQRTHTGKKTS

SEQ ID NO: 726 - DNA, 27 bp, synthetic construct
  misc_feature 1..27: note = Synthetic Sequence
  source 1..27: mol_type = other DNA
gcaggaacgaagccgtgggcccagggc

SEQ ID NO: 727 - protein, 260 aa, synthetic construct
  REGION 1..260: note = Synthetic Sequence
LEPGEKPYKCPECGKSFSDPGHLVRHQRTHTGEKPYKCPECGKSFSRADNLTEHQRTHTGEKPYKCPECGKSFSDCRDLARHQRTHTGEKPYKCPECGKSFSRSDHLTTHQRTHTGEKPYKCPECGKSFSRNDTLTEHQRTHTGEKPYKCPECGKSFSRKDNLKNHQRTHTGEKPYKCPECGKSFSRTDTLRDHQRTHTGEKPYKCPECGKSFSQRAHLERHQRTHTGEKPYKCPECGKSFSQSGDLRRHQRTHTGKKTS

SEQ ID NO: 728 - DNA, 18 bp, synthetic construct
  misc_feature 1..18: note = Synthetic Sequence
  source 1..18: mol_type = other DNA
ggaaaccaccccagcaga

SEQ ID NO: 729 - protein, 176 aa, synthetic construct
  REGION 1..176: note = Synthetic Sequence
LEPGEKPYKCPECGKSFSQLAHLRAHQRTHTGEKPYKCPECGKSFSERSHLREHQRTHTGEKPYKCPECGKSFSSKKHLAEHQRTHTGEKPYKCPECGKSFSSKKALTEHQRTHTGEKPYKCPECGKSFSDSGNLRVHQRTHTGEKPYKCPECGKSFSQRAHLERHQRTHTGKKTS

SEQ ID NO: 730 - DNA, 33 bp, synthetic construct
  misc_feature 1..33: note = Synthetic Sequence
  source 1..33: mol_type = other DNA
aagggtcaagctcggaaaccaccccagcagata

SEQ ID NO: 731 - protein, 316 aa, synthetic construct
  REGION 1..316: note = Synthetic Sequence
LEPGEKPYKCPECGKSFSQKSSLIAHQRTHTGEKPYKCPECGKSFSRADNLTEHQRTHTGEKPYKCPECGKSFSRADNLTEHQRTHTGEKPYKCPECGKSFSSKKHLAEHQRTHTGEKPYKCPECGKSFSTSHSLTEHQRTHTGEKPYKCPECGKSFSQRANLRAHQRTHTGEKPYKCPECGKSFSRSDKLTEHQRTHTGEKPYKCPECGKSFSTSGELVRHQRTHTGEKPYKCPECGKSFSQSGNLTEHQRTHTGEKPYKCPECGKSFSTSGHLVRHQRTHTGEKPYKCPECGKSFSRKDNLKNHQRTHTGKKTS

SEQ ID NO: 732 - DNA, 18 bp, synthetic construct
  misc_feature 1..18: note = Synthetic Sequence
  source 1..18: mol_type = other DNA
ctttgatgaacagtcaca

SEQ ID NO: 733 - protein, 176 aa, synthetic construct
  REGION 1..176: note = Synthetic Sequence
LEPGEKPYKCPECGKSFSSPADLTRHQRTHTGEKPYKCPECGKSFSDPGALVRHQRTHTGEKPYKCPECGKSFSSPADLTRHQRTHTGEKPYKCPECGKSFSQAGHLASHQRTHTGEKPYKCPECGKSFSQAGHLASHQRTHTGEKPYKCPECGKSFSTTGALTEHQRTHTGKKTS

SEQ ID NO: 734 - DNA, 18 bp, synthetic construct
  misc_feature 1..18: note = Synthetic Sequence
  source 1..18: mol_type = other DNA
cttccaattagtcctacc

SEQ ID NO: 735 - protein, 176 aa, synthetic construct
  REGION 1..176: note = Synthetic Sequence
LEPGEKPYKCPECGKSFSDKKDLTRHQRTHTGEKPYKCPECGKSFSTKNSLTEHQRTHTGEKPYKCPECGKSFSHRTTLTNHQRTHTGEKPYKCPECGKSFSHKNALQNHQRTHTGEKPYKCPECGKSFSTSHSLTEHQRTHTGEKPYKCPECGKSFSTTGALTEHQRTHTGKKTS

SEQ ID NO: 736 - DNA, 21 bp, synthetic construct
  misc_feature 1..21: note = Synthetic Sequence
  source 1..21: mol_type = other DNA
atactaggaagaaatacaata

SEQ ID NO: 737 - protein, 204 aa, synthetic construct
  REGION 1..204: note = Synthetic Sequence
LEPGEKPYKCPECGKSFSQKSSLIAHQRTHTGEKPYKCPECGKSFSSPADLTRHQRTHTGEKPYKCPECGKSFSTTGNLTVHQRTHTGEKPYKCPECGKSFSQLAHLRAHQRTHTGEKPYKCPECGKSFSQRAHLERHQRTHTGEKPYKCPECGKSFSQNSTLTEHQRTHTGEKPYKCPECGKSFSQKSSLIAHQRTHTGKKTS

SEQ ID NO: 738 - DNA, 18 bp, synthetic construct
  misc_feature 1..18: note = Synthetic Sequence
  source 1..18: mol_type = other DNA
gctcttgtcatttgagat

SEQ ID NO: 739 - protein, 176 aa, synthetic construct
  REGION 1..176: note = Synthetic Sequence
LEPGEKPYKCPECGKSFSTSGNLVRHQRTHTGEKPYKCPECGKSFSQAGHLASHQRTHTGEKPYKCPECGKSFSHKNALQNHQRTHTGEKPYKCPECGKSFSDPGALVRHQRTHTGEKPYKCPECGKSFSTTGALTEHQRTHTGEKPYKCPECGKSFSTSGELVRHQRTHTGKKTS

SEQ ID NO: 740 - DNA, 33 bp, synthetic construct
  misc_feature 1..33: note = Synthetic Sequence
  source 1..33: mol_type = other DNA
ccaagctgaaatgacacaaaagttaaaacaaag

SEQ ID NO: 741 - protein, 316 aa, synthetic construct
  REGION 1..316: note = Synthetic Sequence
LEPGEKPYKCPECGKSFSRKDNLKNHQRTHTGEKPYKCPECGKSFSSPADLTRHQRTHTGEKPYKCPECGKSFSQRANLRAHQRTHTGEKPYKCPECGKSFSTSGSLVRHQRTHTGEKPYKCPECGKSFSQRANLRAHQRTHTGEKPYKCPECGKSFSSPADLTRHQRTHTGEKPYKCPECGKSFSDPGNLVRHQRTHTGEKPYKCPECGKSFSTTGNLTVHQRTHTGEKPYKCPECGKSFSQAGHLASHQRTHTGEKPYKCPECGKSFSERSHLREHQRTHTGEKPYKCPECGKSFSTSHSLTEHQRTHTGKKTS

SEQ ID NO: 742 - DNA, 21 bp, synthetic construct
  misc_feature 1..21: note = Synthetic Sequence
  source 1..21: mol_type = other DNA
cttataccagttaattagcac

SEQ ID NO: 743 - protein, 204 aa, synthetic construct
  REGION 1..204: note = Synthetic Sequence
LEPGEKPYKCPECGKSFSSKKALTEHQRTHTGEKPYKCPECGKSFSREDNLHTHQRTHTGEKPYKCPECGKSFSTTGNLTVHQRTHTGEKPYKCPECGKSFSTSGSLVRHQRTHTGEKPYKCPECGKSFSTSHSLTEHQRTHTGEKPYKCPECGKSFSQKSSLIAHQRTHTGEKPYKCPECGKSFSTTGALTEHQRTHTGKKTS

SEQ ID NO: 744 - DNA, 24 bp, synthetic construct
  misc_feature 1..24: note = Synthetic Sequence
  source 1..24: mol_type = other DNA
aacgctattgcacacatagttaca

SEQ ID NO: 745 - protein, 232 aa, synthetic construct
  REGION 1..232: note = Synthetic Sequence
LEPGEKPYKCPECGKSFSSPADLTRHQRTHTGEKPYKCPECGKSFSTSGSLVRHQRTHTGEKPYKCPECGKSFSQKSSLIAHQRTHTGEKPYKCPECGKSFSSKKALTEHQRTHTGEKPYKCPECGKSFSQSGDLRRHQRTHTGEKPYKCPECGKSFSHKNALQNHQRTHTGEKPYKCPECGKSFSTSGELVRHQRTHTGEKPYKCPECGKSFSDSGNLRVHQRTHTGKKTS

SEQ ID NO: 746 - DNA, 21 bp, synthetic construct
  misc_feature 1..21: note = Synthetic Sequence
  source 1..21: mol_type = other DNA
tgaattcaggaacaaagtata

SEQ ID NO: 747 - protein, 204 aa, synthetic construct
  REGION 1..204: note = Synthetic Sequence
LEPGEKPYKCPECGKSFSQKSSLIAHQRTHTGEKPYKCPECGKSFSHRTTLTNHQRTHTGEKPYKCPECGKSFSQSGNLTEHQRTHTGEKPYKCPECGKSFSQSSNLVRHQRTHTGEKPYKCPECGKSFSRADNLTEHQRTHTGEKPYKCPECGKSFSHKNALQNHQRTHTGEKPYKCPECGKSFSQAGHLASHQRTHTGKKTS

SEQ ID NO: 748 - DNA, 27 bp, synthetic construct
  misc_feature 1..27: note = Synthetic Sequence
  source 1..27: mol_type = other DNA
gctggtatgtgacacagccatcaacaa

SEQ ID NO: 749 - protein, 260 aa, synthetic construct
  REGION 1..260: note = Synthetic Sequence
LEPGEKPYKCPECGKSFSQSGNLTEHQRTHTGEKPYKCPECGKSFSQSGNLTEHQRTHTGEKPYKCPECGKSFSTSGNLTEHQRTHTGEKPYKCPECGKSFSERSHLREHQRTHTGEKPYKCPECGKSFSSKKALTEHQRTHTGEKPYKCPECGKSFSQAGHLASHQRTHTGEKPYKCPECGKSFSRRDELNVHQRTHTGEKPYKCPECGKSFSTSGHLVRHQRTHTGEKPYKCPECGKSFSTSGELVRHQRTHTGKKTS

SEQ ID NO: 750 - DNA, 24 bp, synthetic construct
  misc_feature 1..24: note = Synthetic Sequence
  source 1..24: mol_type = other DNA
cttcctgaaagcaagagatgaaat

SEQ ID NO: 751 - protein, 232 aa, synthetic construct
  REGION 1..232: note = Synthetic Sequence
LEPGEKPYKCPECGKSFSTTGNLTVHQRTHTGEKPYKCPECGKSFSQAGHLASHQRTHTGEKPYKCPECGKSFSQLAHLRAHQRTHTGEKPYKCPECGKSFSRKDNLKNHQRTHTGEKPYKCPECGKSFSERSHLREHQRTHTGEKPYKCPECGKSFSQSSNLVRHQRTHTGEKPYKCPECGKSFSTKNSLTEHQRTHTGEKPYKCPECGKSFSTTGALTEHQRTHTGKKTS

SEQ ID NO: 752 - DNA, 23 bp, synthetic construct
  misc_feature 1..23: note = Synthetic Sequence
  source 1..23: mol_type = other DNA
ctgaaagcaagagatgaaattcc

SEQ ID NO: 753 - protein, 232 aa, synthetic construct
  REGION 1..232: note = Synthetic Sequence
LEPGEKPYKCPECGKSFSTSHSLTEHQRTHTGEKPYKCPECGKSFSHKNALQNHQRTHTGEKPYKCPECGKSFSQSSNLVRHQRTHTGEKPYKCPECGKSFSTSGNLVRHQRTHTGEKPYKCPECGKSFSQLAHLRAHQRTHTGEKPYKCPECGKSFSQSGDLRRHQRTHTGEKPYKCPECGKSFSQRANLRAHQRTHTGEKPYKCPECGKSFSRNDALTEHQRTHTGKKTS

SEQ ID NO: 754 - DNA, 21 bp, synthetic construct
  misc_feature 1..21: note = Synthetic Sequence
  source 1..21: mol_type = other DNA
atacgaggagaaaattagcat

SEQ ID NO: 755 - protein, 204 aa, synthetic construct
  REGION 1..204: note = Synthetic Sequence
LEPGEKPYKCPECGKSFSTSGNLTEHQRTHTGEKPYKCPECGKSFSREDNLHTHQRTHTGEKPYKCPECGKSFSTTGNLTVHQRTHTGEKPYKCPECGKSFSQSSNLVRHQRTHTGEKPYKCPECGKSFSQRAHLERHQRTHTGEKPYKCPECGKSFSQSGHLTEHQRTHTGEKPYKCPECGKSFSQKSSLIAHQRTHTGKKTS

SEQ ID NO: 756 - DNA, 36 bp, synthetic construct
  misc_feature 1..36: note = Synthetic Sequence
  source 1..36: mol_type = other DNA
catccatggcaggaagttgaagccaaaataaatctg

SEQ ID NO: 757 - protein, 344 aa, synthetic construct
  REGION 1..344: note = Synthetic Sequence
LEPGEKPYKCPECGKSFSRNDALTEHQRTHTGEKPYKCPECGKSFSTTGNLTVHQRTHTGEKPYKCPECGKSFSQKSSLIAHQRTHTGEKPYKCPECGKSFSQRANLRAHQRTHTGEKPYKCPECGKSFSDCRDLARHQRTHTGEKPYKCPECGKSFSQSSNLVRHQRTHTGEKPYKCPECGKSFSTSGSLVRHQRTHTGEKPYKCPECGKSFSQSSNLVRHQRTHTGEKPYKCPECGKSFSRADNLTEHQRTHTGEKPYKCPECGKSFSRSDHLTTHQRTHTGEKPYKCPECGKSFSTSHSLTEHQRTHTGEKPYKCPECGKSFSTSGNLTEHQRTHTGKKTS

SEQ ID NO: 758 - DNA, 27 bp, synthetic construct
  misc_feature 1..27: note = Synthetic Sequence
  source 1..27: mol_type = other DNA
atggcaggaagttgaagccaaaataaa

SEQ ID NO: 759 - protein, 260 aa, synthetic construct
  REGION 1..260: note = Synthetic Sequence
LEPGEKPYKCPECGKSFSQRANLRAHQRTHTGEKPYKCPECGKSFSTTGNLTVHQRTHTGEKPYKCPECGKSFSQSGNLTEHQRTHTGEKPYKCPECGKSFSERSHLREHQRTHTGEKPYKCPECGKSFSQAGHLASHQRTHTGEKPYKCPECGKSFSHRTTLTNHQRTHTGEKPYKCPECGKSFSQRAHLERHQRTHTGEKPYKCPECGKSFSQSGDLRRHQRTHTGEKPYKCPECGKSFSRRDELNVHQRTHTGKKTS

SEQ ID NO: 760 - DNA, 33 bp, synthetic construct
  misc_feature 1..33: note = Synthetic Sequence
  source 1..33: mol_type = other DNA
gaaaagaagactcaaggaaacagagccaaacac

SEQ ID NO: 761 - protein, 316 aa, synthetic construct
  REGION 1..316: note = Synthetic Sequence
LEPGEKPYKCPECGKSFSSKKALTEHQRTHTGEKPYKCPECGKSFSQRANLRAHQRTHTGEKPYKCPECGKSFSDCRDLARHQRTHTGEKPYKCPECGKSFSQLAHLRAHQRTHTGEKPYKCPECGKSFSDSGNLRVHQRTHTGEKPYKCPECGKSFSQRAHLERHQRTHTGEKPYKCPECGKSFSQSGNLTEHQRTHTGEKPYKCPECGKSFSTHLDLIRHQRTHTGEKPYKCPECGKSFSRKDNLKNHQRTHTGEKPYKCPECGKSFSRKDNLKNHQRTHTGEKPYKCPECGKSFSQSSNLVRHQRTHTGKKTS

SEQ ID NO: 762 - DNA, 24 bp, synthetic construct
  misc_feature 1..24: note = Synthetic Sequence
  source 1..24: mol_type = other DNA
aggaaacagagccaaacacttaca

SEQ ID NO: 763 - protein, 232 aa, synthetic construct
  REGION 1..232: note = Synthetic Sequence
LEPGEKPYKCPECGKSFSSPADLTRHQRTHTGEKPYKCPECGKSFSTTGALTEHQRTHTGEKPYKCPECGKSFSSPADLTRHQRTHTGEKPYKCPECGKSFSQSGNLTEHQRTHTGEKPYKCPECGKSFSERSHLREHQRTHTGEKPYKCPECGKSFSRADNLTEHQRTHTGEKPYKCPECGKSFSQRANLRAHQRTHTGEKPYKCPECGKSFSRSDHLTNHQRTHTGKKTS

SEQ ID NO: 764 - DNA, 60 bp, synthetic construct
  misc_feature 1..60: note = Synthetic Sequence
  source 1..60: mol_type = other DNA
atgcagatttggacacagagtagtaaactgtgaaaacgtgacaaggcaaagtggcgtggg

SEQ ID NO: 765 - protein, 568 aa, synthetic construct
  REGION 1..568: note = Synthetic Sequence
LEPGEKPYKCPECGKSFSRSDKLVRHQRTHTGEKPYKCPECGKSFSSRRTCRAHQRTHTGEKPYKCPECGKSFSRSDHLTTHQRTHTGEKPYKCPECGKSFSRKDNLKNHQRTHTGEKPYKCPECGKSFSQSGDLRRHQRTHTGEKPYKCPECGKSFSRKDNLKNHQRTHTGEKPYKCPECGKSFSDPGNLVRHQRTHTGEKPYKCPECGKSFSSRRTCRAHQRTHTGEKPYKCPECGKSFSQRANLRAHQRTHTGEKPYKCPECGKSFSQAGHLASHQRTHTGEKPYKCPECGKSFSRNDALTEHQRTHTGEKPYKCPECGKSFSQRANLRAHQRTHTGEKPYKCPECGKSFSHRTTLTNHQRTHTGEKPYKCPECGKSFSHRTTLTNHQRTHTGEKPYKCPECGKSFSRADNLTEHQRTHTGEKPYKCPECGKSFSSPADLTRHQRTHTGEKPYKCPECGKSFSRSDHLTTHQRTHTGEKPYKCPECGKSFSHKNALQNHQRTHTGEKPYKCPECGKSFSRADNLTEHQRTHTGEKPYKCPECGKSFSRRDELNVHQRTHTGKKTS

SEQ ID NO: 766 - DNA, 18 bp, synthetic construct
  misc_feature 1..18: note = Synthetic Sequence
  source 1..18: mol_type = other DNA
ggacacagagtagtaaac

SEQ ID NO: 767 - protein, 176 aa, synthetic construct
  REGION 1..176: note = Synthetic Sequence
LEPGEKPYKCPECGKSFSDSGNLRVHQRTHTGEKPYKCPECGKSFSQSSSLVRHQRTHTGEKPYKCPECGKSFSQSSSLVRHQRTHTGEKPYKCPECGKSFSQLAHLRAHQRTHTGEKPYKCPECGKSFSSKKALTEHQRTHTGEKPYKCPECGKSFSQRAHLERHQRTHTGKKTS

SEQ ID NO: 768 - DNA, 18 bp, synthetic construct
  misc_feature 1..18: note = Synthetic Sequence
  source 1..18: mol_type = other DNA
aaagctagcagcatggca

SEQ ID NO: 769 - protein, 176 aa, synthetic construct
  REGION 1..176: note = Synthetic Sequence
LEPGEKPYKCPECGKSFSQSGDLRRHQRTHTGEKPYKCPECGKSFSRRDELNVHQRTHTGEKPYKCPECGKSFSERSHLREHQRTHTGEKPYKCPECGKSFSERSHLREHQRTHTGEKPYKCPECGKSFSTSGELVRHQRTHTGEKPYKCPECGKSFSQRANLRAHQRTHTGKKTS

SEQ ID NO: 770 - DNA, 24 bp, synthetic construct
  misc_feature 1..24: note = Synthetic Sequence
  source 1..24: mol_type = other DNA
cctcttataaggcccaagaggata

SEQ ID NO: 771 - protein, 232 aa, synthetic construct
  REGION 1..232: note = Synthetic Sequence
LEPGEKPYKCPECGKSFSQKSSLIAHQRTHTGEKPYKCPECGKSFSRSDHLTNHQRTHTGEKPYKCPECGKSFSRKDNLKNHQRTHTGEKPYKCPECGKSFSSKKHLAEHQRTHTGEKPYKCPECGKSFSRSDHLTNHQRTHTGEKPYKCPECGKSFSQKSSLIAHQRTHTGEKPYKCPECGKSFSTTGALTEHQRTHTGEKPYKCPECGKSFSTKNSLTEHQRTHTGKKTS

SEQ ID NO: 772 - DNA, 21 bp, synthetic construct
  misc_feature 1..21: note = Synthetic Sequence
  source 1..21: mol_type = other DNA
caacatccttgacttaatcac

SEQ ID NO: 773 - protein, 204 aa, synthetic construct
  REGION 1..204: note = Synthetic Sequence
LEPGEKPYKCPECGKSFSSKKALTEHQRTHTGEKPYKCPECGKSFSTTGNLTVHQRTHTGEKPYKCPECGKSFSTTGALTEHQRTHTGEKPYKCPECGKSFSQAGHLASHQRTHTGEKPYKCPECGKSFSTKNSLTEHQRTHTGEKPYKCPECGKSFSTSGNLTEHQRTHTGEKPYKCPECGKSFSQSGNLTEHQRTHTGKKTS

SEQ ID NO: 774 - DNA, 18 bp, synthetic construct
  misc_feature 1..18: note = Synthetic Sequence
  source 1..18: mol_type = other DNA
ggtagcaaaaaggtaacc

SEQ ID NO: 775 - protein, 176 aa, synthetic construct
  REGION 1..176: note = Synthetic Sequence
LEPGEKPYKCPECGKSFSDKKDLTRHQRTHTGEKPYKCPECGKSFSQSSSLVRHQRTHTGEKPYKCPECGKSFSRKDNLKNHQRTHTGEKPYKCPECGKSFSQRANLRAHQRTHTGEKPYKCPECGKSFSERSHLREHQRTHTGEKPYKCPECGKSFSTSGHLVRHQRTHTGKKTS

SEQ ID NO: 776 - DNA, 36 bp, synthetic construct
  misc_feature 1..36: note = Synthetic Sequence
  source 1..36: mol_type = other DNA
tggggtgcaagaggccaggccagagttgttctggtc

SEQ ID NO: 777 - protein, 344 aa, synthetic construct
  REGION 1..344: note = Synthetic Sequence
LEPGEKPYKCPECGKSFSDPGALVRHQRTHTGEKPYKCPECGKSFSRNDALTEHQRTHTGEKPYKCPECGKSFSTSGSLVRHQRTHTGEKPYKCPECGKSFSTSGSLVRHQRTHTGEKPYKCPECGKSFSQLAHLRAHQRTHTGEKPYKCPECGKSFSDCRDLARHQRTHTGEKPYKCPECGKSFSRADNLTEHQRTHTGEKPYKCPECGKSFSDPGHLVRHQRTHTGEKPYKCPECGKSFSQLAHLRAHQRTHTGEKPYKCPECGKSFSQSGDLRRHQRTHTGEKPYKCPECGKSFSTSGHLVRHQRTHTGEKPYKCPECGKSFSRSDHLTTHQRTHTGKKTS

SEQ ID NO: 778 - DNA, 24 bp, synthetic construct
  misc_feature 1..24: note = Synthetic Sequence
  source 1..24: mol_type = other DNA
cgcatgctgattcagcctcctgac

SEQ ID NO: 779 - protein, 232 aa, synthetic construct
  REGION 1..232: note = Synthetic Sequence
LEPGEKPYKCPECGKSFSDPGNLVRHQRTHTGEKPYKCPECGKSFSTKNSLTEHQRTHTGEKPYKCPECGKSFSTKNSLTEHQRTHTGEKPYKCPECGKSFSRADNLTEHQRTHTGEKPYKCPECGKSFSHKNALQNHQRTHTGEKPYKCPECGKSFSRNDALTEHQRTHTGEKPYKCPECGKSFSRRDELNVHQRTHTGEKPYKCPECGKSFSHTGHLLEHQRTHTGKKTS

SEQ ID NO: 780 - DNA, 18 bp, synthetic construct
  misc_feature 1..18: note = Synthetic Sequence
  source 1..18: mol_type = other DNA
agtcaagcaacaggatga

SEQ ID NO: 781 - protein, 176 aa, synthetic construct
  REGION 1..176: note = Synthetic Sequence
LEPGEKPYKCPECGKSFSQAGHLASHQRTHTGEKPYKCPECGKSFSQRAHLERHQRTHTGEKPYKCPECGKSFSSPADLTRHQRTHTGEKPYKCPECGKSFSQSGDLRRHQRTHTGEKPYKCPECGKSFSQSGNLTEHQRTHTGEKPYKCPECGKSFSHRTTLTNHQRTHTGKKTS

SEQ ID NO: 782 - DNA, 30 bp, synthetic construct
  misc_feature 1..30: note = Synthetic Sequence
  source 1..30: mol_type = other DNA
gtcaagcaacaggatgatccaaatgctatt

SEQ ID NO: 783 - protein, 288 aa, synthetic construct
  REGION 1..288: note = Synthetic Sequence
LEPGEKPYKCPECGKSFSHKNALQNHQRTHTGEKPYKCPECGKSFSTSGELVRHQRTHTGEKPYKCPECGKSFSTTGNLTVHQRTHTGEKPYKCPECGKSFSTSHSLTEHQRTHTGEKPYKCPECGKSFSTSGNLVRHQRTHTGEKPYKCPECGKSFSTSGNLVRHQRTHTGEKPYKCPECGKSFSRADNLTEHQRTHTGEKPYKCPECGKSFSQSGNLTEHQRTHTGEKPYKCPECGKSFSRKDNLKNHQRTHTGEKPYKCPECGKSFSDPGALVRHQRTHTGKKTS

SEQ ID NO: 784 - DNA, 18 bp, synthetic construct
  misc_feature 1..18: note = Synthetic Sequence
  source 1..18: mol_type = other DNA
gtagaaactcgccttatg

SEQ ID NO: 785 - protein, 176 aa, synthetic construct
  REGION 1..176: note = Synthetic Sequence
LEPGEKPYKCPECGKSFSRRDELNVHQRTHTGEKPYKCPECGKSFSTTGALTEHQRTHTGEKPYKCPECGKSFSHTGHLLEHQRTHTGEKPYKCPECGKSFSTHLDLIRHQRTHTGEKPYKCPECGKSFSQSSNLVRHQRTHTGEKPYKCPECGKSFSQSSSLVRHQRTHTGKKTS

SEQ ID NO: 786 - DNA, 21 bp, synthetic construct
  misc_feature 1..21: note = Synthetic Sequence
  source 1..21: mol_type = other DNA
tgaatgagtcctgtccatctt

SEQ ID NO: 787 - protein, 204 aa, synthetic construct
  REGION 1..204: note = Synthetic Sequence
LEPGEKPYKCPECGKSFSTTGALTEHQRTHTGEKPYKCPECGKSFSTSGNLTEHQRTHTGEKPYKCPECGKSFSDPGALVRHQRTHTGEKPYKCPECGKSFSTKNSLTEHQRTHTGEKPYKCPECGKSFSHRTTLTNHQRTHTGEKPYKCPECGKSFSRRDELNVHQRTHTGEKPYKCPECGKSFSQAGHLASHQRTHTGKKTS

SEQ ID NO: 788 - DNA, 21 bp, synthetic construct
  misc_feature 1..21: note = Synthetic Sequence
  source 1..21: mol_type = other DNA
aagattagaacaaatgtccag

SEQ ID NO: 789 - protein, 204 aa, synthetic construct
  REGION 1..204: note = Synthetic Sequence
LEPGEKPYKCPECGKSFSRADNLTEHQRTHTGEKPYKCPECGKSFSDPGALVRHQRTHTGEKPYKCPECGKSFSTTGNLTVHQRTHTGEKPYKCPECGKSFSSPADLTRHQRTHTGEKPYKCPECGKSFSQLAHLRAHQRTHTGEKPYKCPECGKSFSHKNALQNHQRTHTGEKPYKCPECGKSFSRKDNLKNHQRTHTGKKTS

SEQ ID NO: 790 - DNA, 18 bp, synthetic construct
  misc_feature 1..18: note = Synthetic Sequence
  source 1..18: mol_type = other DNA
actctaagcagcaatgta

SEQ ID NO: 791 - protein, 176 aa, synthetic construct
  REGION 1..176: note = Synthetic Sequence
LEPGEKPYKCPECGKSFSQSSSLVRHQRTHTGEKPYKCPECGKSFSTTGNLTVHQRTHTGEKPYKCPECGKSFSERSHLREHQRTHTGEKPYKCPECGKSFSERSHLREHQRTHTGEKPYKCPECGKSFSQNSTLTEHQRTHTGEKPYKCPECGKSFSTHLDLIRHQRTHTGKKTS

SEQ ID NO: 792 - DNA, 18 bp, synthetic construct
  misc_feature 1..18: note = Synthetic Sequence
  source 1..18: mol_type = other DNA
tgggatagtgaaaatgtc

SEQ ID NO: 793 - protein, 176 aa, synthetic construct
  REGION 1..176: note = Synthetic Sequence
LEPGEKPYKCPECGKSFSDPGALVRHQRTHTGEKPYKCPECGKSFSTTGNLTVHQRTHTGEKPYKCPECGKSFSQSSNLVRHQRTHTGEKPYKCPECGKSFSHRTTLTNHQRTHTGEKPYKCPECGKSFSTSGNLVRHQRTHTGEKPYKCPECGKSFSRSDHLTTHQRTHTGKKTS

SEQ ID NO: 794 - DNA, 27 bp, synthetic construct
  misc_feature 1..27: note = Synthetic Sequence
  source 1..27: mol_type = other DNA
aaaacttgggtcactaaaatagatgat

SEQ ID NO: 795 - protein, 260 aa, synthetic construct
  REGION 1..260: note = Synthetic Sequence
LEPGEKPYKCPECGKSFSTSGNLVRHQRTHTGEKPYKCPECGKSFSTSGNLVRHQRTHTGEKPYKCPECGKSFSQKSSLIAHQRTHTGEKPYKCPECGKSFSQRANLRAHQRTHTGEKPYKCPECGKSFSTHLDLIRHQRTHTGEKPYKCPECGKSFSDPGALVRHQRTHTGEKPYKCPECGKSFSRSDHLTTHQRTHTGEKPYKCPECGKSFSTHLDLIRHQRTHTGEKPYKCPECGKSFSQRANLRAHQRTHTGKKTS

SEQ ID NO: 796 - DNA, 27 bp, synthetic construct
  misc_feature 1..27: note = Synthetic Sequence
  source 1..27: mol_type = other DNA
aaacatggaaaaggtcaaaaacttggg

SEQ ID NO: 797 - protein, 260 aa, synthetic construct
  REGION 1..260: note = Synthetic Sequence
LEPGEKPYKCPECGKSFSRSDKLVRHQRTHTGEKPYKCPECGKSFSTTGALTEHQRTHTGEKPYKCPECGKSFSQRANLRAHQRTHTGEKPYKCPECGKSFSQSGNLTEHQRTHTGEKPYKCPECGKSFSTSGHLVRHQRTHTGEKPYKCPECGKSFSQRANLRAHQRTHTGEKPYKCPECGKSFSQRAHLERHQRTHTGEKPYKCPECGKSFSTSGNLTEHQRTHTGEKPYKCPECGKSFSQRANLRAHQRTHTGKKTS

SEQ ID NO: 798 - DNA, 24 bp, synthetic construct
  misc_feature 1..24: note = Synthetic Sequence
  source 1..24: mol_type = other DNA
aatgactagaatgaagtcctactg

SEQ ID NO: 799 - protein, 232 aa, synthetic construct
  REGION 1..232: note = Synthetic Sequence
LEPGEKPYKCPECGKSFSRNDALTEHQRTHTGEKPYKCPECGKSFSQNSTLTEHQRTHTGEKPYKCPECGKSFSDPGALVRHQRTHTGEKPYKCPECGKSFSQSSNLVRHQRTHTGEKPYKCPECGKSFSTTGNLTVHQRTHTGEKPYKCPECGKSFSREDNLHTHQRTHTGEKPYKCPECGKSFSDPGNLVRHQRTHTGEKPYKCPECGKSFSTTGNLTVHQRTHTGKKTS

SEQ ID NO: 800 - DNA, 20 bp, synthetic construct
  misc_feature 1..20: note = Synthetic Sequence
  source 1..20: mol_type = other DNA
ggagccacgaaaacagatcc

SEQ ID NO: 801 - DNA, 20 bp, synthetic construct
  misc_feature 1..20: note = Synthetic Sequence
  source 1..20: mol_type = other DNA
ggagccacgaaaacagatcc

SEQ ID NO: 802 - DNA, 20 bp, synthetic construct
  misc_feature 1..20: note = Synthetic Sequence
  source 1..20: mol_type = other DNA
agatccagggacacggtgct

SEQ ID NO: 803 - DNA, 20 bp, synthetic construct
  misc_feature 1..20: note = Synthetic Sequence
  source 1..20: mol_type = other DNA
gacacggtgctaggacagtg

SEQ ID NO: 804 - DNA, 20 bp, synthetic construct
  misc_feature 1..20: note = Synthetic Sequence
  source 1..20: mol_type = other DNA
gaaaatgacccaacagcctc

SEQ ID NO: 805 - DNA, 20 bp, synthetic construct
  misc_feature 1..20: note = Synthetic Sequence
  source 1..20: mol_type = other DNA
gcctggccggcctgaccact

SEQ ID NO: 806 - DNA, 20 bp, synthetic construct
  misc_feature 1..20: note = Synthetic Sequence
  source 1..20: mol_type = other DNA
ctgagcactgaaggcctggc

SEQ ID NO: 807 - DNA, 20 bp, synthetic construct
  misc_feature 1..20: note = Synthetic Sequence
  source 1..20: mol_type = other DNA
tggtttccactgagcactga

SEQ ID NO: 808 - DNA, 20 bp, synthetic construct
  misc_feature 1..20: note = Synthetic Sequence
  source 1..20: mol_type = other DNA
gatagccaggagtcctttcg

SEQ ID NO: 809 - DNA, 20 bp, synthetic construct
  misc_feature 1..20: note = Synthetic Sequence
  source 1..20: mol_type = other DNA
gcgcttccagtgctcagact

SEQ ID NO: 810 - DNA, 20 bp, synthetic construct
  misc_feature 1..20: note = Synthetic Sequence
  source 1..20: mol_type = other DNA
cagtgctcagactagggaag

SEQ ID NO: 811 - DNA, 20 bp, synthetic construct
  misc_feature 1..20: note = Synthetic Sequence
  source 1..20: mol_type = other DNA
gcccctcctccttcagagcc

SEQ ID NO: 812 - DNA, 20 bp, synthetic construct
  misc_feature 1..20: note = Synthetic Sequence
  source 1..20: mol_type = other DNA
tccttcagagccaggagtcc

SEQ ID NO: 813 - DNA, 20 bp, synthetic construct
  misc_feature 1..20: note = Synthetic Sequence
  source 1..20: mol_type = other DNA
tggtttccgagcttgaccct

SEQ ID NO: 814 - DNA, 20 bp, synthetic construct
  misc_feature 1..20: note = Synthetic Sequence
  source 1..20: mol_type = other DNA
ctgcagagtatctgctgggg

SEQ ID NO: 815 - DNA, 20 bp, synthetic construct
  misc_feature 1..20: note = Synthetic Sequence
  source 1..20: mol_type = other DNA
cgttcctgcagagtatctgc